(19) United States
(12) Patent Application Publication
(10) Pub. No.: US 2023/0414777 A1
(43) Pub. Date: Dec. 28, 2023

(54) Antigen Presenting Polypeptide Complexes and Methods of Use Thereof

(71) Applicant: Cue Biopharma, Inc., Boston, MA (US)

(72) Inventors: Ronald D. SEIDEL, III, Natick, MA (US); Rodolfo J. CHAPARRO, Cambridge, MA (US); John F. ROSS, Arlington, MA (US); Chee Meng LOW, Boston, MA (US); Saso CEMERSKI, Norfolk, MA (US)

(21) Appl. No.: 18/035,731

(22) PCT Filed: Nov. 8, 2021
(86) PCT No.: PCT/US2021/058493

§ 371 Date: May 5, 2023

Related U.S. Application Data
(60) Provisional application No. 63/110,937, filed on Nov. 6, 2020.

(51) Int. Cl. A61K 47/68; C07K 14/74; C07K 16/28; C07K 16/24

(52) U.S. Cl. CPC A61K 47/6849; C07K 14/70539; C07K 16/2827; C07K 2319/03; C07K 2317/52; C07K 2317/10; C07K 16/246

(57) ABSTRACT

The present disclosure provides Multimeric Antigen Presenting Polypeptides (MAPPs) for the presentation of KRAS antigens in the context of a class I MHC receptor. The present disclosure provides nucleic acids comprising nucleotide sequences encoding those MAPPs, as well as cells genetically modified with the nucleic acids. MAPPs of the present disclosure are useful for selectively modulating activity of T cells having T cell receptors that recognize the antigens. Thus, the present disclosure provides compositions and methods for modulating the activity of T cells, as well as compositions and methods for treating persons who have diseases and/or disorders including cancers.

This application claims the benefit of U.S. Provisional Application No. 62/814,842 filed on Mar. 6, 2019, which is incorporated herein by reference in its entirety. This application contains a sequence listing submitted electronically via EFS-web, which serves as both the paper copy and the computer readable form (CRF) and consists of a file entitled “2910-19PCT_seglist.txt”, which was created on Nov. 8, 2021, which is 457,736 bytes in size, and which is herein incorporated by reference in its entirety.

### INTRODUCTION

An adaptive immune response involves the engagement of the T cell receptor (TCR), present on the surface of a T cell, with a small peptide antigen non-covalently presented on the surface of an antigen presenting cell (APC) by a major histocompatibility complex (MHC; also referred to in humans as a human leukocyte antigen (“HLA”) complex). This engagement represents the immune system's targeting mechanism and is a requisite molecular interaction for T cell modulation (activation or inhibition) and effector function. In addition to epitope-specific cell targeting, the targeted T cells are activated through engagement of costimulatory proteins found, for example, on the APC with counterpart costimulatory proteins (e.g., receptors) on the T cells. Both signals—epitope/TCR binding and engagement of APC costimulatory proteins with T cell costimulatory proteins—are required to drive T cell specificity and activation or inhibition. The TCR is specific for a given epitope; however, costimulatory proteins are not epitope specific, and instead are generally expressed on all T cells or on subsets of T cells.

APCs serve to capture and break the proteins from foreign organisms, or abnormal proteins (e.g., from genetic mutation in cancer cells), into smaller fragments suitable as signals for scrutiny by the larger immune system, including T cells. In particular, APCs break down proteins into small peptide fragments, which are then paired with proteins of the major histocompatibility complex (“MHC”) and displayed on the cell surface. Cell surface display of an MHC together with a peptide fragment, also known as a T cell epitope, provides the underlying scaffold surveilled by T cells, allowing for specific recognition. The peptide fragments can be pathogen-derived (infectious agent-derived), tumor-derived, or derived from natural host proteins (self-proteins). Moreover, APCs can recognize other foreign components, such as bacterial toxins, viral proteins, viral DNA, viral RNA, etc., whose presence denotes an escalated threat level. The APCs relay this information to T cells through additional costimulatory signals in order to generate a more effective response.

T cells recognize peptide-major histocompatibility complex (“pMHC”) complexes through a specialized cell surface receptor, the T cell receptor (“TCR”). The TCR is unique to each T cell; as a consequence, each T cell is highly specific for a particular pMHC target. In order to adequately address the universe of potential threats, a very large number (˜10,000,000) of distinct T cells with distinct TCRs exist in the human body. Further, any given T cell, specific for a particular T cell peptide, is initially a very small fraction of the total T cell population. Although normally dormant and in limited numbers, T cells bearing specific TCRs can be readily activated and amplified by APCs to generate highly potent T cell responses that involve many millions of T cells. Such activated T cell responses are capable of attacking and clearing viral infections, bacterial infections, and other cellular threats including tumors, as discussed below. Conversely, the broad, non-specific activation of overly active T cell responses against self or shared antigens can give rise to T cells inappropriately attacking and destroying healthy tissues or cells.

MHC proteins are referred to as human leukocyte antigens (HLA) in humans. HLA proteins are divided into two major classes, class I and class II proteins encoded by separate loci. HLA class I proteins comprise a heavy chain (sometimes denoted “MHC-H”) encoded by gene loci including the classical HLA-A, HLA-B, HLA-C, and non-classical HLA-E, HLA-F and HLA-G loci. HLA Class I proteins also include a light chain protein, the β-2 microglobulin (β2M) polypeptide.

### I. SUMMARY

The present disclosure provides multimeric antigen-presenting polypeptide complexes (“MAPP” singular and “MAPPs” plural) that include at least one framework polypeptide and at least one dimerization polypeptide. Framework polypeptides comprise one or more polypeptide dimerization sequence that permits specific binding with other polypeptides (dimerization polypeptides) having a counterpart dimerization sequence thereby forming at least a heterodimer (See FIG. 1A). Framework polypeptides also comprise a multimerization sequence(s) that permits two or more framework polypeptides to associate, thereby forming a higher order structure (e.g., a duplex of the two or more heterodimers, a “duplex MAPP” see e.g., FIGS. 1A and 1B). Neither the dimerization sequence nor the multimerization sequence of the framework polypeptide (or the counterpart dimerization sequence) comprises an MHC-H polypeptide sequence or a β2M polypeptide sequence; and as such, interaction brought about by those sequences are not consider dimerization or multimerization of framework and/or dimerization peptides. Accordingly, the framework polypeptides provide a structure upon which other polypeptides can be organized by interactions at the dimerization sequences, and which can interact with other framework polypeptides by way of multimerization sequences.

The epitopes presented by MAPPs, duplex MAPPs, and higher order MAPPs of the present disclosure are epitopes presented by KRAS peptides, phosphopeptides, and/or lipopeptides and variants thereof, which may simply be referred to as “epitopes,” “epitope peptides” the purpose of this disclosure. The framework and dimerization peptide containing MAPPs, duplex MAPPs, and MAPPs of higher order (e.g., triplex MAPPs) described herein provide a means by which epitope may be delivered in the context of an MHC (e.g., HLA) to a target T cell displaying a TCR specific for the epitope, while at the same time permitting for the flexible presentation of one or more immunomodulatory polypeptides (“MODs”). Accordingly, the MAPPs, duplex MAPPs, and higher order MAPPs permit deliver of one or more MODs in an KRAS epitope selective (e.g., dependent/specific) manner that permits formation of an active immune synapse with a target T cell selective for the KRAS epitope, and control/regulation of the target T cell's response to the KRAS epitope.

MHC polypeptide portions of MAPPs presenting epitopes may be divided into single polypeptide chain presenting sequences, or presenting complexes comprising two or more polypeptide chains.

Where the epitope peptide, an MHC-H and β2M polypeptide sequences, and optionally one or more MODs are provided in one polypeptide, it is termed an “epitope presenting sequence” or “presenting sequence.” See, e.g., FIG. 12. MAPPs typically contain one or two presenting sequence, and accordingly, duplex MAPPS typically comprise one, two, three, four presenting sequences. MAPPs and duplex MAPPs may comprise more presenting sequences depending on, for example, the number of dimerization sequences in the framework polypeptide. The presenting sequences may be integrated into a MAPP as part of the a framework polypeptide or a dimerization polypeptide. A MAPP may have presenting sequences as part of either or both of framework or dimerization polypeptide. Compare, for example, FIG. 6 structures A-D and FIG. 7 structures A-D.

As an alternative to utilizing a single polypeptide to present an epitope, the MHC components (MHC-H polypeptide sequences) and the epitope may be divided (split) into two separate polypeptide sequences, which together are denoted herein as an “epitope presenting complex” or “presenting complex.” A presenting complex is integrated into a MAPP by having a presenting complex first amino acid sequence (“presenting complex 1^{st} sequence”) as part of a framework or dimerization polypeptide. The remaining MHC sequence(s) are part of a polypeptide termed the presenting complex second amino acid sequence (“presenting complex 2^{nd} sequence”). The epitope peptide and any independently selected MODs that are present may be part of the polypeptide comprising either the presenting complex 1^{st} sequence or the presenting complex 2^{nd} sequence. The presenting complex 1^{st} sequence and presenting complex 2^{nd} sequence generally associate through non-covalent interactions between the MHC-H chain and the β2M polypeptide sequence, and may be stabilized by disulfide bonds between either the MHC sequences or peptide/polypeptide linkers attached to the N- or C-t terminus of the MHC sequences. The presenting complex 1^{st} sequence and presenting complex 2^{nd} sequence may also associate through dimerization or interspecific dimerization sequences if present in those polypeptides. MAPPs with presenting complexes typically contain one or two presenting complexes, and accordingly, duplex MAPPs with presenting complexes typically comprise two or four presenting complexes, but may contain one, two, three, or four presenting complexes. As discussed above, MAPPs and duplex MAPPs may comprise more presenting complexes depending on, for example, the number of dimerization sequences in the framework polypeptide.

MAPPs and accordingly their higher order complexes (duplexes, triplexes etc.) comprise MHC Class I polypeptide sequences that bind an epitope for presentation to a TCR, and accordingly may present peptides to T cells (e.g., CD8+ T cells). The effect of MAPPs on T cells with TCRs specific to the epitope depends on which, if any, MODs are present in the MAPP. In the absence of any stimulatory MOD, prolonged exposure to the MAPP may result in T cell anergy or suppression of T cell stimulation.

MOD-containing MAPPs can function as a means of selectively delivering the MODs to T cells specific for an epitope, resulting in MOD-driven T cell responses (e.g., proliferation of epitope specific T cells). The incorporation of one or more MODs with affinity for their cognate receptor on T cells (“co-MOD”) can reduce the specificity of MAPPs and duplex MAPPs for epitope specific T cells where MOD-co-MOD binding interactions significantly compete with MHC/epitope binding to target cell TCR. The inclusion of MODs with reduced affinity for their co-MOD(s), and the affinity of the epitope for a TCR, provides for enhanced selectivity of MAPPs and duplex MAPPs, while retaining the activity of the MODs.

The ability of MAPPs and duplex MAPPs of the present disclosure to modulate T cells provides methods of modulating the activity of T cells in vitro and in vivo, and accordingly the use of MAPPs and/or duplex MAPPs as therapeutics in methods of treatment.

The MAPPs and duplex MAPPs of the present disclosure are useful for modulating activity of T cells selective for specific epitope peptides by delivering to those T cells the desired immunomodulators. Thus, the present disclosure provides methods of modulating activity of a T cell in vitro, ex vivo, and in vivo, as well as methods of treating diseases, such as cancers and benign (non-malignant) neoplasms associated with KRAS sequence variations.

The present disclosure provides nucleic acids comprising nucleotide sequences encoding individual MAPP polypeptides and MAPPs (e.g., all polypeptides of the MAPP), as well as cells genetically modified with the nucleic acids and producing MAPP polypeptides and/or MAPP proteins (e.g., duplex MAPPs). The present disclosure provides methods of producing MAPPs, duplex MAPPs, and higher order MAPPs utilizing such cells.

### II. BRIEF DESCRIPTION OF THE DRAWINGS

FIG. 1A is provided to illustrate of the terminology used to describe MAPPs and duplex MAPPs with presenting sequences. The peptides are oriented from N-terminus (left) to C-terminus (right). The figure shows first and second framework polypeptides, which in this case are different and have specific multimerization sequences comprising a knob and counterpart hole (KiH). Also shown are first and second dimerization polypeptides having an N-terminal epitope peptide and counterpart dimerization sequences. The dashed circles indicate five potential locations for the addition of peptide sequences, including sequences of MODs (discussed below). The figure depicts the formation of a first and second heterodimer MAPPs each comprising a framework polypeptide and dimerization polypeptide. The heterodimers may interact through the multimerization sequence to form a multimer (a duplex MAPP as shown). The use of knob-in-hole sequences permits the assembly of an asymmetric interspecific duplex MAPP where, for example, different MOD sequences are provided at positions 1 and 1′ and/or positions 3 and 3′. While interactions between polypeptide chains through peptide interaction sequences may initially be non-covalent in nature, interchain disulfide bond formation reactions may occur thereby providing covalently linked polypeptides at, for example, either dimerization sequences or multimerization sequences. Throughout the figures, lines connecting various elements of MAPP polypeptides are optional aas serving as linkers (e.g., peptide linkers).

FIG. 1B parallels FIGS. 1A and 1s provided to illustrate of the terminology used to describe MAPPs and duplex MAPPs with presenting complexes.

FIG. 2 provides a multiple aa sequence alignment of β2M precursors (i.e., including the leader sequence) from Homo sapiens (NP_004039.1; SEQ ID NO:1), Pan troglodytes (NP_001009066.1; SEQ ID NO:2), Macaca mulatta (NP_001040602.1; SEQ ID NO:3), Bos Taurus (NP_776318.1; SEQ ID NO:4) and Mus musculus (NP_033865.2; SEQ ID NO:5). Underlined aas 1-20 are the signal peptide (sometime referred to as a leader sequence) which is absent in the mature protein, and unless stated otherwise is not present in MAPP polypeptides.

FIGS. 3A, 3B and 3C provide aa sequences of HLA class I heavy chain polypeptides. Signal sequences, aas 1-24, are bolded and underlined. FIG. 3A entry: 3A.1 is the HLA-A heavy chain (HLA-A*01:01:01:01 or A*0101) (NCBI accession NP_001229687.1), SEQ ID NO:6; entry 3A.2 is ‘HLA-A*1101, SEQ ID NO:7; entry 3A.3 is HLA-A*2402, SEQ ID NO:8; and entry 3A.4 is HLA-A*3303, SEQ ID NO:9. FIG. 3B provides the sequence for HLA-B*07:02:01 (HLA-B*0702) (NCBI GenBank Accession NP_005505.2 (see, also GenBank Accession AUV50118.1), SEQ ID NO:10). FIG. 3C provides the sequence for HLA-C*0701 (GenBank Accession NP_001229971.1) (HLA-C*07:01:01:01 or HLA-Cw*070101), SEQ ID NO:11, (HLA-Cw*07) (see GenBank Accession CA078194.1).

FIG. 3D provides an alignment of eleven mature MHC Class I heavy chain peptide sequences without all, or substantially all, of their leader, transmembrane and intracellular domain regions. The aligned sequences include human HLA-A*0101, SEQ ID NO:12 (see also SEQ ID NO:6); HLA-B*0702, SEQ ID NO:13 (see SEQ ID NO:10); HLA-C, SEQ ID NO:14 (see SEQ ID NO:11); HLA-A*0201, SEQ ID NO:15; a mouse H2K protein sequence, SEQ ID NO:16; three allelic variants of HLA-A (var.2, var.2C, and var.2CP, SEQ ID NOs:17, 18, and 19); and 3 human HLA-A polypeptides (HLA-A*1101 (HLA-A11), SEQ ID NO:20; HLA-A*2402 (HLA-A24), SEQ ID NO:21; and HLA-A*3303 (HLA-A33, SEQ ID NO:22). HLA-A*0201 is an allelic variant of the sequence marked as HLA-A. Marked as HLA-A (var. 2) is the Y84A and A236C variant of HLA-A. The seventh HLA-A sequence, marked as HLA-A (var. 2C), shows the HLA-A sequence substituted with C residues at positions 84, 139 and 236, and the 8th sequence adds one additional proline to the C-terminus of the preceding sequence. The 9th through the 11th sequences are from HLA-A11 (HLA-A*1101), HLA-A24 (HLA-A*2402); and HLA-A33 (HLA-A*3303), respectively, which are prevalent in certain Asian populations. Indicated in the alignment are aa locations (aas 84 and 139 of the mature proteins) where cysteine residues may be inserted in place of the wt. aas to form an intrachain stabilizing disulfide bond that stabilizes the MHC-H epitope peptide binding pocket, particularly when the epitope peptide is not present or does not bind the MHC pocket with high affinity. Also shown in the alignment is position 236 (of the mature polypeptide), which may be replaced by a cysteine residue that can form an interchain disulfide bond with β2M (e.g., at aa 12 of the mature polypeptide). An arrow appears above each of those locations and the residues are bolded. The boxes flanking residues 84, 139 and 236 show the groups of five aas on either side of those six sets of five residues, denoted aa cluster 1, aa cluster 2, aa cluster 3, aa cluster 4, aa cluster 5, and aa cluster 6 (shown in the figure as aac1 through aac6, respectively). Any one or more of those groups of aas may be replaced by 1 to 5 amino acids selected independently from (i) any naturally occurring amino acid or (ii) any naturally occurring amino acid except proline or glycine.

FIGS. 3E-3G provide alignments of the amino acid sequences of mature HLA-A, -B, and, -C class I heavy chains, respectively. The sequences are provided for a portion of the mature proteins (without all or substantially all of their leader sequences, transmembrane domains or intracellular domains). As described in FIG. 3D, the positions of aa residues 84, 139, and 236 and their flanking residues (aac1 to aac6) that may be replaced by 1 to 5 amino acids selected independently from (i) any naturally occurring amino acid or (ii) any naturally occurring amino acid except proline or glycine are also shown. A consensus sequence is also provided for each group of HLA alleles provided in the figures showing the variable aa positions as “X” residues sequentially numbered and the locations of aas 84, 139 and 236 double underlined.

FIG. 3H provides a consensus sequence for each of HLA-E, -F, and -G with the variable aa positions indicated as “X” residues sequentially numbered and the locations of aas 84, 139 and 236 double underlined.

FIG. 3I provides an alignment of the consensus aa sequences for HLA-A, -B, -C, -E, -F, and -G, which are given in FIGS. 3E to 3H. The alignment shows the correspondence of amino acids between the different sequences. Variable residues in each sequence are listed as “X” with the sequential numbering removed. The permissible amino acids at each variable residue can be determined by reference to FIGS. 3E-3H As indicated in FIG. 3D, the locations of aas 84, 139 and 236 with their flanking five-amino acid clusters that may be replaced by 1 to 5 amino acids selected independently from (i) any naturally occurring amino acid or (ii) any naturally occurring amino acid except proline or glycine are also shown.

FIGS. 4A-4H provides partial amino acid sequences of immunoglobulin polypeptides including their heavy chain constant regions (“Ig Fc” or “Fc”, e.g., the CH2-CH3 domain of IgG1) SEQ ID NOs:54 to 66).

FIG. 4I provides the sequence of an immunoglobulin heavy chain region 1 (CH1) or Ig CH1 domain (SEQ ID NO:67).

FIG. 4J provides the sequence of a human immunoglobulin J chain precursor, NCBI accession No. NP_653247.1 (SEQ ID NO:68).

FIG. 5A provides the sequence of an immunoglobulin (“Ig”) light chain constant region (“C_{L}”) or “Ig C_{L}” from a human kappa light chain (Ig C_{L} κ chain) (SEQ ID NO:69).

FIG. 5B provides the sequence of an Ig lambda light chain constant region Ig C_{L}λ chain (SEQ ID NO:70).

FIG. 6 provides a series of MAPP structures based on framework polypeptides having both a multimerization sequence and first and second dimerization sequences that may be the same or different. The structure is shown generically in A with locations 1-5 and 1′-5′ indicating locations for additional peptide sequences (e.g., MOD polypeptide sequences). The MHC/epitope can be either a presenting sequence (see, e.g., FIG. 12), or presenting complexes (see FIGS. 13-15). Locations 4 and 4′ are shown at the N-terminus of the presenting sequence or complex and locations 5 and 5′ are shown at the C-terminus. Locations 1 and 1′ are shown at the N-terminus of the framework peptide and locations 3 and 3′ at the C-terminus of the framework polypeptide. In B the framework polypeptides are multimerized to form a duplex of heterodimers using an immunoglobulin Fc region knob-in-hole moti-f. In C the duplexes contain heterodimers in which two different asymmetric interspecific dimerization sequences bind together the framework peptides and their associated dimerization peptides. In D the framework peptides are joined together by a knob-in-hole Fc motif and the dimerization peptide and framework peptide are joined together by different dimerization sequences to form a duplex of heterodimers.

FIG. 7 provides in A to D a series of MAPP structures as in FIG. 6, with the addition of presenting sequences or presenting complexes at the N-terminus of the framework peptides. Positions 4 and 4′ may still serve as locations for peptide addition (e.g., MOD polypeptide addition).

FIG. 8 provides in A to D a series of MAPP structures as in FIG. 6, where the dimerization sequences are Ig CH1 sequences (CH1) that pair with Ig light chain κ or λ sequences (CL). The framework peptides are multimerized (dimers in this instance) through the interaction of Ig Fc (e.g., CH2 and CH3) regions, with the structures in B and D having knob-in-hole motifs to permit heteroduplexes to be formed. The peptides are also joined by disulfide bonds (e.g., those that form between Ig Fc region peptides).

FIG. 9 provides a series of MAPP structures as in FIG. 8, with the addition of presenting sequences or presenting complexes at the N-terminus of the framework peptides. Positions 4 and 4′ may still serve as locations for peptide addition (e.g., MOD polypeptide addition).

FIG. 10 provides in A to J a series of MAPP structures as in FIG. 8. In each instance a presentation sequence lacking a MOD sequence is present on the dimerization peptide (marked as a single chain MHC and epitope). Locations 2, 2′, 4, 4′, 5 and 5′ are unfiled and not shown. Locations 1 and 1′ are substituted with one or more MODs selected from IL-2b, PD L1, and CD80. Positions 3 and 3′ are shown for orientation in A to G. In H to J the 3 and 3′ locations are either unfiled or, for example, a TGF-β or 4-1BBL MOD may be located there.

FIG. 11 provides examples of duplex MAPPs epitope presenting sequences detailed (the “Single Chain MHC” with the epitope as part of the structure. Although shown with the specific epitope ELAGIGILTV, that peptide epitope may be replaced by any other translatable epitope peptide.

FIG. 12 shows in A and B two different MHC Class I presenting sequences (from the epitope at the N-terminus to C-terminus. In the figures (e.g., FIGS. 12 to 15) the symbol “-//-” indicates a point of connection with a dimerization peptide or a framework peptide to form a single peptide sequence. The sequences optionally comprise one or more independently selected MODs (including MODs in tandem) at the locations indicated (e.g. at the N-terminus or between the recited elements, such as in a linker polypeptide sequences).

FIGS. 13 to 15 show a series of MHC class I presenting complexes from N- to C-terminus. The sequence bearing the symbol “-//-” is the presenting complex 1^{st} polypeptide sequence (presenting complex 1^{st} sequence). The other sequence the presenting complex 2^{nd} sequence that is associated with the presenting complex 1^{st} sequence. The symbol “-//-” also indicates a point of connection with a dimerization peptide or a framework peptide. In FIG. 15 the presenting complex 1^{st} sequence and its associated presenting complex 2^{nd} sequence are covalently attached by the formation of a disulfide bond.

FIG. 16A-16D provide the aa sequence of the constructs employed in Example 1.

### III. DETAILED DESCRIPTION

### A. Definitions

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “polypeptide,” and “protein” are used interchangeably herein, and refer to a polymeric form of amino acids, which unless stated otherwise are the naturally occurring proteinogenic L-amino acids that are incorporated biosynthetically into proteins during translation in a mammalian cell. Furthermore, as used herein, these terms may refer to polypeptides or proteins that include modifications, such as deletions, additions, and substitutions (generally conservative in nature as would be known to a person in the art) to the native sequence, as long as the protein maintains the desired activity. These modifications can be deliberate, as through site-directed mutagenesis, or can be accidental, such as through mutations of hosts that produce the proteins, or errors due to polymerase chain reaction (PCR) amplification or other recombinant DNA methods. References herein to a specific residue or residue number in a known polypeptide, e.g., a human MHC class I polypeptide, are understood to refer to the amino acid at that position in the wild-type polypeptide. To the extent that the sequence of the wild-type polypeptide is altered, either by addition or deletion of one or more amino acids, one of ordinary skill will understand that a reference to the specific residue or residue number will be correspondingly altered so as to refer to the same specific amino acid in the altered polypeptide, which would be understood to reside at an altered position number. A reference to a “non-naturally occurring Cys residue” in a polypeptide, e.g., an MHC class I polypeptide, means that the polypeptide comprises a Cys residue in a location where there is no Cys in the corresponding wild-type polypeptide. This can be accomplished through routine protein engineering in which a cysteine is substituted for the amino acid that occurs in the wild-type sequence.

A nucleic acid or polypeptide has a certain percent “sequence identity” to another nucleic acid or polypeptide, meaning that, when aligned, that percentage of bases or amino acids are the same, and in the same relative position, when comparing the two sequences. Sequence identity can be determined in a number of different ways. To determine sequence identity, sequences can be aligned using various convenient methods and computer programs (e.g., BLAST, T-COFFEE, MUSCLE, MAFFT, etc.), available over the world wide web at sites including blast.ncbi.nlm.nih.gov/Blast.cgi for BLAST+2.10.0, ebi.ac.uk/Tools/msa/tcoffee/, ebi.ac.uk/Tools/msa/muscle/, and mafft.cbrc.jp/alignment/software/. See, e.g., Altschul et al. (1990), J. Mol. Biol. 215:403-10.

As used herein amino acid (“aa” singular or “aas” plural) means the naturally occurring proteogenic amino acids incorporated into polypeptides and proteins in mammalian cell translation. Unless stated otherwise: L (Leu, leucine), A (Ala, alanine), G (Gly, glycine), S (Ser, serine), V (Val, valine), F (Phe, phenylalanine), Y (Tyr, tyrosine), H (His, histidine), R (Arg, arginine), N (Asn, asparagine), E (Glu, glutamic acid), D (Asp, asparagine), C (Cys, cysteine), Q (Gln, glutamine), I (Ile, isoleucine), M (Met, methionine), P (Pro, proline), T (Thr, threonine), K (Lys, lysine), and W (Trp, tryptophan). Amino acid also includes the amino acids hydroxyproline and selenocysteine, which appear in some proteins found in mammalian cells, however, unless their presence is expressly indicated they are not understood to be included.

The term “conservative amino acid substitution” refers to the interchangeability in proteins of aa residues having similar side chains. For example, a group of aas having aliphatic side chains consists of glycine, alanine, valine, leucine, and isoleucine; a group of aas having aliphatic-hydroxyl side chains consists of serine and threonine; a group of aas having amide containing side chains consists of asparagine and glutamine; a group of aas having aromatic side chains consists of phenylalanine, tyrosine, and tryptophan; a group of aas having basic side chains consists of lysine, arginine, and histidine; a group of aas having acidic side chains consists of glutamate and aspartate; and a group of aas having sulfur containing side chains consists of cysteine and methionine. Exemplary conservative aa substitution groups are: valine-leucine-isoleucine, phenylalanine-tyrosine, lysine-arginine, alanine-valine-glycine, and asparagine-glutamine.

The term “binding” refers to a direct association between molecules and/or atoms, due to, for example, covalent, electrostatic, hydrophobic, and ionic and/or hydrogen-bond interactions, including interactions such as salt bridges and water bridges.

The term “binding,” as used with reference to a MAPP to a polypeptide (e.g., a T cell receptor on a T cell), refers to a non-covalent interaction between two molecules. Non-covalent interactions/binding refers to a direct association between two molecules, due to, for example, electrostatic, hydrophobic, ionic, and/or hydrogen-bond interactions, including interactions such as salt bridges and water bridges. Non-covalent binding interactions are generally characterized by a dissociation constant (K_{D}) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Covalent bonding,” or “covalent binding” as used herein, refers to the formation of one or more covalent chemical bonds between two different molecules.

“Affinity” as used herein generally refers to the strength of non-covalent binding, increased binding affinity being correlated with a lower K_{D}. As used herein, the term “affinity” may be described by the dissociation constant (K_{D}) for the reversible binding of two agents (e.g., an antibody and an antigen. Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 40-fold greater, at least 60-fold greater, at least 80-fold greater, at least 100-fold greater, or at least 1,000-fold greater, or more, than the affinity of an antibody or receptor for an unrelated aa sequence (e.g., ligand). Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution.

The term “immunological synapse” or “immune synapse” as used herein generally refers to the natural interface between two interacting immune cells of an adaptive immune response including, e.g., the interface between an antigen-presenting cell (APC) or target cell and an effector cell, e.g., a lymphocyte, an effector T cell, a natural killer cell, and the like. An immunological synapse between an APC and a T cell is generally initiated by the interaction of a T cell antigen receptor and major histocompatibility complex molecules, e.g., as described in Bromley et al., Ann. Rev. Immunol. 2001; 19:375-96; the disclosure of which is incorporated herein by reference in its entirety.

“T cell” includes all types of immune cells expressing CD3, including T-helper cells (CD4⁺ cells), cytotoxic T cells (CD8⁺ cells), T-regulatory cells (Treg), and NK-T cells.

The term “immunomodulatory polypeptide” (also referred to as a “costimulatory polypeptide” or, as noted above, “MOD”), as used herein includes a polypeptide or portion thereof (e.g., an ectodomain) on an APC (e.g., a dendritic cell, a B cell, and the like), or otherwise available to interact with the T cell, that specifically binds a cognate co-immunomodulatory polypeptide (“co-MOD”), present on a T cell, thereby providing a signal. The signal provided by the MOD engaging its co-MOD in addition to the primary signal provided by, for instance, binding of a TCR/CD3 complex with a MHC polypeptide loaded with peptide, mediates (e.g., directs) a T cell response. The responses include, but are not limited to, proliferation, activation, differentiation, and the like. A MOD can include, but is not limited to, CD7, B7-1 (CD80), B7-2 (CD86), PD-L1, PD-L2, 4-1BBL, OX40L, Fas ligand (FasL), inducible costimulatory ligand (ICOS-L), intercellular adhesion molecule (ICAM), CD30L, CD40, CD70, CD83, HLA-G, lymphotoxin beta receptor, 3/TR6, ILT3, ILT4, HVEM, an agonist or antibody that binds Toll ligand receptor and a ligand that specifically binds with B7-H3. A MOD also encompasses, inter alia, an antibody or antibody fragment that specifically binds with and activates a cognate co-stimulatory (co-MOD) molecule present on a T cell, such as, but not limited to antibodies against the receptors for any of IL-2, CD27, CD28, 4-1BB, OX40, CD30, CD40, PD-1, ICOS, lymphocyte function-associated antigen-1 (LFA-1), CD2, LIGHT, NKG2C, B7-DC, B7-H2, B7-H3, and CD83.

“Heterologous,” as used herein, means a nucleotide or polypeptide that is not found in the native nucleic acid or protein, respectively.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system.

The terms “recombinant expression vector,” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

The terms “treatment,” “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting the disease or symptom, i.e., arresting its development; and/or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient” are used interchangeably herein and refer to any mammalian subject for whom diagnosis, treatment, or therapy is desired. Mammals include humans and non-human primates, and in addition include rodents (e.g., rats; mice), lagomorphs (e.g., rabbits), ungulates (e.g., cows, sheep, pigs, horses, goats, and the like), felines, canines, etc.

Unless indicated otherwise, the term “substantially” is intended to encompass both “wholly” and “largely but not wholly”. For example, an Ig Fc that “substantially does not induce cell lysis” means an Ig Fc that induces no cell lysis at all or that largely does not induce cell lysis.

As used herein, the term “about” used in connection with an amount indicates that the amount can vary by 10% of the stated amount. For example, “about 100” means an amount of from 90-110. Where about is used in the context of a range, the “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

As used herein the term “in vivo” refers to any process or procedure occurring inside of the body, e.g., of a patient having a cancer caused by a KRAS mutation.

As used herein, “in vitro” and “ex vivo” refer to processes or procedures occurring outside of the body. Although the terms may be used interchangeably, the term ex vivo is generally used to indicate a process where an animal-derived (e.g., human-derived) tissue is subjected to a process outside of the body and then the acted-upon tissue is re-inserted into the animal.

Before the present invention is further described, it is to be understood that this invention is not limited to the particular embodiments described, as such may, of course, vary. It is also to be understood that the terminology used herein is for the purpose of describing particular embodiments only, and is not intended to be limit the scope of the invention.

Where a range of values is provided, it is understood that each intervening value between the upper and lower limit of that range to a tenth of the lower limit of the range is encompassed within the disclosure along with any other stated or intervening value in the range. The upper and lower limits of these smaller ranges may independently be included in smaller ranges, that are also encompassed within the disclosure subject to any specifically excluded limit in the stated range. Where the stated range a value (e.g., an upper or lower limit), ranges excluding those values are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a Treg” includes a plurality of such Tregs and reference to “the MHC Class I heavy chain” includes reference to one or more MHC Class I heavy chains and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### B. Description

1. MAPP Structure And The Role of Framework and Dimerization Peptides

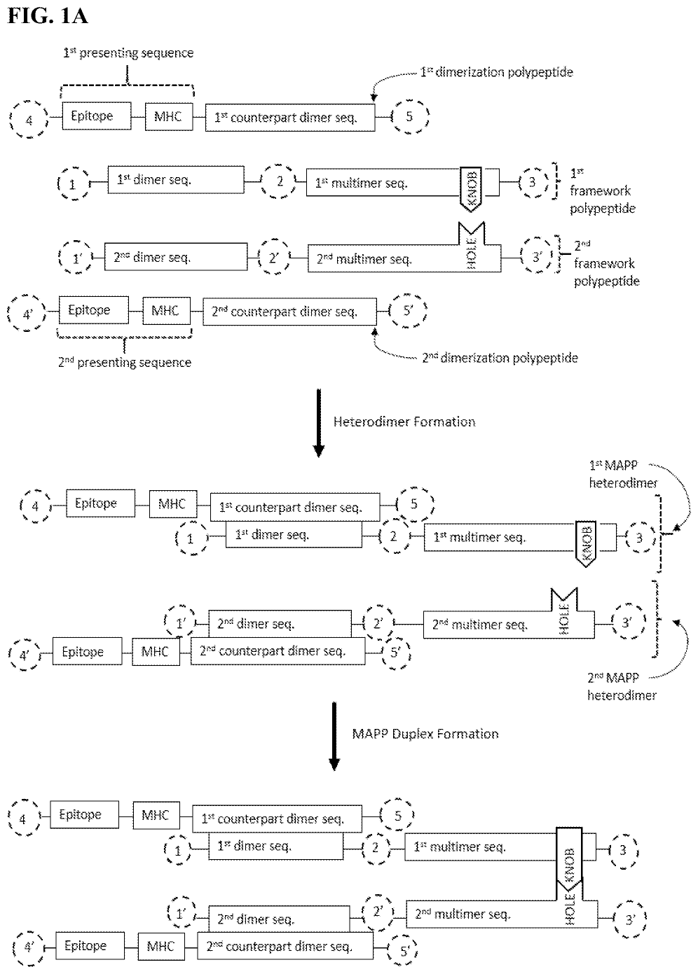

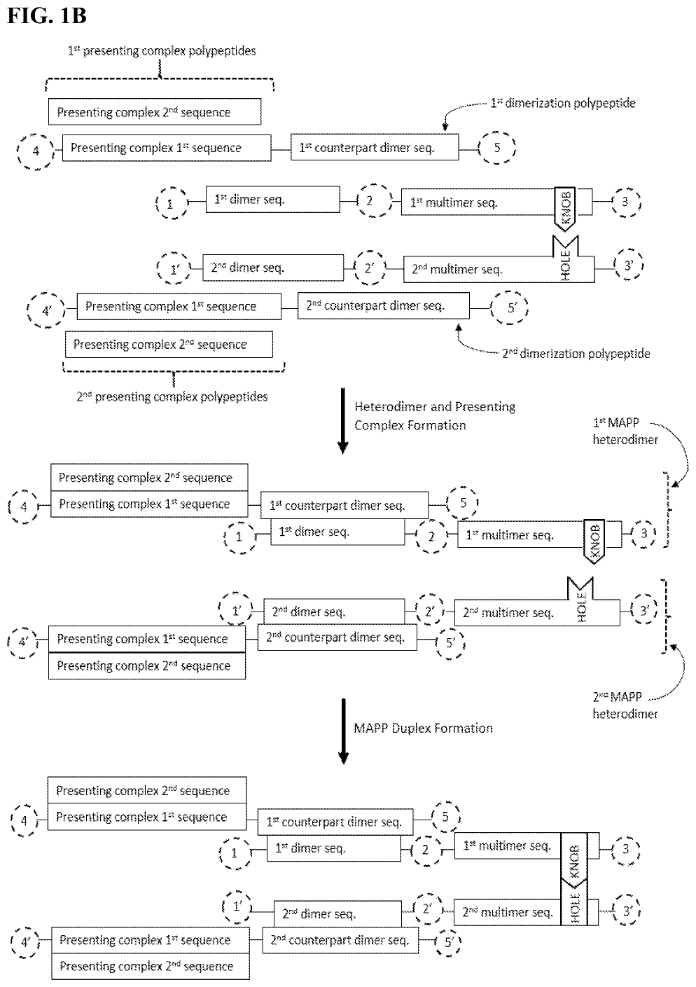

The present disclosure provides multimeric antigen-presenting polypeptide complexes (“MAPP” singular and “MAPPs” plural) that include at least one framework polypeptide and at least one dimerization polypeptide. Framework polypeptides comprise one or more polypeptide dimerization sequence that permits specific binding with other polypeptides (dimerization polypeptides) having a counterpart dimerization sequence thereby forming at least a heterodimer (see FIGS. 1A and 1B). Framework polypeptides also comprise a multimerization sequence(s) that permits two or more framework polypeptides to associate, thereby forming a higher order structure (e.g., a duplex of the two or more heterodimers, a “duplex MAPP” see FIGS. 1A and 1B). Neither the dimerization sequence nor the multimerization sequence of the framework polypeptide (or the counterpart dimerization sequence) comprises an MHC-H polypeptide sequence or a β2M polypeptide sequence; and as such, interaction brought about by those sequences are not consider dimerization or multimerization of framework and/or dimerization peptides. Accordingly, the framework polypeptides provide a structure upon which other polypeptides can be organized by interactions at the dimerization sequences, and which can interact with other framework polypeptides by way of multimerization sequences.

The framework and dimerization peptide containing MAPPs, duplex MAPPs, and MAPPs of higher order (e.g., triplex MAPPs) described herein provide a means by which KRAS epitope presenting molecules, in particular peptides, (“epitope peptides,” “KRAS epitopes” or simply “epitopes” for the purpose of this disclosure) may be delivered in the context of an MHC (e.g., HLA) to a target T cell displaying a TCR specific for the epitope, while at the same time permitting for the flexible presentation of one or more immunomodulatory polypeptides (“MODs”). The MAPPs, duplex MAPPs, and higher order MAPPs thereby permit deliver of one or more MODs in an epitope selective (e.g., dependent/specific) manner that permits formation of an active immune synapse with a target T cell selective for the epitope, and control/regulation of the target T cell's response to the epitope. Accordingly, where MAPPs comprise stimulatory or activating MODs (e.g., IL-2, CD80, CD86, and/or 4-1BBL) that increase T cell proliferation and/or effector functions in an epitope selective manner. In contrast, where MAPPs comprise inhibitory MODs (e.g., FasL and/or PDL1) they decrease T cell proliferation and/or effector functions in an epitope selective manner.

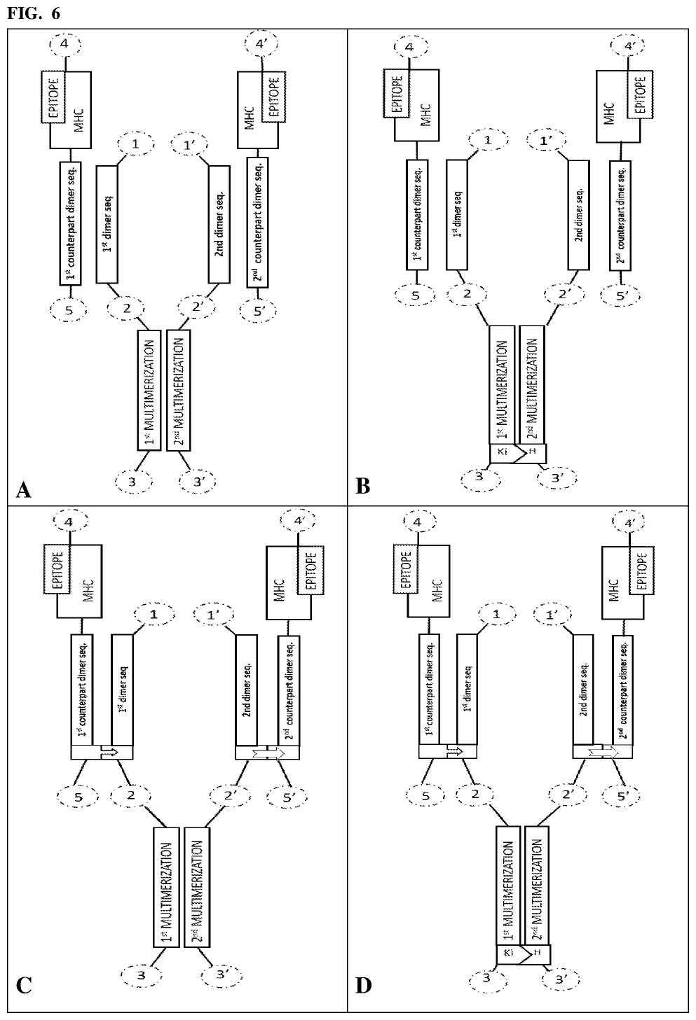

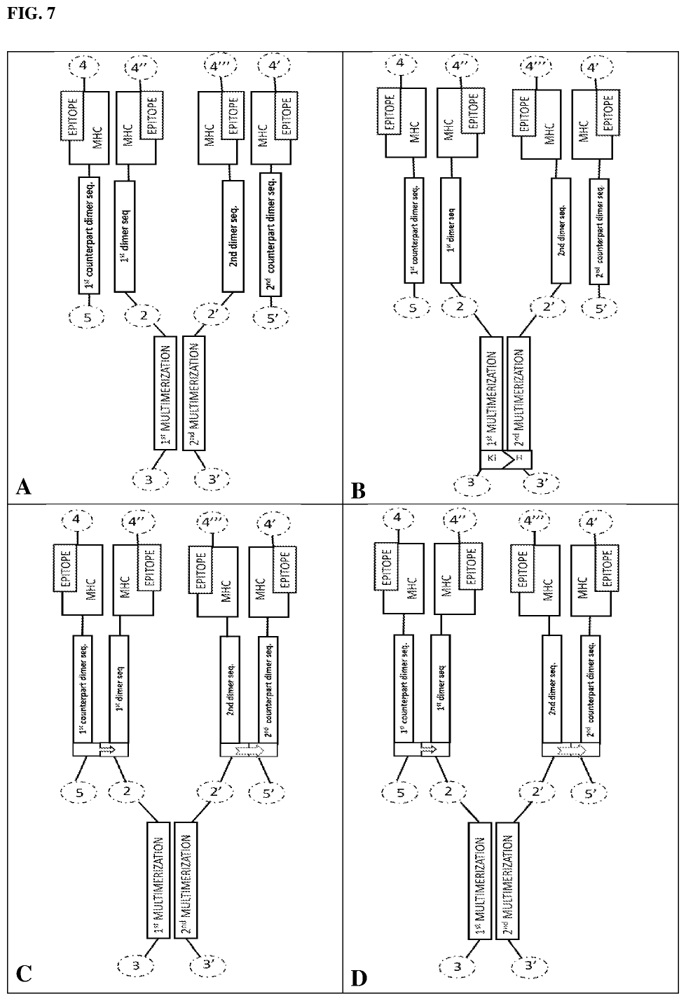

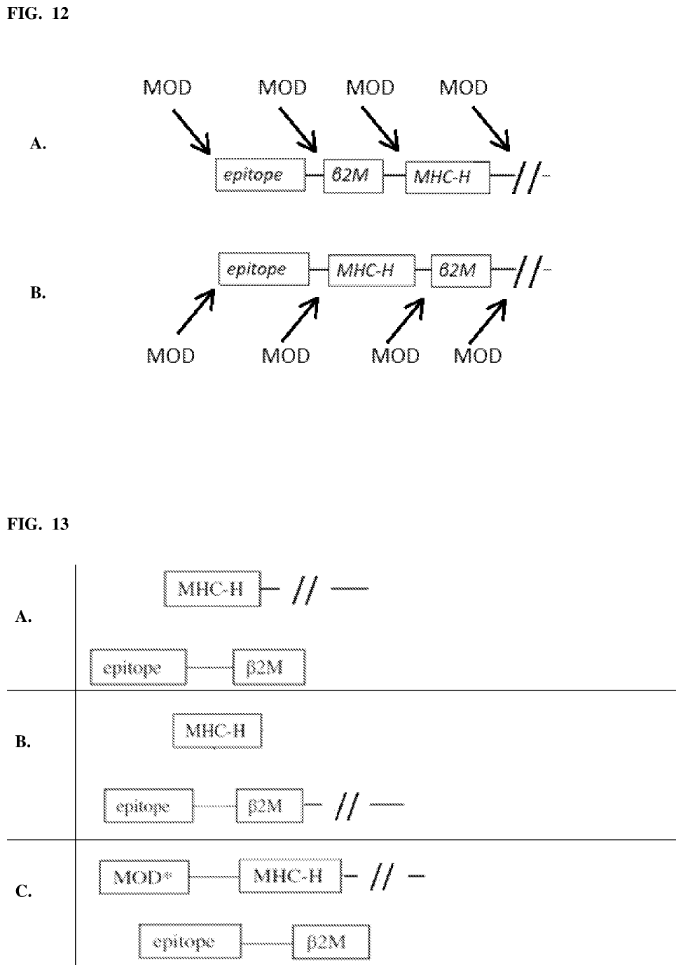

MHC polypeptide portions of MAPP presenting epitopes may be divided into single polypeptide chain presenting sequences, or presenting complexes comprising two or more polypeptide chains. Where the epitope peptide, an MHC-H and β2M polypeptide sequences, and optionally one or more MODs are provided in one polypeptide, it is termed an “epitope presenting sequence” or “presenting sequence.” See, e.g., FIG. 12. MAPPs typically contain one or two presenting sequence, and accordingly, duplex MAPPS typically comprise one, two, three, four presenting sequences. MAPPs and duplex MAPPs may comprise more presenting sequences depending on, for example, the number of dimerization sequences in the framework polypeptide. The presenting sequences may be integrated into a MAPP as part of the a framework polypeptide or a dimerization polypeptide. A MAPP may have presenting sequences as part of either or both of framework or dimerization polypeptide. Compare, for example, FIG. 6 structures A-D and FIG. 7 structures A-D. Presenting sequences may further comprise one or more MOD peptide sequences (see FIG. 12).

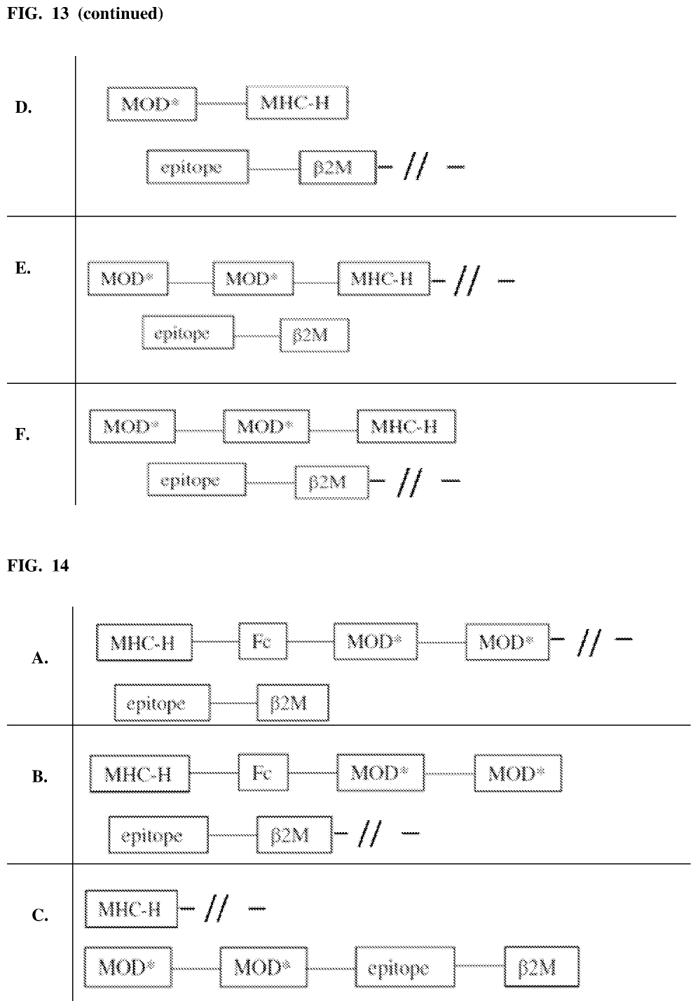

As an alternative to utilizing a single polypeptide to present an epitope, the MHC components (MHC-H polypeptide sequences) and the epitope may be divided (split) into two separate polypeptide sequences, which together are denoted herein as an “epitope presenting complex” or “presenting complex.” A presenting complex is integrated into a MAPP by having a presenting complex first amino acid sequence (“presenting complex 1^{st} sequence”) as part of a framework or dimerization polypeptide. The remaining MHC sequence(s) are part of a polypeptide termed the presenting complex second amino acid sequence (“presenting complex 2^{nd} sequence”). The epitope peptide and any independently selected MODs that are present may be part of the polypeptide comprising either the presenting complex 1^{st} sequence or the presenting complex 2^{nd} sequence. The presenting complex 1^{st} sequence and presenting complex 2^{nd} sequence associate through non-covalent interactions between the MHC-H chain and the β2M polypeptide sequence, and may be stabilized by disulfide bonds between either the MHC sequences or peptide/polypeptide linkers attached to the N- or C-t terminus of the MHC sequences. The presenting complex 1^{st} sequence and presenting complex 2^{nd} sequence may also associate through dimerization or interspecific dimerization sequences if present in those polypeptides. MAPPs with presenting complexes typically contain one or two presenting complexes, and accordingly, duplex MAPPs with presenting complexes typically comprise one, two, three, or four presenting complexes. As discussed above, MAPPs and duplex MAPPs may comprise more presenting complexes depending on, for example, the number of dimerization sequences in the framework polypeptide. Presenting complexes may further comprise one or more MOD peptide sequences as part of the presenting complex 1^{st} sequence or presenting complex 2^{st} sequence. FIGS. 13-15.

MAPPs and their higher order complexes (e.g., duplex MAPPs) are intended to be soluble in aqueous media under physiological conditions (e.g., soluble in human blood plasma at therapeutic levels). Accordingly, they do not contain peptide sequences that would anchor them in a cell membrane, such as an MHC or immunomodulatory protein transmembrane domain, or portion thereof, that would cause a MAPP or duplex MAPP to anchor in a Chinese Hamster Ovary cell during cellular expression.

The framework/dimerization polypeptide architecture of MAPPs and their higher order structures may also be understood to provide flexibility in locating MODs and epitope presenting complexes or epitope presenting sequences. Duplex MAPP and higher order MAPP architecture can be particularly useful when both the MOD and the epitope presenting complexes (or epitope presenting sequences) are most biologically active (e.g., optimally active) when present at the N-terminus of a polypeptide. In such an instance, employing a duplex MAPP the MOD and the epitope presenting complex (or epitope presenting sequence) may each be located at the N-terminus of different framework and/or dimerization polypeptide sequences.

The structure of MAPPs, and particularly higher order MAPPs such as duplexes may be specified by the use of a pairs of polypeptides having different sequences that specifically pair with each other. Multimerization of framework polypeptides results from interactions between multimerization sequences, and dimerization (the interaction of a framework and dimerization polypeptide) results from the interaction of a dimerization sequence on the framework polypeptide and a counterpart dimerization on a dimerization polypeptide. For example, in a duplex MAPP the multimerization sequences may be Ig Fc heavy chain (e.g, CH2-CH3) sequences, and the dimerization sequence and counterpart dimerization sequences may be the same (e.g., all leucine zipper sequences). An additional degree of control may be obtained by utilizing non-identical peptide sequences that specifically/selectively pair with each other that are referred to herein as “interspecific sequences” or “interspecific dimerization sequences” or “interspecific multimerization sequences,” and give rise to asymmetric interspecific pairs of sequences. The structure of MAPPs permit effective placement of each polypeptide into a MAPP (see e.g., FIGS. 6-10). Interspecific sequences include Ig heavy chain Fc (CH2-CH3) region modified with knob-in-hole variations; and Fos peptide sequences paired with Jun peptide sequences. Accordingly, MAPP architectures include, but are not limited to, MAPPs where each, or some, of the dimerization sites are different (permit different peptide pairings). For example, duplex MAPPs where each multimerization and dimerization site is different and provides separate peptide pairings.

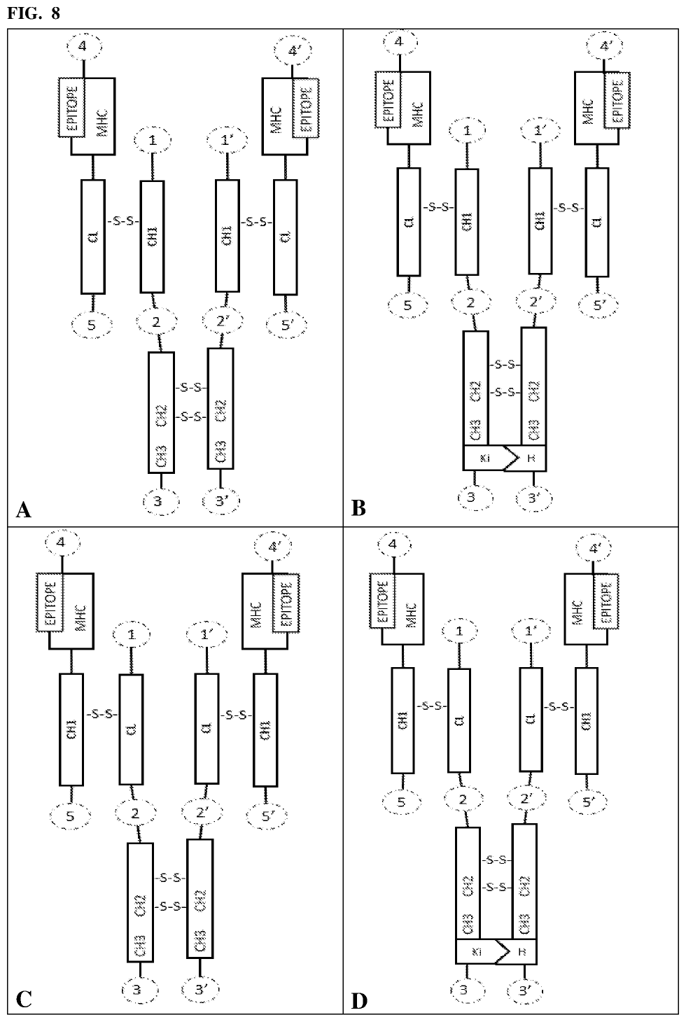

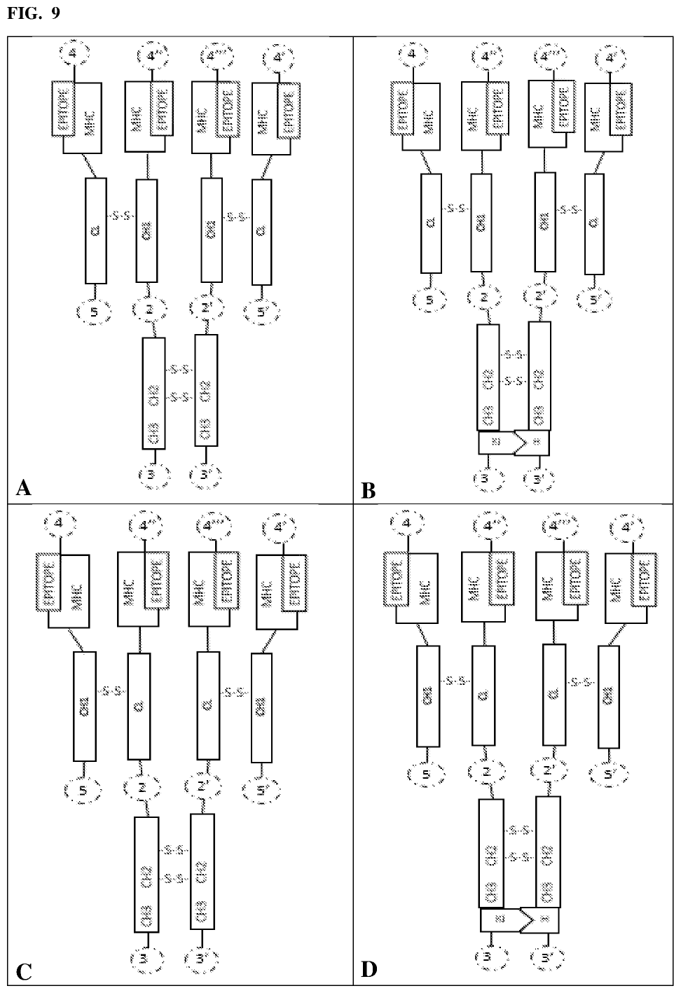

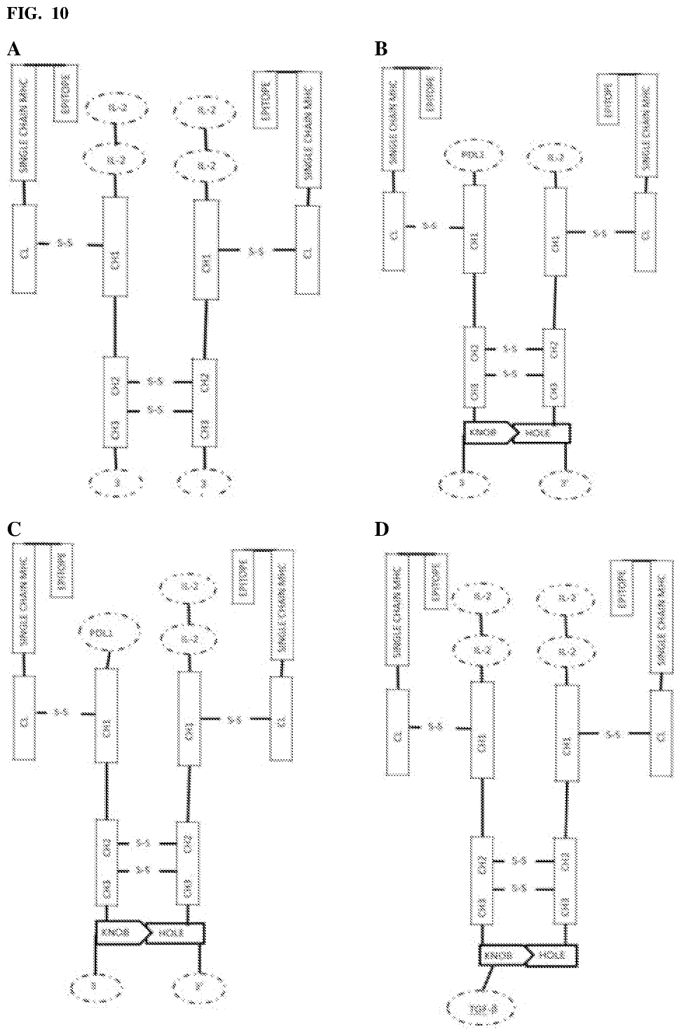

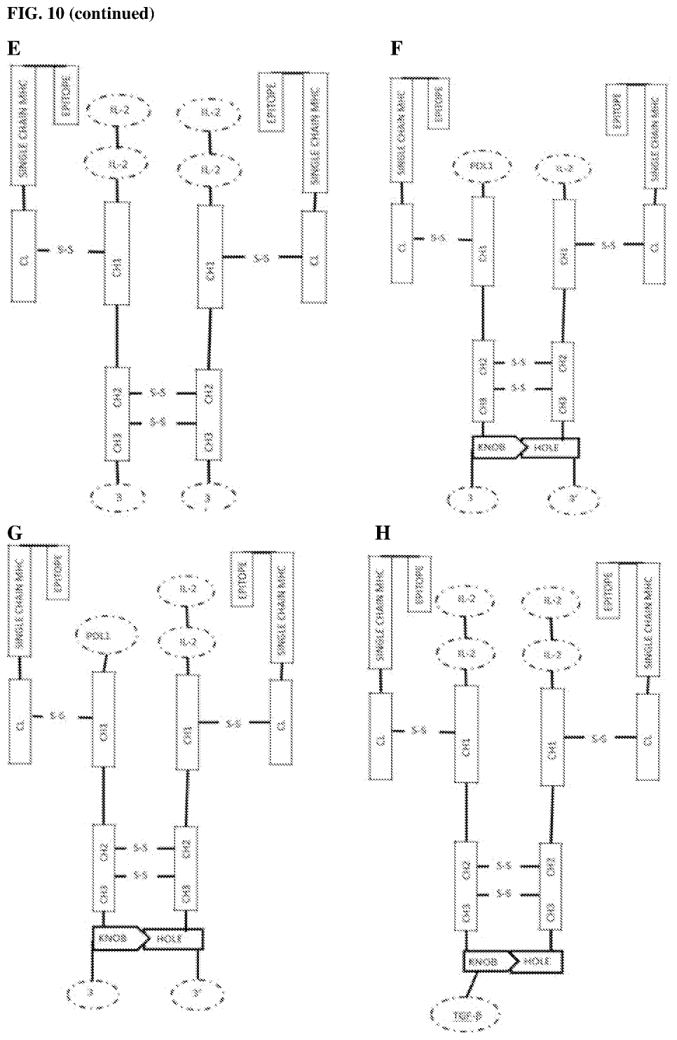

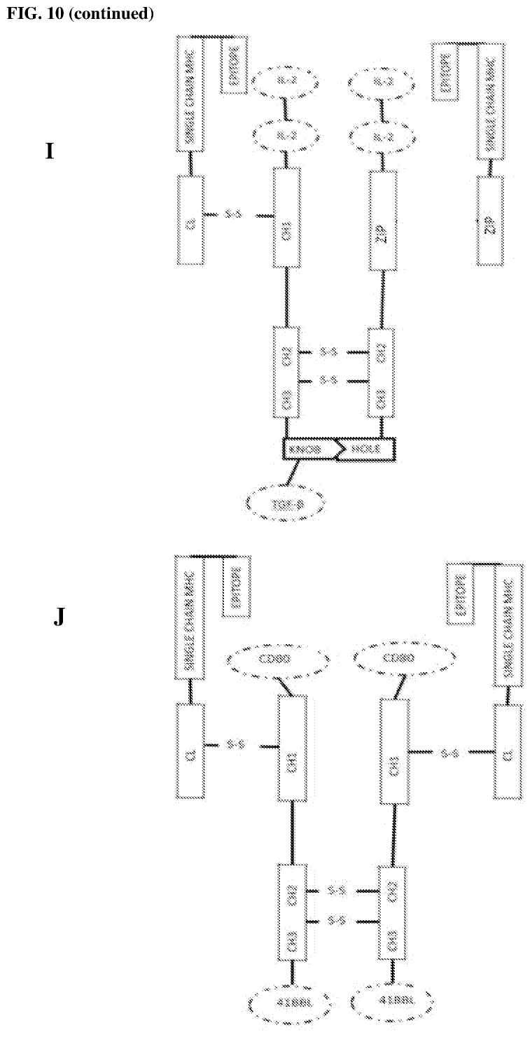

In an embodiment, the framework peptide multimerization site is an Fc heavy chain region (optionally a knob-in hole Fc sequence pair) and the dimerization sequences are the same (e.g., Ig CH1 sequences paired with light chain λ or κ constant region sequences) (see, for example, FIGS. 8 and 9, structures A to D). In another embodiment, the framework peptide multimerization site is an Fc heavy chain region (optionally a knob-in hole Fc sequence pair) and the dimerization sequences are selected to be different (e.g., a dimerization sequence pair comprising an Ig CH1 paired with light chain λ or κ sequence and a dimerization sequence comprising a leucine zipper pair, see for example, FIG. 10, structures E to H). For example, in a duplex MAPP the multimerization sequences may be a knob-in-hole Ig sequence, one dimerization sequence and its counterpart dimerization sequence may be leucine zipper sequences, and second dimerization sequence and its counterpart dimerization sequence may be an Ig CH1 and Ig CL λ domain pair.

MAPPs and accordingly their higher order complexes (duplexes, triplexes etc.) comprise MHC Class I polypeptide sequences that bind an epitope for presentation to a TCR, and accordingly may present peptides to T cells (e.g., CD8+ T cells). The effect of MAPPs on T cells with TCRs specific to the epitope depends on which, if any, MODs are present in the MAPP. As noted above, MAPPs, duplex MAPPS and higher order MAPPs comprising MOD(s) permit MOD delivery to T cells in an epitope selective manner and the MODs principally dictate the effect of MAPP-T cell engagement in light of the specific cell type stimulated and the environment. The effect of MAPP (e.g., duplex MAPP) presentation of MOD(s) and epitope to a T cells in some cases is enhanced relative to the situation encountered in antigen presenting cells (APC) where epitope can diffuse away from the MHC-H/β2M complex and any MODs the APC is presenting. This is the situation where the epitope and MOD are part of a MAPP polypeptide and cannot diffuse away even if the epitope's affinity for the MHC-H/β2M complex would normally permit it to leave the comparable cell complex. The inability of epitope to diffuse away from MHC-H/β2M and MOD components of a MAPP, duplex MAPP, or higher order MAPP is further limited where the polypeptide(s) of the MAPP (e.g, framework, dimerization sequence, and if present, the presenting complex 2^{nd} sequence) are covalently attached to each other (e.g., by disulfide bonds). Consequently, MAPPs and their higher order structures can prolong delivery of MOD(s) to T cells in an epitope selective manner relative to systems where epitope can diffuse away from the presenting MHC. In the absence of any MOD or any stimulatory MOD, prolonged exposure to the MAPP may result in T cell anergy or suppression of T cell stimulation.

Incorporation of one or more MODs with affinity for their cognate receptor on T cells (“co-MOD”) can reduce the specificity of MAPPs (e.g., duplex MAPPs) for epitope selective/specific T cells. The reduction in epitope selectivity/specificity of the MAPPs becomes more pronounced where MOD/co-MOD binding interactions increase in strength (binding energy) and significantly compete with MHC/epitope binding to target cell TCR. The inclusion of variant MODs with reduced affinity for their co-MOD(s) provides a lower contribution of MOD binding energy, thereby permitting MHC-epitope interactions with the TCR to dominate the binding and provide epitope selective interactions with T cells while retaining the activity of the MODs. Variant MODs with one or more substitutions (or deletions or insertions) that reduced the affinity of the MOD for their co-MOD may be incorporated into MAPPs and their higher order complexes alone or in combination with wild-type MODs polypeptide sequences. Wild-type and variant MODs are described further below.

The ability of MAPPs of the present disclosure to modulate T cells in an epitope selective/specific manner provides methods of modulating activity of a T cell in vitro, ex vivo, and in vivo, and accordingly, methods of treating disease such as cancers and benign neoplasms.

The present disclosure provides nucleic acids comprising nucleotide sequences encoding MAPP polypeptides, cells genetically modified with the nucleic acids and capable of producing the MAPP, and methods of producing MAPPs and their higher order complexes utilizing such cells.

Each presenting sequence or presenting complex present in a MAPP comprises MHC class I heavy chain and β2M polypeptide sequences (e.g., human MHC class I sequences) sufficient to bind an epitope peptide and present it to a TCR. MHC Class I peptides, may include sequence variations that are designed to stabilize the MHC, stabilize the MHC epitope peptide complex, and/or stabilize the MAPP. Sequence variations may also serve to enhance cellular expression of MAPPs prepared in cell-based systems as well as the stability (e.g., thermal stability) of MAPPs and their higher order complexes such as duplex MAPPs. Some MHC class I sequences suitable for use in MAPPs are described below.

As indicated in the description of the drawings, MAPPs may comprise one or more independently selected peptide sequences or (one or more “linker” or “linkers”) between any two or more components of the MAPP, which in the figures may be shown as a line between peptide and/or polypeptide elements of the MAPPs. The same sequences used as linkers may also be located at the N- and/or C-termini of the MAPP peptides to prevent, for example, proteolytic degradation. Linker sequences include but are not limited to polypeptides comprising: glycine; glycine and serine; glycine and alanine; alanine and serine; and glycine, alanine and serine; any one which may comprise a cysteine for formation of an intra or interpeptide disulfide bond. Various linkers are described in more detail below.

2. MAPP Architecture

MAPPs of the present disclosure comprise framework polypeptides with a multimerization sequence and at least one dimerization sequence, and dimerization polypeptides with a counterpart dimerization sequence that binds with the dimerization sequence. MAPP further comprise one or more epitope presenting sequences and/or one or more epitope presenting complexes.

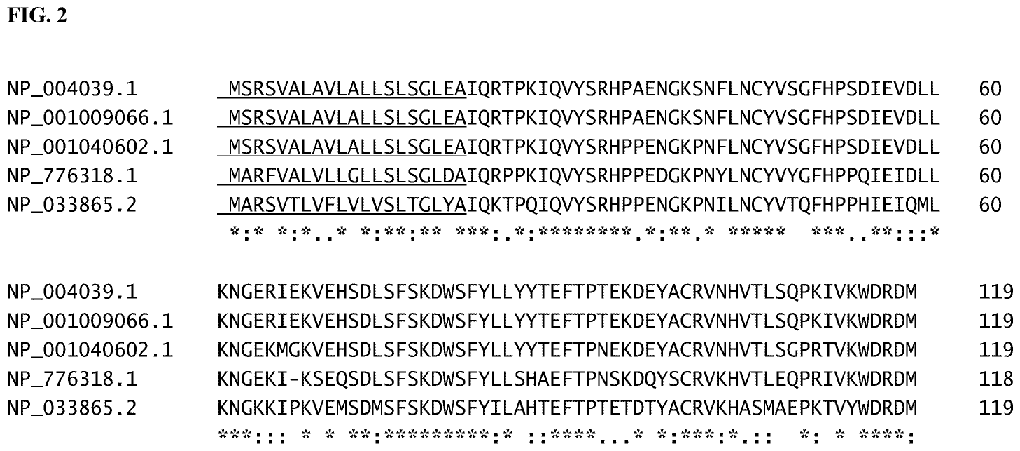

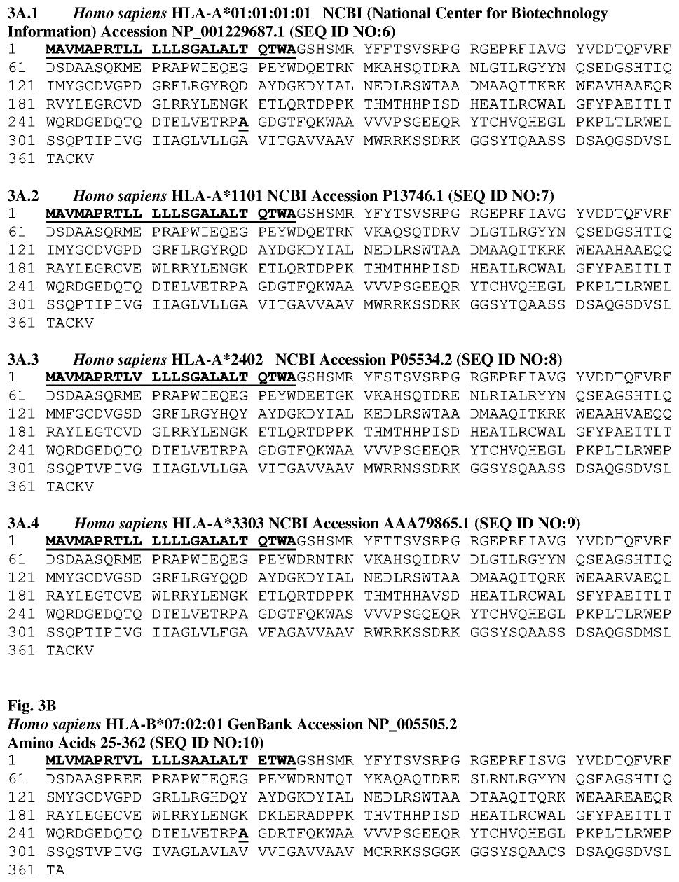

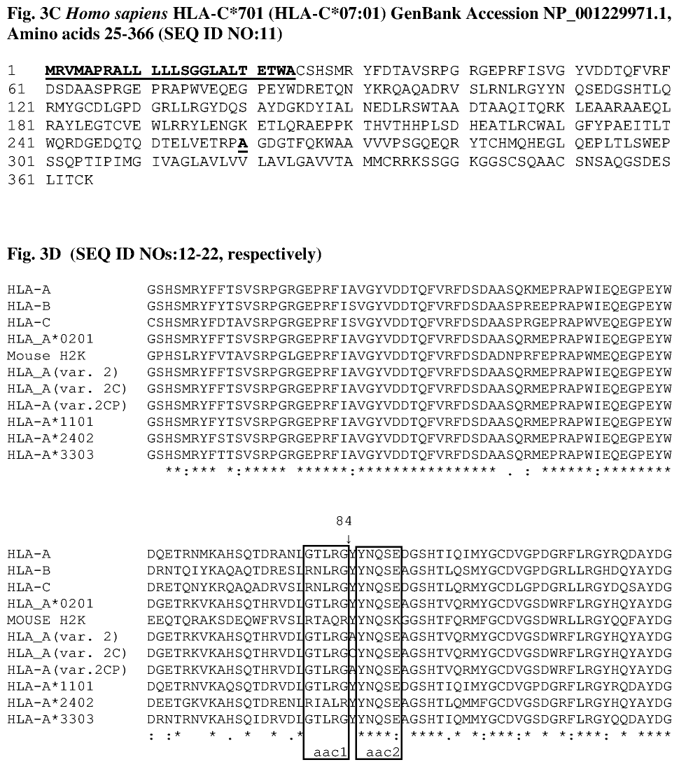

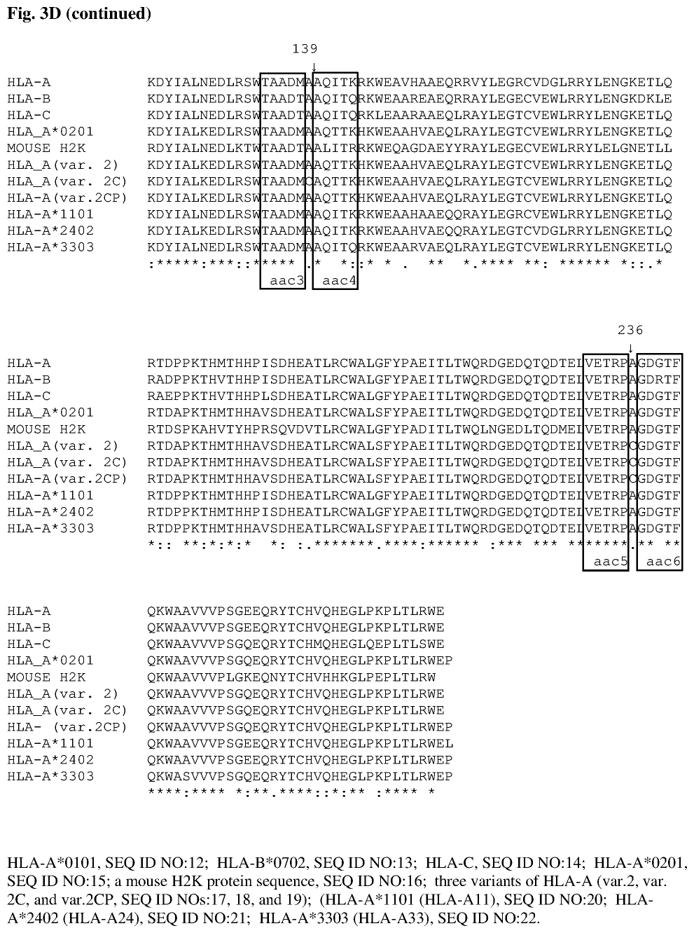

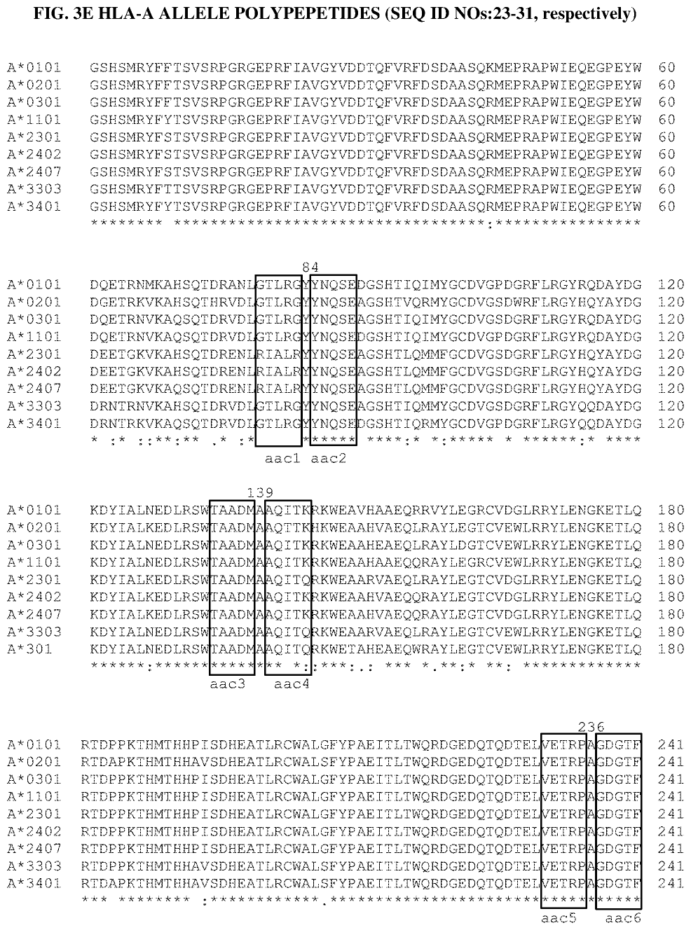

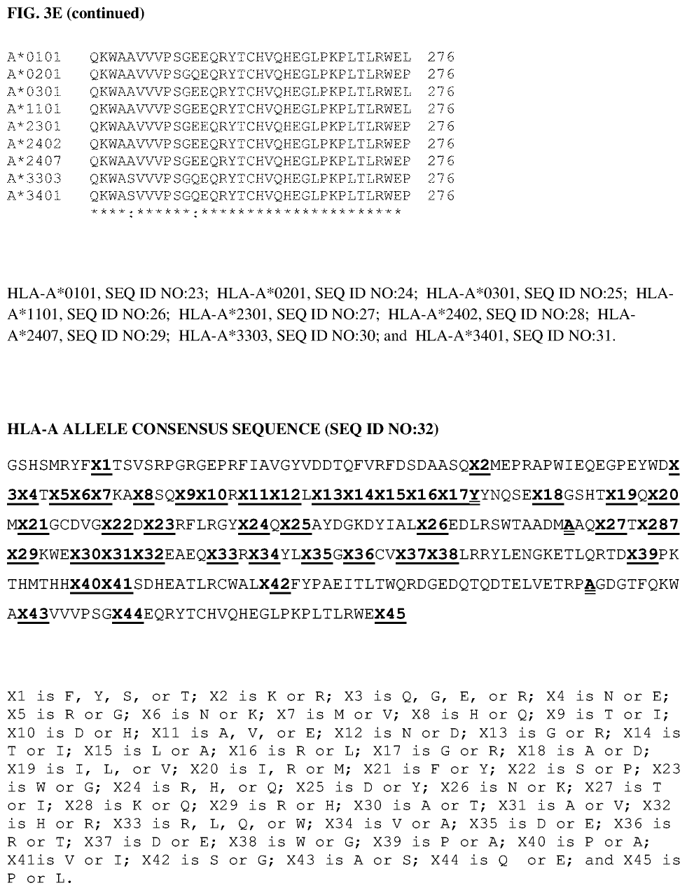

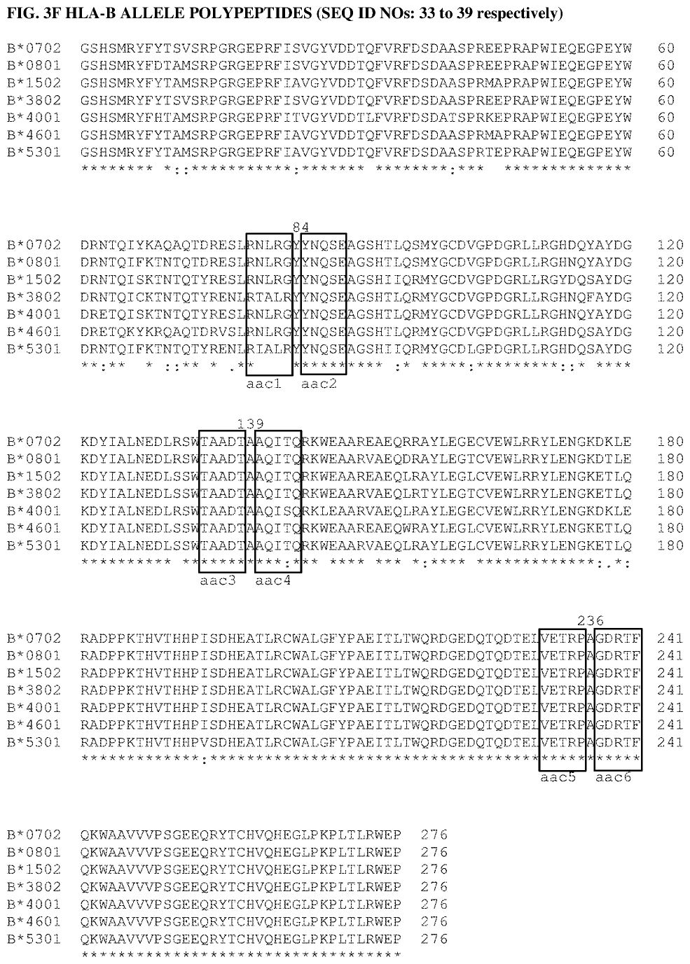

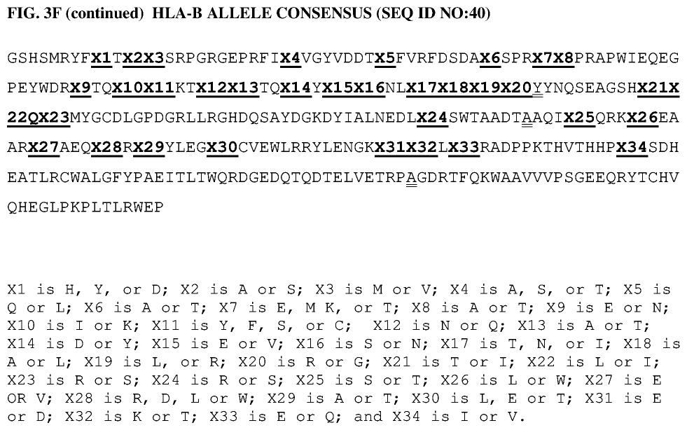

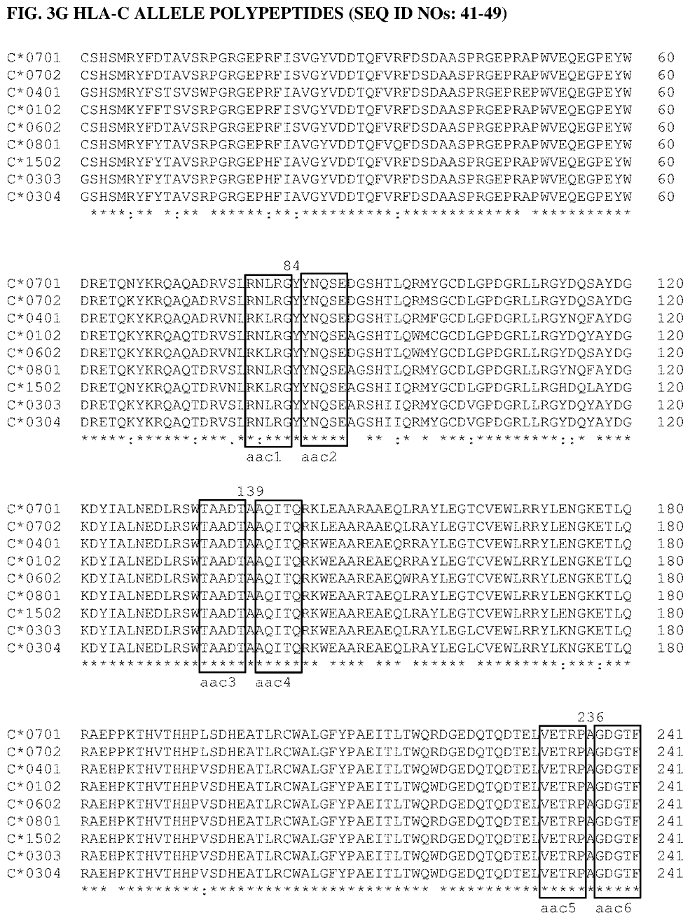

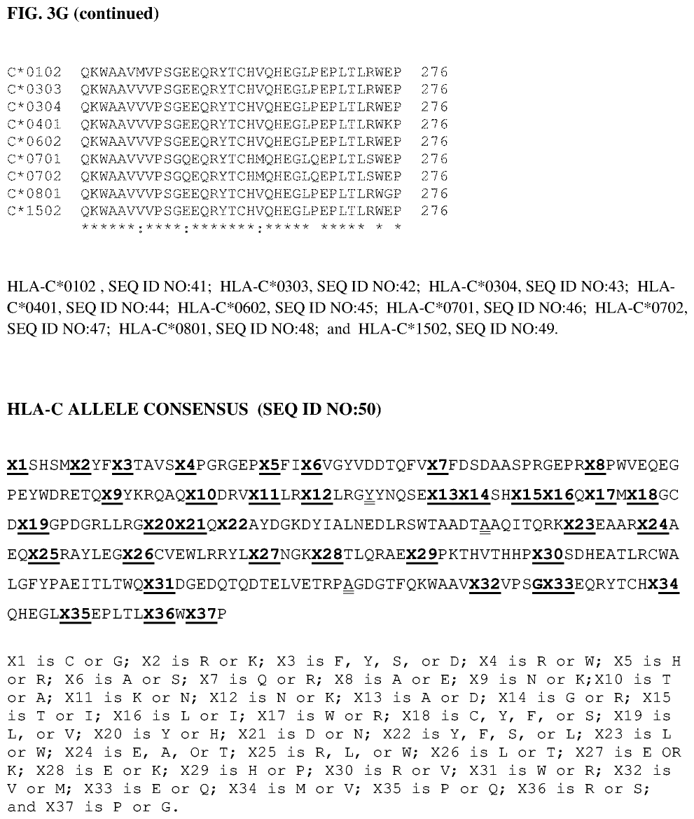

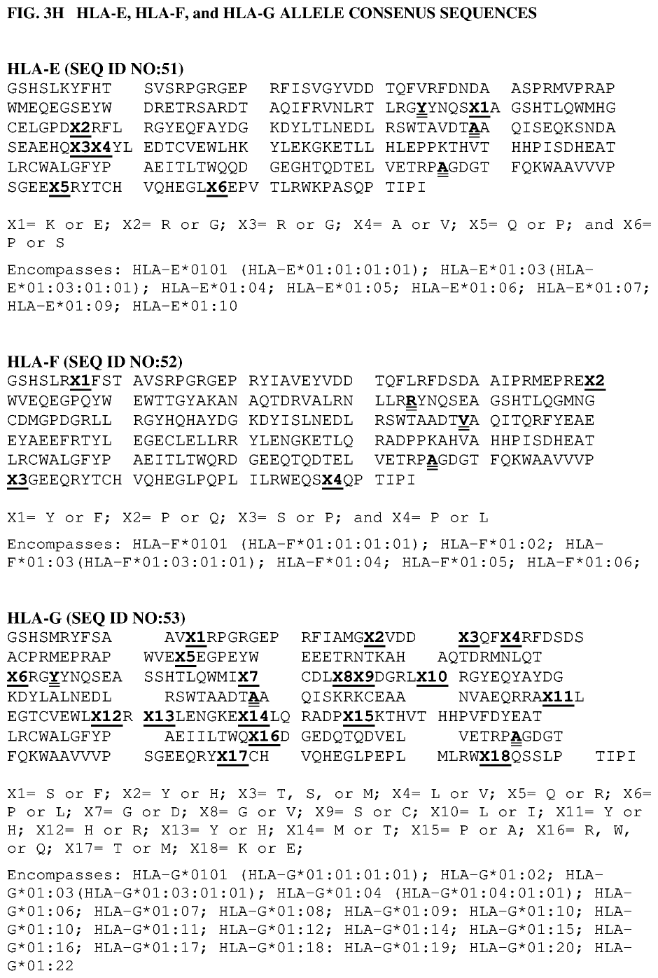

Interactions of MHC-H and β2M are not considered to result in multimerization and/or dimerization. In and embodiment, neither the dimerization sequence nor the multimerization sequence of the framework polypeptide, nor the counterpart dimerization sequence of the dimerization polypeptide comprises a class I MHC-H or β2M polypeptide sequence having at least 90% (e.g. 95% or 98%) sequence identity to at least 15 (e.g., at least 20, 30, 40, 50, 60 or 70) contiguous aas of a MHC class I polypeptide (e.g., a polypeptide in any of FIG. 2 or 3A to 3I). In an embodiment, MAPPs comprise at least one, or at least two, dimerization peptides that comprise an epitope presenting sequence.

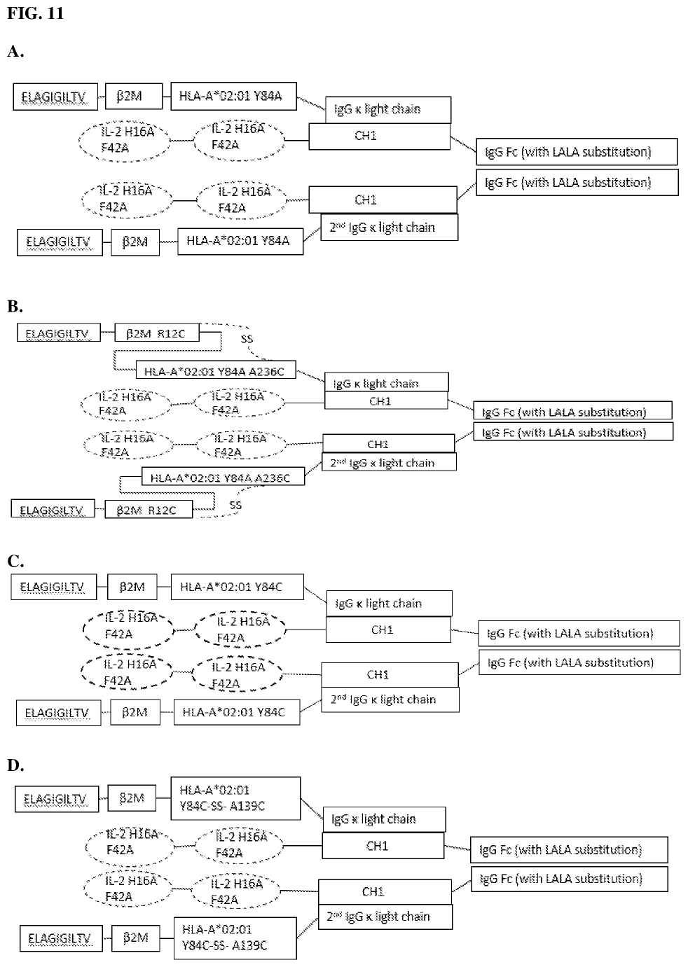

Exemplary structures for such MAPPs and their duplexes appear in FIG. 1A and FIGS. 6 to 11. The structure depicted in FIG. 11 represent duplex MAPPs with epitope presenting sequences. In FIGS. 1A and in FIGS. 6 to 9 the structures may comprise either epitope presenting sequences or epitope presenting complexes.

One group of MAPPs, those having epitope presenting sequences, comprise a framework polypeptide having, from N-terminus to C-terminus, a dimerization sequence and multimerization sequence; and a dimerization polypeptide comprising a counterpart dimerization sequence complementary to the dimerization sequence of the framework polypeptide and dimerizing therewith through covalent and/or non-covalent interactions to form a heterodimer; wherein at least one (e.g., one, or both) of a dimerization polypeptide and the framework polypeptide comprise a presenting sequence located on the N-terminal side of their dimerization or counterpart dimerization sequences. In such a MAPP the presenting sequence may comprise an epitope peptide and one or more MHC polypeptide sequences, with the epitope peptide sequence located: (i) at or within 10 aa, 15 aa, 20 aa, or 25 aa of the N-terminus of the presenting sequence, or (ii) in a polypeptide located at the N-terminus of the presenting sequence comprising, from N-terminus to C-terminus, a MOD, one or more optional linkers, and the epitope peptide; optionally at least one (e.g., one, two or each) of the framework polypeptide, dimerization peptide, and presenting sequence comprises one or more independently selected MODs located at their N-terminus or C-terminus (or on the N-terminal or C-terminal side of the dimerization or counterpart dimerization sequences). The MHC polypeptide sequences are MHC class I polypeptide sequences comprising a MHC class I heavy chain (MHC-H) sequence and a β2M sequence. In an embodiment, neither the dimerization sequence nor the multimerization sequence of the framework polypeptide comprises a MHC-H peptide or β2M sequence having at least 90% (e.g. 95% or 98%) sequence identity to at least 15 (e.g., at least 20, 30, 40, 50, 60 or 70) contiguous aas of a MHC class I polypeptide in any of FIGS. 2 to 3I.

Another group of MAPPs, those having epitope presenting complexes, comprise a framework polypeptide having, from N-terminus to C-terminus, a dimerization sequence and multimerization sequence; and a dimerization polypeptide comprising a counterpart dimerization sequence complementary to the dimerization sequence of the framework polypeptide and dimerizing therewith through covalent and/or non-covalent interactions to form a heterodimer; wherein at least one (e.g., one, or both) of a dimerization polypeptides and/or at least one (e.g., one or both) of the framework polypeptide comprise a presenting complex 1^{st} sequence located on the N-terminal side of their dimerization sequence. A presenting complex 2^{nd} sequence is associated with the presenting complex 1^{st} sequence (e.g., non-covalently or covalently such as by one or two interchain disulfide bonds) to form a presenting complex. In such a MAPP, each of the presenting complex 1^{st} sequence and presenting complex 2^{nd} sequence comprise one of the MHC-H and β2M polypeptide sequences, with one of the sequences further comprising the epitope peptide. The epitope peptide may be located (i) at or within 10 or 15 aa of the N-terminus of the presenting complex 1^{st} sequence or presenting complex 2^{nd} sequence, or (ii) in a polypeptide located at the N-terminus of the presenting complex 1^{st} sequence or presenting complex 2^{nd} sequence, with the polypeptide comprising, from N-terminus to C-terminus, a MOD, one or more optional linkers, and the epitope peptide. Optionally, at least one (e.g., one, two or each) of the framework polypeptide, dimerization peptide, or the peptides of a presenting complex comprise one or more independently selected MODs located at their N-terminus or C-terminus (or on the N-terminal or C-terminal side of the dimerization sequences).

In an embodiment, neither the dimerization sequence nor the multimerization sequence of the framework polypeptide comprises a class II MHC peptide sequence having at least 90% (e.g. 95% or 98%) sequence identity to at least 15 (e.g., at least 20, 30, 40, 50, 60 or 70) contiguous aas of a MHC class I polypeptide in any of FIGS. 2 to 3I.

As discussed above, a dimerization sequence of a framework polypeptide may interact with dimerization peptides to form heterodimers. The multimerization sequence of the framework polypeptide may associate with another framework polypeptide multimerization sequence forming a duplex (or higher order structure, such as a triplex, quadraplex or pentaplex) of the heterodimers. Where the multimerization sequences are interspecific (e.g., a knob-in-hole Fc peptide pair), and at least one heterodimer comprises an interspecific dimerization and counterpart dimerization pair, two different heterodimers may be formed. When the different heterodimers are combined to form a duplex MAPP, any one or more component (e.g., MODs) may differ (e.g., in type or location) between the two heterodimers.

### C. MAPP Components

1. Framework Polypeptides and Dimerization Polypeptides

As may be understood from the preceding section, framework polypeptides serve as the structural basis or skeleton of MAPPs, permitting the organization of other elements in the MAPP complex. Framework peptides interact with other peptides through binding interactions, principally at dimerization and multimerization sequences. Interactions at dimerization sequences permit association of non-framework peptides (e.g., dimerization peptides) with framework peptides. In contrast, multimerization sequences are involved in the interaction of two or more framework peptides.

The framework polypeptide of MAPPs comprise at least one multimerization sequence, and at least one independently selected dimerization sequence that is not identical to or of the same type (e.g., not both leucine zipper variants) as the multimerization sequence. By utilizing different types of sequences for the interactions at multimerization and dimerization sequences, it becomes possible to control the interactions of the framework polypeptide with other framework polypeptides and with dimerization polypeptides. In an embodiment, framework polypeptides comprise one multimerization sequence and one dimerization sequence. In an embodiment, framework polypeptides comprise at least one multimerization sequence and at least two independently selected dimerization sequences. Framework peptides may contain peptide sequences (e.g., linker sequences and/or MOD sequences) between any of the elements of the framework polypeptide or at the ends of the framework polypeptide including the multimerization sequences and dimerization sequences.

In addition to providing for the structural organization of MAPPS through their multimerization and dimerization sequences, framework peptides, and particularly their N- and C-termini, may also serve as locations for placement of elements such as MOD sequences, an epitope, presenting sequences, and/or a presenting complex 1^{st} sequence (one polypeptide of an epitope presenting complexes). When placed at the N- and/or C-termini of a framework polypeptide, such polypeptide elements are part of the framework polypeptide (e.g., a single translation product formed in a cell).

Within a MAPP, all of the dimerization sequence may be non-interspecific (such as leucine zipper pairs) while the multimerization sequences is either interspecific or non-interspecific (see e.g., structures A & B of FIGS. 6 and 7). For example, in a duplex MAPP with first and second framework polypeptides, the multimerization sequences may be a non-interspecific (e.g., IgFc (CH2, CH3) or leucine zipper) or the multimerization sequences may be an interspecific knob-in-hole sequence pair; with the dimerization sequences of the first and second framework polypeptide as a non-interspecific leucine zipper polypeptide. Where an Fc polypeptide is employed it may be, for example, from an IgA, IgD, IgE, IgG, or IgM, which may be a human polypeptide sequence, a humanized polypeptide sequence, a Fc region polypeptide of a synthetic heavy chain constant region, or a consensus heavy chain constant region.

Within a MAPP, all of the dimerization sequence may interspecific, while the multimerization sequences is not interspecific (see e.g., FIG. 10 A). For example, a duplex MAPP with first and second framework polypeptides, the multimerization sequences may be an IgFc (e.g., CH2, CH3) sequence, with the a ZW1 sequence or its counterpart employed as the dimerization sequence of the first framework polypeptide and an Ig CH1 domain or its counterpart Ig C_{L} κ sequence as the dimerization sequence of the second framework polypeptide.

All of the dimerization sequences or all of the dimerization and multimerization sequences, in a MAPP may differ in that they bind only specific binding partners present in the MAPP (e.g., each are part of a different interspecific sequence pair) (see e.g., FIG. 10 structures A to D). For example, in a duplex MAPP with first and second framework polypeptides, the multimerization sequences may be a pair of knob-in-hole IgFc sequences, with the a ZW1 sequence or its counterpart employed as the dimerization sequence of the first framework polypeptide, and a Ig CH1 or its counterpart Ig C_{L}sequence as the dimerization sequence of the second framework polypeptide.

2. Multimerization and Dimerization Polypeptide Sequences

Amino acid sequences that permit polypeptides to interact may be utilized as dimerization sequences or counterpart dimerization sequences when they are involved in the formation of dimers between a framework polypeptide and a dimerization polypeptide. The same type of aa sequences may be utilized as multimerization sequences when they are used to form duplex or form higher order structures (trimers, tetramers, pentamer, etc.) between framework polypeptides. In any given MAPP, sequences that can interact with each other are not utilized as dimerization and multimerization sequences. Stated another way, the same aa sequence pair may serve as either dimerization or multimerization sequences depending on whether they: bring together two or more framework peptides, in which case they are multimerization sequences; or they bring together a dimerization and multimerization sequence, in which case they are designated as dimerization sequences.

Where dimerization or multimerization sequences employ identical sequences that pair or multimerize (e.g., some leucine zipper sequences), they can form symmetrical pairs or multimers (e.g., homodimers) as shown in FIG. 6 structure A. In contrast, where dimerization or multimerization sequences that pair are not identical and require a specific complementary counterpart sequence to form a dimer, they are interspecific binding sequences and can form asymmetric pairs. Both immunoglobulin and non-immunobloblin (e.g., IgFc) polypeptides can be interspecific or non-interspecific in nature. For example, both Fos/Jun binding pairs and Ig CH1 polypeptide sequences and light chain constant region C_{L} sequences form interspecific binding paris. Natural Ig Fc regions tend to be non-intersepcifc, but, as discussed below, can be made to form interspecif pairs (e.g., KiH pairs). Coiled-coil sequences, including leucine zipper sequences, can be either interspecific leucine zipper or non-interspecific leucine zipper sequences. See e.g., Zeng et al., (1997) PNAS (USA) 94:3673-3678; and Li et al., (2012), Nature Comms. 3:662.

Interspecific binding sequences may in some instances form some amount of homodimers, but preferentially dimerize by binding more strongly with their counterpart interspecific binding sequence. Accordingly, specific heterodimers tend to be formed when an interspecific dimerization sequence and its counterpart interspecific binding sequence are incorporated into a pair of polypeptides. By way of example, where an interspecific dimerization sequence and its counterpart are incorporated into a pair of polypeptides, they may selectively form greater than 70%, 80%, 90%, 95%, 98% or 99% heterodimers when an equimolar mixture of the polypeptides are combined. The remainder of the polypeptides may be present as monomers or homodimers, which may be separated from the heterodimer. See, for example, FIG. 6, structure B, with an interspecifc multimerization sequence and structure C with two different interspecific dimerization sequences. Moreover, because interspecific sequences are selective for their counterpart sequence, they can limit the interaction with other proteins expressed by cells (e.g., in culture or in a subject) particularly where the interspecific sequences are not .naturally occurring or are variants of naturally occurring protein sequences.

Sequence are considered orthogonal to other sequences when they do not form complexes (bind) with each other's counterpart sequences. See FIG. 6 structure D where the MAPP comprises an interspecific multimerization sequence and two independently selected interspecific dimerization sequences, all of which are orthogonal to each other. Any of the MAPPS described herein may have two or more (e.g., three, four or more) orthogonal dimerization sequences. In an embodiment, MAPPs with multimerizing framework peptides may have orthogonal multimerization and dimerization domains (where the dimerization domains may or may not be orthogonal to each other).

Some sequences permitting polypeptides to interact with sufficient affinity to be used as dimerization and/or multimerization sequences are provided for example in U.S. Patent Publication No. 2003/0138440. The sequences may be of relatively compact size (e.g., such as less than about 300, 250, 225, 200, 175, 150, 125, 100, 75, 60, 50, 40, or 30 aa). In an embodiment, at least one (e.g., at least two or all) dimerization and/or multimerization sequence is less than 300 aa. In an embodiment, at least one (e.g., at least two or all) dimerization and/or multimerization sequence is less than 200 aa. In an embodiment, at least one (e.g., at least two or all) dimerization and/or multimerization sequence is less than 100 aa. In an embodiment, at least one (e.g., at least two or all) dimerization and/or multimerization sequence is less than 75 aa. In another embodiment, at least one (e.g., at least two or all) dimerization and/or multimerization sequence is less than are less than 50 aa. In an embodiment, at least one (e.g., at least two or all) dimerization and/or multimerization sequence is less than 30 aa.

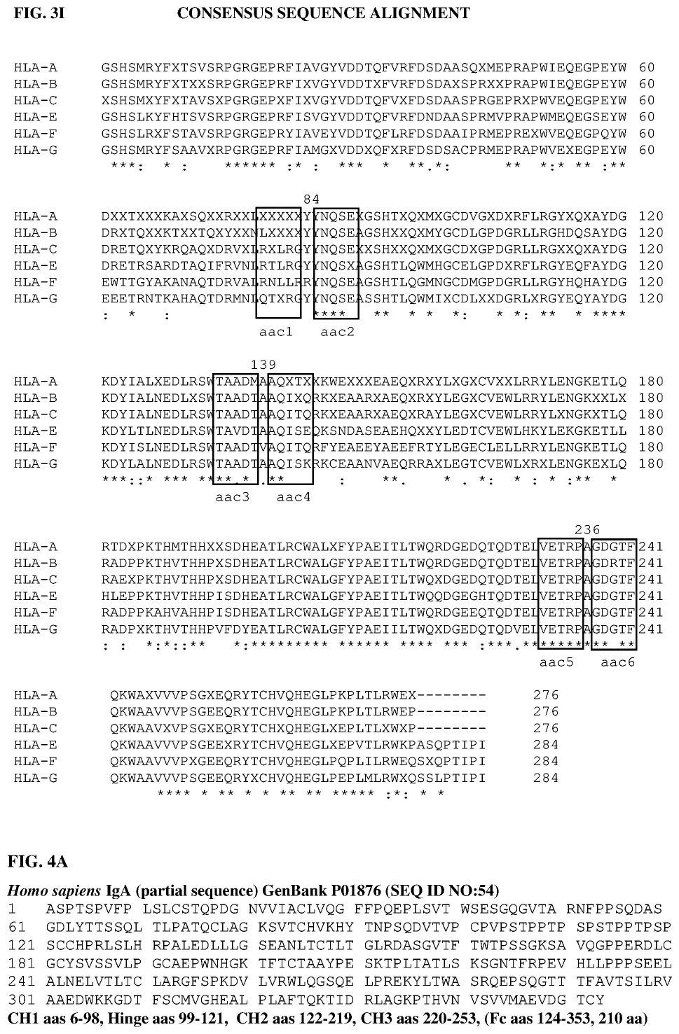

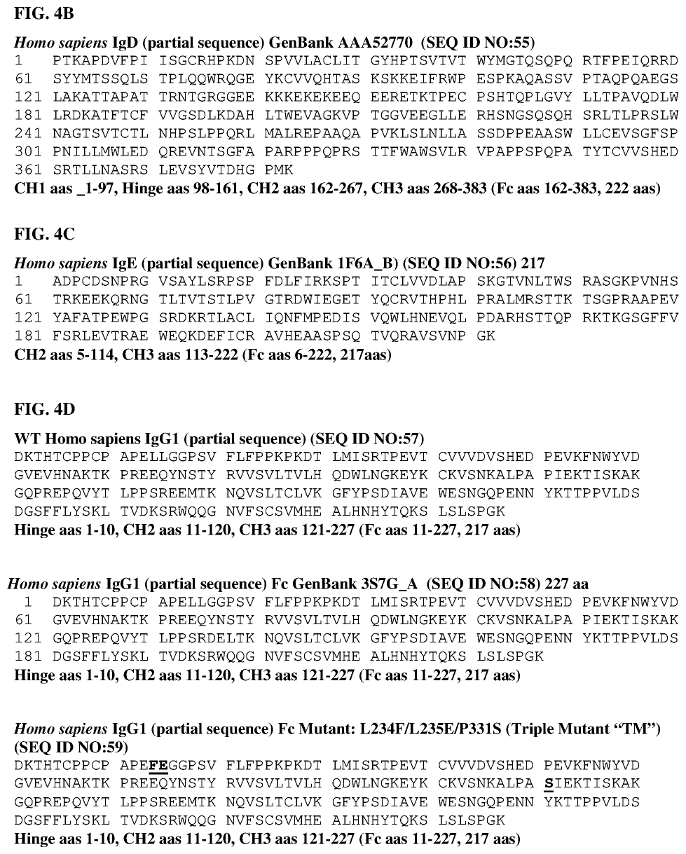

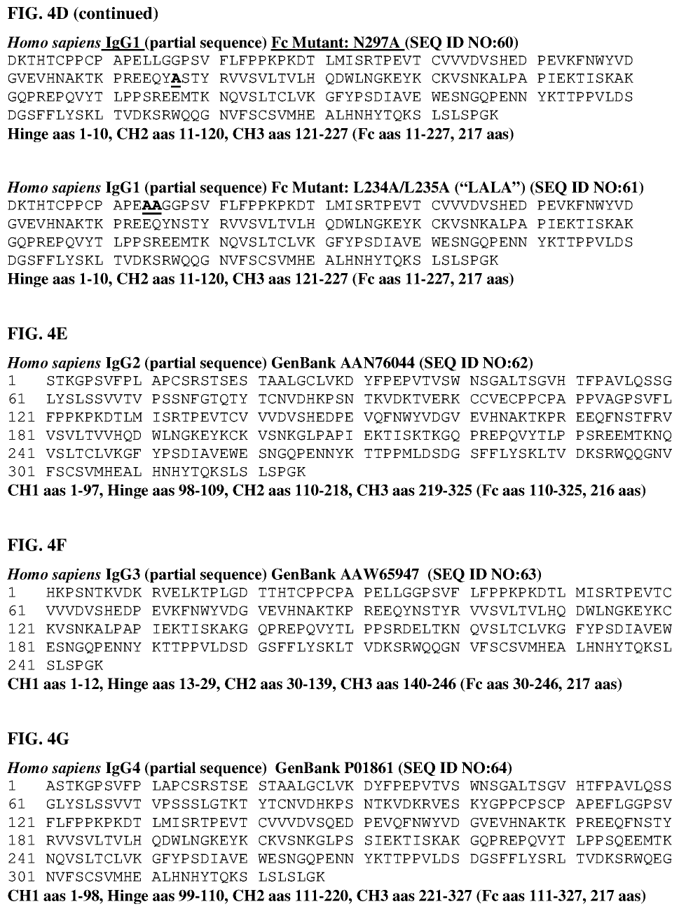

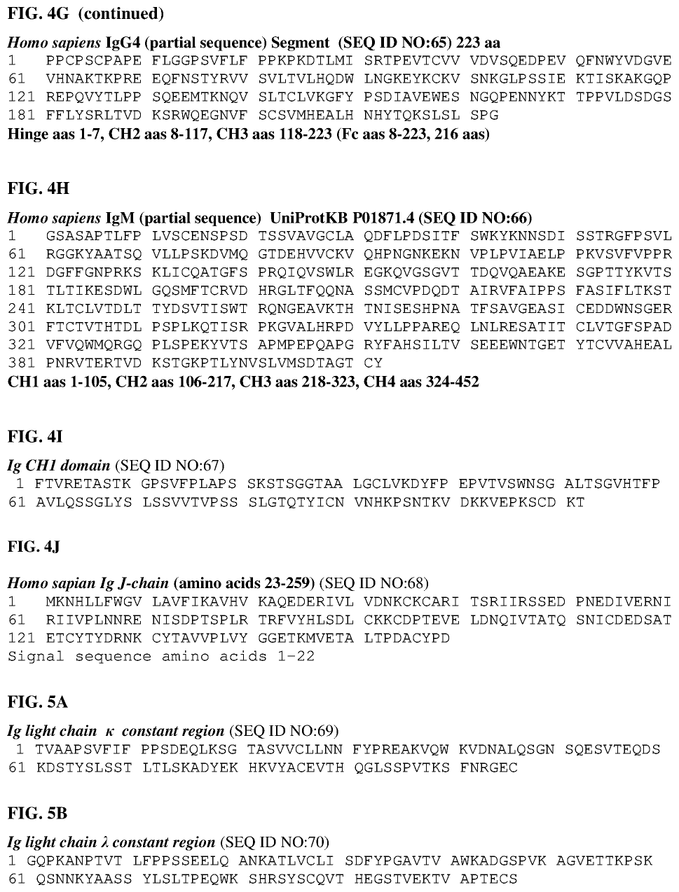

Dimerization/multimerization sequences include but are not limited to: immunoglobulin heavy chain constant region (Ig Fc) polypeptide sequences (e.g., sequences comprising CH2-CH3 regions of immunoglobulins such as those provided in FIGS. 4A-4H and SEQ ID NOs:54 to 66); polypeptides of the collectin family (e.g., ACRP30 or ACRP30-like proteins) that contain collagen domains consisting of collagen repeats Gly-Xaa-Yaa and/or Gly-Xaa-Pro (which may be repeated from 10-40 times); coiled-coil domains; leucine-zipper domains; interspecific Ig Fc heavy chain constant regions (such as knob-in-hole sequences described in more detail below); Fos/Jun binding pairs; immunoglobulin heavy chain constant region (CH2-CH3) sequences, and; Ig CH1 and light chain constant region CL sequences (Ig CH1/CL pairs such as a Ig CH1 sequence paired with a Ig C_{L} κ or λ light chain constant region sequence).

Framework and/or dimerization polypeptides of a MAPP may comprise an immunoglobulin heavy chain constant region (e.g., CH2-CH3 domains) polypeptide sequence that functions as a dimerization or multimerization sequence. Where the framework polypeptide comprises a IgFc multimerization sequence, and a CH1 dimerization sequence it may comprise all or part a native or variant immunoglobin sequence set forth in any of FIGS. 4A to 4H that comprise the CH1, CH2 and CH3 domains and any hinge sequences that may be present. In some cases, the Fc sequence has at least about 70%, at least about 80%, at least about 90%, at least about 95%, at least about 98%, at least about 99%, or 100% aa sequence identity to an aa sequence of an Fc region depicted in FIGS. 4A-4H. Such immunoglobulin sequences can covalently link the polypeptides of MAPP complex together by forming one or two interchain disulfide bonds, thereby stabilizing MAPPs, particularly where a pair of interspecific Ig sequence such as knob-in-hole polypeptide pairs are employed. Where an Fc polypeptide sequence, alone or in combination with a CH1 polypeptide sequence, is employed they may be, for example, from an IgA, IgD, IgE, IgG, or IgM, which may be a human polypeptide sequence, a humanized polypeptide sequence, a Fc region polypeptide of a synthetic heavy chain constant region, or a consensus heavy chain constant region.

Framework and/or dimerization polypeptides of a MAPP may comprise a sequence that has at least about 70%, at least about 80%, at least about 90%, at least about 95%, at least about 98%, at least about 99%, or 100% aa sequence identity to at least 150 contiguous aas (at least 175, at least 200, at least 225, at least 250, at least 275, at least 300, at least 325, or at least 350 contiguous aas), or all aas, of the IgA Fc sequence depicted in FIG. 4A (SEQ ID NO:54). Framework and/or dimerization polypeptides of a MAPP may comprise a sequence that has at least about 70%, at least about 80%, at least about 90%, at least about 95%, at least about 98%, at least about 99%, or 100% aa sequence identity to at least 150 contiguous aas (at least 175, at least 200, at least 225, at least 250, at least 275, at least 300, at least 325, or at least 350 contiguous aas), or all aas, of the IgD Fc sequence depicted in FIG. 4B (SEQ ID NO:55). Framework and/or dimerization polypeptides of a MAPP may comprise a sequence that has at least about 70%, at least about 80%, at least about 90%, at least about 95%, at least about 98%, at least about 99%, or 100% aa sequence identity to at least 125 contiguous aas (at least 150, at least 175, or at least 200 contiguous aas), or all aas, of the IgE Fc sequence depicted in FIG. 4C (SEQ ID NO:56).

A MAPP may comprise one or more IgG Fc sequences as dimerization and/or multimerization sequences. The Fc polypeptide of a MAPP of the present disclosure can be a human IgG1 Fc, a human IgG2 Fc, a human IgG3 Fc, a human IgG4 Fc, etc. In some cases, the Fc sequence has at least about 70%, at least about 80%, at least about 90%, at least about 95%, at least about 98%, at least about 99%, or 100% aa sequence identity to an aa sequence of an Fc region depicted in FIG. 4D-4G. Framework and/or dimerization polypeptides of a MAPP may comprise a sequence that has at least about 70%, at least about 80%, at least about 90%, at least about 95%, at least about 98%, at least about 99%, or 100% aa sequence identity to at least 125 contiguous aas (e.g., at least 150, at least 175, at least 200, or at least 220 contiguous aas), or all aas, of the wt. IgG1 Fc polypeptide sequence depicted in FIG. 4D (SEQ ID NO: 57). Framework and/or dimerization polypeptides of a MAPP may comprise a sequence that has at least about 70%, at least about 80%, at least about 90%, at least about 95%, at least about 98%, at least about 99%, or 100% aa sequence identity to at least 125 (e.g., at least 150, at least 175, at least 200, or at least 225) contiguous aas, or all aas, of the IgG2 Fc polypeptide sequence depicted in FIG. 4E (SEQ ID NO:62). Framework and/or dimerization polypeptides of a MAPP may comprise a sequence that has at least about 70%, at least about 80%, at least about 90%, at least about 95%, at least about 98%, at least about 99%, or 100% aa sequence identity to at least 125 (e.g., at least 150, at least 175, at least 200, at least 225, or at least 240) contiguous aas, or all aas, of the IgG3 Fc sequence depicted in FIG. 4F (SEQ ID NO:63). Framework and/or dimerization polypeptides of a MAPP may comprise a sequence that has at least about 70%, at least about 80%, at least about 90%, at least about 95%, at least about 98%, at least about 99%, or 100% aa sequence identity to at least 125 (e.g., at least 150, at least 175, at least 200, or at least 220) contiguous aas, or all aas, of the IgG4 Fc sequence depicted in FIG. 4G (SEQ ID NO:64 or 65).

Framework and/or dimerization polypeptides of a MAPP may comprise a sequence that has at least about 70%, at least about 80%, at least about 90%, at least about 95%, at least about 98%, at least about 99%, or 100% aa sequence identity to at least 125 (at least 150, at least 175, at least 200, at least 225, or at least 250) contiguous aas, or all aas, of the IgM Fc polypeptide sequence depicted in FIG. 4H (SEQ ID NO:66).

Framework and/or dimerization polypeptides of a MAPP comprising immunoglobulin sequences (e.g., depicted in FIGS. 4A-4H) can be covalently linked together by formation of one or two interchain disulfide bonds between cysteines that are adjacent to the immunoglobin hinge regions. Such disulfide bonds can stabilize the interaction of framework and dimerization polypeptide heterodimers, or, for example, duplexes of such heterodimers when the disulfide bonds are between framework multimerization sequences.

A framework or dimerization polypeptide may comprise an aa sequence having 100% aa sequence identity to the wt. human IgG1 Fc polypeptide depicted in FIG. 4D. A framework or dimerization polypeptide may comprise an aa sequence (e.g., as a multimerization sequence) having at least about 70% (e.g., at least about 80%, 90%, 95%, 98%, or 99%) aa sequence identity to the wt. human IgG1 Fc polypeptide depicted in FIG. 4D, that includes a substitution of N297 (N77 as numbered in FIG. 4D, SEQ ID NO:60) with an aa other than asparagine. In one case, N297 is substituted by alanine, (N297A). Substitutions at N297 lead to the removal of carbohydrate modifications and result antibody sequences with reduced complement component 1q (“C1q”) binding compared to the wt. protein, and accordingly a reduction in complement dependent cytotoxicity.

L234 and other aas in the lower hinge region (e.g., aas 234 to 239, which correspond to aas 14-19 of SEQ ID NO:57) of IgG are involved in binding to the Fc lambda receptor (FcγR), and accordingly, mutations at that location reduce binding to the receptor (relative to the wt. protein). A framework or dimerization polypeptide with a substitution in the lower hinge region may comprise an aa sequence (e.g., as a multimerization sequence) having at least about 70% (e.g., at least about 80%, 90%, 95%, 98%, or 99%) aa sequence identity to the wt. human IgG1 Fc polypeptide depicted in FIG. 4D, that includes a substitution of L234 (L14 of the aa sequence depicted in FIG. 4D) with an aa other than leucine.

A framework or dimerization polypeptide with a substitution in the lower hinge region may comprise an aa sequence (e.g., as a multimerization sequence) having at least about 70% (e.g., at least about 80%, 90%, 95%, 98%, or 99%) aa sequence identity to the wt. human IgG1 Fc polypeptide depicted in FIG. 4D, that includes a substitution of L235 (L15 of the aa sequence depicted in FIG. 4D) with an aa other than leucine. In some cases, the framework and/or dimerization polypeptide present in a MAPP with substitutions in the lower hinge region includes L234A and L235A (“LALA”) substitutions (the positions corresponding to positions 14 and 15 of the wt. aa sequence depicted in FIG. 4D; see, e.g., SEQ ID NO:61).

A framework or dimerization polypeptide with a substitution in the lower hinge region may comprise an aa sequence (e.g., as a multimerization sequence) having at least about 70% (e.g., at least about 80%, 90%, 95%, 98%, or 99%) aa sequence identity to the wt. human IgG1 Fc polypeptide depicted in FIG. 4D, that includes a substitution of P331 (P111 of the aa sequence depicted in FIG. 4D) with an aa other than proline. Substitutions at P331, like those at N297, lead to reduced binding to C1q relative to the wt. protein, and thus a reduction in complement dependent cytotoxicity. In one embodiment, the substitution is a P331S substitution. In another embodiment, the substitution is a P331A substitution.

A framework or dimerization polypeptide may comprise an aa sequence (e.g., as a multimerization sequence) having at least about 70% (e.g., at least about 80%, 90%, 95%, 98%, or 99%) aa sequence identity to the wt. human IgG1 Fc polypeptide depicted in FIG. 4D, and include substitutions of D270, K322, and/or P329 (corresponding to D50, K102, and P109 of SEQ ID NO:57 in FIG. 4D) that reduce binding to C1q protein relative to the wt protein.

A framework or dimerization polypeptide may comprise an aa sequence (e.g., as a multimerization sequence) having at least about 70% (e.g., at least about 80%, 90%, 95%, 98%, or 99%) aa sequence identity to the wt. human IgG1 Fc polypeptide depicted in FIG. 4D, including substitutions at L234 and/or L235 (L14 and/or L15 of the aa sequence depicted in FIG. 4D) with aas other than leucine such as L234A and L235A, and a substitution of P331 (P111 of the aa sequence depicted in FIG. 4D) with an aa other than proline such as P331S. In one instance, a framework or dimerization polypeptide present in a MAPP comprises the “Triple Mutant” aa sequence (SEQ ID NO:59) depicted in FIG. 4D (human IgG1 Fc) having L234F, L235E, and P331S substitutions (corresponding to aa positions 14, 15, and 111 of the aa sequence depicted in FIG. 4D).

Where an asymmetric pairing between two polypeptides of a MAPP is desired, a framework or dimerization polypeptide present in a MAPP may comprise, consist essentially of, or consist of an interspecific binding sequence. Interspecific binding sequences favor formation of heterodimers with their cognate polypeptide sequence (i.e., the interspecific sequence and its counterpart interspecific sequence), particularly those based on immunoglobulin Fc (Ig Fc) sequence variants. Such interspecific polypeptide sequences include knob-in-hole without (KiH) or with (KiHs-s) a stabilizing disulfide bond, HA-TF, ZW-1, 7.8.60, DD-KK, EW-RVT, EW-RVTs-s, and A107 sequences. One interspecific binding pair comprises a T366Y and Y407T mutant pair in the CH3 domain interface of IgG1, or the corresponding residues of other immunoglobulins. See Ridgway et al., Protein Engineering 9:7, 617-621 (1996). A second interspecific binding pair involves the formation of a knob by a T366W substitution, and a hole by the triple substitutions T366S, L368A and Y407V on the complementary Ig Fc sequence. See Xu et al. mAbs 7:1, 231-242 (2015). Another interspecific binding pair has a first Ig Fc polypeptide with Y349C, T366S, L368A, and Y407V substitutions and a second Ig Fc polypeptide with S354C, and T366W substitutions (disulfide bonds can form between the Y349C and the S354C). See e.g., Brinkmann and Konthermann, mAbs 9:2, 182-212 (2015). Ig Fc polypeptide sequences, either with or without knob-in-hole modifications, can be stabilized by the formation of disulfide bonds between the Ig Fc polypeptides (e.g., the hinge region disulfide bonds). Several interspecific binding sequences based upon immunoglobulin sequences are summarized in the table that follows, with cross reference to the numbering of the aa positions as they appear in the wt. IgG1 sequence (SEQ ID NO:57) set forth in FIG. 4D shown in brackets “{ }”.

TABLE 1
Interspecific immunoglobulin sequences and their cognate counterpart interspecific
sequences

| | Substitutions in the first | Substitutions in the second | |
|---|---|---|---|
| Interspecific | interspecific polypeptide | (counterpart) interspecific | |
| Pair Name | sequence | polypeptide sequence | Comments |
| KiH | T366W | T366S/L368A/Y407V | Hydrophobic/steric |
| | {T146W} | {T146S/L148A/Y187V} | complementarity |
| KiHs-s | T366W/S354C* | T366S/L368A/Y407V/Y349C | KiH + inter-CH3 |
| | {T146W/S134C*} | {T146S/L148A/Y187V/Y129C} | domain S—S bond |
| HA-TF | S364H/F405A | Y349T/T394F | Hydrophobic/steric |
| | {S144H/F185A} | {Y129T/T174F} | complementarity |
| ZW1 | T350V/L351Y/F405A/Y407V | T350V/T366L/K392L/T394W | Hydrophobic/steric |
| | {T130V/L131Y/F185A/ | {T130V/T146L/K172L/T174W} | complementarity |
| | Y187V} | | |
| 7.8.60 | K360D/D399M/Y407A | E345R/Q347R/T366V/K409V | Hydrophobic/steric |
| | {K140D/D179M/Y187A} | {E125R/Q127R/T146V/K189V} | complementarity + |
| | | | electrostatic |
| | | | complementarity |
| DD-KK | K409D/K392D | D399K/E356K | Electrostatic |
| | {K189D/K172D} | {D179K/E136K} | complementarity |
| EW-RVT | K360E/K409W | Q347R/D399V/F405T | Hydrophobic/steric |
| | {K140E/K189W} | {Q127R/D179V/F185T} | complementarity & |
| | | | long-range electro- |
| | | | static interaction |
| EW-RVTs-s | K360E/K409W/Y349C* | Q347R/D399V/F405T/S354C | EW-RVT + inter- |
| | {K140E/K189W/Y129C*} | {Q127R/D179V/F185T/S134C} | CH3 domain S-S |
| | | | bond |
| A107 | K370E/K409W | E357N/D399V/F405T | Hydrophobic/steric |
| | {K150E/K189W} | {E137N/D179V/F185T} | complementarity + |
| | | | hydrogen bonding |
| | | | complementarity |

Table 1 modified from Ha et al., Frontiers in Immunol. 7: 1-16 (2016).
*aa forms a stabilizing disulfide bond.

In addition to the interspecific pairs of sequences in Table 1, framework and/or dimerization polypeptides may include interspecific “SEED” sequences having 45 residues derived from IgA in an IgG1 CH3 domain of the interspecific sequence, and 57 residues derived from IgG1 in the IgA CH3 in its counterpart interspecific sequence. See Ha et al., Frontiers in Immunol. 7:1-16 (2016).

In an embodiment, a framework or dimerization polypeptide found in a MAPP may comprise an interspecific binding sequence or its counterpart interspecific binding sequence selected from the group consisting of: knob-in-hole (KiH); knob-in-hole with a stabilizing disulfide (KiHs-s); HA-TF; ZW-1; 7.8.60; DD-KK; EW-RVT; EW-RVTs-s; A107; or SEED sequences.

In an embodiment, a MAPP comprises a framework or dimerization polypeptide comprising an IgG1 sequence with a T146W KiH sequence substitution, and its counterpart interspecific binding partner polypeptide comprises an IgG1 sequence having T146W, L148A, and Y187V KiH sequence substitutions, where the framework and/or dimerization polypeptides comprises a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 4D. One or both of the framework, or both of dimerization polypeptides optionally comprising substitutions at one of more of: L234 and L235 (e.g., L234A/L235A “LALA” or L234F/L235E); N297 (e.g., N297A); P331 (e.g. P331S); L351 (e.g., L351K); T366 (e.g., T366S); P395 (e.g., P395V); F405 (e.g., F405R); Y407 (e.g., Y407A); and K409 (e.g., K409Y). Those substitutions appear at: L14 and L15 (e.g., L14A/L15A “LALA” or L14F/L15E); N77 (e.g., N77A); P111 (e.g. P111S) L131 (e.g., L131K); T146 (e.g., T146S); P175 (e.g., P175V); F185 (e.g., F185R); Y187 (e.g., Y187A); and K189 (e.g., K189Y) in the wt. IgG1 sequence of FIG. 4D.

In an embodiment, a MAPP comprises a framework or dimerization polypeptide comprising an IgG1 sequence with a T146W KiH sequence substitution, and its counterpart interspecific binding partner polypeptide comprises an IgG1 sequence having T146S, L148A, and Y187V KiH sequence substitutions, where the framework and/or dimerization polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 4D; where one or both of the framework and/or dimerization polypeptide sequences may comprise additional substitutions such as L14 and/or L15 substitutions (e.g., “LALA” substitutions L234A and L235A), and/or N77 (N297 e.g., N297A or N297G). See e.g., the N297A and LALA sequences in FIG. 4D.

In an embodiment, a MAPP comprises a framework or dimerization polypeptide comprising an IgG1 sequence with a T146W and S134C KiHs-s substitutions, and its counterpart interspecific binding partner polypeptide comprises an IgG1 sequence having T146S, L148A, Y187V and Y129C KiHs-s substitutions, where the framework and/or dimerization polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 4D; where one or both of the framework and/or dimerization polypeptide sequences may comprise additional substitutions such as L14 and/or L15 substitutions (e.g., “LALA” substitutions L234A and L235A), and/or N77 (N297 e.g., N297A or N297G).

In an embodiment, a MAPP comprises a framework or dimerization polypeptide comprising an IgG1 sequence with a S144H and F185A HA-TF substitutions, and its counterpart interspecific binding partner polypeptide comprises an IgG1 sequence having Y129T and T174F HA-TF substitutions, where the framework and/or dimerization polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 4D; with none, one, or both of the sequences comprising L14 and L15 substitutions (e.g., “LALA” substitutions L234A and L235A), and/or N77 (N297 e.g., N297A or N297G).

In an embodiment, a MAPP comprises a framework or dimerization polypeptide comprising an IgG1 sequence with a T130V, L131Y, F185A, and Y187V ZW1 substitutions, and its counterpart interspecific binding partner polypeptide comprises an IgG1 sequence having T130V, T146L, K172L, and T174W ZW1 substitutions, where the framework and/or dimerization polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 4D; where one or both of the framework and/or dimerization polypeptide sequences may comprise additional substitutions such as L14 and/or L15 substitutions (e.g., “LALA” substitutions L234A and L235A), and/or N77 (N297 e.g., N297A or N297G).

In an embodiment, a MAPP comprises a framework or dimerization polypeptide comprising an IgG1 sequence with a K140D, D179M, and Y187A 7.8.60 substitutions, and its counterpart interspecific binding partner polypeptide comprises an IgG1 sequence having T130V E125R, Q127R, T146V, and K189V 7.8.60 substitutions, where the framework and/or dimerization polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 4D; where one or both of the framework and/or dimerization polypeptide sequences may comprise additional substitutions such as L14 and/or L15 substitutions (e.g., “LALA” substitutions L234A and L235A), and/or N77 (N297 e.g., N297A or N297G).

In an embodiment, a MAPP comprises a framework or dimerization polypeptide comprising an IgG1 sequence with a K189D, and K172D DD-KK substitutions, and its counterpart interspecific binding partner polypeptide comprises an IgG1 sequence having T130V D179K and E136K DD-KK substitutions, where the framework and/or dimerization polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 4D; where one or both of the framework and/or dimerization polypeptide sequences may comprise additional substitutions such as L14 and/or L15 substitutions (e.g., “LALA” substitutions L234A and L235A), and/or N77 (N297 e.g., N297A or N297G).

In an embodiment, a MAPP comprises a framework or dimerization polypeptide comprising an IgG1 sequence with a K140E and K189W EW-RVT substitutions, its counterpart interspecific binding partner polypeptide comprises an IgG1 sequence having T130V Q127R, D179V, and F185T EW-RVT substitutions, where the framework and/or dimerization polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 4D; where one or both of the framework and/or dimerization polypeptide sequences may comprise additional substitutions such as L14 and/or L15 substitutions (e.g., “LALA” substitutions L234A and L235A), and/or N77 (N297 e.g., N297A or N297G).

In an embodiment, a MAPP comprises a framework or dimerization polypeptide comprising an IgG1 sequence with a K140E, K189W, and Y129C EW-RVTs-s substitutions, its counterpart interspecific binding partner polypeptide comprises an IgG1 sequence having T130V Q127R, D179V, F185T, and S134C EW-RVTs-s substitutions, where the framework and/or dimerization polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 4D; where one or both of the framework and/or dimerization polypeptide sequences may comprise additional substitutions such as L14 and/or L15 substitutions (e.g., “LALA” substitutions L234A and L235A), and/or N77 (N297 e.g., N297A or N297G).

In an embodiment, a MAPP comprises a framework or dimerization polypeptide comprising an IgG1 sequence with a K150E and K189W A107 substitutions, its counterpart interspecific binding partner polypeptide comprises an IgG1 sequence having T130V E137N, D179V, and F185T A107 substitutions, where the framework and/or dimerization polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 4D; where one or both of the framework and/or dimerization polypeptide sequences may comprise additional substitutions such as L14 and/or L15 substitutions (e.g., “LALA” substitutions L234A and L235A), and/or N77 (N297 e.g., N297A or N297G).

As an alternative to the use of immunoglobulin CH2 and CH3 heavy chain constant regions as dimerization or multimerization sequences, immunoglobulin light chain constant regions (See FIGS. 5A and 5B) can be paired with Ig CH1 sequences (See FIG. 4I) as multimerization or dimerization sequences and their counterpart sequences of a framework polypeptide.

In an embodiment, a MAPP framework or dimerization polypeptide comprises an Ig CH1 domain (e.g., the polypeptide of FIG. 4I), and the sequence with which it will form a complex (its counterpart binding partner) comprises an Ig κ chain constant region sequence, where the framework or dimerization polypeptide comprise a sequence having at least 80%, 85%, 90%, 95%, 98%, 99%, or 100% sequence identity to at least 70, at least 80, at least 90, at least 100, or at least 110 contiguous aas of SEQ ID NOs:67 and/or 69 respectively. See FIGS. 41 and 5A. The Ig CH1 and Ig κ sequences may be modified to increase their affinity for each other, and accordingly the stability of any heterodimer formed utilizing them as a dimerization or multimerization sequences. Among the substitutions that increase the stability of CH1-Ig κ heterodimers are those identified as the MD13 combination in Chen et al., MAbs, 8(4):761-774 (2016). In the MD13 combination two substitutions are introduced into to each of the IgCH1 and Ig κ sequences. The Ig CH1 sequence is modified to contain S64E and S66V substitutions (S70E and S72V of the sequence shown in FIG. 4I). The Ig κ sequence is modified to contain S69L and T71S substitutions (S68L and T70S of the sequence shown in FIG. 5A).

In another embodiment, a framework or dimerization polypeptide of a MAPP comprises an Ig CH1 domain (e.g., the polypeptide of FIG. 4I SEQ ID NO:67), and its counterpart sequence comprises an Ig λ chain constant region sequence such as is shown in FIG. 5B (SEQ ID NO:70), where the framework or dimerization polypeptide comprises a sequence having at least 80%, 85%, 90%, 95%, 98%, 99%, or 100% sequence identity to at least 70 (e.g., at least 80, at least 90, or at least 100) contiguous aas of the sequences shown in FIG. 5B.

Framework and/or dimerization polypeptide of a MAPP each comprise a leucine zipper polypeptide as a dimerization or multimerization sequence. The leucine zipper polypeptides bind to one another to form dimer (e.g., homodimer). Non-limiting examples of leucine-zipper polypeptides include a peptide comprising any one of the following aa sequences: RMKQIEDKIEEILSKIYHIENEIAR-IKKLIGER (SEQ ID NO:71); LSSIEKKQEEQTSWLIWISNELTLIRNELAQS (SEQ ID NO:72); LSSIEKKLEEITSQLIQISNELTLIRNELAQ (SEQ ID NO:73); LSSIKKLEEITSQLIQIRNELTL-IRNELAQ (SEQ ID NO:74); LSSIEKKLEEITSQLQQIRNELTLIRNELAQ (SEQ ID NO:75); LSSLEKKLEELTSQLIQLRNELTLLRNELAQ (SEQ ID NO:76); ISSLEKKIEELTSQIQQLR-NEITLLRNEIAQ (SEQ ID NO:77). In some cases, a leucine zipper polypeptide comprises the following aa sequence: LEIEAAFLERENTALETRVAELRQRVQRLRNRVSQYRTRYGPLGGGK (SEQ ID NO:78). Additional leucine-zipper polypeptides are known in the art, a number of which are suitable for use as multimerization or dimerization sequences.

The a framework and/or dimerization polypeptide of a MAPP may comprise a coiled-coil polypeptide sequence that forms a dimer. Non-limiting examples of coiled-coil polypeptides include, for example, a peptide of any one of the following aa sequences: LKSVENRLAVVENQLKTVIEELK-TVKDLLSN (SEQ ID NO:79); LARIEEKLKTIKAQLSEIASTLNMIREQLAQ (SEQ ID NO:100); VSRLEEKVKTLKSQVTELASTVSLLREQVAQ (SEQ ID NO:80); IQSEKKIEDISSLIG-QIQSEITLIRNEIAQ (SEQ ID NO:81); and LMSLEKKLEELTQTLMQLQNELSMLKNELAQ (SEQ ID NO:82).

A MAPP may comprise a pair of two framework polypeptides and/or a framework and dimerization polypeptide that each have an aa sequence comprising at least one cysteine residue that can form a disulfide bond permitting homodimerization or heterodimerization of those polypeptides stabilized by disulfide bond between the cysteine residues. Examples of such aa sequences include: VDLEGSTSNGRQCAGIRL (SEQ ID NO:83); EDDVTTTEELAPALVPPPKGTCAGWMA (SEQ ID NO:84); and GHDQETTTQGPGVLLPLPKGACTGQMA (SEQ ID NO:85).

Some aa sequences suitable as multimerization (oligomerization) sequences permit formation of MAPPs capable of forming structures greater than duplexes of a heterodimers comprising a framework and dimerization polypeptide. In some instances, triplexes, tetraplexes, pentaplexes may be formed. Such aa sequences include, but are not limited to, IgM constant regions (see e.g., FIG. 4H) which forms hexamer, or pentamers (particularly when combined with a mature j-chain peptide lacking a signal sequence such as that provided in FIG. 4J. Collagen domains, which form trimers, can also be employed. Collagen domains may comprise the three aa sequence Gly-Xaa-Xaa and/or GlyXaaYaa, where Xaa and Yaa are independently any aa, with the sequence appear or are repeated multiple times (e.g., from 10 to 40 times). In Gly-Xaa-Yaa sequences, Xaa and Yaa are frequently proline and hydroxyproline respectively in greater than 25%, 50%, 75%, 80% 90% or 95% of the Gly-Xaa-Yaa occurrences, or in each of the Gly-Xaa-Yaa occurrences. In some cases, a collagen domain comprises the sequence Gly-Xaa-Pro repeated from 10 to 40 times. A collagen oligomerization peptide can comprise the following aa sequence: VTAFSNMDDMLQKAHLVIEGTFIYLRDSTEFFIRVRDGW-KKLQLGELIPIPADSPPPPALSSNP (SEQ ID NO:86).

Suitable framework polypeptides e.g., those with an Ig Fc multimerization sequence) will, in some cases, be half-life extending polypeptides. Thus, in some cases, a suitable framework polypeptide increases the in vivo half-life (e.g., the serum half-life) of the MAPPs, compared to a control MAPP having a framework polypeptide with a different aa sequence. For example, in some cases, a framework polypeptide increases the in vivo half-life (e.g., the serum half-life) of the MAPP, compared to a control MAPP having a framework polypeptide with a different aa sequence, by at least about 10%, at least about 15%, at least about 25%, at least about 50%, at least about 2-fold, at least about 5-fold, at least about 10-fold, at least about 25-fold, at least about 50-fold, at least about 100-fold, or more than 100-fold. As an example, in some cases, an Ig Fc polypeptide sequence (e.g., utilized as a multimerization sequence to form a duplex of MAPP heterodimers comprising a framework and dimerization polypeptide) increases the stability and/or in vivo half-life (e.g., the serum half-life) of a MAPP duplex compared to a control MAPP lacking the Ig Fc polypeptide sequence by at least about 10%, at least about 15%, at least about 25%, at least about 50%, at least about 2-fold, at least about 2.5-fold, at least about 5-fold, at least about 10-fold, at least about 25-fold, at least about 50-fold, at least about 100-fold, or more than 100-fold.

3. Presenting Sequence and Presenting Complexes

Epitope presenting sequences (presenting sequences) and epitope presenting complexes (presenting complexes) comprise MHC class I polypeptides (MHC-H and β2M) sufficient to bind and present an epitope to a TCR. Presenting sequences and complexes may also comprise additional protein (peptide) elements including one or more independently selected MODs and/or one or more independently selected linkers (e.g., linkers placed between various domains). As discussed herein, unless stated otherwise, neither presenting sequences nor presenting complexes comprise a MHC transmembrane domain (or intracellular domain such as a cytoplasmic tail) sufficient to anchor MAPP molecules (e.g., more than 50% of the MAPP molecules) in a mammalian cell membrane (e.g., a CHO cell membrane) when expressed therein.

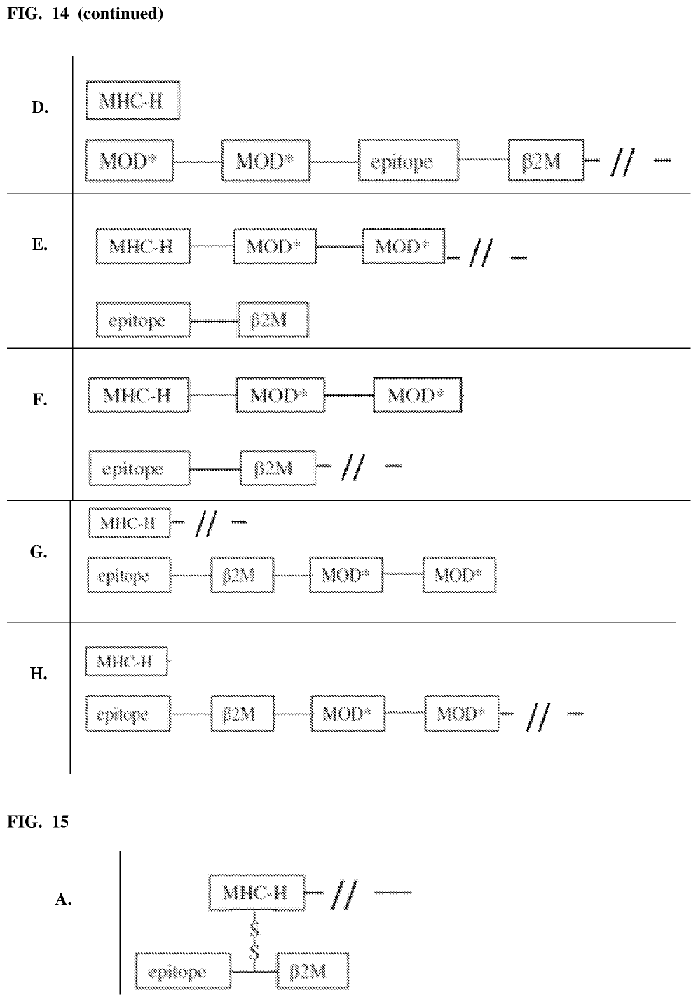

Conceptually, each of the presenting sequences and presenting complexes may be considered a “soluble MHC” that is fully capable of binding and presenting an epitope peptide (e.g., and epitope peptide that is part of a polypeptide comprising an MHC sequence) to a TCR. In presenting sequences all of the MHC sequences and optionally the epitope peptide sequence are present in a single polypeptide chain (single linear sequence of aas produced by translation). See e.g., FIG. 12 structures A and B.

Where the MHC-H and β2M polypeptides are divided among two or more polypeptide chains the “soluble MHC” is termed a presenting complex. The presenting complex has one chain that is part of a framework peptide or dimerization peptide, referred to as a “presenting complex 1^{st} sequence.” The second chain of the presenting complex is termed the “presenting complex 2^{nd} sequence.” The presenting complex 2^{nd} sequence may be associated non-covalently with the MHC components present in the presenting complex 1^{st} sequence (through binding interactions between MHC-H and β2M components as in FIGS. 13 and 14), in addition, one or more disulfide bonds between the presenting complex 1^{st} sequence and the presenting complex 2^{nd} sequence (See e.g., FIGS. 13-15.

In some cases, one or more presenting sequence of a MAPP comprises all of the class I components required for binding and presenting the epitope of interest to a TCR; e.g., the MHC-H and β2M and epitope in a single polypeptide sequence. In some cases, one or more presenting sequences of a MAPP, comprises presenting complexes, where the epitope is part of the presenting complex 1^{st} sequence or the presenting complex 2^{nd} sequence. See Aspect 1 in Section V of this disclosure.

Although subject to dissociation from the MAPPs, epitopes may also be separate peptides (e.g., phosphopeptide, lipopeptides glycosylated peptides, etc.).

Peptide presenting sequences and peptide presenting complexes in MAPPs may contain one or more substitutions in, for example, the MOD sequences and/or MHC sequences. By way of example, the presenting proteins or complexes may contain one or more reduced affinity variant MODs. The presenting sequences or complexes may also comprise substitutions in their MHC sequences, such as a class I MHC containing a Y84A substitution (as discussed above, that opens the end of the binding pocket). A class I MHC containing presenting sequence or presenting complex may also contain substitutions leading to stabilizing disulfide bonds, such as a cysteine substituted into the carboxyl end portion of the α1 helix and a cysteine in the amino end portion of the α2-1 helix (e.g., a disulfide bond formed between positions 84 and 139 in a Y84C and A139C substituted MHC-H as previously discussed). A class I MHC containing presenting sequence or presenting complex bearing cysteine substitutions at MHC-H aa 236 (A236C) and β2M aa 12 (e.g., R12C) may form a disulfide bond between those aas. In presenting sequences or presenting complexes having epitope peptides bound by a linker to the N-terminal side of the β2M sequence(s), a stabilizing disulfide bond joining a cysteine residue present in the linker (e.g., at position 2 of a the linker such as a G2C substitutions in the linkers of SEQ ID NOs:112-115 provided below) and a cysteine residue in the MHC-H polypeptide (e.g., a Cys introduced at position 84 such as a Y84C substitution) may also be introduced. A class I MHC containing presenting sequence or presenting complex may also contain paired substitutions forming two disulfide bonds such as between the R12C/Y84C and the Y84C/linker (e.g., G2C) substitution pairs.

4. MHC Class I Polypeptides

As noted above, MAPPs include MHC polypeptide sequences. For the purposes of this disclosure, the term “major histocompatibility complex (MHC) polypeptides” is meant to include MHC class I polypeptides of various species, including human MHC polypeptides (HLA polypeptides, and the MHC polypeptides of other mammalian species (e.g., lagomorphs, non-human primates, rodents canines, felines, ungulates (e.g., equines, bovines, ovines, caprines, etc.), and the like. Both the β2M and MHC-H chain sequences in a MAPP may be of human origin.

The HLA locus is highly polymorphic in nature, and class I MHC polypeptides include allelic forms. The HLA Nomenclature site maintained by the Anthony Nolan Research Institute, available on the world wide web at hla.alleles.org/nomenclature/index.html indicates that as of Apr. 8, 2019, there are 5,018 HLA-A alleles, 6,096, HLA-B alleles, 4,852 HLA-C alleles, 30 HLA-E alleles, 44 HLA-F alleles, and 68 HLA-G alleles.

MAPPs are not intended to include sequences (e.g., transmembrane domains) sufficient to anchor MAPPs in the membrane of a cell (e.g., a eukaryotic cell such as a mammalian cell such as a Chinese Hamster Ovary or “CHO” cell) in which the MAPP is expressed. Accordingly, the MAPPs described herein do not include a transmembrane domain and/or intracellular portions (e.g., cytoplasmic domain). Similarly, when mature (full process by an expressing cell) the polypeptides of the MAPPs described herein do not generally include MHC polypeptide leader sequences after cellular processing.

The term “MHC polypeptide” includes both 3-2 microglobulin and MHC class I heavy chains and/or portions thereof. More specifically, MHC class I polypeptides include a β2M polypeptide and the α1, α2, and α3 domains of class I MHC heavy chain, which together represent all or most of the extracellular Class I protein required for presentation of an epitope peptide. Accordingly, the MHC Class I heavy chain present in a MAPP of this disclosure may include only MHC class I heavy chain α1, α2, and α3 domains. In some cases, the MHC Class I heavy chain sequence present in a MAPP of the present disclosure has a length from about 270 aa to about 290 aa. In some cases, the MHC Class I heavy chain present in a MAPP of the present disclosure has a length of 270 aa, 271 aa, 272 aa, 273 aa, 274 aa, 275 aa, 276 aa, 277 aa, 278 aa, 279 aa, 280 aa, 281 aa, 282 aa, 283 aa, 284 aa, 285 aa, 286 aa, 287 aa, 288 aa, 289 aa, or 290 aa.

In some cases, a MHC polypeptide of a MAPP is a human MHC polypeptide, where human MHC polypeptides are also referred to as “human leukocyte antigen” (“HLA”) polypeptides, more specifically, a Class I HLA polypeptide, e.g., a β2M polypeptide, or a Class I HLA heavy chain polypeptide. Class I HLA heavy chain polypeptides that can be included in MAPPs include, for example, HLA-A, -B, -C, -E, -F, and/or -G heavy chain polypeptide extracellular domains (e.g., fully process and lacking the signal sequence). In an embodiment, the Class I HLA heavy chain polypeptides of MAPPs comprise polypeptides having a sequence having at least 75%, at least 80%, at least 85%, at least 90%, at least 95%, at least 98%, at least 99%, or 100% aa sequence identity to all or part (e.g., 50, 75, 100, 150, 200, or 250 contiguous aa) of the aa sequence of any of the human HLA heavy chain polypeptides depicted in FIGS. 3A-3I. For example, they may comprise 1-30, 1-5, 5-10, 10-15, 15-20, 20-25 or 25-30 aa insertions, deletions, and/or substitutions (in addition to those locations indicated as being variable in the heavy chain consensus sequences of FIGS. 3E-3I).

As an example, a MHC Class I heavy chain polypeptide of a MAPP can comprise an aa sequence having at least 75%, at least 80%, at least 85%, at least 90%, at least 95%, at least 98%, at least 99%, or 100% aa sequence identity to aas 25-300 (lacking all, or substantially all, of the leader, transmembrane and cytoplasmic sequences), or aas 25-365 (lacking the leader), of the human HLA-A heavy chain polypeptides depicted in FIGS. 3A, 3B and/or 3C.

(i) MHC Class I Heavy Chains

Class I human MHC polypeptides may be drawn from the classical HLA alleles (HLA-A, B, and C), or the non-classical HLA alleles (e.g., HLA-E, F and G). The following are non-limiting examples of MHC-H alleles and variants of those alleles that may be incorporated into MAPPs.

(a) HLA-A Heavy Chains

The HLA-A heavy chain peptide sequences, or portions thereof, that may be incorporated into a MAPP include, but are not limited to, the alleles: A*0101 (FIG. 3D SEQ ID NO:12, FIG. 3E SEQ ID NO:23), A*0201 (FIG. 3D SEQ ID NO:15, FIG. 3E SEQ ID NO:24), A*0301 (FIG. 3E SEQ ID NO:25), A*1101 (FIG. 3D SEQ ID NO:20, FIG. 3E SEQ ID NO:26), A*2301 (FIG. 3E SEQ ID NO:27), A*2402 (FIG. 3D SEQ ID NO:21, FIG. 3E SEQ ID NO:28), A*2407 (FIG. 3E SEQ ID NO:29), A*3303 (FIG. 3D SEQ ID NO:22, FIG. 3E SEQ ID NO:30), and A*3401 (FIG. 3E SEQ ID NO:31), which are aligned without all, or substantially all, of the leader, transmembrane and cytoplasmic sequences in FIG. 3E. Any of those alleles may comprise a substitution at one or more of positions 84, 139 and/or 236 (as shown in FIG. 3E) selected from: a tyrosine to alanine at position 84 (Y84A); a tyrosine to cysteine at position 84 (Y84C); an alanine to cysteine at position 139 (A139C); and an alanine to cysteine at position 236 (A236C). In addition, a HLA-A sequence having at least 75% (e.g., at least 80%, at least 85%, at least 90%, at least 95%, at least 98%, at least 99% or 100%) aa sequence identity to all or part (e.g., 50, 75, 100, 150, 200, or 250 contiguous aas) of the sequence of any of those HLA-A alleles may also be employed (e.g., it may comprise 1-30, 1-5, 5-10, 10-15, 15-20, 20-25, or 25-30 aa insertions, deletions, and/or substitutions). The HLA-A heavy chain polypeptide sequence of a MAPP may comprise the Y84C and A139C substitutions.

HLA-A*0101 (HLA-A*01:01:01:01)

In an embodiment, a MHC Class I heavy chain polypeptide of a MAPP comprises an aa sequence of HLA-A*01:01:01:01 (SEQ ID NOs: 12 or 23), or a sequence having at least 75% (at least 80%, at least 85%, at least 90%, at least 95%, at least 98%, at least 99% or 100%) aa sequence identity to all or part (e.g., 50, 75, 100, 150, 200, or 250 contiguous aas) of that sequence (e.g., it may comprise 1-30, 1-5, 5-10, 10-15, 15-20, 20-25, or 25-30 aa insertions, deletions, and/or substitutions). In an embodiment, where the HLA-A heavy chain polypeptide of a MAPP has less than 100% identity to the sequence labeled HLA-A in FIG. 3D, it may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). The HLA-A*0101 heavy chain polypeptide sequence of a MAPP may comprise the Y84C and A139C substitutions.

HLA-A*0201 (HLA-A*02:01)

In an embodiment, a MHC Class I heavy chain polypeptide of a MAPP comprises an aa sequence of HLA-A*0201 (SEQ ID NOs:15 or 24) provided in FIG. 3D or FIG. 3E, or a sequence having at least 75% (e.g., at least 80%, at least 85%, at least 90%, at least 95%, at least 98%, at least 99% or 100%) aa sequence identity to all or part (e.g., 50, 75, 100, 150, 200, or 250 contiguous aas) of that sequence (e.g., it may comprise 1-30, 1-5, 5-10, 10-15, 15-20, 20-25, or 25-30 aa insertions, deletions, and/or substitutions). In an embodiment, where the HLA-A*0201 heavy chain polypeptide of a MAPP has less than 100% identity to the sequence labeled HLA-A*0201 in FIG. 3D or 3E, it may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). The HLA-A*0201 heavy chain polypeptide of a MAPP may comprise the Y84A and A236C substitutions. In an embodiment, the HLA-A*0201 heavy chain polypeptide of a MAPP comprises the Y84C and A139C substitutions. In an embodiment, the HLA-A*0201 heavy chain polypeptide of a MAPP comprises the Y84C, A139C and A236C substitutions.

HLA-A*1101 (HLA-A*11:01)

In an embodiment, a MHC Class I heavy chain polypeptide of a MAPP comprises an aa sequence of HLA-A*1101 (SEQ ID NOs:20 or 26) provided in FIG. 3D or in FIG. 3E, or a sequence having at least 75% (e.g., at least 80%, at least 85%, at least 90%, at least 95%, at least 98%, at least 99% or 100%) aa sequence identity to all or part (e.g., 50, 75, 100, 150, 200, or 250 contiguous aas) of that sequence (e.g., it may comprise 1-30, 1-5, 5-10, 10-15, 15-20, 20-25, or 25-30 aa insertions, deletions, and/or substitutions). The HLA-A*1101 heavy chain allele may be prominent in Asian populations, including populations of individuals of Asian descent.

In an embodiment, where the HLA-A*1101 heavy chain polypeptide of a MAPP has less than 100% identity to the sequence labeled HLA-A*1101 in FIG. 3D or 3E, it may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). The HLA-A*1101 heavy chain polypeptide of a MAPP may comprise the Y84A and A236C substitutions. In an embodiment, the HLA-A*1101 heavy chain polypeptide of a MAPP comprises the Y84C and A139C substitutions. In an embodiment, the HLA-A*1101 heavy chain polypeptide of a MAPP comprises the Y84C, A139C and A236C substitutions.

HLA-A*2402 (HLA-A*24:02)

In an embodiment, a MHC Class I heavy chain polypeptide of a MAPP comprises an aa sequence of HLA-A*2402 (SEQ ID NOs:21 or 28) provided in FIG. 3D or 3E, or a sequence having at least 75% (e.g., at least 80%, at least 85%, at least 90%, at least 95%, at least 98%, at least 99% or 100%) aa sequence identity to all or part (e.g., 50, 75, 100, 150, 200, or 250 contiguous aas) of that sequence (e.g., it may comprise 1-30, 1-5, 5-10, 10-15, 15-20, 20-25, or 25-30 aa insertions, deletions, and/or substitutions). The HLA-A*2402 heavy chain allele may be prominent in Asian populations, including populations of individuals of Asian descent.

In an embodiment, where the HLA-A*2402 heavy chain polypeptide of a MAPP has less than 100% identity to the sequence labeled HLA-A*2402 in FIG. 3D or 3E, it may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). The HLA-A*2402 heavy chain polypeptide of a MAPP may comprise the Y84A and A236C substitutions. The HLA-A*2402 heavy chain polypeptide of a MAPP may comprise the Y84C and A139C substitutions. In an embodiment, the HLA-A*2402 heavy chain polypeptide of a MAPP comprises the Y84C, A139C and A236C substitutions.

HLA-A*3303 (HLA-A*33:03)

In an embodiment, a MHC Class I heavy chain polypeptide of a MAPP comprises an aa sequence of HLA-A*3303 (SEQ ID NOs:22 or 30) provided in FIG. 3D or 3E, or a sequence having at least 75% (e.g., at least 80%, at least 85%, at least 90%, at least 95%, at least 98%, at least 99% or 100%) aa sequence identity to all or part (e.g., 50, 75, 100, 150, 200, or 250 contiguous aas) of that sequence (e.g., it may comprise 1-25, 1-5, 5-10, 10-15, 15-20, 20-25, or 25-30 aa insertions, deletions, and/or substitutions). The HLA-A*3303 heavy chain allele may be prominent in Asian populations, including populations of individuals of Asian descent.

In an embodiment, where the HLA-A*3303 heavy chain polypeptide of a MAPP has less than 100% identity to the sequence labeled HLA-A*3303 in FIG. 3D, it may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine substitution at position 236 (A236C). The HLA-A*3303 heavy chain polypeptide of a MAPP may comprise the Y84A and A236C substitutions. The HLA-A*3303 heavy chain polypeptide of a MAPP may comprise the Y84C and A139C substitutions. The HLA-A*3303 heavy chain polypeptide of a MAPP may comprise the Y84C, A139C and A236C substitutions.

(b) HLA-B heavy chains

The HLA-B heavy chain peptide sequences, or portions thereof, that may be incorporated into a MAPP include, but are not limited to, the alleles B*0702 shown in FIGS. 3D and 3F (SEQ ID NOs:13 in FIG. 3D or 33 in FIG. 3F), or in FIG. 3F as B*0801 (SEQ ID NO:34), B*1502 (SEQ ID NO:35), B27 (subtypes B*2701-2759), B*3802 (SEQ ID NO:36), B*4001 (SEQ ID NO:37), B*4601 (SEQ ID NO:38), and B*5301 (SEQ ID NO:39), which are aligned without all, or substantially all, of the leader, transmembrane and cytoplasmic sequences in FIG. 3F. Any of those alleles may comprise a substitution at one or more of positions 84, 139 and/or 236 (as shown in FIG. 3F) selected from: a tyrosine to alanine at position 84 (Y84A); a tyrosine to cysteine at position 84 (Y84C); an alanine to cysteine at position 139 (A139C); and an alanine to cysteine at position 236 (A236C). In addition, an HLA-B sequence having at least 75% (e.g., at least 80%, at least 85%, at least 90%, at least 95%, at least 98%, at least 99% or 100%) aa sequence identity to all or part (e.g., 50, 75, 100, 150, 200, or 250 contiguous aas) of the sequence of any of those HLA-B alleles may also be employed (e.g., it may comprise 1-30, 1-5, 5-10, 10-15, 15-20, 20-25, or 25-30 aa insertions, deletions, and/or substitutions). The HLA-B heavy chain polypeptide sequence of a MAPP may comprise the Y84C and A139C substitutions.

HLA-B*0702 (HLA-B*07:02)

In an embodiment, a MHC Class I heavy chain polypeptide of a MAPP comprises an aa sequence of HLA-B*0702 (SEQ ID NO:33 and SEQ ID NO:13 in FIG. 3F labeled HLA-B in FIG. 3D), or a sequence having at least 75% (e.g., at least 80%, at least 85%, at least 90%, at least 95%, at least 98%, at least 99% or 100%) aa sequence identity to all or part (e.g., 50, 75, 100, 150, 200, or 250 contiguous aas) of that sequence (e.g., it may comprise 1-30, 1-5, 5-10, 10-15, 15-20, 20-25, or 25-30 aa insertions, deletions, and/or substitutions). In an embodiment, where the HLA-B heavy chain polypeptide of a MAPP has less than 100% identity to the sequence labeled HLA-B in FIG. 3D, it may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). The HLA-B*0702 heavy chain polypeptide of a MAPP may comprise the Y84A and A236C substitutions. The HLA-B*0702 heavy chain polypeptide of a MAPP may comprise the Y84C and A139C substitutions. The HLA-B*0702 heavy chain polypeptide of a MAPP may comprise the Y84C, A139C and A236C substitutions.

(c) HLA-C Heavy Chains

The HLA-C heavy chain peptide sequences, or portions thereof, that may be incorporated into a MAPP include, but are not limited to, the alleles: C*0102 (SEQ ID NO:41), C*0303 (SEQ ID NO:42), C*0304 (SEQ ID NO:43), C*0401 (SEQ ID NO:44), C*0602 (SEQ ID NO:45), C*0701 (SEQ ID NO:46), C*0702 (SEQ ID NO:47), C*0801 (SEQ ID NO:48), and C*1502 (SEQ ID NO:49), which are aligned without all, or substantially all, of the leader, transmembrane and cytoplasmic sequences in FIG. 3G. Any of those alleles may comprise a substitution at one or more of positions 84, 139 and/or 236 (as shown in FIG. 3G) selected from: a tyrosine to alanine at position 84 (Y84A); a tyrosine to cysteine at position 84 (Y84C); an alanine to cysteine at position 139 (A139C); and an alanine to cysteine at position 236 (A236C). In addition, an HLA-C sequence having at least 75% (e.g., at least 80%, at least 85%, at least 90%, at least 95%, at least 98%, at least 99% or 100%) aa sequence identity to all or part (e.g., 50, 75, 100, 150, 200, or 250 contiguous aas) of the sequence of any of those HLA-C alleles may also be employed (e.g., it may comprise 1-30, 1-5, 5-10, 10-15, 15-20, 20-25, or 25-30 aa insertions, deletions, and/or substitutions). The HLA-C heavy chain polypeptide sequence of a MAPP may comprise the Y84C and A139C substitutions.

HLA-C*701 (HLA-C*07:01) and HLA-C*702 (HLA-C*07:02)

The MHC Class I heavy chain polypeptide of a MAPP may comprise an aa sequence of HLA-C*701 (FIG. 3C SEQ ID NO:11 or FIG. 3G SEQ ID NO:46) or HLA-C*702, FIG. 3G (SEQ ID NO:47) in FIG. 3D labeled HLA-C, or a sequence having at least 75% (e.g., at least 80%, at least 85%, at least 90%, at least 95%, at least 98%, at least 99% or 100%) aa sequence identity to all or part (e.g., 50, 75, 100, 150, 200, or 250 contiguous aas) of one of those sequences (e.g., it may comprise 1-30, 1-5, 5-10, 10-15, 15-20, 20-25, or 25-30 aa insertions, deletions, and/or substitutions). In an embodiment, where the HLA-C heavy chain polypeptide of a MAPP has less than 100% identity to the sequence labeled HLA-C in FIG. 3D, it may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). The HLA-C*701 heavy chain polypeptide of a MAPP may comprise the Y84A and A236C substitutions. The HLA-C*701 heavy chain polypeptide of a MAPP may comprise the Y84C and A139C substitutions. The HLA-C*701 heavy chain polypeptide of a MAPP may comprise the Y84C, A139C and A236C substitutions.

(d) Non-Classical HLA-E, F and G Heavy Chains

The non-classical HLA heavy chain peptide sequences, or portions thereof, that may be incorporated into a MAPP include, but are not limited to, those of the HLA-E, F, and/or G alleles. Sequences for those alleles (and the HLA-A, B and C alleles) may be found on the world wide web at, for example, hla.alleles.org/nomenclature/index.html, the European Bioinformatics Institute (www.ebi.ac.uk), which is part of the European Molecular Biology Laboratory (EMBL), and at the National Center for Bioecology Information (www.ncbi.nlm.nih.gov).

Some suitable HLA-E alleles include, but are not limited to, HLA-E*0101 (HLA-E*01:01:01:01), HLA-E*01:03 (HLA-E*01:03:01:01), HLA-E*01:04, HLA-E*01:05, HLA-E*01:06, HLA-E*01:07, HLA-E*01:09, and HLA-E*01:10. Some suitable HLA-F alleles include, but are not limited to, HLA-F*0101 (HLA-F*01:01:01:01), HLA-F*01:02, HLA-F*01:03 (HLA-F*01:03:01:01), HLA-F*01:04, HLA-F*01:05, and HLA-F*01:06. Some suitable HLA-G alleles include, but are not limited to, HLA-G*0101 (HLA-G*01:01:01:01), HLA-G*01:02, HLA-G*01:03 (HLA-G*01:03:01:01), HLA-G*01:04 (HLA-G*01:04:01:01), HLA-G*01:06, HLA-G*01:07, HLA-G*01:08, HLA-G*01:09: HLA-G*01:10, HLA-G*01:11, HLA-G*01:12, HLA-G*01:14, HLA-G*01:15, HLA-G*01:16, HLA-G*01:17, HLA-G*01:18: HLA-G*01:19, HLA-G*01:20, and HLA-G*01:22. Consensus sequences for those HLA-E, -F, and -G alleles without all, or substantially all, of the leader, transmembrane and cytoplasmic sequences are provided in FIG. 3H as SEQ ID NOs:51, 52 and 53 respectively. Those consensus sequences are aligned in FIG. 3I with consensus sequences of the HLA-A, -B, and -C alleles (from FIGS. 3E-G).

Any of the above-mentioned HLA-E, F and/or G alleles may comprise a substitution at one or more of positions 84, 139 and/or 236 as shown in FIG. 3I for the consensus sequences. In an embodiment, the substitutions may be selected from: a position 84 tyrosine to alanine (Y84A) or cysteine (Y84C) or, in the case of HLA-F, a R84A or R84C substitution; a position 139 alanine to cysteine (A139C) or, in the case of HLA-F, a V139C; and an alanine to cysteine substitution at position 236 (A236C). In addition, an HLA-E, -F, and/or -G sequence having at least 75% (e.g., at least 80%, at least 85%, at least 90%, at least 95%, at least 98%, at least 99% or 100%) aa sequence identity to all or part (e.g., 50, 75, 100, 150, 200, or 250 contiguous aas) of any of the consensus sequences set forth in FIG. 3I may also be employed (e.g., the sequences may comprise 1-30, 1-5, 5-10, 10-15, 15-20, 20-25, or 25-30 aa insertions, deletions, and/or substitutions in addition to changes at variable residues listed therein). The HLA-E, F, or G heavy chain polypeptide sequence of a MAPP may comprise a cysteine at both position 84 and 139.

(e) Mouse H2K

In an embodiment, a MHC Class I heavy chain polypeptide of a MAPP comprises an aa sequence of MOUSE H2K (SEQ ID NO:16) (MOUSE H2K in FIG. 3D), or a sequence having at least 75%, at least 80%, at least 85%, at least 90%, at least 95%, at least 98%, at least 99%, or 100% aa sequence identity to all or part (e.g., 50, 75, 100, 150, 200, or 250 contiguous aas) of that sequence (e.g., it may comprise 1-30, 1-5, 5-10, 10-15, 15-20, 20-25, or 25-30 aa insertions, deletions, and/or substitutions). In an embodiment, where the MOUSE H2K heavy chain polypeptide of a MAPP has less than 100% identity to the sequence labeled MOUSE H2K in FIG. 3D, it may comprise a substitution at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine at position 236 (A236C). In an embodiment, the MOUSE H2K heavy chain polypeptide of a MAPP comprises the Y84A and A236C substitutions. In an embodiment, the MOUSE H2K heavy chain polypeptide of a MAPP comprises the Y84C and A139C substitutions. In an embodiment, the MOUSE H2K heavy chain polypeptide of a MAPP comprises the Y84C, A139C and A236C substitutions.

(f) The Effect of Substitutions and Combinations of Substitutions In Class I Polypeptides on MAPPS

Substitution of position 84, particularly when it is a tyrosine residue, with a small aa such as alanine (Y84A) or glycine tends to open one end of the MHC binding pocket, allowing a linker (e.g., attached to an epitope peptide) to “thread” through the end of the MHC class I binding pocket. A Y84A substitution permits greater variation in epitope sizes and allows longer peptides bearing epitope sequences to fit into the binding pocket and be presented by the MAPP. As with aa position 84 substitutions that open one end of the MHC-H binding pocket, substitution of an alanine or glycine at position 167 or its equivalent (e.g., a W167A or W167G substitutions or their equivalent) open the other end of the MHC binding pocket, creating a groove that permits greater variation (e.g., longer length) in the epitope peptides that may be present. In an embodiment, the HLA-A heavy chain polypeptide of a MAPP comprises the Y84A and A236C substitutions. In an embodiment, the HLA-A heavy chain polypeptide of a MAPP comprises the Y84C and A139C substitutions. When aas 84 and 139 are both cysteines, they may form an intrachain disulfide bond which can stabilize the MHC Class I protein, thereby permitting translation and excretion of the MAPP by eukaryotic cells (e.g., CHO cells), even when not loaded with an epitope peptide. When position 84 is a C residue, it can also form an interchain disulfide bond with a linker attached to the N-terminus of a β2M polypeptide (e.g., epitope-GCGGS (G₄S) (SEQ ID NO:87 (where the G₄S may be repeated 1-10 times)-mature β2M polypeptide (e.g., lacking its signal sequence), see FIG. 2 and SEQ ID NOs:1 to 5). When aa 236 is a cysteine, it can form an interchain disulfide bond with cysteine at aa 12 of a variant β2M polypeptide that comprises a R12C substitution at that position. Some possible combinations of MHC Class 1 heavy chain sequence modifications that may be incorporated into a MAPP are shown in the Table that follows.

The Table below lists some MHC heavy chain sequence modifications that may be incorporated into a MAPP.

Table of MHC Class I heavy chain mutations

| | HLA Heavy Chain | | |
|---|---|---|---|
| | Sequence | Sequence | Specific |
| | From | Identity | Substitutions at aa positions 84, |
| Entry | FIGS. 3D-H | Range□ | 139 and/or 236 |
| 1 | HLA-A | 75%-99.8%, 80%-99.8%, 85%-99.8%, | None; Y84C; Y84A; A139C; |
| | Consensus | 90%-99.8%, 95%-99.8%, 98%-99.8%, | A236C; (Y84A & A236C); Y84C & |
| | FIG. 3E | or 99%-99.8%; or 1-25, 1-5, 5-10, 10- | A236C; (Y84C & A139C); or |
| | | 15, 15-20, 20-25 or 25-30 aa insertions, | (Y84C, A139C & A236C) |
| | | deletions, and/or substitutions (not | |
| | | counting variable residues) | |
| 2 | A*0101, A*0201, | 75%-99.8%, 80%-99.8%, 85%-99.8%, | None; Y84C; Y84A; A139C; |
| | A*0301, A*1101, | 90%-99.8%, 95%-99.8%, 98%-99.8%, | A236C; (Y84A & A236C); Y84C & |
| | A*2402, A*2301, | or 99%-99.8%; or 1-30, 1-5, 5-10, 10- | A236C; (Y84C & A139C); or |
| | A*2402, A*2407, | 15, 15-20, 20-25 or 25-30 aa insertions, | (Y84C, A139C & A236C) |
| | A*3303, or A*3401 | deletions, and/or substitutions | |
| 3 | HLA-B | 75%-99.8%, 80%-99.8%, 85%-99.8%, | None; Y84C; Y84A; A139C; |
| | Consensus | 90%-99.8%, 95%-99.8%, 98%-99.8%, | A236C; (Y84A & A236C); Y84C & |
| | FIG. 3F | or 99%-99.8%; or 1-30, 1-5, 5-10, 10- | A236C; (Y84C & A139C); or |
| | | 15, 15-20, 20-25 or 25-30 aa insertions, | (Y84C, A139C & A236C) |
| | | deletions, and/or substitutions (not | |
| | | counting variable residues) | |
| 4 | B*0702, B*0801, | 75%-99.8%, 80%-99.8%, 85%-99.8%, | None; Y84C; Y84A; A139C; |
| | B*1502, B*3802, | 90%-99.8%, 95%-99.8%, 98%-99.8%, | A236C; (Y84A & A236C); Y84C & |
| | B*4001, B*4601, or | or 99%-99.8%; or 1-30, 1-5, 5-10, 10- | A236C; (Y84C & A139C); or |
| | B*5301 | 15, 15-20, 20-25 or 25-30 aa insertions, | (Y84C, A139C & A236C) |
| | | deletions, and/or substitutions | |
| 5 | HLA-C | 75%-99.8%, 80%-99.8%, 85%-99.8%, | None; Y84C; Y84A; A139C; |
| | Consensus | 90%-99.8%, 95%-99.8%, 98%-99.8%, | A236C; (Y84A & A236C); Y84C & |
| | FIG. 3G | or 99%-99.8%; or 1-30, 1-5, 5-10, 10- | A236C; (Y84C & A139C); or |
| | | 15, 15-20, 20-25 or 25-30 aa insertions, | (Y84C, A139C & A236C) |
| | | deletions, and/or substitutions (not | |
| | | counting variable residues) | |
| 6 | C*0102, C*0303, | 75%-99.8%, 80%-99.8%, 85%-99.8%, | None; Y84C; Y84A; A139C; |
| | C*0304, C*0401, | 90%-99.8%, 95%-99.8%, 98%-99.8%, | A236C; (Y84A & A236C); Y84C & |
| | C*0602, C*0701, | or 99%-99.8%; or 1-30, 1-5, 5-10, 10- | A236C; (Y84C & A139C); or |
| | C*702, C*0801, or | 15, 15-20, 20-25 or 25-30 aa insertions, | (Y84C, A139C & A236C) |
| | C*1502 | deletions, and/or substitutions | |
| 7 | HLA-E, F, or G | 75%-99.8%, 80%-99.8%, 85%-99.8%, | None; Y84C; Y84A; A139C; |
| | Consensus FIG. 3H | 90%-99.8%, 95%-99.8%, 98%-99.8%, | A236C; (Y84A & A236C); Y84C & |
| | | or 99%-99.8%; or 1-30, 1-5, 5-10, 10- | A236C; (Y84C & A139C); or |
| | | 15, 15-20, 20-25 or 25-30 aa insertions, | (Y84C, A139C & A236C) |
| | | deletions, and/or substitutions (not | Note that in HLA F contains an R84 |
| | | counting variable residues) | and V139, but can be substituted |
| | | | with C or A giving the |
| | | | corresponding R84C, R84A and/or |
| | | | V139C substitutions |
| 8 | MOUSE H2K | 75%-99.8%, 80%-99.8%, 85%-99.8%, | None; Y84C; Y84A; A139C; |
| | | 90%-99.8%, 95%-99.8%, 98%-99.8%, | A236C; (Y84A & A236C); Y84C & |
| | | or 99%-99.8%; or 1-30, 1-5, 5-10, 10- | A236C; (Y84C & A139C); or |
| | | 15, 15-20, 20-25 or 25-30 aa insertions, | (Y84C, A139C & A236C) |
| | | deletions, and/or substitutions | |

□The Sequence Identity Range is the permissible range in sequence identity of a MHC-H polypeptide sequence incorporated into a MAPP relative to the corresponding portion of sequences listed in FIGS. 3D-3H not counting the variable residues in the consensus sequences.

(g) 02-Microglobin Polypeptides and Their Combination with MHC-H Polypeptides

A 032M polypeptide of a MAPP can be a human 032M polypeptide, a non-human primate 032M polypeptide, a murine 032M polypeptide, and the like. In some instances, a 032M polypeptide comprises an aa sequence having at least 75%, at least 80%, at least 85%, at least 90%, at least 95%, at least 98%, at least 99%, or 100% aa sequence identity to a 032M aa sequence depicted in FIG. 2. In some instances, a 032M polypeptide comprises an aa sequence having at least 75%, at least 80%, at least 85%, at least 90%, at least 95%, at least 98%, at least 99%, or 100% aa sequence identity to aas 21 to 119 of a β2M aa sequence depicted in FIG. 2.

In some cases, a MHC class I polypeptide comprises an aa substitution replacing another aa with a cysteine (Cys) residue. Such cysteine residues can form a disulfide bond with a cysteine residue present in a second polypeptide chain.

In some cases, a β2M polypeptide sequence and an MHC-H polypeptide sequence comprise naturally occurring cysteines substituted for another aa. Such cysteines permit formation of a disulfide bond between those sequences. For example, in some cases MAPPs may contain a disulfide bond between one of following pairs of residues in a HLA β2M (see FIG. 2) and a HLA Class I heavy chains (see FIGS. 3D-3I) (where residue numbers are those of the mature polypeptide): 1) β2M residue 12, HLA Class I heavy chain residue 236; 2) β2M residue 12, HLA Class I heavy chain residue 237; 3) β2M residue 8, HLA Class I heavy chain residue 234; 4) β2M residue 10, HLA Class I heavy chain residue 235; 5) β2M residue 24, HLA Class I heavy chain residue 236; 6) β2M residue 28, HLA Class I heavy chain residue 232; 7) β2M residue 98, HLA Class I heavy chain residue 192; 8) β2M residue 99, HLA Class I heavy chain residue 234; 9) β2M residue 3, HLA Class I heavy chain residue 120; 10) β2M residue 31, HLA Class I heavy chain residue 96; 11) β2M residue 53, HLA Class I heavy chain residue 35; 12) β2M residue 60, HLA Class I heavy chain residue 96; 13) β2M residue 60, HLA Class I heavy chain residue 122; 14) β2M residue 63, HLA Class I heavy chain residue 27; 15) β2M residue Arg3, HLA Class I heavy chain residue Gly120; 16) β2M residue His31, HLA Class I heavy chain residue Gln96; 17) β2M residue Asp53, HLA Class I heavy chain residue Arg35; 18) β2M residue Trp60, HLA Class I heavy chain residue Gln96; 19) β2M residue Trp60, HLA Class I heavy chain residue Asp122; 20) β2M residue Tyr63, HLA Class I heavy chain residue Tyr27; 21) β2M residue Lys6, HLA Class I heavy chain residue Glu232; 22) β2M residue Gln8, HLA Class I heavy chain residue Arg234; 23) β2M residue Tyr10, HLA Class I heavy chain residue Pro235; 24) β2M residue Ser11, HLA Class I heavy chain residue Gln242; 25) β2M residue Asn24, HLA Class I heavy chain residue Ala236; 26) β2M residue Ser28, HLA Class I heavy chain residue Glu232; 27) β2M residue Asp98, HLA Class I heavy chain residue His192; and 28) β2M residue Met99, HLA Class I heavy chain residue Arg234. The aa numbering of the MHC/HLA Class I heavy chain is in reference to the mature MHC/HLA Class I heavy chain, without a signal peptide. For example, in some cases, residue 236 of the mature HLA-A, -B, or -C aa sequence (i.e., residue 260 of the aa sequence depicted in FIGS. 3A-3C respectively) is substituted with a Cys. In some cases, residue 32 (corresponding to Arg-12 of mature β2M) of an aa sequence depicted in FIG. 2 is substituted with a Cys.

Separately, or in addition to, the pairs of cysteine residues in a β2M and HLA Class I heavy chain polypeptide that may be used to form interchain disulfide bonds between the first and second polypeptides of a MAPP (discussed above), the HLA-heavy chain of a MAPP or its epitope conjugate may be substituted with cysteines to form an intrachain disulfide bond between a cysteine substituted into the carboxyl end portion of the α1 helix and a cysteine in the amino end portion of the α2-1 helix. Such disulfide bonds stabilize the MAPP and permit its cellular processing and excretion from eukaryotic cells in the absence of a bound epitope peptide (or null peptide). In one embodiment, a disulfide bond is formed between the carboxyl end portion of the α1 helix is from about aa position 79 to about aa position 89 and the amino end portion of the α2-1 helix is from about aa position 134 to about aa position 144 of the MHC Class I heavy chain (the aa positions are determined based on the sequence of the heavy chains without their leader sequence (see, e.g., FIGS. 3D-3H). In one such embodiment the disulfide bond is between a cysteine located at positions 83, 84, or 85 and a cysteine located at any of positions 138, 139 or 140 of the MHC Class I heavy chain. For example, a disulfide bond may be formed from cysteines incorporated into the MHC Class I heavy chain at aa 83 and a cysteine at an aa located at any of positions 138, 139 or 140. Alternatively, a disulfide bond may be formed between a cysteine inserted at position 84 and a cysteine inserted at any of positions 138, 139 or 140, or between a cysteine inserted at position 85 and a cysteine at any one of positions 138, 139 or 140. In an embodiment, the MHC class I heavy chain intrachain disulfide bond is between cysteines substituted into a heavy chain sequence at positions 84 and 139 (e.g., the heavy chain sequence may be one of the heavy chain sequences set forth in FIGS. 3D-3H). As noted above, any of the MHC Class I intrachain disulfide bonds, including a disulfide bond between cysteines at 84 and 139, may be combined with other interchain disulfide bonds including a bond between MHC Class 1 heavy position 236 and position 12 of a mature β2M polypeptide sequence (lacking its leader) as shown, for example, in FIG. 2.

In another embodiment, an intrachain disulfide bond may be formed in a MHC-H sequence of a MAPP between a cysteine substituted into the region between aa positions 79 and 89 and a cysteine substituted into the region between aa positions 134 and 144 of the sequences given in FIGS. 3D-3H. In such an embodiment, the MHC Class I heavy chain sequence may have insertions, deletions and/or substitutions of 1 to 5 aas preceding or following the cysteines forming the disulfide bond between the carboxyl end portion of the α1 helix and the amino end portion of the α2-1 helix. Any inserted aas may be selected from the naturally occurring aas or the naturally occurring aas except proline and alanine.

In an embodiment, the β2M polypeptide sequence of a MAPP comprises a mature β2M polypeptide sequence (aas 21-119) of any one of NP_004039.1, NP_001009066.1, NP_001040602.1, NP_776318.1, or NP_033865.2 (SEQ ID NOs:1 to 5).

In some cases, a HLA Class I heavy chain polypeptide of a MAPP or its epitope conjugate comprises any one of the HLA-A, -B, -C, -E, -F, or -G sequences in FIGS. 3D-3H. Any of the heavy chain sequences may further comprise cysteine substitutions at positions 84 and 139, which may form an intrachain disulfide bond.

In an embodiment, the β2M polypeptide of a MAPP comprises a mature β2M polypeptide sequence (aas 21-119) of any one of the sequences in FIG. 2, which further comprises a R12C substitution.

In an embodiment, a MAPP comprises a mature β2M polypeptide sequence (e.g., aas 21-119 of any one of the sequences in FIG. 2) having a R12C substitution, and any one of the HLA-A, -B, -C, -E, -F, or -G sequences in FIGS. 3D-3H having or substituted with a cysteine at position 236. In such embodiments a disulfide bond may form between the cysteines at positions 12 and 236. In addition, any of the heavy chain sequences may further comprise cysteine substitutions at positions 84 and 139, which may also form a disulfide bond.

In an embodiment, a MAPP has a β2M peptide sequence comprising aas 21-119 of a β2M sequence in FIG. 2 (e.g., NP_004039.1) with a R12C substitution, and an HLA Class I heavy chain polypeptide comprises the aa sequence: GSHSMRYFFTSVSRPGRGEPRFIAVGYVDDTQFVRFDSDAASQRMEPRAPWIEQEGPEYWDGE TRKVKAHSQTHRVDL(aa cluster 1){C}(aa cluster 2)AGSHTVQRMYGCDVGSDWRFLRGYHQYA YDGKDYIALKEDLRSW(aa cluster 3){C}(aa cluster 4)HKWEAAHVAEQLRAYLEGTCVEWLRRY LENGKETLQRTDAPKTHMTHHAVSDHEATLRCWALSFYPAEITLTWQRDGEDQTQDTELVET RPAGDGTFQKWAAVVVPSGQEQRYTCHVQHEGLPKPLTLRWEP (SEQ ID NO:88); or, the first polypeptide comprises the sequence, IQRTPKIQVYSCHPAENGKSNFLNCYVSGFHPSDIEVDLLKNGERIEKVEHSDLSFSKDWSFYLL YYTEFTPTEKDEYACRVNHVTLSQPKIVKWDRDM (SEQ ID NO:89), and the second polypeptide comprises the aa sequence, GSHSMRYFFTSVSRPGRGEPRFIAVGYVDDTQFVRFDSDAASQRMEPRAPWIEQEGPEYWDGE TRKVKAHSQTHRVDL(aa cluster 1){C}(aa cluster 2)AGSHTVQRMYGCDVGSDWRFLRGYHQYA YDGKDYIALKEDLRSW(aa cluster 3){C}(aa cluster 4))HKWEAAHVAEQLRAYLEGTCVEWLRR YLENGKETLQRTDAPKTHMTHHAVSDHEATLRCWALSFYPAEITLTWQRDGEDQTQDTEL(aa cluster 5)(C)(aa cluster 6)QKWAAVVVPSGQEQRYTCHVQHEGLPKPLTLRWEP (SEQ ID NO:90);
where the cysteine residues indicated as {C} form a disulfide bond between the α1 and α2-1 helices and the (C) residue forms a disulfide bond with the mature β2M polypeptide cysteine at position 12.

Each occurrence of aa cluster 1, aa cluster 2, aa cluster 3, aa cluster 4, aa cluster 5, and aa cluster 6 is independently selected to be 1-5 aa residues, wherein the aa residues are each selected independently from i) any naturally occurring (proteogenic) aa or ii) any naturally occurring aa except proline or glycine.

In an embodiment where the MHC Class I heavy chain is an HLA-A chain:
aa cluster 1 may be the aa sequence GTLRG or that sequence with one or two aas deleted or substituted with other naturally occurring aas (e.g., L replaced by I, V, A or F);
aa cluster 2 may be the aa sequence YNQSE or that sequence with one or two aas deleted or substituted with other naturally occurring aas (e.g., N replaced by Q, Q replaced by N, and/or E replaced by D);
aa cluster 3 may be the aa sequence TAADM or that sequence with one or two aas deleted or substituted with other naturally occurring aas (e.g., T replaced by S, A replaced by G, D replaced by E, and/or M replaced by L, V, or I);
aa cluster 4 may be the aa sequence AQTTK or that sequence with one or two aas deleted or substituted with other naturally occurring aas (e.g., A replaced by G, Q replaced by N, or T replaced by S, and or K replaced by R or Q);
aa cluster 5 may be the aa sequence VETRP or that sequence with one or two aas deleted or substituted with other naturally occurring aas (e.g., V replaced by I or L, E replaced by D, T replaced by S, and/or R replaced by K); and/or
aa cluster 6 may be the aa sequence GDGTF or that sequence with one or two aas deleted or substituted with other naturally occurring aas (e.g., D replaced by E, T replaced by S, or F replaced by L, W, or Y).

In some cases, the β2M polypeptide sequence of a MAPP comprises the aa sequence:

(SEQ ID NO: 89)
IQRTPKIQVYSCHPAENGKSNFLNCYVSGFHPSDIEVDLLKNGERIEKV
EHSDLSFSKDWSFYLLYYTEFTPTEKDEYACRVNHVTLSQPKIVKWDRD
M.

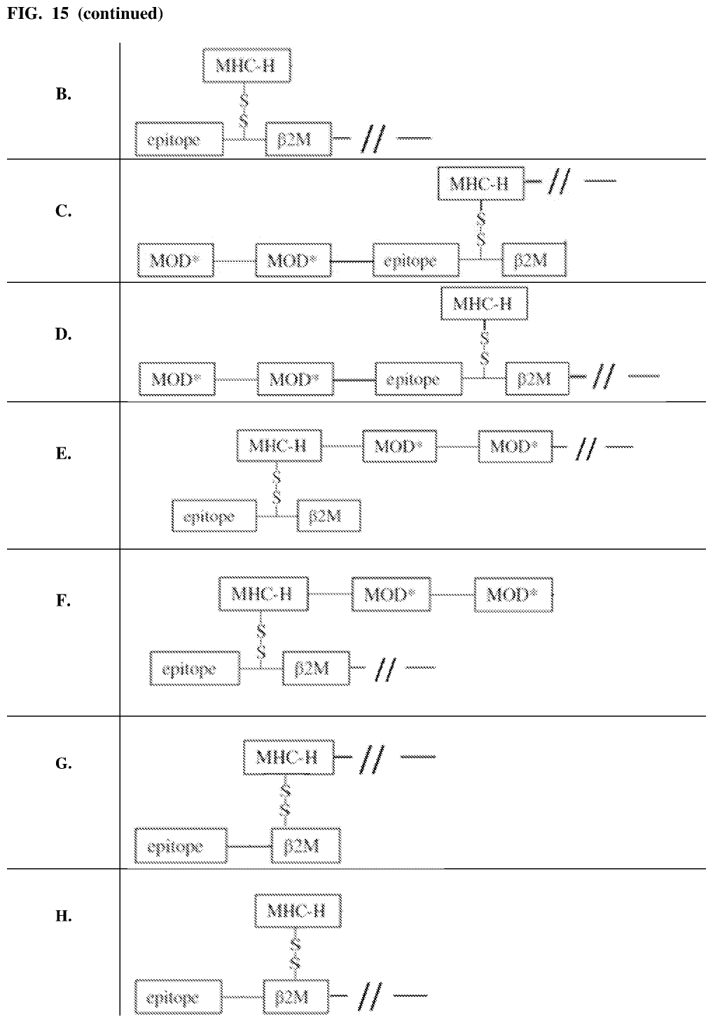

In some cases, such as where a MAPP comprises a presenting complex with an epitope and β2M sequence on one of the presenting complex 1^{st} sequence and the presenting complex 2^{nd} sequence, with the MHC-H sequences on the other of presenting complex 1^{st} sequence and the presenting complex 2^{nd} sequence, one or more disulfide bond may be used to link the presenting complex 1^{st} sequence and the presenting complex 2^{nd} sequence (see, e.g., FIG. 15). In one instance the disulfide bond joining the presenting complex 1^{st} sequence and the presenting complex 2^{nd} sequence is between a Cys residue present in a linker connecting the epitope and a β2M polypeptide sequence and a Cys residue in the MHC-H polypeptide (see, e.g., FIG. 15, structures A to F). In such an instance the Cys residue present in the MHC Class I heavy chain may be a Cys introduced as a Y84C substitution and the linker connecting the peptide epitope and the β2M polypeptide in the first polypeptide chain is GCGGS(G₄S) where the G₄S may be repeated from 1 to 9 times (SEQ ID NO:92) (e.g., epitope-GCGGS(G₄S)-mature β2M polypeptide). For example, in some cases, the linker comprises the aa sequence GCGGSGGGGSGGGGSGGGGS (SEQ ID NO:93). As another example, the linker comprises the aa sequence GCGGSGGGGSGGGGS (SEQ ID NO:94). In another instance, the disulfide bond joining the presenting complex 1^{st} sequence and the presenting complex 2^{nd} sequence is between a Cys residue in a β2M polypeptide sequence and a Cys residue present in a MHC Class I heavy sequence (see, e.g., FIG. 15, structures G and H). In such an instance the Cys residue present in the MHC Class I heavy chain may be a Cys introduced as a A-236C substitution and the Cys residue present in the β2M polypeptide sequence is a Cys introduced as a R12C substitution.

5. Immunomodulatory Polypeptides (“MODs”)

MODs that are suitable for inclusion in a MAPP of the present disclosure include, but are not limited to, IL-1, IL-2, IL-4, IL-6, IL-7, IL-10, IL-12, IL-15, IL-17, IL-21, IL-23, CD7, CD30L, CD40, CD70, CD80, (B7-1), CD83, CD86 (B7-2), HVEM (CD270), ILT3 (immunoglobulin-like transcript 3), ILT4 (immunoglobulin-like transcript 4), Fas ligand (FasL), ICAM (intercellular adhesion molecule), ICOS-L (inducible costimulatory ligand), JAG1 (CD339), lymphotoxin beta receptor, 3/TR6, OX40L (CD252), PD-L1, PD-L2, TGF-β1, TGF-β2, TGF-β3, 4-1BBL, and fragments of any thereof, such as ectodomain fragments, capable of engaging and signaling through their cognate receptor). MODs polypeptides suitable for inclusion in a MAPP of the present disclosure, and their “co-MODS (“co-immunomodulatory polypeptides” or cognate costimulatory receptors) include polypeptide sequences with T cell modulatory activity from the protein pairs recited in the following table:

Exemplary Pairs of MODs and Co-MODs

a) 4-1BBL (MOD) and 4-1BB (Co-MOD);
b) PD-L1 (MOD) and PD1 (Co-MOD);
c) IL-2 (MOD) and IL-2 receptor (Co-MOD);
d) CD80 (MOD) and CD28 (Co-MOD);
e) CD86 (MOD) and CD28 (Co-MOD);
f) OX40L (CD252) (MOD) and OX40 (CD134)
(Co-MOD);
g) Fas ligand (MOD) and Fas (Co-MOD);
h) ICOS-L (MOD) and ICOS (Co-MOD);
i) ICAM (MOD) and LFA-1 (Co-MOD);
j) CD30L (MOD) and CD30 (Co-MOD);
k) CD40 (MOD) and CD40L (Co-MOD);
l) CD83 (MOD) and CD83L (Co-MOD);
m) HVEM (CD270) (MOD) and CD160 (Co-
MOD);
n) JAG1 (CD339) (MOD) and Notch (Co-
MOD);
o) JAG1 (CD339) (MOD) and CD46 (Co-
MOD);
p) CD70 (MOD) and CD27 (Co-MOD);
q) CD80 (MOD) and CTLA4 (Co-MOD);
r) CD86 (MOD) and CTLA4 (Co-MOD);
s) PD-L1(MOD) and CD-80 (Co-MOD); and
t) TGF-β1, TGF-β2, and/or TGF-β3 (MODs)
and TGF-β Receptor (e.g., TGFBR1
and/or TGFBR2) (Co-MOD)

Typically, however, the chosen MOD(s) for the MAPPs of this disclosure will be MODs that cause T cell activation that provides one or more of the properties discussed above, i.e., an increase the activity of ZAP70 protein kinase activity, induction in the proliferation of the T-cell(s), granule-dependent effector actions (e.g., the release of granzymes, perforin, and/or granulysin from cytotoxic T-cells), and/or release of T cell cytokines (e.g., interferon γ from CD8+ cells). In some cases, the MOD is selected from an IL-2 polypeptide, a 4-1BBL polypeptide, a B7-1 polypeptide; a B7-2 polypeptide, an ICOS-L polypeptide, an OX-40L polypeptide, a CD80 polypeptide, a CD86 polypeptide and a TGFβ polypeptide. In some cases, the MAPP or duplex MAPP comprises two different MODs, such as an IL-2 MOD or IL-2 variant MOD polypeptide and either a CD80 or CD86 MOD polypeptide. In another instance, the MAPP or duplex MAPP comprises an IL-2 MOD or IL-2 variant MOD polypeptide. In some case MODs, which may be the same or different, are present in a MAPP or duplex MAPP in tandem. When MODs are presented in tandem, their sequences are immediately adjacent to each other on a single polypeptide, either without any intervening sequence or separated by only a linker polypeptide (e.g., no MHC sequences or epitope sequences intervene). The MOD polypeptide may comprise all or part of the extracellular portion of a full-length MOD. Thus, for example, the MOD can in some cases exclude one or more of a signal peptide, a transmembrane domain, and an intracellular domain normally found in a naturally-occurring MOD. Unless stated otherwise, a MOD present in a MAPP or duplex MAPP does not comprise the signal peptide, intracellular domain, or a sufficient portion of the transmembrane domain to anchor a substantial amount (e.g., more than 5% or 10%) of a MAPP or duplex MAPP into a mammalian cell membrane.

In some cases, a MOD suitable for inclusion in a MAPP of the present disclosure comprises all or a portion of (e.g., an extracellular portion of) the aa sequence of a naturally-occurring MOD. In other instances, a MOD suitable for inclusion in a MAPP of the present disclosure is a variant MOD that comprises at least one aa substitution compared to the aa sequence of a naturally-occurring MOD. In some instances, a variant MOD exhibits a binding affinity for a co-MOD that is lower than the affinity of a corresponding naturally-occurring MOD (e.g., a MOD not comprising the aa substitution(s) present in the variant) for the co-MOD. Suitable variations in MOD polypeptide sequence that alter affinity may be identified by scanning (making aa substitution e.g., alanine substitutions or “alanine scanning” or charged residue changes) along the length of a peptide and testing its affinity. Once key aa positions altering affinity are identified those positions can be subject to a vertical scan in which the effect of one or more aa substitutions other than alanine are tested.

a. MODs and Variant MODs with Reduced Affinity

Suitable immunomodulatory domains that exhibit reduced affinity for a co-immunomodulatory domain can have from 1 aa (aa) to 20 aa differences from a wild-type immunomodulatory domain. For example, in some cases, a variant MOD present in a MAPP of the present disclosure differs in aa sequence by 1 aa to 10 aa, or by 11 aa to 20 aa from a corresponding wild-type MOD. A variant MOD present in a MAPP of the present disclosure may include a single aa substitution compared to a corresponding reference (e.g., wild-type) MOD. A variant MOD present in a MAPP of the present disclosure may include 2 aa substitutions compared to a corresponding reference (e.g., wild-type) MOD. A variant MOD present in a MAPP of the present disclosure may include 3 aa substitutions compared to a corresponding reference (e.g., wild-type) MOD. A variant MOD present in a MAPP of the present disclosure may include 4 aa substitutions compared to a corresponding reference (e.g., wild-type) MOD. A variant MOD present in a MAPP of the present disclosure may include 5 aa substitutions compared to a corresponding reference (e.g., wild-type) MOD. A variant MOD present in a MAPP of the present disclosure may include 6 aa or 7 aa substitutions compared to a corresponding reference (e.g., wild-type) MOD. A variant MOD present in a MAPP of the present disclosure may include 8 aa, 9 aa, or 10 aa substitutions compared to a corresponding reference (e.g., wild-type) MOD.

A variant MOD present in a MAPP of the present disclosure may include 11 or 12 aa substitutions compared to a corresponding reference (e.g., wild-type) MOD. A variant MOD present in a MAPP of the present disclosure may include 13, 14, or 15 aa substitutions compared to a corresponding reference (e.g., wild-type) MOD. A variant MOD present in a MAPP of the present disclosure may include 16, 17, 18, 19, or 20 aa substitutions compared to a corresponding reference (e.g., wild-type) MOD.

As discussed above, a variant MOD suitable for inclusion in a MAPP of the present disclosure may exhibit reduced affinity for a cognate co-MOD, compared to the affinity of a corresponding wild-type MOD for the cognate co-MOD.

In some cases, a variant MOD present in a MAPP of the present disclosure has a binding affinity for a cognate co-MOD that is from 100 nM to 100 μM. For example, in some cases, a variant MOD present in a MAPP of the present disclosure has a binding affinity for a cognate co-MOD that is from about 100 nM to about 200 nM, from about 200 nM to about 300 nM, from about 300 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, from about 1 μM to about 5 μM, from about 5 μM to about 10 μM, from about M to about 20 μM, from about 20 μM to about 30 μM, from about 30 μM to about 50 μM, from about 50 μM to about 75 μM, or from about 75 μM to about 100 μM.

(i) Determining Binding Affinity

Binding affinity between a MOD and its cognate Co-MOD can be determined by bio-layer interferometry (BLI) using purified MOD and purified cognate Co-MOD, following the procedure set forth in published PCT Application WO 2020/132138 A1. Unless otherwise stated herein, the affinity of a MOD for a Co-MOD is determined using BLI following the procedure set forth in published PCT Application WO 2020/132138 A1.

In some cases, the ratio of: i) the binding affinity of a control MAPP (where the control MAPP comprises a wild-type MOD) to a cognate co-MOD to ii) the binding affinity of a MAPP of the present disclosure comprising a variant of the wild-type MOD to the cognate co-MOD, when measured by BLI (as described above), is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at least 10⁵:1, or at least 10⁶:1. In some cases, the ratio of: i) the binding affinity of a control MAPP (where the control MAPP comprises a wild-type MOD) to a cognate co-MOD to ii) the binding affinity of a MAPP of the present disclosure comprising a variant of the wild-type MOD to the cognate co-MOD, when measured by BLI, is in a range of from 1.5:1 to 10⁶:1, e.g., from 1.5:1 to 10:1, from 10:1 to 50:1, from 50:1 to 10²:1, from 10²:1 to 103:1, from 10³:1 to 10⁴:1, from 10⁴:1 to 10⁵:1, or from 10⁵:1 to 10⁶:1.

The epitope present in a MAPP of the present disclosure may bind to a T cell receptor (TCR) on a T cell with an affinity of at least 100 μM (e.g., at least 10 μM, at least 1 μM, at least 100 nM, at least 10 nM, or at least 1 nM). In some cases, the epitope present in a MAPP of the present disclosure binds to a TCR on a T cell with an affinity of from about 10⁻⁴ M to about 5×10⁻⁴ M, from about 5×10⁻⁴ M to about 10⁻⁵ M, from about 10⁻⁵ M to 5×10⁻⁵ M, from about 5×10⁻⁵ M to 10⁻⁶ M, from about 10⁻⁶ M to about 5×10⁻⁶ M, from about 5×10⁻⁶ M to about 10⁻⁷ M, from about 10⁻⁷ M to about 5×10⁻⁷ M, from about 5×10⁻⁷ M to about 10⁻⁸ M, or from about 10⁻⁸ M to about 10⁻⁹ M. Expressed another way, in some cases, the epitope present in a MAPP of the present disclosure binds to a TCR on a T cell with an affinity of from about 1 nM to about 5 nM, from about 5 nM to about 10 nM, from about 10 nM to about 50 nM, from about 50 nM to about 100 nM, from about 0.1 μM to about 0.5 μM, from about 0.5 μM to about 1 μM, from about 1 μM to about 5 μM, from about 5 μM to about 10 μM, from about 10 μM to about 25 μM, from about 25 μM to about 50 μM, from about 50 μM to about 75 μM, or from about 75 M to about 100 μM.

In some cases, a variant MOD present in a MAPP of the present disclosure has a binding affinity for a cognate co-MOD that is from 1 nM to 100 nM, or from 100 nM to 100 μM. For example, in some cases, a variant MOD present in a MAPP of the present disclosure has a binding affinity for a cognate co-MOD that is from about 100 nM to about 200 nM, from about 200 nM to about 300 nM, from about 300 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, from about 1 μM to about 5 μM, from about 5 μM to about 10 μM, from about 10 μM to about 20 μM, from about 20 μM to about 30 μM, from about 30 M to about 50 μM, from about 50 μM to about 75 μM, or from about 75 μM to about 100 μM. In some cases, a variant MOD present in a MAPP of the present disclosure has a binding affinity for a cognate co-MOD that is from about 1 nM to about 5 nM, from about 5 nM to about 10 nM, from about 10 nM to about 50 nM, or from about 50 nM to about 100 nM.

Immunomodulatory polypeptides and variants, including reduced affinity variants, such as CD80, CD86, 4-1BBL and IL-2 are described in the published literature, e.g., published PCT application WO2020132138A1, the disclosure of which as it pertains to immunomodulatory polypeptides and specific variant immunomodulatory polypeptides of CD80, CD86, 4-1BBL, IL-2 are expressly incorporated herein by reference, including specifically paragraphs [00260]-[00455] of WO2020132138A1.

Of specific interest are MODs that are variants of the cytokine IL-2, discussed below in further detail. Wild-type IL-2 binds to IL-2 receptor (IL-2R) on the surface of a T cell. Wild-type IL-2 has a strong affinity for IL-2R and will bind to activate most or substantially all CD8+ T cells. For this reason, synthetic forms of wt. Il-2 such as the drug Aldesleukin (trade name Proleukin®) are known to have severe side-effects when administered to humans for the treatment of cancer because the IL-2 indiscriminately activates both target and non-target T cells.

An IL-2 receptor is in some cases a heterotrimeric polypeptide comprising an alpha chain (IL-2Rα; also referred to as CD25), a beta chain (IL-2Rβ; also referred to as CD122: and a gamma chain (IL-2Rγ; also referred to as CD132). Amino acid sequences of human IL-2, human IL-2Rα, IL2Rβ, and IL-2Rγ are known. See, e.g., published PCT application WO2020132138A1, discussed above.

In some cases, an IL-2 variant MOD of this disclosure exhibits substantially reduced or no binding to IL-2Rα, thereby minimizing or substantially reducing the activation of Tregs by the IL-2 variant. In some cases, an IL-2 variant MOD of this disclosure has reduced affinity to IL-2Rβ and/or IL-2Rγ such that the IL-2 variant MOD exhibits an overall reduced affinity for IL-2R. In some cases, an IL-2 variant MOD of this disclosure exhibits both properties, i.e., it exhibits substantially reduced or no binding to IL-2Rα, and also has reduced affinity to IL-2Rβ and/or IL-2Rγ such that the IL-2 variant polypeptide exhibits an overall reduced affinity for IL-2R. MAPPs comprising such variants, including variants that substantially do not bind IL-2Rα and have reduced affinity to IL-2Rβ, have shown the ability to preferentially bind to and activate IL-2 receptors on T cells that contain the target TCR that is specific for the peptide epitope on the MAPP, and are thus less likely to deliver IL-2 to non-target T cells, i.e., T cells that do not contain a TCR that specifically binds the KRAS peptide epitope on the MAPP. That is, the binding of the IL-2 variant MOD to its co-MOD on the T cell is substantially driven by the binding of the MHC-epitope moiety rather than by the binding of the Il -2. Suitable IL-2 variant MODs thus include a polypeptide that comprises an amino acid sequence having at least 90%, at least 95%, at least 98%, at least 99% amino acid sequence identity to the amino acid sequence set forth in SEQ ID NO:95 for an IL-2. One such MOD is a variant IL-2 polypeptide comprises the sequence of wild-type IL-2 but with H16A and F42A substitutions, discussed below.

b. IL-2 and its Variants

As one non-limiting example, a MOD or variant MOD present in a MAPP is an IL-2 or variant IL-2 polypeptide. In some cases, a variant MOD present in a MAPP is a variant IL-2 polypeptide. Wild-type IL-2 binds to an IL-2 receptor (IL-2R). A wild-type IL-2 aa sequence can be as follows:

APTSSSTKKT QLQLEHLLLD LQMILNGINN YKNPKLTRML
TFKFYMPKKA TELKHLQCLE EELKPLEEVL NLAQSKNFHL
RPRDLISNIN VIVLELKGSE TTFMCEYADE TATIVEFLNR
WITFCQSIIS TLT (aa 21-153 of UniProt P60568,
SEQ ID NO: 95).

Wild-type IL2 binds to an IL2 receptor (IL2R) on the surface of a cell. An IL2 receptor is in some cases a heterotrimeric polypeptide comprising an alpha chain (IL-2Rα; also referred to as CD25), a beta chain (IL-2Rβ; also referred to as CD122) and a gamma chain (IL-2Rγ; also referred to as CD132). Amino acid sequences of human IL-2Rα, IL2Rβ, and IL-2Rγ can be as follows.

Human IL-2Rα:
(SEQ ID NO: 96)
ELCDDDPPE IPHATFKAMA YKEGTMLNCE CKRGERRIKS
GSLYMLCTGN SSHSSWDNQC QCTSSATRNT TKQVTPQPEE QKERKTTEMQ SPMQPVDQAS
LPGHCREPPP WENEATERIY HFVVGQMVYY QCVQGYRALH RGPAESVCKM THGKTRWTQP
QLICTGEMET SQFPGEEKPQ ASPEGRPESE TSCLVTTTDF QIQTEMAATM ETSIFTTEYQ
VAVAGCVFLL ISVLLLSGLT WQRRQRKSRR TI.
Human IL-2Rβ:
(SEQ ID NO: 97)
VNG TSQFTCFYNS RANISCVWSQ DGALQDTSCQ VHAWPDRRRW
NQTCELLPVS QASWACNLIL GAPDSQKLTT VDIVTLRVLC REGVRWRVMA IQDFKPFENL
RLMAPISLQV VHVETHRCNI SWEISQASHY FERHLEFEAR TLSPGHTWEE APLLTLKQKQ
EWICLETLTP DTQYEFQVRV KPLQGEFTTW SPWSQPLAFR TKPAALGKDT IPWLGHLLVG
LSGAFGFIIL VYLLINCRNT GPWLKKVLKC NTPDPSKFFS QLSSEHGGDV QKWLSSPFPS
SSFSPGGLAP EISPLEVLER DKVTQLLLQQ DKVPEPASLS SNHSLTSCFT NQGYFFFHLP
DALEIEACQV YFTYDPYSEE DPDEGVAGAP TGSSPQPLQP LSGEDDAYCT FPSRDDLLLF
SPSLLGGPSP PSTAPGGSGA GEERMPPSLQ ERVPRDWDPQ PLGPPTPGVP DLVDFQPPPE
LVLREAGEEV PDAGPREGVS FPWSRPPGQG EFRALNARLP LNTDAYLSLQ ELQGQDPTHL V.
Human IL-2Rγ:
(SEQ ID NO: 98)
LNTTILTP NGNEDTTADF FLTTMPTDSL SVSTLPLPEV QCFVENVEYM
NCTWNSSSEP QPTNLTLHYW YKNSDNDKVQ KCSHYLFSEE ITSGCQLQKK EIHLYQTFVV
QLQDPREPRR QATQMLKLQN LVIPWAPENL TLHKLSESQL ELNWNNRFLN HCLEHLVQYR
TDWDHSWTEQ SVDYRHKFSL PSVDGQKRYT FRVRSRENPL CGSAQHWSEW SHPIHWGSNT
SKENPFLFAL EAVVISVGSM GLIISLLCVY FWLERTMPRI PTLKNLEDLV TEYHGNFSAW
SGVSKGLAES LQPDYSERLC LVSEIPPKGG ALGEGPGASP CNQHSPYWAP PCYTLKPET.

In some cases, where a MAPP comprises a variant IL-2 polypeptide, a cognate co-MOD is an IL-2R comprising polypeptides comprising the aa sequences of any one of SEQ ID NO:96, SEQ ID NO:97, and SEQ ID NO:98.

In some cases, a variant IL-2 polypeptide exhibits reduced binding affinity to IL-2R, compared to the binding affinity of an IL-2 polypeptide comprising the aa sequence set forth in SEQ ID NO:95. For example, in some cases, a variant IL-2 polypeptide binds IL-2R with a binding affinity that is at least 10% less, at least 20% less, at least 30% less, at least 40% less, at least 50% less, at least 60% less, at least 70% less, at least 80% less, at least 90% less, at least 95% less, or more than 95% less, than the binding affinity of an IL-2 polypeptide comprising the aa sequence set forth in SEQ ID NO:95 for an IL-2R (e.g., an IL-2R comprising polypeptides comprising the aa sequence set forth in SEQ ID NOs:96-98), when assayed under the same conditions.

In some cases, a variant IL-2 polypeptide (e.g., a variant of SEQ ID NO:95) has a binding affinity to IL-2R (e.g., of SEQ ID NOs:96-98) that is from 100 nM to 100 μM. As another example, in some cases, a variant IL-2 polypeptide (e.g., a variant of SEQ ID NO:95) has a binding affinity for IL-2R (e.g., an IL-2R comprising polypeptides comprising the aa sequence set forth in SEQ ID NOs:96-98) that is from about 100 nM to about 200 nM, from about 200 nM to about 400 nM, from about 400 nM to about 600 nM, from about 600 nM to about 800 nM, from about 800 nM to about 1 μM, from about 1 M to about 5 μM, from about 5 μM to about 10 μM, from about 10 μM to about 20 μM, from about 20 M to about 40 μM, from about 40 μM to about 75 μM, or from about 75 μM to about 100 μM.

In some cases, a variant IL-2 polypeptide has a single aa substitution compared to the IL-2 aa sequence set forth in SEQ ID NO:95. In some cases, a variant IL-2 polypeptide has from 2 to 10 aa substitutions compared to the IL-2 aa sequence set forth in SEQ ID NO:95. In some cases, a variant IL-2 polypeptide has 2 aa substitutions compared to the IL-2 aa sequence set forth in SEQ ID NO:95. In some cases, a variant IL-2 polypeptide has 3 aa substitutions compared to the IL-2 aa sequence set forth in SEQ ID NO:95. In some cases, a variant IL-2 polypeptide has 4 aa substitutions compared to the IL-2 aa sequence set forth in SEQ ID NO:95. In some cases, a variant IL-2 polypeptide has 5 aa substitutions compared to the IL-2 aa sequence set forth in SEQ ID NO:95. In some cases, a variant IL-2 polypeptide has 6 or 7 aa substitutions compared to the IL-2 aa sequence set forth in SEQ ID NO:95. In some cases, a variant IL-2 polypeptide has 8, 9, or 10 aa substitutions compared to the IL-2 aa sequence set forth in SEQ ID NO:95.

Suitable variant IL-2 polypeptide sequences include polypeptide sequences comprising an aa sequence having at least 80% (e.g., at least 85%, at least 90%, at least 95%, at least 98%, at least 99%, or 100%) aa sequence identity to at least 80 (e.g., 90, 100, 110, 120, 130 or 133) contiguous aas of SEQ ID NO:95.

IL-2 variants include polypeptides having at least 90% (e.g., at least 95%, 98%, or 99%) aa sequence identity to at least 80 (e.g., at least 90, 100, 110, 120, or 130) contiguous aas of SEQ ID NO:95, wherein the aa at position 15 is an aa other than E. In one case, the position of H16 is substituted by Ala. In one case, the position of E15 is substituted by Ala.

IL-2 variants include polypeptides having at least 90% (e.g., at least 95%, 98%, or 99%) aa sequence identity to at least 80 (e.g., at least 90, 100, 110, 120, or 130) contiguous aas of SEQ ID NO:95, wherein the aa at position 16 is an aa other than H. In one case, the position of H16 is substituted by Asn, Cys, Gln, Met, Val, or Trp. In one case, the position of H16 is substituted by Ala. In another case, the position of H16 is substituted by Thr.

IL-2 variants include polypeptides having at least 90% (e.g., at least 95%, 98%, or 99%) aa sequence identity to at least 80 (e.g., at least 90, 100, 110, 120, or 130) contiguous aas of SEQ ID NO:95, wherein the aa at position 20 is an aa other than D. In one case, the position of D20 is substituted by Ala.

IL-2 variants include polypeptides having at least 90% (e.g., at least 95%, 98%, or 99%) aa sequence identity to at least 80 (e.g., at least 90, 100, 110, 120, or 130) contiguous aas of SEQ ID NO:95, wherein the aa at position 42 is an aa other than F. In one case, the position of F42 is substituted by Met, Pro, Ser, Thr, Trp, Tyr, Val, or His. In one case, the position of F42 is substituted by Ala.

IL-2 variants include polypeptides having at least 90% (e.g., at least 95%, 98%, or 99%) aa sequence identity to at least 80 (e.g., at least 90, 100, 110, 120, or 130) contiguous aas of SEQ ID NO:95, wherein the aa at position 45 is an aa other than Y. In one case, the position of Y45 is substituted by Ala.

IL-2 variants include polypeptides having at least 90% (e.g., at least 95%, 98%, or 99%) aa sequence identity to at least 80 (e.g., at least 90, 100, 110, 120, or 130) contiguous aas of SEQ ID NO:95, wherein the aa at position 88 is an aa other than N. In one case, the position of N88 is substituted by Ala. In another case, the position of N88 is substituted by Arg.

IL-2 variants include polypeptides having at least 90% (e.g., at least 95%, 98%, or 99%) aa sequence identity to at least 80 (e.g., at least 90, 100, 110, 120, or 130) contiguous aas of SEQ ID NO:95, wherein the aa at position 126 is an aa other than Q. In one case, the position of Q126 is substituted by Ala.

IL-2 variants include polypeptides having at least 90% (e.g., at least 95%, 98%, or 99%) aa sequence identity to at least 80 (e.g., at least 100, 110, 120, or 130) contiguous aas of SEQ ID NO:95, wherein the aa at position 16 is an aa other than H and the aa at position 42 is other than F. In one case, the position of H16 is substituted by Ala or Thr and the position of F42 is substituted by Ala or Thr. In one case, the position of H16 is substituted by Ala and the position of F42 is substituted by Ala (an H16A and F42A variant). In one case, the position of H16 is substituted by Thr and the position of F42 is substituted by Ala (an H16T and F42A variant).

An IL-2 variant may comprise an aa sequence having at least 80%, at least 85%, at least 90%, at least 95%, or at least 98% aa sequence identity to the sequence: APTSSSTKKT QLQLEX₁LLLD LQMILNGINN YKNPKLTRML TX₂KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:99), wherein position 16 and 42 are substituted as follows: X₁ is any aa other than His; and X₂ is any aa other than Phe. A second IL-2 variant comprises the substitutions X₁ is Ala and X₂ is Ala (an H16A and F42A variant) APTSSSTKKTQLQLEALLLDLQMILNGINNY-KNPKLTRMLTAKFYMPKKATELKHLQCLEEELKPLEEVLNLAQSKNFHLRPRDLISNINVIVLEL KGSETTFMCEYADETATIVEFLNRWITFCQSIISTLT (SEQ ID NO:91). A third IL-2 variant comprise the substitutions X₁ is Thr and X₂ is Ala (an H16T and F42A variant).

IL-2 variants include polypeptides having at least 90% (e.g., at least 95%, 98%, or 99%) aa sequence identity to at least 80 (e.g., at least 100, 110, 120, or 130) contiguous aas of SEQ ID NO:95, wherein the aa at position 20 is an aa other than D and the aa at position 42 is other than F. In one case, the position of D20 is substituted by Ala and the position of F42 is substituted by Ala (D20A, F42A substitutions).

IL-2 variants include polypeptides having at least 90% (e.g., at least 95%, 98%, or 99%) aa sequence identity to at least 80 (e.g., at least 100, 110, 120, or 130) contiguous aas of SEQ ID NO:95, wherein the aa at position 15 is other than E, the aa at position 20 is an aa other than D, and the aa at position 42 is other than F. In one case, the position of E15 is substituted by Ala, the position of D20 is substituted by Ala and the position of F42 is substituted by Ala (E15A, D20A, F42A substitutions).

IL-2 variants include polypeptides having at least 90% (e.g., at least 95%, 98%, or 99%) aa sequence identity to at least 80 (e.g., at least 100, 110, 120, or 130) contiguous aas of SEQ ID NO:95, wherein the aa at position 16 is other than H, the aa at position 20 is an aa other than D, and the aa at position 42 is other than F. In one case, the position of H16 is substituted by Ala, the position of D20 is substituted by Ala and the position of F42 is substituted by Ala (an H16A, D20A, F42A substitution). In another case, the position H16 is substituted by Thr, the position of D20 is substituted by Ala and the position of F42 is substituted by Ala (H16T, D20A, F42A substitutions).

IL-2 variants include polypeptides having at least 90% (e.g., at least 95%, 98%, or 99%) aa sequence identity to at least 80 (e.g., at least 100, 110, 120, or 130) contiguous aas of SEQ ID NO:95, wherein the aa at position 16 is other than H, the aa at position 42 is other than F, and the aa at position 88 is other than N. In one case, the position of H16 is substituted by Ala or Thr, the position of F42 is substituted by Ala, and the position of N88 is substituted by Arg (H16A, F42A, N88R substitution or H16T, F42A, N88R substitutions).

IL-2 variants include polypeptides having at least 90% (e.g., at least 95%, 98%, or 99%) aa sequence identity to at least 80 (e.g., at least 100, 110, 120, or 130) contiguous aas of SEQ ID NO:95, wherein the aa at position 16 is other than H, the aa at position 42 is other than F, and the aa at position 126 is other than Q. Such IL-2 variants include those wherein, the position of H16 is substituted by Ala or Thr, the position of F42 is substituted by Ala, and the position of Q126 is substituted by Ala (an H16A, F42A, Q126A substitution or an H16T, F42A, Q126A substitutions).

IL-2 variants include polypeptides having at least 90% (e.g., at least 95%, 98%, or 99%) aa sequence identity to at least 80 (e.g., at least 100, 110, 120, or 130) contiguous aas of SEQ ID NO:95, wherein the aa at position 20 is other than D, the aa at position 42 is other than F, and the aa at position 126 is other than Q. In one case, the position D20 is substituted by Ala, the position of F42 is substituted by Ala, and the position of Q126 is substituted by Ala (D20A, F42A, Q126A substitutions).

IL-2 variants include polypeptides having at least 90% (e.g., at least 95%, 98%, or 99%) aa sequence identity to at least 80 (e.g., at least 100, 110, 120, or 130) contiguous aas of SEQ ID NO:95, wherein the aa at position 20 is other than D, the aa at position 42 is other than F, and the aa at position 45 is other than Y. In one case, the position D20 is substituted by Ala, the position of F42 is substituted by Ala, and the position of Y45 is substituted by Ala (D20A, F42A, and Y45A substitutions).

IL-2 variants include polypeptides having at least 90% (e.g., at least 95%, 98%, or 99%) aa sequence identity to at least 80 (e.g., at least 100, 110, 120, or 130) contiguous aas of SEQ ID NO:95, wherein the aa at position 16 is other than H, the aa at position 20 is other than D, the aa at position 42 is other than F, and the aa at position 45 is other than Y. Such IL-2 variants include those in which the position of H16 is substituted by Ala or Thr, the position D20 is substituted by Ala, the position of F42 is substituted by Ala, and the position of Y45 is substituted by Ala (H16A, D20A, F42A, and Y45A substitution, or H16T, D20A, F42A, and Y45A substitution,).

IL-2 variants include polypeptides having at least 90% (e.g., at least 95%, 98%, or 99%) aa sequence identity to at least 80 (e.g., at least 100, 110, 120, or 130) contiguous aas of SEQ ID NO:95, wherein the aa at position 20 is other than D, the aa at position 42 is other than F, the aa at position 45 is other than Y, and the aa at position 126 is other than Q. In one case, the position D20 is substituted by Ala, the position of F42 is substituted by Ala, the position of Y45 is substituted by Ala, and the position of Q126 is substituted by Ala (D20A, F42A, Y45A, Q126A substitutions).

IL-2 variants include polypeptides having at least 90% (e.g., at least 95%, 98%, or 99%) aa sequence identity to at least 80 (e.g., at least 100, 110, 120, or 130) contiguous aas of SEQ ID NO:95, wherein the aa at position 16 is other than H, the aa at position 20 is other than D, the aa at position 42 is other than F, the aa at position 45 is other than Y, and the aa at position 126 is other than Q. In one case, the position of H16 is substituted by Ala or Thr, the position D20 is substituted by Ala, the position of F42 is substituted by Ala, the position of Y45 is substituted by Ala, and the position of Q126 is substituted by Ala (H16A, D20A, F42A, Y45A, Q126A substitutions or H16T, D20A, F42A, Y45A, Q126A substitutions).

6. Linkers

As noted above, a MAPP of the present disclosure can include a linker sequence (aa, peptide, or polypeptide sequence) or “linker” interposed between any two elements of a MAPP, e.g., an epitope and an MHC polypeptide; between an MHC polypeptide and an Ig Fc polypeptide; between a first MHC polypeptide and a second MHC polypeptide; etc. Although termed “linkers,” sequences employed for linkers may also be placed at the N- and/or C-terminus of a MAPP polypeptide to stabilize the MAPP polypeptide or protect it from proteolytic degradation.

Suitable polypeptide linkers (also referred to as “spacers”) can be readily selected and can be of any of a number of suitable lengths, such as from 1 aa to 35 aa, from 3 aa to 20 aa, from 3 aa to 20 aa, from 2 aa to 15 aa, from 3 aa to 12 aa, from 4 aa to 10 aa, from 4 aa to 35 aa, from 5 aa to 35 aa, from 5 aa to 20 aa, from 6 aa to 25 aa, or from 7 aa to 35 aa. In embodiments, a suitable linker can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 aa in length. A linker may have a length of from 25 aa to 50 aa, e.g., from 20-35, from 25 to 30, from 30 to 35, from 35 to 40, from 40 to 45, or from 45 to 50 aa in length. A suitable linker can be 45, 46, 47, 48, 49, or 50 aa in length.

Polypeptide linkers in the MAPP may include, for example, polypeptides that comprise, consist essentially of, or consists of: i) Gly and Ser; ii) Ala and Ser; iii) Gly, Ala, and Ser; iv) Gly, Ser, and Cys (e.g, a single Cys residue); v) Ala, Ser, and Cys (e.g., a single Cys residue); and vi) Gly, Ala, Ser, and Cys (e.g., a single Cys residue). Exemplary linkers may comprise glycine polymers, glycine-serine polymers, glycine-alanine polymers; alanine-serine polymers, (including, for example polymers comprising the sequences GA, AG, AS, SA, GS, GSGGS (SEQ ID NO:101) or GGGS (SEQ ID NO:102), any of which may be repeated from 1 to 10 times (e.g., repeated 1, 2, 3, 4, 5, 6, 7, 8, 9, or 10 times); and other flexible linkers known in the art. Glycine and glycine-serine polymers can both be used as both Gly and Ser are relatively unstructured and therefore can serve as a neutral tether between components. Glycine polymers access significantly more phi-psi space than even alanine, and are much less restricted than residues with longer side chains (see Scheraga, Rev. Computational Chem. 11173-142 (1992)). Exemplary linkers may also comprise an aa sequence comprising, but not limited to, GGSG (SEQ ID NO:103), GGSGG (SEQ ID NO:104), GSGSG (SEQ ID NO:105), GSGGG (SEQ ID NO:106), GGGSG (SEQ ID NO:107), GSSSG (SEQ ID NO:108), any of which may be repeated from 1 to 10 times (e.g., repeated 1, 2, 3, 4, 5, 6, 7, 8, 9, or 10 times), or combinations thereof, and the like. Linkers can also comprise the sequence Gly(Ser)₄ (SEQ ID NO:109) or (Gly)₄Ser (SEQ ID NO:110), either of which may be repeated from 1 to 10 times (e.g., 1, 2, 3, 4, 5, 6, 7, 8, 9, or 10 times). In one embodiment the linker comprises the aa sequence AAAGG (SEQ ID NO:111), which may be repeated from 1 to 10 times.

In some cases, a linker polypeptide, present in a polypeptide of a MAPP (e.g., in a 1st or 2nd presenting complex sequence) includes a cysteine residue that can form a disulfide bond with a cysteine residue present in another polypeptide of the MAPP. In some cases, for example, the linker comprises the aa sequence GCGGS(G₄S) (SEQ ID NO:112) where the G₄S unit may be repeated from 1 to 10 times (e.g., repeated 1, 2, 3, 4, 5, 6, 7, 8, 9, or 10 times), GCGASGGGGSGGGGS (SEQ ID NO:113), the sequence GCGGSGGGGSGGGGSGGGGS (SEQ ID NO:114) or the sequence GCGGSGGGGSGGGGS (SEQ ID NO:115).

Rigid polypeptide linkers comprise a sequence of amino acids that effectively separates protein domains by maintaining a substantially fixed distance/spatial separation between the domains, thereby reducing or substantially eliminating unfavorable interactions between such domains. Rigid polypeptide linkers thus may be employed where it is desired to minimize the interaction between the domains of the MAPP, in particular the interactions between MOD aa sequences (e.g., IL-2) and other aas sequences of the MAPP. Rigid peptide linkers include peptide linkers rich in proline, and peptide linkers having an inflexible helical structure, such as an α-helical structure. Examples of rigid peptide linkers include, e.g., (EAAAK) (SEQ ID NO:205), A(EAAAK)A (SEQ ID NO:206), A(EAAAK)ALEA(EAAAK)A (SEQ ID NO:207), (Lys-Pro), (Glu-Pro), (Thr-Pro-Arg), and (Ala-Pro) where the bracketed sequences may be repeated or appear from 1 to 20 (e.g., n is 1, 2, 3, 4, 5, 6, 7, 8, 9, 10, 11, 12, 13, 14, 15, 16, 17, 18, 19, or 20). Non-limiting examples of suitable rigid linkers comprising EAAAK (SEQ ID NO:208) include EAAAK (SEQ ID NO:208), (EAAAK)2 (SEQ ID NO:209), (EAAAK)3 (SEQ ID NO:210), A(EAAAK)ALEA(EAAAK)A where the EAAAK sequence may be repeated or appear 1-4 times (SEQ ID NO:211), and AEAAAKEAAAKA (SEQ ID NO:212). Non-limiting examples of suitable rigid linkers comprising (AP)n include APAP (SEQ ID NO:213; also referred to herein as “(AP)2”); APAPAPAP (SEQ ID NO:214; also referred to herein as “(AP)4”); APAPAPAPAPAP (SEQ ID NO:215; also referred to herein as “(AP)6”); APAPAPAPAPAPAPAP (SEQ ID NO:216; also referred to herein as “(AP)8”); and APAPAPAPAPAPAPAPAPAP (SEQ ID NO:217; also referred to herein as “(AP)10”). Non-limiting examples of suitable rigid linkers comprising (KP)n include KPKP (SEQ ID NO:218; also referred to herein as “(KP)2”); KPKPKPKP (SEQ ID NO:219; also referred to herein as “(KP)4”); KPKPKPKPKPKP (SEQ ID NO:220; also referred to herein as “(KP)6”); KPKPKPKPKPKPKPKP (SEQ ID NO:221; also referred to herein as “(KP)8”); and KPKPKPKPKPKPKPKPKPKP (SEQ ID NO:222; also referred to herein as “(KP)10”). Non-limiting examples of suitable rigid linkers comprising (EP)n include EPEP (SEQ ID NO:223; also referred to herein as “(EP)2”); EPEPEPEP (SEQ ID NO:224; also referred to herein as “(EP)4”); EPEPEPEPEPEP (SEQ ID NO:225; also referred to herein as “(EP)6”); EPEPEPEPEPEPEPEP (SEQ ID NO:226; also referred to herein as “(EP)8”); and EPEPEPEPEPEPEPEPEPEP (SEQ ID NO:227; also referred to herein as “(EP)10”).

7. Epitopes

As indicated above, a variety of KRAS epitope presenting molecules, in particular peptides, (“epitope peptides,” “KRAS epitopes,” or simply “epitopes” for the purpose of this disclosure) including peptide epitopes, phosphopeptide epitopes, and/or lipopeptide epitopes may be present in a MAPP or higher order complexes of MAPPs (such as duplex MAPPs of the present disclosure), and presentable to a TCR on the surface of a T cell. An epitope peptide can have a length of from about 4 aas to about 25 aas (aa), e.g., the epitope can have a length of from 4 aa to 10 aa, from 10 aa to 15 aa, from 15 aa to 20 aa, or from 20 aa to 25 aa. For example, an epitope present in a MAPP of the present disclosure can have a length of 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa. In some cases, an epitopepeptide present in a MAPP of the present disclosure has a length of from 5 aa to 10 aa, e.g., 5 aa, 6 aa, 7 aa, 8 aa, 9 aa, or 10 aa.

An epitope peptide present in a MAPP (e.g., a duplex MAPP) with class I MHC presenting sequences or complexes may be bound in an epitope specific manner by a T cell (i.e., the epitope is specifically bound by an epitope-specific T cell). An epitope-specific T cell binds an epitope peptide having a reference aa sequence, but does not substantially bind an epitope that differs from the reference aa sequence. For example, an epitope-specific T cell binds an epitopepeptide having a reference aa sequence, may bind an epitope that differs from the reference aa sequence, if at all, with an affinity that is less than 10⁻⁶ M, less than 10⁻⁵ M, or less than 10⁻⁴ M. An epitope-specific T cell can bind an epitope peptide for which it is specific with an affinity of at least 10⁻⁷ M, at least 10⁻⁸ M, at least 10⁻⁹ M, or at least 10⁻¹⁰ M.

a. KRAS Epitope Peptides in MAPPs with Class I MHC Presenting Sequences and Presenting Complexes

An epitope present in a MAPP may be bound in an epitope-specific manner by a T cell (i.e., the epitope is specifically bound by an epitope-specific T cell whose TCR recognizes the peptide). An epitope-specific T cell binds an epitope having a reference aa sequence in the context of a specific MHC-H allele polypeptide/β2M complex, but does not substantially bind an epitope that differs from the reference aa sequence presented in the same context. For example, an epitope-specific T cell may bind an epitope in the context of a specific MHC-H allele polypeptide/β2M complex having a reference aa sequence, and may bind an epitope that differs from the reference aa sequence presented in the same context, if at all, with an affinity that is less than 10⁻⁶ M, less than 10⁻⁵ M, or less than 10⁻⁴ M. An epitope-specific T cell may bind an epitope (e.g., a peptide presenting an epitope of interest) for which it is specific with an affinity of at least 10⁻⁷ M, at least 10⁻⁸ M, at least 10⁻⁹ M, or at least 10⁻¹⁰ M.

In some cases, the KRAS epitope present in a MAPP presents an epitope-specific to an HLA-A, -B, -C, -E, -F or -G allele. In an embodiment, the peptide epitope present in a MAPP presents an epitope restricted to HLA-A*0101, A*0201, A*0203, A*0301, A*1101, A*2301, A*2402, A*2407, A*3303, A*3401, and/or A*5801. In an embodiment, the peptide epitope present in a MAPP presents an epitope restricted to HLA-B*0702, B*0801, B*1502, B*2705, B*3802, B*3901, B*3902, B*4001, B*4601, and/or B*5301. In an embodiment, the peptide epitope present in a MAPP presents an epitope restricted to HLA-C*0102, C*0303, C*0304, C*0401, C*0602, C*0701, C*702, C*0801, and/or C*1502.

The present disclosure provides MAPPs comprising a KRAS peptide that, when bound to major histocompatibility complex (MHC) polypeptides, presents an KRAS epitope to a T-cell receptor (TCR). As used herein, the term “KRAS peptide” means a peptide having a length of at least 4 amino acids, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 amino acids, from 6 to 18 amino acids, from 8 to 15 amino acids, from 8 to 12 amino acids, from 5 to 10 amino acids, from 10 to 20 amino acids, and from 15 to 25 amino acids in length) that presents a KRAS epitope to a TCR when the KRAS peptide is bound to an MHC complex. As used herein, the term “KRAS epitope” means an epitope found on a KRAS protein. As used herein, the terms “KRAS” and “KRAS protein” are synonymous and mean a protein having an amino acid sequence present in one of the following: (i) a KRAS4A polypeptide; (ii) a KRAS4B; and (iii) variants of (i) and (ii) that occur in human cancers, including, e.g., mutated forms. As used herein, the term “KRAS polypeptide” means a polypeptide having a sequence of amino acids found in all or a part of a KRAS protein, or where specified, a polypeptide having at least 80% (e.g., at least 90%, 95%, 98% or more) amino acid sequence identity to a sequence of amino acids found in all or a part of a KRAS protein. KRAS epitopes of interest include peptides that have sequence variations (e.g., substitutions, deletions, insertions etc.) not found in wild type RAS proteins and that have been associated neoplastic behavior when RAS/KRAS proteins bearing those sequence variations are introduced into mammalian cells. The peptides may include posttranslational modifications including phosphorylation, glycosylation, and/or lipidation (e.g., palmitoylation, glycosylation, and/or farnesylation).

KRAS (also known as “KRAS proto-oncogene, GTPase,” Kirsten rat sarcoma viral oncogene homolog,” and “K-Ras P21 protein”) is a GTPase that controls cell proliferation. When mutated, KRAS can fail to control cell proliferation, leading to cancer.

A wild-type (normal; non-cancer-associated) KRAS polypeptide can have the following amino acid sequence: MTEYKLVVVG A [symbol] VGKSALT IQLIQNHFVD EYDP [symbol] EDSY RKQVVIDG [symbol] T CLWDILDTAG [symbol] EEYSAMRDQ YMRTGEGFLC VFAINNTKSF EDIHHYREQI KRVKDSEDVP MVLVGN [symbol] CDL PSRTVDTKQA QDLARSYGIP FIETS [symbol] KTRQ GVDDAFYTLV REIRKHKEKM SKDGKKKKKK SKTKCVIM (SEQ ID NO:116).

A wild-type (normal; non-cancer-associated) KRAS polypeptide can have the following amino acid sequence: MTEYKLVVVG AGGVGKSALT IQLIQNHFVD EYDPTIEDSY RKQVVIDGET CLLDILDTAG QEEYSAMRDQ YMRTGEGFLC VFAINNTKSF EDIHHYREQI KRVKDSEDVP MVLVGNKCDL PSRTVDTKQA QDLARSYGIP FIETSAKTRQ RVEDAFYTLV REIRQYRLKK ISKEEKTPGC VKIKKCIIM (SEQ ID NO:117).

Mutated forms of KRAS are associated with certain cancers; and at least a portion of the mutated form of KRAS is present on the surface of certain cancer cells. See, e.g., Prior et al. (2012) Cancer Res. 72:2457; and Warren and Holt (2010) Human Immunology 71:245. In SEQ ID NO:116 and SEQ ID NO:117, amino acids G12, G13, T35, 136, E49, Q61, K127, and A156 are in bold and underlined; substitutions of one or more of these residues can be present in a cancer-associated form of a KRAS polypeptide. A cancer-associated KRAS polypeptide can include one or more of: i) a substitution of G12 (e.g. G12C, G12V, G125, G12A, G12R, G12F, or G12D); ii) a substitution of G13 (e.g. G13C, G13D, G13R, G13V, G13S, or G13A); iii) a substitution of T35 (e.g., T35I); iv) a substitution of 136 (e.g., I36L or I36M); v) a substitution of E49 (e.g., E49K); vi) a substitution of Q61 (e.g. Q61H, Q61R, Q61P, Q61E, Q61K, Q61L, or Q61K); vii) a substitution of K117 (e.g., K117N); and viii) a substitution of A146 (e.g. A146T or A146V); where the amino acid numbering is as set out in SEQ ID NO:116 and SEQ ID NO:117. See, e.g., U.S. 2019/0194192. Peptides bearing such substitutions may be incorporated into MAPPS.

For example, a cancer-associated, mutated form of a KRAS polypeptide, or peptides that acts as the presented epitope in a MAPP, can have one or more amino acid substitutions compared to the amino acid sequence set forth in SEQ ID NO:116 or SEQ ID NO:117. In some cases, a cancer-associated, mutated form of a KRAS polypeptide or peptide epitope has only a single amino acid substitution compared to the amino acid sequence set forth in SEQ ID NO:116 or SEQ ID NO:117. In some cases, a cancer-associated, mutated form of a KRAS polypeptide or peptide epitope has only two amino acid substitutions compared to the amino acid sequence set forth in SEQ ID NO:116 or SEQ ID NO:117. In some cases, a cancer-associated, mutated form of a KRAS polypeptide or peptide epitope has only three amino acid substitutions compared to the amino acid sequence set forth in SEQ ID NO:116 or SEQ ID NO:117. In some cases, a cancer-associated, mutated form of a KRAS polypeptide or peptide epitope has only four amino acid substitutions compared to the amino acid sequence set forth in SEQ ID NO:116 or SEQ ID NO:117. In some cases, a cancer-associated, mutated form of a KRAS polypeptide or peptide epitope has only five amino acid substitutions compared to the amino acid sequence set forth in SEQ ID NO:116 or SEQ ID NO:117.

For example, KRAS(G12D) (a KRAS polypeptide having a G-to-D substitution at amino acid position 12, based on the amino acid numbering set forth in SEQ ID NO:116) is associated with pancreatic ductal adenocarcinoma (PDAC). KRAS(G12V) (a KRAS polypeptide having a G-to-V substitution at amino acid position 12, based on the amino acid numbering set forth in SEQ ID NO:116 or SEQ ID NO:117) is also associated with pancreatic cancer. KRAS(G12R) (a KRAS polypeptide having a G-to-R substitution at amino acid position 12, based on the amino acid numbering set forth in SEQ ID NO:116 or SEQ ID NO:117) is also associated with pancreatic cancer. See, e.g., Waters and Der (2018) Cold Spring Harb. Perspect. Med. 8:(9). pii: a031435. doi: 10.1101/cshperspect.a031435. As another example, KRAS(G12C) (a KRAS polypeptide having a G-to-C substitution at amino acid position 12, based on the amino acid numbering set forth in SEQ ID NO:116 or SEQ ID NO:117) is associated with lung cancer, e.g., non-small cell lung cancer. See, e.g., Román et al. (2018) Mol. Cancer 17:33. Other mutated forms of KRAS (e.g., G12A; G12C; G12D; G12R; G12S; G12V; G13A; G13C; G13D; G13R; G13S; G13V) are associated with various cancers; where such cancers include, e.g., bile duct carcinoma, gall bladder carcinoma, adenocarcinoma, rectal adenocarcinoma, endometrial carcinoma, hematopoietic neoplasms, and lung cancer. See, e.g., Prior et al. (20120 Cancer Res. 72:2457.

As another example, a cancer-associated, mutated form of a KRAS polypeptide can have an amino acid substitution at amino acid 61 of a KRAS polypeptide (e.g., a KRAS polypeptide having the amino acid sequence set forth in SEQ ID NO:116 or SEQ ID NO:117). For example, a cancer-associated, mutated form of a KRAS polypeptide can have an amino acid substitution such as Q61H, Q61L, Q61E, Q61R, or Q61K.

As discussed above, MAPPs of the present disclosure comprises a KRAS peptide that is typically at least about 4 amino acids in length, and presents a KRAS epitope to a T cell when in an MHC/peptide complex (e.g., an HLA/peptide complex). The KRAS epitope may include one or more aa substitutions associated with a benign neoplasm or cancer (malignant neoplasm).

A KRAS epitope present in a MAPP of the present disclosure is a peptide specifically bound by a T-cell, i.e., the epitope is specifically bound by an epitope-specific T cell. An epitope-specific T cell binds an epitope having a reference amino acid sequence, but does not substantially bind an epitope that differs from the reference amino acid sequence. For example, an epitope-specific T cell binds an epitope having a reference amino acid sequence, and binds an epitope that differs from the reference amino acid sequence, if at all, with an affinity that is less than 10⁻⁶ M, less than 10⁻⁵ M, or less than 10⁻⁴ M. An epitope-specific T cell can bind an epitope for which it is specific with an affinity of at least 10⁻⁷ M, at least 10⁻⁸ M, at least 10⁻⁹ M, or at least 10⁻¹⁰ M.

In some cases, a suitable KRAS peptide is a peptide of at least 4 amino acids in length, (e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 amino acids, from 6 to 18 amino acids, from 8 to 15 amino acids, from 8 to 12 amino acids, from 9-10 amino acids, from 5 to 10 amino acids, from 10 to 20 amino acids, and from 15 to 25 amino acids in length) of a KRAS polypeptide comprising an amino acid sequence having at least 80%, at least 85%, at least 90%, at least 95%, at least 98%, or at least 99%, amino acid sequence identity to a portion of the KRAS sequence provided in SEQ ID NO:116, where the KRAS polypeptide comprises one or more (e.g., 1, 2, 3, 4, or 5) amino acid substitutions compared to the amino acid sequence forth in SEQ ID NO:116. The one or more amino acid substitutions can include substitutions associated with cancer; e.g., substitutions that are found in a KRAS polypeptide in a cancer cell.

In some cases, a suitable KRAS peptide is a peptide of at least 4 amino acids in length, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 amino acids, from 6 to 18 amino acids, from 8 to 15 amino acids, from 8 to 12 amino acids, from 9-10 amino acids, from 5 to 10 amino acids, from 10 to 20 amino acids, and from 15 to 25 amino acids in length) of a KRAS polypeptide comprising an amino acid sequence having at least 80%, at least 85%, at least 90%, at least 95%, at least 98%, or at least 99%, amino acid sequence identity to a portion of the KRAS sequence provided in SEQ ID NO:117, where the KRAS polypeptide comprises one or more (e.g., 1, 2, 3, 4, or 5) amino acid substitutions compared to the amino acid sequence forth in SEQ ID NO:116. The one or more amino acid substitutions can include substitutions associated with cancer; e.g., substitutions that are found in a KRAS polypeptide in a cancer cell.

In some cases, a suitable KRAS peptide is a peptide of at least 4 amino acids in length, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 amino acids, from 6 to 18 amino acids, from 8 to 15 amino acids, from 8 to 12 amino acids, from 9-10 amino acids, from 5 to 10 amino acids, from 10 to 20 amino acids, and from 15 to 25 amino acids in length) of a KRAS polypeptide comprising an amino acid sequence having at least 80%, at least 85%, at least 90%, at least 95%, at least 98%, or at least 99%, amino acid sequence identity to a portion of the following KRAS amino acid sequence: MTEY(X1)L(X2)(X3)(X4)GA(X5)(X6)VGKSALT IQLIQNHFVD EYDPTIEDSY RKQVVIDGET CLWDILDTAG QEEYSAMRDQ YMRTGEGFLC VFAINNTKSF EDIHHYREQI KRVKDSEDVP MVLVGNKCDL PSRTVDTKQA QDLARSYGIP FIETSAKTRQ GVDDAFYTLV REIRKHKEKM SKDGKKKKKK SKTKCVIM (SEQ ID NO:118), where X1 is Lys, Phe, or Leu; X2 is Val or Leu; X3 is Val or Thr; X4 is Val or Thr; X5 is Gly, Asp, Cys, Val, or Ser; and X6 is Gly, Cys, or Asp; where one or both of X5 and X6 is not a Cys.

Non-limiting examples of suitable KRAS peptides for incorporation into MAPPs include: VVGADGVGK (SEQ ID NO:119), VVGACGVGK (SEQ ID NO:120), VVGAVGVGK (SEQ ID NO:121), VVVGADGVGK (SEQ ID NO:122), VVVGAVGVGK (SEQ ID NO:123), VVVGACGVGK (SEQ ID NO:124), VTGADGVGK (SEQ ID NO:125), VTGAVGVGK (SEQ ID NO:126), VTGACGVGK (SEQ ID NO:127), VTVGADGVGK (SEQ ID NO:128), VTVGAVGVGK (SEQ ID NO:129), and VTVGACGVGK (SEQ ID NO:130); where the KRAS peptide has a length of 9 amino acids or 10 amino acids.

Additional non-limiting examples of suitable KRAS peptides for incorporation into MAPPs include: VVVGAGDVGK (SEQ ID NO:131); VVGAGDVGK (SEQ ID NO:132); VVVGARGVGK (SEQ ID NO:133); and VVGARGVGK (SEQ ID NO:134); where the KRAS peptide has a length of 9 amino acids or 10 amino acids.

Non-limiting examples of suitable KRAS peptides for incorporation into MAPPs also include: LVVVGADGV (SEQ ID NO:135), LVVVGAVGV (SEQ ID NO:136), LVVVGACGV (SEQ ID NO:137), KLVVVGADGV (SEQ ID NO:138), KLVVVGAVGV (SEQ ID NO:139), KLVVVGACGV (SEQ ID NO:140), LLVVGADGV (SEQ ID NO:141), LLVVGAVGV (SEQ ID NO:142), LLVVGACGV (SEQ ID NO:143), FLVVVGADGV (SEQ ID NO:144), FLVVVGAVGV (SEQ ID NO:145), and FLVVVGACGV (SEQ ID NO:146); where the KRAS peptide has a length of 9 amino acids or 10 amino acids.

Additional non-limiting examples of suitable KRAS peptides for incorporation into MAPPs include: KLVVVGAGDV (SEQ ID NO:161); and KLVVVGARGV (SEQ ID NO:162); where the KRAS peptide has a length of 9 amino acids or 10 amino acids.

Another group of KRAS peptides suitable as epitopes includes KLVVGADGV (SEQ ID NO:163), KLVVGAVGV (SEQ ID NO:164), KLVVVAVGV (SEQ ID NO:165), and KLVVVADGV (SEQ ID NO:166).

Other non-limiting examples of suitable KRAS peptides for incorporation into MAPPs include:

(SEQ ID NO: 147)
GAGDVGKSAL;
(SEQ ID NO: 148)
AGDVGKSAL;
(SEQ ID NO: 149)
DVGKSALTI;
(SEQ ID NO: 150)
GAVGVGKSAL;
(SEQ ID NO: 151)
AVGVGKSAL;
(SEQ ID NO: 152)
YKLVVVGAV;
(SEQ ID NO: 153)
ARGVGKSAL;
(SEQ ID NO: 154)
GARGVGKSAL;
(SEQ ID NO: 155)
EYKLVVVGAR;
(SEQ ID NO: 156)
RGVGKSALTI;
(SEQ ID NO: 157)
LVVVGARGV;
(SEQ ID NO: 160)
GADGVGKSAL;
(SEQ ID NO: 158)
ACGVGKSAL;
and
(SEQ ID NO: 159)
GACGVGKSAL.

In some cases, a MAPP of the present disclosure modulates the activity of a T cell that comprises a TCR that is specific for a G12V form of a KRAS polypeptide, as described above. In such cases, the KRAS peptide present in a MAPP of the present disclosure can comprise, for example, one of the following amino acid sequences: VVGAVGVGK (SEQ ID NO:121), VVVGAVGVGK (SEQ ID NO:123), VGAVGVGKS (SEQ ID NO:167), VGAVGVGKSA (SEQ ID NO:168), AVGVGKSAL (SEQ ID NO:151), AVGVGKSALT (SEQ ID NO:169), GAVGVGKSAL (SEQ ID NO:150), GAVGVGKSA (SEQ ID NO:170), LVVVGAVGVG (SEQ ID NO:171), LVVVGAVGV (SEQ ID NO:136), KLVVVGAVGV (SEQ ID NO:139), and KLVVVGAVG (SEQ ID NO:172); where the KRAS peptide has a length of 9 amino acids or 10 amino acids.

In some cases, the KRAS peptide present in a MAPP of the present disclosure presents an epitope specific to an HLA-A, -B, -C, -E, -F, or -G allele. In an embodiment, the KRAS peptide present in a MAPP presents an epitope restricted to HLA-A*0101, A*0201, A*0203, A*0301, A*1101, A*2301, A*2402, A*2407, A*3101, A*3303, A*3401, and/or A*6801. In an embodiment, the KRAS epitope peptide present in a MAPP presents an epitope restricted to HLA-B*0702, B*0801, B*1502, B*2705, B*3802, B*3802, B*3901, B*3902, B*4001, B*4601, B*5101, and/or B*5301. In an embodiment, the KRAS epitope peptide present in a MAPP presents an epitope restricted to C*0102, C*0303, C*0304, C*0401, C*0602, C*0701, C*702, C*0801, and/or C*1502.

As non-limiting examples, the KRAS peptides VVGADGVGK (SEQ ID NO:119), VVGACGVGK (SEQ ID NO:120), VVGAVGVGK (SEQ ID NO:121), VVVGADGVGK (SEQ ID NO:122), VVVGAVGVGK (SEQ ID NO:123), VVVGACGVGK (SEQ ID NO:124), VTGADGVGK (SEQ ID NO:125), VTGAVGVGK (SEQ ID NO:126), VTGACGVGK (SEQ ID NO:127), VTVGADGVGK (SEQ ID NO:128), VTVGAVGVGK (SEQ ID NO:129), VTVGACGVGK (SEQ ID NO:130), VVVGAGDVGK (SEQ ID NO:131), VVGAGDVGK (SEQ ID NO:132), VVVGARGVGK (SEQ ID NO:133), and VVGARGVGK (SEQ ID NO:134) present an epitope when bound to an HLA complex comprising a β2M polypeptide and an A*1101 HLA-A heavy chain. Such peptides may also be presented in complex with an HLA complex comprising a β2M polypeptide and an A*6801 HLA-A heavy chain.

As non-limiting examples, the KRAS peptides LVVVGADGV (SEQ ID NO:135), LVVVGAVGV (SEQ ID NO:136), LVVVGACGV (SEQ ID NO:137), KLVVVGADGV (SEQ ID NO:138), KLVVVGAVGV (SEQ ID NO:139), KLVVVGACGV (SEQ ID NO:140), LLVVGADGV (SEQ ID NO:141), LLVVGAVGV (SEQ ID NO:142), LLVVGACGV (SEQ ID NO:143), FLVVVGADGV (SEQ ID NO:144), FLVVVGAVGV (SEQ ID NO:145), and FLVVVGACGV (SEQ ID NO:146) present an epitope when bound to an HLA complex comprising a β2M polypeptide and an A*0201 HLA-A heavy chain.

As additional examples, the following KRAS peptides can present an epitope when bound to an HLA complex comprising a β2M polypeptide and an HLA-A heavy chain as follows: GAGDVGKSAL (SEQ ID NO:147), which can present an epitope when bound to an HLA complex comprising a β2M polypeptide and a B*3801 HLA-A heavy chain; AGDVGKSAL (SEQ ID NO:148), which can present an epitope when bound to an HLA complex comprising a β2M polypeptide and a B*0702, a B*3801, or a B*3901 HLA-A heavy chain; DVGKSALTI (SEQ ID NO:149), which can present an epitope when bound to an HLA complex comprising a β2M polypeptide and a B*5101 HLA-A heavy chain; GAVGVGKSAL (SEQ ID NO:150), which can present an epitope when bound to an HLA complex comprising a β2M polypeptide and a B*0702 or a B*3801 HLA-A heavy chain; AVGVGKSAL (SEQ ID NO:151), which can present an epitope when bound to an HLA complex comprising a β2M polypeptide and a B*0702 HLA-A heavy chain; YKLVVVGAV (SEQ ID NO:152), which can present an epitope when bound to an HLA complex comprising a β2M polypeptide and an A*0203 or a B*3902 HLA-A heavy chain; ARGVGKSAL (SEQ ID NO:153), which can present an epitope when bound to an HLA complex comprising a β2M polypeptide and a B*0702, a B*2705, or a B*3901 HLA-A heavy chain; GARGVGKSAL (SEQ ID NO:154), which can present an epitope when bound to an HLA complex comprising a β2M polypeptide and a B*0702 HLA-A heavy chain; EYKLVVVGAR (SEQ ID NO:155), which can present an epitope when bound to an HLA complex comprising a β2M polypeptide and an A*3101 HLA-A heavy chain; RGVGKSALTI (SEQ ID NO:156), which can present an epitope when bound to an HLA complex comprising a β2M polypeptide and a B*0702 HLA-A heavy chain; LVVVGARGV (SEQ ID NO:157), which can present an epitope when bound to an HLA complex comprising a β2M polypeptide and an A*0203 HLA-A heavy chain; GADGVGKSAL (SEQ I DNO:160), which can present an epitope when bound to an HLA complex comprising a β2M polypeptide and a B*3801 HLA-A heavy chain; ACGVGKSAL (SEQ ID NO:158), which can present an epitope when bound to an HLA complex comprising a β2M polypeptide and a B*0702 HLA-A heavy chain; and GACGVGKSAL (SEQ ID NO:159), which can present an epitope when bound to an HLA complex comprising a β2M polypeptide and a B*3801 HLA-A heavy chain.

b. HLA/Peptide Binding Assays

Whether a given peptide (e.g., WT-1 peptide) binds a class I HLA (comprising an HLA heavy chain and a β2M polypeptide), and, when bound to the HLA complex, can effectively present an epitope to a TCR, can be determined using any of a number of well-known methods. Assays include binding assays and T cell activation assays.

(i) Cell-Based Binding Assay

As one example, a cell-based peptide-induced stabilization assay can be used to determine peptide-HLA class I binding. In this assay, a peptide of interest is allowed to bind to a TAP-deficient cell, i.e., a cell that has defective transporter associated with antigen processing (TAP) machinery, and consequently, few surface class I molecules. Such cells include, e.g., the human T2 cell line (T2 (174 x CEM.T2; American Type Culture Collection (ATCC) No. CRL-1992). Henderson et al. (1992) Science 255:1264. Without efficient TAP-mediated transport of cytosolic peptides into the endoplasmic reticulum, assembled class I complexes are structurally unstable, and retained only transiently at the cell surface. However, when T2 cells are incubated with an exogenous peptide capable of binding class I, surface peptide-HLA class I complexes are stabilized and can be detected by flow cytometry with, e.g., a pan anti-class I monoclonal antibody. The stabilization and resultant increased life-span of peptide-HLA complexes on the cell surface by the addition of a peptide validates their identity. Analysis can be carried out using flow cytometry, e.g., where the pan-HLA class I antibody comprises a fluorescent label. Binding of the peptide to various allelic forms of HLA H chains can be tested by genetically modifying the T2 cells to express an allelic HLA H chain of interest.

The following is a non-limiting example of use of a T2 assay to assess peptide binding to HLA A*0201. T2 cells are washed in cell culture medium, and concentrated to 10⁶ cells/ml. Peptides of interest are prepared in cell culture medium and serially diluted providing concentrations of 200 μM, 100 μM, 20 μM and 2 μM. The cells are mixed 1:1 with each peptide dilution to give a final volume of 200 L and final peptide concentrations of 100 μM, 50 μM, 10 μM and 1 μM. A HLA A*0201 binding peptide, GILGFVFTL, and a non-HLA A*0201-restricted peptide, HPVGEADYF (HLA-B*3501), are included as positive and negative controls, respectively. The cell/peptide mixtures are kept at 37° C. 5% CO₂ for ten minutes; then incubated at room temperature overnight. Cells are then incubated for 2 hours at 37° C. and stained with a fluorescently-labeled anti-human HLA antibody. The cells are washed twice with phosphate-buffered saline and analyzed using flow cytometry. The average mean fluorescence intensity (MFI) of the anti-HLA antibody staining is used to measure the strength of binding.

(ii) Biochemical Binding Assay

HLA polypeptides (HLA heavy chain polypeptide complexed with β2M polypeptide) can be tested for binding to a peptide of interest in a cell-free in vitro assay system. For example, a labeled reference peptide (e.g., fluorescently labeled) is allowed to bind to HLA polypeptides (HLA heavy chain polypeptide complexed with β2M polypeptide), to form an HLA-reference peptide complex. The ability of a test peptide of interest to displace the labeled reference peptide from the HLA-reference peptide complex is tested. The relative binding affinity is calculated as the amount of test peptide needed to displace the bound reference peptide. See, e.g., van der Burg et al. (1995) Human Immunol. 44:189.

As another example, a peptide of interest can be incubated with an HLA molecule (HLA heavy chain complexed with a β2M polypeptide), and the stabilization of the HLA/peptide complex can be measured in an immunoassay format. The ability of a peptide of interest to stabilize an HLA molecule is compared to that of a control peptide presenting a known T cell epitope. Detection of stabilization is based on the presence or absence of the native conformation of the HLA/peptide complex, detected using an anti-HLA antibody. See, e.g., Westrop et al. (2009) J. Immunol. Methods 341:76; Steinitz et al. (2012) Blood 119:4073; and U.S. Pat. No. 9,205,144.

(iii) T Cell Activation Assays

Whether a given peptide binds a class I HLA (comprising an HLA heavy chain and a β2M polypeptide), and, when bound to the HLA complex, can effectively present an epitope to a TCR, can be determined by assessing T cell response to the peptide-HLA complex. T cell responses that can be measured include, e.g., interferon-gamma (IFNγ) production, cytotoxic activity, and the like.

(iv) ELISPOT Assay

Suitable assays include, e.g., an enzyme linked immunospot (ELISPOT) assay. In this assay, production of IFNγ by CD8⁺ T cells is measured following with an antigen-presenting cell (APC) that presents a peptide of interest complexed with HLA class I. Antibody to IFNγ is immobilized on wells of a multi-well plate. APCs are added to the wells, and incubated for a period of time with a peptide of interest, such that the peptide binds HLA class I on the surface of the APCs. CD8⁺ T cells specific for the peptide are added to the wells, and the plate is incubated for about 24 hours. The wells are then washed, and any IFNγ bound to the immobilized anti-IFNγ antibody is detected using a detectably labeled anti-IFNγ antibody. A colorimetric assay can be used. For example, the detectably labeled anti-IFNγ antibody can be a biotin-labeled anti-IFNγ antibody, which can be detected using, e.g., streptavidin conjugated to alkaline phosphatase. A BCIP/NBT (5-bromo-4-chloro-3-indolyl phosphate/nitro blue tetrazolium) solution is added, to develop the assay. The presence of IFNγ-secreting T cells is identified by colored spots. Negative controls include APCs not contacted with the peptide. APCs expressing various HLA H chain alleles can be used to determine whether a peptide of interest effectively binds to a HLA class I molecule comprising a particular HLA H chain.

(v) Cytotoxicity Assays

Whether a given peptide binds to a particular HLA class I H chain and, when bound to a HLA class I complex comprising the H chain, can effectively present an epitope to a TCR, can also be determined using a cytotoxicity assay. A cytotoxicity assay involves incubation of a target cell with a cytotoxic CD8⁺ T cell. The target cell displays on its surface a peptide/HLA class I complex comprising a peptide of interest and an HLA class I molecule comprising an HLA H chain to be tested. The target cells can be radioactively labeled, e.g., with ⁵¹Cr. Whether the target cell effectively presents an epitope to a TCR on the cytotoxic CD8⁺ T cell, thereby inducing cytotoxic activity by the CD8⁺ T cell toward the target cell, is determined by measuring release of ⁵¹Cr from the lysed target cell. Specific cytotoxicity can be calculated as the amount of cytotoxic activity in the presence of the peptide minus the amount of cytotoxic activity in the absence of the peptide.

(vi) Detection of Antigen-Specific T Cells with Peptide-HLA Tetramers

As another example, multimers (e.g., tetramers) of peptide-HLA complexes are generated with fluorescent or heavy metal tags. The multimers can then be used to identify and quantify specific T cells via flow cytometry (FACS) or mass cytometry (CyTOF). Detection of epitope-specific T cells provides direct evidence that the peptide-bound HLA molecule is capable of binding to a specific TCR on a subset of antigen-specific T cells. See, e.g., Klenerman et al. (2002) Nature Reviews Immunol. 2:263.

8. Additional Polypeptides

A polypeptide chain of a MAPP of the present disclosure (e.g., a dimerization or framework polypeptide) may include one or more polypeptides in addition to those described above. Suitable additional polypeptides include epitope tags and affinity domains. The one or more additional polypeptides can be included at the N-terminus of a polypeptide chain of a MAPP of the present disclosure, at the C-terminus of a polypeptide chain of a MAPP of the present disclosure, or within (internal to) a polypeptide chain of a MAPP of the present disclosure.

a. Epitope Tags

Suitable epitope tags include, but are not limited to, hemagglutinin (HA; e.g., YPYDVPDYA (SEQ ID NO:173); FLAG (e.g., DYKDDDDK (SEQ ID NO:174); c-myc (e.g., EQKLISEEDL; SEQ ID NO:175), and the like.

b. Affinity Domains

Affinity domains include peptide sequences that can interact with a binding partner, e.g., such as one immobilized on a solid support, useful for identification or purification. DNA sequences encoding multiple consecutive single aas, such as histidine, when fused to the expressed protein, may be used for one-step purification of the recombinant protein by high affinity binding to a resin column, such as nickel sepharose. Exemplary affinity domains include HisX5 (HHHHH) (SEQ ID NO:176), HisX6 (HHHHHH) (SEQ ID NO:177), C-myc (EQKLISEEDL) (SEQ ID NO:175), Flag (DYKDDDDK) (SEQ ID NO:174), StrepTag (WSHPQFEK) (SEQ ID NO:185), hemagglutinin, e.g., HA Tag (YPYDVPDYA) (SEQ ID NO:173), glutathione-S-transferase (GST), thioredoxin, cellulose binding domain, RYIRS (SEQ ID NO:178), Phe-His-His-Thr (SEQ ID NO:179), chitin binding domain, S-peptide, T7 peptide, SH2 domain, C-end RNA tag, WEAAAREACCRECCARA (SEQ ID NO:180), metal binding domains, e.g., zinc binding domains or calcium binding domains such as those from calcium-binding proteins, e.g., calmodulin, troponin C, calcineurin B, myosin light chain, recoverin, S-modulin, visinin, VILIP, neurocalcin, hippocalcin, frequenin, caltractin, calpain large-subunit, S100 proteins, parvalbumin, calbindin D9K, calbindin D28K, calretinin, inteins, biotin, streptavidin, MyoD, Id, leucine zipper sequences, and maltose binding protein.

c. Targeting Sequences

MAPPs of the present disclosure may include, as part of any one or more framework and/or any one or more dimerization polypeptide, a targeting polypeptide or “targeting sequence.” Targeting sequences may be located, for example at or near the carboxyl terminal end of a framework or dimerization peptide (e.g., in place of a C-terminal MOD in FIG. 1A or 1B or at position 3, 3′, 5 and/or 5′ of the MAPP in any of FIG. 1A, 1B or 6-9). In an embodiment the targeting sequence may be located at position 3 and/or 3′. Targeting sequences serve to bind or “localize” MAPPs to cells and tissue displaying the protein (or other molecule) which the targeting sequence binds. In some cases, a targeting sequence is an antibody or antigen binding fragment thereof. In some cases, a targeting sequence is a single-chain T cell receptor (scTCR). Targeting sequences may be translated as part of the MAPP (e.g., part of the framework polypeptide) or incorporated by covalent attachment (e.g., using a crosslinker) of a targeting sequence, where the targeting sequence effectively becomes a payload-like molecule attached to the MAPP. Targeting sequences may also be non-covalently bound to a MAPP. For example, a MAPP having a biotin labeled framework polypeptide may be non-covalently attached to an avidin labeled targeting antibody or Fab directed to, for example, a cancer antigen). A bispecific antibody (e.g., a bispecific IgG or humanized antibody) having a first antigen binding site directed to a part of the MAPP (e.g., the framework polypeptide) may also be employed to non-covalently attach a MAPP to a targeting sequence (the second bispecific antibody binding site) directed to a target (e.g., a cancer antigen). Targeting sequences serve to bind or “localize” MAPPs to cells and/or tissues displaying the protein (or other molecule) to which the targeting sequence binds.

9. Payloads—Drug and Other Conjugates

A polypeptide chain of a MAPP can comprise a payload such as a therapeutic (e.g, a small molecule drug or therapeutic) a label (e.g., a fluorescent label or radio label), or other biologically active agent that is linked (e.g., covalently attached) to the polypeptide chain. For example, where a MAPP comprises an Fc polypeptide, the Fc polypeptide may comprise a covalently linked payload such as an agent that treats a cancer or benign neoplasmautoim or is an agent that relieves a symptom of such diseases.

A payload can be linked directly or indirectly to a polypeptide chain of a MAPP of the present disclosure (e.g., to an Ig Fc polypeptide in the MAPP). Direct linkage can involve linkage directly to an aa side chain. Indirect linkage can be linkage via a cross-linker, such as a bifunctional cross-linker. A payload can be linked to a MAPP of the present disclosure by any acceptable chemical linkage including, but not limited to a thioether bond, an amide bond, a carbamate bond, a disulfide bond, or an ether bond, including those formed by reaction with a crosslinking agent.

Crosslinkers (crosslinking agents) include cleavable cross-linkers and non-cleavable cross-linkers. In some cases, the cross-linker is a protease-cleavable cross-linker. Suitable cross-linkers may include, for example, peptides (e.g., from 2 to 10 aas in length; e.g., 2, 3, 4, 5, 6, 7, 8, 9, or 10 aas in length), alkyl chains, poly(ethylene glycol), disulfide groups, thioether groups, acid labile groups, photolabile groups, peptidase labile groups, and esterase labile groups. Non-limiting example of suitable cross-linkers are: N-succinimidyl-[(N-maleimidopropionamido)-tetraethyleneglycol]ester (NHS-PEG4-maleimide); N-succinimidyl 4-(2-pyridyldithio)butanoate (SPDB); N-succinimidyl 4-(2-pyridyldithio)2-sulfobutanoate (sulfo-SPDB); N-succinimidyl 4-(2-pyridyldithio) pentanoate (SPP); N-succinimidyl-4-(N-maleimidomethyl)-cyclohexane-1-carboxy-(6-amidocaproate) (LC-SMCC); κ-maleimidoundecanoic acid N-succinimidyl ester (KMUA); γ-maleimide butyric acid N-succinimidyl ester (GMBS); ε-maleimidocaproic acid N-hydroxysuccinimide ester (EMCS); m-maleimide benzoyl-N-hydroxysuccinimide ester (MBS); N-(α-maleimidoacetoxy)-succinimide ester (AMAS); succinimidyl-6-(β-maleimidopropionamide)hexanoate (SMPH); N-succinimidyl 4-(p-maleimidophenyl)butyrate (SMPB); N-(p-maleimidophenyl)isocyanate (PMPI); N-succinimidyl 4(2-pyridylthio)pentanoate (SPP); N-succinimidyl(4-iodo-acetyl)aminobenzoate (SIAB); 6-maleimidocaproyl (MC); maleimidopropanoyl (MP); p-aminobenzyloxycarbonyl (PAB); N-succinimidyl 4-(maleimidomethyl)cyclohexanecarboxylate (SMCC); N-succinimidyl-4-(N-maleimidomethyl)-cyclohexane-1-carboxy-(6-amidocaproate), a “long chain” analog of SMCC (LC-SMCC); 3-maleimidopropanoic acid N-succinimidyl ester (BMPS); N-succinimidyl iodoacetate (SIA); N-succinimidyl bromoacetate (SBA); and N-succinimidyl 3-(bromoacetamido)propionate (SBAP).

MAPP payload conjugates may be formed by reaction of a MAPP polypeptide (e.g., an IgFc polypeptide) with a crosslinking reagent to introduce 1-10 reactive groups. The polypeptide is then reacted with the molecule to be conjugated (e.g., a thiol-containing payload drug, label or agent) to produce a MAPP-payload conjugate. For example, where a MAPP of the present disclosure comprises an IgFc polypeptide, the conjugate can be of the form (A)-(L)-(C), where (A) is the polypeptide chain comprising the IgFc polypeptide; where (L), if present, is a cross-linker; and where (C) is a payload. (L), if present, links (A) to (C). In some cases, the MAPP includes an IgFc polypeptide that comprises one or more (e.g., 2, 3, 4, 5, or more than 5) molecules of a payload. Introducing payloads into a MAPP using an excess of crosslinking agents can result in multiple molecules of payload being incorporated into the MAPP.

Suitable payloads (e.g., drugs) include virtually any small molecule (e.g., less than 2,000 Daltons in molecular weight) approved by the U.S. Food and Drug Administration, and/or listed in the 2020 U.S. Pharmacopeia or National Formulary. In an embodiment, those drugs are less than 1,000 molecular weight. Suitable drugs include antibiotics, and chemotherapeutic (antineoplastic). Suitable chemotherapeutics may be alkylating agents, cytoskeletal disruptors (taxanes), epothilone, histone deacetylase inhibitors, topoisomerase I inhibitors, topoisomerase II inhibitors, kinase inhibitors, nucleotide analog or precursor analogs, peptide antineoplastic antibiotics (e.g. bleomycin or actinomycin), platinum-based agents, retinoids, vinca alkaloids, cyclophosphamide. Suitable drugs also include non-steroidal anti-inflammatory drugs and glucocorticoids, and the like.

### D. Nucleic Acids

The present disclosure provides a nucleic acid comprising a nucleotide sequence encoding one or more polypeptides of a MAPP of the present disclosure. In some cases, the nucleic acid is a recombinant expression vector; thus, the present disclosure provides a recombinant expression vector comprising a nucleotide sequence encoding a MAPP of the present disclosure.

1. Nucleic Acids Encoding a MAPP or MAPP Forming a Higher Order Complex, Such as a Duplex MAPP, that Comprises at Least One Dimerization Sequence and a Multimerization Sequence

The present disclosure provides a nucleic acid comprising a nucleotide sequence encoding a MAPPs having a framework polypeptide that comprises at least one dimerization sequence and at least one multimerization sequence that permits two molecules of the framework polypeptide to form dimers or higher order complexes. The nucleic acid may additionally comprise a nucleotide sequence encoding a dimerization peptide. The nucleic acid encoding either or both of the framework polypeptide and/or dimerization peptide may include a sequence of encoding a presenting sequence. Where the MAPP comprises a presenting complex, the nucleic acid may further comprise a sequence encoding a presenting complex 2^{nd} sequence. The nucleic acid sequences encoding MAPPs may also encode an epitope peptide. The nucleotide sequence(s) comprising any of the MAPP polypeptides can be operably linked to a transcription control element(s), e.g., a promoter. It will be apparent that individual polypeptides of a MAPP (e.g., a framework polypeptide and dimerization polypeptide) may be encoded on a single nucleic acid under the control of separate promoters, or alternatively, may be located on separate nucleic acids (e.g., plasmids).

2. Recombinant Expression Vectors

The present disclosure provides recombinant expression vectors comprising nucleic acids encoding one or more polypeptides of a MAPP or its higher order complexes. In some cases, the recombinant expression vector is a non-viral vector. In some cases, the recombinant expression vector is a viral construct, e.g., a recombinant adeno-associated virus construct (see, e.g., U.S. Pat. No. 7,078,387), a recombinant adenoviral construct, a recombinant lentiviral construct, a recombinant retroviral construct, a non-integrating viral vector, etc.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Virol. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. Numerous suitable expression vectors are known to those of skill in the art, and many are commercially available.

Depending on the host/vector system utilized, any of a number of suitable transcription and translation control elements, including constitutive and inducible promoters, transcription enhancer elements, transcription terminators, etc. may be used in the expression vector (see, e.g., Bitter et al. (1987) Methods in Enzymology, 153:516-544).

In some cases, a nucleotide sequence encoding one or more polypeptides of a MAPP is operably linked to a control element, e.g., a transcriptional control element, such as a promoter. The transcriptional control element may be functional in either a eukaryotic cell, e.g., a mammalian cell such as a human, hamster, or mouse cell; or a prokaryotic cell (e.g., bacterial). In some cases, a nucleotide sequence encoding a DNA-targeting RNA and/or a site-directed modifying polypeptide is operably linked to multiple control elements that allow expression of the nucleotide sequence encoding a DNA-targeting RNA and/or a site-directed modifying polypeptide in both prokaryotic and eukaryotic cells.

Non-limiting examples of suitable eukaryotic promoters (promoters functional in a eukaryotic cell) include the cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

### E. Genetically Modified Host Cells

The present disclosure provides a genetically modified host cell, where the host cell is genetically modified with a nucleic acid(s) of the present disclosure that encode, or encode and express, MAPP proteins or higher order complexes of MAPPs (e.g., duplex MAPPs).

Suitable host cells include eukaryotic cells, such as yeast cells, insect cells, and mammalian cells. In some cases, the host cell is a cell of a mammalian cell line. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2™), CHO cells (e.g., ATCC Nos. CRL-9618™, CCL-61™, CRL9096), 293 cells (e.g., ATCC No. CRL-1573™), Vero cells, NIH 3T3 cells (e.g., ATCC No. CRL-1658), Huh-7 cells, BHK cells (e.g., ATCC No. CCL-10™), PC12 cells (ATCC No. CRL-1721™), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like. In some cases, the host cell is a mammalian cell that has been genetically modified such that it does not synthesize endogenous MHC β2M and/or such that it does not synthesize endogenous MHC Class I heavy chains (MHC-H).

Genetically modified host cells can be used to produce a MAPP and higher order complexes of MAPPs of the present disclosure. For example, a genetically modified host cell can be used to produce a duplex MAPP of the present disclosure. For example, an expression vector(s) comprising nucleotide sequences encoding the MAPP polypeptide(s) is/are introduced into a host cell, generating a genetically modified host cell, which genetically modified host cell produces the polypeptide(s) (e.g., as an excreted soluble protein).

### F. Compositions

1. Compositions Comprising a MAPP

The present disclosure provides compositions, including pharmaceutical compositions, comprising a MAPP and/or higher order complexes of MAPPs (e.g., duplex MAPPs) of the present disclosure. The present disclosure provides compositions, including pharmaceutical compositions, comprising a nucleic acid or a recombinant expression vector of the present disclosure. A composition of the present disclosure can comprise, in addition to a MAPP of the present disclosure, one or more of: a salt, e.g., NaCl, MgCl₂, KCl, MgSO₄, etc.; a buffering agent, e.g., a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a protease inhibitor; glycerol; and the like.

The composition may comprise a pharmaceutically acceptable excipient, a variety of which are known in the art and need not be discussed in detail herein. Pharmaceutically acceptable excipients have been amply described in a variety of publications, including, for example, “Remington: The Science and Practice of Pharmacy”, 19^{th} Ed. (1995), or latest edition, Mack Publishing Co; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

A pharmaceutical composition can comprise: i) a MAPP (or higher order MAPP complex such as a duplex MAPP) of the present disclosure; and ii) a pharmaceutically acceptable excipient. In some cases, a subject pharmaceutical composition will be suitable for administration to a subject, e.g., will be sterile. For example, in some embodiments, a subject pharmaceutical composition will be suitable for administration to a human subject, e.g., where the composition is sterile and is free of detectable pyrogens and/or other toxins.

The protein compositions may comprise other components, such as pharmaceutical grades of mannitol, lactose, starch, magnesium stearate, sodium saccharin, talcum, cellulose, glucose, sucrose, magnesium, carbonate, and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate, hydrochloride, sulfate salts, solvates (e.g., mixed ionic salts, water, organics), hydrates (e.g., water), and the like.

For example, compositions may include (e.g., be in the form of) aqueous or other solutions, powders, granules, tablets, pills, suppositories, capsules, suspensions, sprays, and the like. The composition may be formulated according to the various routes of administration described below.

Where a MAPP or higher order MAPP complex (e.g., duplex MAPP) of the present disclosure is administered as an injectable (e.g. subcutaneously, intraperitoneally, intramuscularly, intralymphatically, and/or intravenously) directly into a tissue, a formulation can be provided as a ready-to-use dosage form, or as non-aqueous form (e.g. a reconstitutable storage-stable powder) or an aqueous form, such as liquid composed of pharmaceutically acceptable carriers and excipients. MAPPs may also be provided so as to enhance serum half-life of the subject protein following administration. For example, the protein may be provided in a liposome formulation, prepared as a colloid, or other conventional techniques for extending serum half-life. A variety of methods are available for preparing liposomes, as described in, e.g., Szoka et al. 1980 Ann. Rev. Biophys. Bioeng. 9:467, U.S. Pat. Nos. 4,235,871, 4,501,728 and 4,837,028. The preparations may also be provided in controlled release or slow-release forms.

In some cases, a MAPP composition comprises: a) a MAPP higher order MAPP complex (e.g., a duplex MAPP) of the present disclosure; and b) saline (e.g., 0.9% NaCl). In some cases, the composition is sterile. In some cases, the composition is suitable for administration to a human subject, e.g., where the composition is sterile and is free of detectable pyrogens and/or other toxins. Thus, the present disclosure provides a composition comprising: a) a MAPP or higher order MAPP complex (e.g., duplex MAPP) of the present disclosure; and b) saline (e.g., 0.9% NaCl), where the composition is sterile and is free of detectable pyrogens and/or other toxins. In such formulations, the epitope peptide may be part of one of the MAPP polypeptides, or alternatively, may be a separate peptide or other epitope presenting molecule (e.g., a phosphopeptide).

Other examples of formulations suitable for parenteral administration include isotonic sterile injection solutions, anti-oxidants, bacteriostats, and solutes that render the formulation isotonic with the blood of the intended recipient, suspending agents, solubilizers, thickening agents, stabilizers, and preservatives. For example, a subject pharmaceutical composition can be present in a container, e.g., a sterile container, such as a syringe. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

The concentration of a MAPP of the present disclosure in a formulation can vary widely. For example, a MAPP or higher order MAPP complex (e.g., duplex MAPP) may be present from less than about 0.1% (usually at least about 2%) to as much as 20% to 50% or more by weight (e.g., from 1% to 10%, 5% to 15%, 10% to 20% by weight, or 20-50% by weight) by weight. The concentration will usually be selected primarily based on fluid volumes, viscosities, and patient-based factors in accordance with the particular mode of administration selected and the patient's needs.

The present disclosure provides a container comprising a composition of the present disclosure, e.g., a liquid composition. The container can be, e.g., a syringe, an ampoule, and the like. In some cases, the container is sterile. In some cases, both the container and the composition are sterile.

2. Compositions Comprising a Nucleic Acid or a Recombinant Expression Vector

The present disclosure provides compositions (e.g., pharmaceutical compositions) comprising a nucleic acid or a recombinant expression vector of the present disclosure that comprise one or more nucleic acid sequences encoding any one or more MAPP polypeptide (or each of the polypeptides of a MAPP). A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

A composition of the present disclosure can include: a) one or more nucleic acids or one or more recombinant expression vectors comprising nucleotide sequences encoding a MAPP polypeptide (or all polypeptides of a MAPP) of the present disclosure; and b) one or more of: a buffer, a surfactant, an antioxidant, a hydrophilic polymer, a dextrin, a chelating agent, a suspending agent, a solubilizer, a thickening agent, a stabilizer, a bacteriostatic agent, a wetting agent, and a preservative. Suitable buffers include, but are not limited to, (such as N,N-bis(2-hydroxyethyl)-2-aminoethanesulfonic acid (BES), bis(2-hydroxyethyl)amino-tris(hydroxymethyl)methane (BIS-Tris), N-(2-hydroxyethyl)piperazine-N′3-propanesulfonic acid (EPPS or HEPPS), glycylglycine, N-2-hydroxyehtylpiperazine-N′-2-ethanesulfonic acid (HEPES), 3-(N-morpholino)propane sulfonic acid (MOPS), piperazine-N,N′-bis(2-ethane-sulfonic acid) (PIPES), sodium bicarbonate, 3-(N-tris(hydroxymethyl)-methyl-amino)-2-hydroxy-propanesulfonic acid) TAPSO, (N-tris(hydroxymethyl)methyl-2-aminoethanesulfonic acid (TES), N-tris(hydroxymethyl)methyl-glycine (Tricine), tris(hydroxymethyl)-aminomethane (Tris), etc.). Suitable salts include, e.g., NaCl, MgCl₂, KCl, MgSO₄, etc.

A pharmaceutical formulation of the present disclosure can include a nucleic acid or recombinant expression vector of the present disclosure in an amount of from about 0.001% to about 90% (w/w). In the description of formulations, below, “subject nucleic acid or recombinant expression vector” will be understood to include a nucleic acid or recombinant expression vector of the present disclosure. For example, in some cases, a subject formulation comprises a nucleic acid or recombinant expression vector of the present disclosure.

A subject nucleic acid or recombinant expression vector can be admixed, encapsulated, conjugated or otherwise associated with other compounds or mixtures of compounds; such compounds can include, e.g., liposomes or receptor-targeted molecules. A subject nucleic acid or recombinant expression vector can be combined in a formulation with one or more components that assist in uptake, distribution and/or absorption.

A subject nucleic acid or recombinant expression vector composition can be formulated into any of many possible dosage forms such as, but not limited to, tablets, capsules, gel capsules, liquid syrups, soft gels, suppositories, and enemas. A subject nucleic acid or recombinant expression vector composition can also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

A formulation comprising a subject nucleic acid or recombinant expression vector can be a liposomal formulation. As used herein, the term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes that can interact with negatively charged DNA molecules to form a stable complex. Liposomes that are pH sensitive or negatively charged are believed to entrap DNA rather than complex with it. Both cationic and noncationic liposomes can be used to deliver a subject nucleic acid or recombinant expression vector.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860, which is incorporated herein by reference in its entirety.

The formulations and compositions of the present disclosure may also include surfactants. The use of surfactants in drug products, formulations and in emulsions is well known in the art. Surfactants and their uses are further described in U.S. Pat. No. 6,287,860.

In one embodiment, various penetration enhancers are included to effect the efficient delivery of nucleic acids. In addition to aiding the diffusion of non-lipophilic drugs across cell membranes, penetration enhancers also enhance the permeability of lipophilic drugs. Penetration enhancers may be classified as belonging to one of five broad categories, i.e., surfactants, fatty acids, bile salts, chelating agents, and non-chelating non-surfactants. Penetration enhancers and their uses are further described in U.S. Pat. No. 6,287,860, which is incorporated herein by reference in its entirety.

Compositions and formulations for oral administration include powders or granules, microparticulates, nanoparticulates, suspensions or solutions in water or non-aqueous media, capsules, gel capsules, sachets, tablets, or minitablets. Thickeners, flavoring agents, diluents, emulsifiers, dispersing aids or binders may be desirable. Suitable oral formulations include those in which a subject antisense nucleic acid is administered in conjunction with one or more penetration enhancers surfactants and chelators. Suitable surfactants include, but are not limited to, fatty acids and/or esters or salts thereof, bile acids and/or salts thereof. Suitable bile acids/salts and fatty acids and their uses are further described in U.S. Pat. No. 6,287,860. Also suitable are combinations of penetration enhancers, for example, fatty acids/salts in combination with bile acids/salts. An exemplary suitable combination is the sodium salt of lauric acid, capric acid, and UDCA. Further penetration enhancers include, but are not limited to, polyoxyethylene-9-lauryl ether, and polyoxyethylene-20-cetyl ether. Suitable penetration enhancers also include propylene glycol, dimethyl sulfoxide, triethanolamine, N,N-dimethylacetamide, N,N-dimethylformamide, 2-pyrrolidone and derivatives thereof, tetrahydrofurfuryl alcohol, and AZONE™.

### G. Formulations (Pharmaceutical Formulations)

Suitable formulations are described above, where suitable formulations include a pharmaceutically acceptable excipient. In some cases, a suitable formulation comprises: a) a MAPP or higher order MAPP complex (e.g., duplex MAPP) of the present disclosure; and b) a pharmaceutically acceptable excipient. In some cases, a suitable formulation comprises: a) a nucleic acid comprising a nucleotide sequence encoding a MAPP, which may assemble into a higher order MAPP complex (e.g., duplex MAPP) of the present disclosure; and b) a pharmaceutically acceptable excipient; in some instances, the nucleic acid is an mRNA. In some cases, a suitable formulation comprises: a) a first nucleic acid comprising a nucleotide sequence encoding a framework MAPP of the present disclosure; b) a second nucleic acid comprising a nucleotide sequence encoding a dimerization polypeptide of a MAPP of the present disclosure; and c) a pharmaceutically acceptable excipient. In some cases, a suitable formulation comprises: a) a recombinant expression vector comprising a nucleotide sequence encoding a polypeptide of a MAPP of the present disclosure or all polypeptides of a MAPP of the present disclosure; and b) a pharmaceutically acceptable excipient. In some cases, a suitable formulation comprises: a) a first recombinant expression vector comprising a nucleotide sequence encoding a framework polypeptide of a MAPP of the present disclosure; b) a second recombinant expression vector comprising a sequence encoding a dimerization polypeptide of a MAPP of the present disclosure; and c) a pharmaceutically acceptable excipient.

Suitable pharmaceutically acceptable excipients are described above.

### H. Methods

MAPPs and higher order MAPP complexes (e.g., duplex MAPP) of the present disclosure are useful for modulating an activity of a T cell. Thus, the present disclosure provides methods of modulating an activity of a T cell, the methods generally involving contacting a target T cell with a MAPP or a higher order MAPP complex (e.g., duplex MAPP) of the present disclosure.

1. Methods of Modulating T Cell Activity

The present disclosure provides a method of selectively modulating the activity of an epitope-specific T cell (for the purpose of this disclosure, an epitope-specific T cell is a T cell specific/selective for a KRAS epitope of interest), the method comprising contacting the T cell with a MAPP or higher order MAPP complex (e.g., duplex MAPP) of the present disclosure, where the contacting selectively modulates the activity of the epitope-specific T cell. In some cases, the contacting occurs in vivo (e.g., in a mammal such as a human, rat, mouse, dog, cat, pig, horse, or primate). In some cases, the contacting occurs in vitro. In some cases, the contacting occurs ex vivo. Contacting T cells with MAPPs or higher order MAPP complexes (e.g., duplex MAPP) can result in modulating the activity of epitope-specific T cells, which can result in, but is not limited to, one or more of: i) activation or proliferation of a cytotoxic (e.g., CD8+) T cells; ii) inducing a cytotoxic activity of a cytotoxic (e.g., CD8+) T cells; iii) inducing the production and release of one or more cytotoxic molecules (e.g., a perforin; a granzyme; a granulysin) by a cytotoxic (e.g., CD8+) T cells; and the like. MAPPs of this disclosure also can be used to cause proliferation of CAR-T cells in vivo, thereby reducing the number of CAR-T cells that are required to be administered to a patient having a cancer associated with a KRAS mutation.

In some cases a MAPP or higher order MAPP complex (e.g., duplex MAPP) of the present disclosure interacts with an epitope specific CD 8+ T cell and increases granule-dependent and/or granule-independent CD8⁺ effector T cell responses. Granule-independent responses include, but are not limited to, changes in the number or percentage of active epitope-specific CD 8+ T cell (e.g., in a population of cells such as in blood, lymphatics, and/or in a target tissue), changes in the expression of Fas ligand (Fas-L, which can result in activation of caspases and target cell death through apoptosis), and cytokine/chemokine production (e.g., production and release of interferon gamma (IFN-γ). Granule-dependent effector actions include the release of granzymes, perforin, and/or granulysin. Activation of epitope specific CD8⁺ cytotoxic T cells (e.g., CD8⁺ cytotoxic effector T cells) can result in the targeted killing of, for example, cancer cells by epitope specific T cells that recognize the epitope presented by the MAPP or MAPP complex through granule-dependent and/or independent responses.

In some cases, a MAPP or higher order MAPP complex of the present disclosure comprises a cancer-related KRAS epitope, and the MAPP or MAPP complex activates a CD8⁺ T cell response (e.g., a CD8⁺ T cell response to a cancer cell).

The present disclosure provides a method of increasing proliferation and/or number of CD 8+ effector T cells specific to the epitope presented by a MAPP or MAPP complex, the method comprising contacting (e.g., in vitro, in vivo, or ex vivo) T cells with a MAPP or higher order MAPP complex (such as by administering to a subject one or more doses of a epitope-presenting MAPP or MAPP complex with a MOD (e.g., IL-2)). The contacting or administering may increase the number of CD8+ effector T cells having a TCR capable of binding the epitope present in the MAPP or MAPP complex relative to the number (e.g., total number or percentage) of T cells present (e.g., in a population of cells such as in blood, lymphatics, and/or in a target tissue). For example, the number of CD 8+ effector T cells specific to the epitope presented by the MAPP or MAPP complex (e.g., duplex MAPP) can be increased by at least 5%, at least 10%, at least 20%, at least 30%, at least 40%, at least 50%, least 75%, at least 100%, at least 2-fold, at least 2.5-fold, at least 5-fold, at least 10-fold, or more than 10-fold following one or more contacts with doses or administrations of the MAPP or higher order MAPP complex. The increase may be calculated relative the CD+ T cell numbers present prior to the contacting or administrations, or relative to the population of T cells present in a sample (e.g., a sample of blood or tissue) that has not been contacted with the MAPP or MAPP complex.

The present disclosure provides a method of increasing granule-independent and/or granule-independent responses of epitope-specific CD 8+ T cell comprising contacting (e.g., in vitro, in vivo, or ex vivo) T cells with a epitope presenting MAPP or higher order MAPP complex (such as by administering to a subject one or more does of a MAPP or MAPP complex with, for example a CD80 or CD86 MOD). The contacting or administering results in increasing the expression of Fas ligand expression, cytokines/chemokines (e.g., IL-2, IL-4, and/or IL-5), release of interferons (e.g., IFN-γ), release of granzymes, release of perforin, and/or release of granulysin. For example, contacting CD 8+ effector cell with a MAPP or MAPP complex (e.g., duplex MAPP) presenting epitope specific to the effector cell can the increase one or more of Fas ligand expression, interferon gamma (IFN-γ) release, granzyme release, perforin release, and/or granulysin release by at least 5%, at least 10%, at least 20%, at least 30%, at least 40%, at least 50%, least 75%, at least 100%, at least 2-fold, at least 2.5-fold, at least 5-fold, at least 10-fold, or more than 10-fold. The increase may be calculated relative the level of expression or release prior to the contacting or administrations, or relative to the population of T cells present in a sample (e.g., a sample of blood or tissue) that has not been contacted with the MAPP or MAPP complex.

2. Treatment Methods

The present disclosure provides treatment methods, the methods comprising administering to the individual an amount of a MAPP or higher order MAPP complex (e.g., duplex MAPP) of the present disclosure, or one or more nucleic acids or expression vectors encoding a MAPP that may assemble into a higher order complex (e.g., duplex MAPP), effective to selectively modulate the activity of an epitope-specific T cell in an individual, and to thereby treat the individual. In some cases, a treatment method of the present disclosure comprises administering to an individual in need thereof one or more recombinant expression vectors comprising nucleotide sequences encoding a MAPP (e.g. a MAPP that may assemble into a higher order MAPP complex) of the present disclosure. In some cases, a treatment method of the present disclosure comprises administering to an individual in need thereof one or more mRNA molecules comprising nucleotide sequences encoding a MAPP of the present disclosure. In some cases, a treatment method of the present disclosure comprises administering to an individual in need thereof a MAPP or higher order MAPP complex (e.g., duplex MAPP) of the present disclosure. The conditions that can be treated include, but are not limited to, cancers and benign neoplasms (e.g., benign tumors that may be inoperable).

The present disclosure provides a method of selectively modulating the activity of an epitope-specific T cell in an individual, the method comprising administering to the individual an effective amount of a MAPP or higher order MAPP complex (e.g., duplex MAPP) of the present disclosure, or one or more nucleic acids (e.g., expression vectors; mRNA; etc.) comprising nucleotide sequences encoding the MAPP which may assemble into a higher order complex, where the MAPP or its complex selectively modulates the activity of the epitope-specific T cell in the individual. Selectively modulating the activity of an epitope-specific T cell can treat a disease or disorder in the individual. Thus, the present disclosure provides a treatment method comprising administering to an individual in need thereof an effective amount of a MAPP of the present disclosure (e.g., a higher order MAPP construct such as a duplex MAPP) sufficient to effect treatment. The diseases and/or disorders that can be treated include cancers and benign neoplasm.

In some cases, a MAPP comprises an inhibitory MOD polypeptide sequence, and a MAPP of the present disclosure inhibits activity of the epitope-specific T cell (e.g., effector functions or proliferation).

In some cases, the MOD is an activating polypeptide, and the MAPP with its associated epitope activates an epitope-specific T cell that recognizes a cancer or tumor specific KRAS antigen. In some cases, the T cells are cytotoxic T cells (CD8⁺ cells). In some cases, a MAPP with its associated epitope increases the activity of a CD8+ effector T cell specific for a cancer or tumor cell expressing the epitope. Activation of CD8⁺ T cells can include increasing proliferation of CD8⁺ T cells and/or inducing or enhancing release of chemokines and/or cytokines by CD8⁺ T cells.

As noted above, in some cases, in carrying out a subject treatment method, a MAPP or higher order MAPP complex (e.g., duplex MAPP) of the present disclosure is administered to an individual in need thereof, as the polypeptide per se. In other instances, in carrying out a subject treatment method, one or more nucleic acids comprising nucleotide sequences encoding a MAPP is/are administering to an individual in need thereof. Thus, in other instances, one or more nucleic acids of the present disclosure, e.g., one or more recombinant expression vectors of the present disclosure, is/are administered to an individual in need thereof.

A MAPP or higher order MAPP complex (e.g., duplex MAPP), or one or more nucleic acids encoding such molecules may be administered alone or with one or more additional therapeutic agents drugs. The therapeutic agents may be administered before, during, or subsequent to MAPP or higher order MAPP complex (e.g., duplex MAPP) or nucleic acids encoding such molecules. When the additional therapeutic agents are administered with a composition or formulation comprising a MAPP or higher order MAPP complex (e.g., duplex MAPP) or nucleic acids encoding such molecules, the therapeutic agent may be administered concurrently with the MAPP. Alternatively, the therapeutic agents may be co-administered with the MAPP as part of a formulation or composition comprising the MAPP or higher order MAPP complex (e.g., duplex MAPP).

Suitable therapeutic agents or drugs that may be administered with a MAPP or higher order MAPP complex include virtually any therapeutic agent, including small molecule therapeutics (e.g., less than 2,000 Daltons in molecular weight) approved by the U.S. Food and Drug Administration, and/or listed in the 2020 U.S. Pharmacopeia or National Formulary. In an embodiment, those therapeutic agents or drugs are less than 1,000 molecular weight. Suitable drugs include antibiotics, and/or chemotherapeutic (antineoplastic). Suitable chemotherapeutics may be alkylating agents, cytoskeletal disruptors (taxanes), epothilones, histone deacetylase inhibitors, topoisomerase I inhibitors, topoisomerase II inhibitors, kinase inhibitors, nucleotide analog or precursor analogs, peptide antineoplastic antibiotics (e.g. bleomycin or actinomycin), platinum-based agents, retinoids, or vinca alkaloids. Suitable drugs also include non-steroidal anti-inflammatory drugs and glucocorticoids, and the like.

In an embodiment, a suitable therapeutic agent that may be administered with a MAPP or higher order MAPP complex comprises an antiTGF-β antibody, such as Metelimumab (CAT192) directed against TGF-β1 and/or Fresolimub directed against TGF-β1 and TGF-β2, or a TGF-β trap (e.g., Cablivi (caplacizumab-yhdp). Such antibodies would, as a generality, not be administered in conjunction with a MAPP or higher order MAPP complex (e.g, a duplexed MAPP) that comprise a sequence to which the antibodies bind such as a TGF-β1 or TGF-β2 MOD.

In an embodiment, a suitable therapeutic agent that may be administered with a MAPP or higher order MAPP complex comprises one or more antibodies directed against: B lymphocyte antigens (e.g., ibritumomab tiuxetan, obinutuzumab, ofatumumab, rituximab to CD20, brentuximab vedotin directed against CD30, and alemtuzumab to CD52); EGFR (e.g., cetuximab, panitumumab, and necitumumab); VEGF (e.g., bevacizumab); VEGFR2 (e.g., ramucirumab); HER2 (e.g., pertuzumab, trastuzumab, and ado-trastuzumab); PD-1 (e.g., nivolumab and pembrolizumab targeting a check point inhibition); RANKL (e.g., denosumab); CTLA-4 (e.g., ipilimumab targeting check point inhibition); IL-6 (e.g., siltuximab); disialoganglioside (GD2), (e.g., dinutuximab) disialoganglioside (GD2); CD38 (e.g., daratumumab); SLAMF7 (Elotuzumab); both EpCAM and CD3 (e.g., catumaxomab); or both CD19 and CD3 (blinatumomab). Such antibodies would, as a generality, not be administered in conjunction with a MAPP or higher order MAPP complex (e.g, a duplexed MAPP) that comprise a sequence to which any of the administered antibodies bind.

In an embodiment, a suitable therapeutic agent that may be administered with a MAPP or higher order MAPP complex comprises one or more chemotherapeutic agents. Such therapeutic agents may be selected from: alkylating agents, cytoskeletal disruptors (taxane), epothilones, histone deacetylase inhibitors, topoisomerase I inhibitors, topoisomerase II inhibitors, kinase inhibitors, nucleotide analog or precursor analogs, peptide antineoplastic antibiotics (e.g. bleomycin or actinomycin), platinum-based agents, retinoids, or vinca alkaloids and their derivatives. In an embodiment, the chemotherapeutic agents are selected from actinomycin all-trans retinoic acid, azacytidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, and vindesine.

The present disclosure provides treatment methods, the methods comprising administering to the individual an amount of a MAPP or higher order MAPP complex of the present disclosure, or one or more nucleic acids or expression vectors encoding the MAPP, effective to selectively modulate the activity of an epitope-specific T cell in an individual and to treat the individual. In some cases, a treatment method of the present disclosure comprises administering to an individual in need thereof one or more recombinant expression vectors comprising nucleotide sequences encoding a MAPP or higher order MAPP complex of the present disclosure. In some cases, a treatment method of the present disclosure comprises administering to an individual in need thereof one or more mRNA molecules comprising nucleotide sequences encoding a MAPP or higher order MAPP complex of the present disclosure. In some cases, a treatment method of the present disclosure comprises administering to an individual in need thereof a MAPP or higher order MAPP complex of the present disclosure.

The present disclosure provides a method of selectively modulating the activity of an epitope-specific T cell in an individual, the method comprising administering to the individual an effective amount of a MAPP or higher order MAPP complex of the present disclosure, or one or more nucleic acids (e.g., expression vectors; mRNA; etc.) comprising nucleotide sequences encoding the MAPP or higher order MAPP complex, which selectively modulates the activity of the epitope-specific T cell in the individual. Selectively modulating the activity of an epitope-specific T cell can treat a disease or disorder in the individual. Thus, the present disclosure provides a treatment method comprising administering to an individual in need thereof an effective amount of a MAPP or higher order MAPP complex in order to treat, for example, a benign neoplasm or cancer associated with KRAS.

In some cases, the MOD is an inhibitory polypeptide, and a MAPP or higher order MAPP complex of the present disclosure inhibits activity of the epitope-specific T cell. In some cases, the epitope is a self-epitope, and a MAPP or higher order MAPP complex of the present disclosure selectively inhibits the activity of a T cell specific for the self-epitope.

In some cases, the MOD is an activating polypeptide, and the MAPP with its associated epitope activates an epitope-specific T cell that recognizes a cancer or tumor specific KRAS antigen. In some cases, the T cells are cytotoxic T cells (CD8⁺ cells). In some cases MAPP or higher order MAPP complex with its associated epitope increases the activity of a T cell specific for a cancer or tumor cell expressing the epitope (e.g., cytotoxic CD8⁺ T cells). Activation of CD8⁺ T cells can include increasing proliferation of CD8⁺ T cells and/or inducing or enhancing release cytokines by CD8⁺ T cells such as interferon γ CD8+ cells.

A MAPP of this disclosure typically will have a MOD such as wt IL-2 or a variant thereof that causes T cell activation that may result in one or more of the following: an increase the activity of ZAP70 protein kinase activity, induction in the proliferation of the T-cell(s), granule-dependent effector actions (e.g., the release of granzymes, perforin, and/or granulysin from cytotoxic T-cells), and/or release of T cell cytokines (e.g., interferon γ from CD8+ cells). In some cases, however, where a MAPP or higher order MAPP complex of the present disclosure may comprise an inhibitory MOD (e.g., PD-L1, FasL, and the like) it may reduce the proliferation and/or activity of a CD8⁺ regulatory T (Treg) cell (e.g., FoxP3⁺, CD8⁺ T cells) specific to the epitope presented by the MAPP or higher order MAPP complex.

As noted above, in some cases, in carrying out a subject treatment method, a MAPP or higher order MAPP complex of the present disclosure is administered to an individual in need thereof, as the polypeptide per se. In other instances, in carrying out a subject treatment method, one or more nucleic acids comprising nucleotide sequences encoding a MAPP is/are administering to an individual in need thereof. Thus, in other instances, one or more nucleic acids of the present disclosure, e.g., one or more recombinant expression vectors of the present disclosure, is/are administered to an individual in need thereof.

As noted above, one type of therapeutic agent that may be administered in conjunction with a MAAP for the treatment of a cancer or benign neoplasm is an immune checkpoint inhibitor. Exemplary immune checkpoint inhibitors include inhibitors that target immune checkpoint polypeptide such as CD27, CD28, CD40, CD122, CD96, CD73, CD47, OX40, GITR, CSF1R, JAK, PI3K delta, PI3K gamma, TAM, arginase, CD137 (also known as 4-1BB), ICOS, A2AR, B7-H3, B7-H4, BTLA, CTLA-4, LAG3, TIM3, VISTA, CD96, TIGIT, CD122, PD-1, PD-L1 and PD-L2. In some cases, the immune checkpoint polypeptide is a stimulatory checkpoint molecule selected from CD27, CD28, CD40, ICOS, OX40, GITR, CD122 and CD137. In some cases, the immune checkpoint polypeptide is an inhibitory checkpoint molecule selected from A2AR, B7-H3, B7-H4, BTLA, CTLA-4, IDO, KIR, LAG3, PD-1, TIM3, CD96, TIGIT and VISTA.

In some cases, the immune checkpoint inhibitor is an antibody specific for an immune checkpoint, e.g., a monoclonal antibody. The anti-immune checkpoint antibody may be a fully human, humanized, or de-immunized such that the antibody does not substantially elicit an immune response in a human. In some cases, the anti-immune checkpoint antibody inhibits binding of the immune checkpoint polypeptide to a ligand for the immune checkpoint polypeptide. In some cases, the anti-immune checkpoint antibody inhibits binding of the immune checkpoint polypeptide to a receptor for the immune checkpoint polypeptide.

Antibodies, e.g., monoclonal antibodies, that are specific for immune checkpoints and that function as immune checkpoint inhibitors, are known in the art. See, e.g., Wurz et al. (2016) Ther. Adv. Med. Oncol. 8:4; and Naidoo et al. (2015) Ann. Oncol. 26:2375. Suitable anti-immune checkpoint antibodies include, but are not limited to, nivolumab (Bristol-Myers Squibb), pembrolizumab (Merck), pidilizumab (Curetech), AMP-224 (GlaxoSmithKline/Amplimmune), MPDL3280A (Roche), MDX-1105 (Medarex, Inc./Bristol Myer Squibb), MEDI-4736 (Medimmune/AstraZeneca), arelumab (Merck Serono), ipilimumab (YERVOY, (Bristol-Myers Squibb), tremelimumab (Pfizer), pidilizumab (CureTech, Ltd.), IMP321 (Immutep S.A.), MGA271 (Macrogenics), BMS-986016 (Bristol-Meyers Squibb), lirilumab (Bristol-Myers Squibb), urelumab (Bristol-Meyers Squibb), PF-05082566 (Pfizer), IPH2101 (Innate Pharma/Bristol-Myers Squibb), MEDI-6469 (MedImmune/AZ), CP-870,893 (Genentech), Mogamulizumab (Kyowa Hakko Kirin), Varlilumab (CelIDex Therapeutics), Avelumab (EMD Serono), Galiximab (Biogen Idec), AMP-514 (Amplimmune/AZ), AUNP 12 (Aurigene and Pierre Fabre), Indoximod (NewLink Genetics), NLG-919 (NewLink Genetics), INCB024360 (Incyte) and combinations thereof. Suitable anti-LAG3 antibodies include, e.g., BMS-986016 and LAG525. Suitable anti-GITR antibodies include, e.g., TRX518, MK-4166, INCAGN01876, and MK-1248. Suitable anti-OX40 antibodies include, e.g., MED10562, INCAGN01949, GSK2831781, GSK-3174998, MOXR-0916, PF-04518600, and LAG525. Suitable anti-VISTA antibodies are provided in, e.g., WO 2015/097536.

A suitable dosage of an anti-immune checkpoint antibody is from about 1 mg/kg to about 2400 mg/kg per day, such as from about 1 mg/kg to about 1200 mg/kg per day, including from about 50 mg/kg to about 1200 mg/kg per day. Other representative dosages of such agents include about 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, 1000 mg/kg, 1100 mg/kg, 1200 mg/kg, 1300 mg/kg, 1400 mg/kg, 1500 mg/kg, 1600 mg/kg, 1700 mg/kg, 1800 mg/kg, 1900 mg/kg, 2000 mg/kg, 2100 mg/kg, 2200 mg/kg, and 2300 mg/kg per day. The effective dose of the antibody may be administered as two, three, four, five, six or more sub-doses, administered separately at appropriate intervals throughout the day.

In some cases, an immune checkpoint inhibitor is an anti-PD-1 antibody. Suitable anti-PD-1 antibodies include, e.g., nivolumab, pembrolizumab (also known as MK-3475), pidilizumab, SHR-1210, PDR001, and AMP-224. In some cases, the anti-PD-1 monoclonal antibody is nivolumab, pembrolizumab or PDR001. Suitable anti-PD1 antibodies are described in U.S. Patent Publication No. 2017/0044259. For pidilizumab, see, e.g., Rosenblatt et al. (2011) J. Immunother. 34:409-18.

In some cases, the anti-PD1 antibody is pembrolizumab. In some cases, the anti-PD-1 antibody is nivolumab (also known as MDX-1106 or BMS-936558; see, e.g., Topalian et al. (2012) N. Eng. J. Med. 366:2443-2454; and U.S. Pat. No. 8,008,449). In some cases, the anti-CTLA-4 antibody is ipilimumab or tremelimumab. For tremelimumab, see, e.g., Ribas et al. (2013) J. Clin. Oncol. 31:616-22.

In some cases, the immune checkpoint inhibitor is an anti-PD-L1 monoclonal antibody. In some cases, the anti-PD-L1 monoclonal antibody is BMS-935559, MEDI4736, MPDL3280A (also known as RG7446), or MSB0010718C. In some embodiments, the anti-PD-L1 monoclonal antibody is MPDL3280A (atezolizumab) or MEDI4736 (durvalumab). For durvalumab, see, e.g., WO 2011/066389. For atezolizumab, see, e.g., U.S. Pat. No. 8,217,149.

In some cases, the anti-PD-L1 antibody is atezolizumab.

3. Methods of Selectively Delivering a MOD

The present disclosure provides a method of delivering a costimulatory polypeptide such as IL-2, or a reduced-affinity variant of a naturally occurring costimulatory polypeptide such as an IL-2 variant disclosed herein, to a selected T cell or a selected T cell population, e.g., in a manner such that a TCR specific for a given KRAS epitope is targeted. The present disclosure provides a method of delivering a costimulatory polypeptide, or a reduced-affinity variant of a naturally occurring costimulatory polypeptide, selectively to a target T cell bearing a TCR specific for the KRAS epitope present in a MAPP or higher order MAPP complex (e.g., duplex MAPP) of the present disclosure. The method comprises contacting a population of T cells with a MAPP or higher order MAPP complex (e.g., duplex MAPP) of the present disclosure. The population of T cells can be a mixed population that comprises: i) the target T cell specific for the KRAS epitope of the MAPP; and ii) non-target T cells that are not specific for the epitope (e.g., T cells that are specific for an epitope(s) other than the epitope to which the epitope-specific T cell binds). The epitope-specific T cell is specific for the epitope peptide present in the MAPP or higher order MAPP complex, and binds to the peptide MHC complex provided by the MAPP or higher order MAPP complex bringing the MOD into contact with those cells. Accordingly, contacting the population of T cells with the MAPP or higher order MAPP complex delivers the costimulatory polypeptide (e.g., IL-2 or a reduced-affinity variant of IL-2) selectively to the T cell(s) that are specific for the epitope present in the MAPP or higher order complex.

Thus, the present disclosure provides a method of delivering a costimulatory polypeptide (MOD) such as IL-2, or a reduced-affinity variant of a naturally occurring costimulatory polypeptide such as an IL-2 variant disclosed herein, or a combination of both a costimulatory MOD and its reduced-affinity variant, selectively to a target T cell. The method of delivering a MOD comprises contacting a mixed population of T cells with a MAPP or higher order MAPP complex (e.g., duplex MAPP) of the present disclosure. The mixed population of T cells comprises the target T cell and non-target T cells. The target T cell is specific for the epitope present within the MAPP or higher order MAPP complex. Contacting the mixed population of T cells with a MAPP or higher order MAPP complex of the present disclosure delivers the costimulatory polypeptide(s) present within the MAPP or higher order complex to the target T cell.

For example, a MAPP or higher order MAPP complex (e.g., duplex MAPP) of the present disclosure is contacted with a population of T cells comprising: i) a target T cell(s) that is specific for the epitope present in the MAPP or a higher order MAPP complex; and ii) a non-target T cell(s), e.g., a T cell(s) that is specific for a second epitope(s) that is not the epitope present in the MAPP or a higher order MAPP complex. Contacting the population results in selective delivery of the costimulatory polypeptide(s) (e.g., naturally-occurring costimulatory polypeptide (e.g., naturally occurring PD-LI) or reduced-affinity variant of a naturally occurring costimulatory polypeptide (e.g., a PD-L1 variant disclosed herein)), which is present in the MAPP or higher order MAPP complex, to the target T cell. Thus, e.g., less than 50%, less than 40%, less than 30%, less than 25%, less than 20%, less than 15%, less than 10%, less than 5%, or less than 4%, 3%, 2% or 1%, of the MAPP or higher order MAPP complex (e.g., duplex MAPP) binds to non-target T cells and, as a result, the costimulatory polypeptide (e.g., PD-L1 or PD-LI variant) is not selectively delivered to target T cell (and accordingly, not effectively delivered to the non-target T cells).

In some cases, the population of T cells is in vitro. In some cases, the population of T cells is in vitro, and a biological response (e.g., T cell activation and/or expansion and/or phenotypic differentiation) of the target T cell population to the MAPP or higher order MAPP complex (e.g., duplex MAPP) of the present disclosure is elicited in the context of an in vitro culture. For example, a mixed population of T cells can be obtained from an individual, and can be contacted with a MAPP or higher order MAPP complex in vitro. Such contacting can comprise single or multiple exposures of the population of T cells to a defined dose(s) and/or exposure schedule(s). In some cases, said contacting results in selectively binding/activating and/or expanding target T cells within the population of T cells, and results in generation of a population of activated and/or expanded target T cells. As an example, a mixed population of T cells can be peripheral blood mononuclear cells (PBMC). For example, PBMC from a patient can be obtained by standard blood drawing and PBMC enrichment techniques before being exposed to 0.1-1000 nM of a MAPP or higher order MAPP complex (e.g., duplex MAPP) of the present disclosure under standard lymphocyte culture conditions. At time points before, during, and after exposure of the mixed T cell population at a defined dose and schedule, the abundance of target T cells in the in vitro culture can be monitored by specific peptide-MHC multimers and/or phenotypic markers and/or functional activity (e.g. cytokine ELISpot assays). In some cases, upon achieving an optimal abundance and/or phenotype of antigen specific cells in vitro, all or a portion of the population of activated and/or expanded target T cells is administered to the individual (the individual from whom the mixed population of T cells was obtained).

In some cases, the population of T cells is in vitro. For example, a mixed population of T cells is obtained from an individual, and is contacted with a MAPP or higher order MAPP complex (e.g., duplex MAPP) of the present disclosure in vitro. Such contacting, which can comprise single or multiple exposures of the T cells to a defined dose(s) and/or exposure schedule(s) in the context of in vitro cell culture, can be used to determine whether the mixed population of T cells includes T cells that are specific for the epitope presented by the MAPP or higher order MAPP complex. The presence of T cells that are specific for the epitope of the MAPP or higher order MAPP complex can be determined by assaying a sample comprising a mixed population of T cells, which population of T cells comprises T cells that are not specific for the epitope (non-target T cells) and may comprise T cells that are specific for the epitope (target T cells). Known assays can be used to detect activation and/or proliferation of the target T cells, thereby providing an ex vivo assay that can determine whether a particular MAPP or higher order MAPP complex possesses an epitope that binds to T cells present in the individual, and thus whether the MAPP or higher order complex has potential use as a therapeutic composition for that individual. Suitable known assays for detection of activation and/or proliferation of target T cells include, e.g., flow cytometric characterization of T cell phenotype and/or antigen specificity and/or proliferation. Such an assay to detect the presence of epitope-specific T cells, e.g., a companion diagnostic, can further include additional assays (e.g. effector cytokine ELISpot assays) and/or appropriate controls (e.g. antigen-specific and antigen-nonspecific multimeric peptide-HLA staining reagents) to determine whether the MAPP or higher order MAPP complex is selectively binding/modulating (activating or inhibiting) and/or expanding the target T cell. Thus, for example, the present disclosure provides a method of detecting, in a mixed population of T cells obtained from an individual, the presence of a target T cell that binds an epitope of interest, the method comprising: a) contacting in vitro the mixed population of T cells with a MAPP or higher order MAPP complex comprising an epitope of the present disclosure; and b) detecting modulation (activation or inhibition) and/or proliferation of T cells in response to said contacting, wherein modulation of and/or proliferation of T cells indicates the presence of the target T cell. Alternatively, and/or in addition, if activation and/or expansion (proliferation) of the desired T cell population is obtained using a MAPP or higher order MAPP complex (e.g., a duplex MAPP), then all or a portion of the population of T cells comprising the activated/expanded T cells can be administered back to the individual as a therapy.

In some instances, the population of T cells is in vivo in an individual. In such instances, a method of the present disclosure for selectively delivering a costimulatory polypeptide (e.g., IL-2 or a reduced-affinity IL-2) to an epitope-specific T cell comprises administering the MAPP or higher order MAPP complex (e.g., duplex MAPP) to the individual.

### I. Dosages

A suitable dosage of a MAPP, higher order MAPP complex (e.g., duplex MAPP), or nucleic acid(s) encoding a MAPP can be determined by an attending physician or other qualified medical personnel, based on various clinical factors. As is well known in the medical arts, dosages for any one patient depend upon many factors, including the patient's size, body surface area, age, the particular polypeptide or nucleic acid to be administered, sex of the patient, time, and route of administration, general health, and other drugs being administered concurrently. A MAPP or higher order MAPP complex (e.g., duplex MAPP) may be administered in amounts between 1 ng/kg body weight and 20 mg/kg body weight per dose, e.g. between 0.1 mg/kg body weight to 10 mg/kg body weight, e.g. between 0.5 mg/kg body weight to 5 mg/kg body weight; however, doses below or above this exemplary range are envisioned, especially considering the aforementioned factors. If the regimen is a continuous infusion, it can also be in the range of 1 μg to 10 mg per kilogram of body weight per minute. A MAPP or higher order MAPP complex (e.g., duplex MAPP) of the present disclosure can be administered in an amount of from about 1 mg/kg body weight to 50 mg/kg body weight, e.g., from about 1 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, from about 20 mg/kg body weight to about 25 mg/kg body weight, from about 25 mg/kg body weight to about 30 mg/kg body weight, from about 30 mg/kg body weight to about 35 mg/kg body weight, from about 35 mg/kg body weight to about 40 mg/kg body weight, or from about 40 mg/kg body weight to about 50 mg/kg body weight.

In some cases, a suitable dose of a MAPP or higher order MAPP complex (e.g., duplex MAPP) of the present disclosure is from 0.01 μg to 100 mg per kg of body weight, from 0.1 μg to 1 g per kg of body weight, from 1 μg to 10 μg per kg of body weight, from 5.0 to 15.0 mg per kg of body weight, from 10.0 to 15.0 mg per kg of body weight, from 15.0 to 20.0 mg per kg of body weight, from 20-25 mg per kg of body weight, from 10 μg to 100 μg per kg of body weight, from 100 μg to 1.0 mg per kg of body weight, from 1 mg to 10 mg per kg of body weight from 10 mg to 100 mg per kg of body weight, or from 100 mg to 1.0 g per kg of body weight. Persons of ordinary skill in the art can easily estimate repetition rates for dosing based on measured residence times and concentrations of the administered agent in bodily fluids or tissues. Following successful treatment, it may be desirable to have the patient undergo maintenance therapy, i.e., periodic administrations intended to prevent the recurrence of the disease state, wherein a MAPP or higher order MAPP complex (e.g., duplex MAPP) is administered in maintenance doses, ranging from 0.01 μg to 10 g per kg of body weight, from 0.01 μg to 0.1 μg per kg of body weight, from 0.01 μg to 0.10 mg per kg of body weight from 0.1 μg to 1.0 μg per kg of body weight, from 1.0 μg to 10 μg per kg of body weight, from 10 μg to 100 μg per kg of body weight, from 100 μg to 1 mg per kg of body weight, from 1 mg to 10 mg per kg of body weight, from 5.0 to 10.0 mg per kg of body weight, from 5.0 to 15.0 mg per kg of body weight, from 10.0 to 15.0 mg per kg of body weight, from 15.0 to 20.0 mg per kg of body weight, from 20-25 mg per kg of body weight, from 10 mg to 100 mg per kg of body weight, or from 100 mg to 1.0 g per kg of body weight.

Those of skill will readily appreciate that dose levels can vary as a function of the MAPP or higher order MAPP complex (e.g., duplex MAPP), the severity of the symptoms and the susceptibility of the subject to side effects. Preferred dosages for a given compound are readily determinable by those of skill in the art by a variety of means.

In some cases, multiple doses of a MAPP or higher order MAPP complex (e.g., duplex MAPP) of the present disclosure, a nucleic acid of the present disclosure, or a recombinant expression vector of the present disclosure are administered. The frequency of administration of a MAPP or higher order MAPP complex (e.g., duplex MAPP) of the present disclosure, a nucleic acid of the present disclosure, or a recombinant expression vector of the present disclosure can vary depending on any of a variety of factors, e.g., severity of the symptoms, etc. For example, in some cases, a MAPP or higher order MAPP complex (e.g., duplex MAPP) of the present disclosure, a nucleic acid of the present disclosure, or a recombinant expression vector of the present disclosure is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

### J. Routes of Administration

The duration of administration of a MAPP or higher order MAPP complex (e.g., duplex MAPP) of the present disclosure, a nucleic acid of the present disclosure, or a recombinant expression vector of the present disclosure, e.g., the period of time over which a MAPP or higher order MAPP complex (e.g., duplex MAPP) of the present disclosure, a nucleic acid of the present disclosure, or a recombinant expression vector of the present disclosure is administered, can vary, depending on any of a variety of factors, e.g., patient response, etc. For example, a MAPP or higher order MAPP complex of the present disclosure, a nucleic acid of the present disclosure, or a recombinant expression vector of the present disclosure can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

An active agent (a MAPP or higher order MAPP complex of the present disclosure, a nucleic acid of the present disclosure, or a recombinant expression vector of the present disclosure) is administered to an individual using any available method and route suitable for drug delivery, including in vivo and ex vivo methods, as well as systemic and localized routes of administration.

Conventional and pharmaceutically acceptable routes of administration include intratumoral, peritumoral, intramuscular, intratracheal, intralymphatic, intracranial, subcutaneous, intradermal, topical application, intravenous, intraarterial, rectal, nasal, oral, and other enteral and parenteral routes of administration. Routes of administration may be combined, if desired, or adjusted depending upon the MAPP or higher order MAPP complex (e.g., duplex MAPP) and/or the desired effect. A MAPP or higher order MAPP complex of the present disclosure, or a nucleic acid or recombinant expression vector of the present disclosure, can be administered in a single dose or in multiple doses.

In some cases, a MAPP or higher order MAPP complex of the present disclosure, a nucleic acid of the present disclosure, or a recombinant expression vector of the present disclosure is administered intravenously. In some cases, a MAPP or higher order MAPP complex of the present disclosure, a nucleic acid of the present disclosure, or a recombinant expression vector of the present disclosure is administered intramuscularly. In some cases, a MAPP or higher order MAPP complex of the present disclosure, a nucleic acid of the present disclosure, or a recombinant expression vector of the present disclosure is administered intralymphatically. In some cases, a MAPP or higher order MAPP complex of the present disclosure, a nucleic acid of the present disclosure, or a recombinant expression vector of the present disclosure is administered locally. In some cases, a MAPP or higher order MAPP complex of the present disclosure, a nucleic acid of the present disclosure, or a recombinant expression vector of the present disclosure is administered intratumorally. In some cases, a MAPP or higher order MAPP complex of the present disclosure, a nucleic acid of the present disclosure, or a recombinant expression vector of the present disclosure is administered peritumorally. In some cases, a MAPP or higher order MAPP complex of the present disclosure, a nucleic acid of the present disclosure, or a recombinant expression vector of the present disclosure is administered intracranially. In some cases, a MAPP or higher order MAPP complex of the present disclosure, a nucleic acid of the present disclosure, or a recombinant expression vector of the present disclosure is administered subcutaneously.

In some cases, a MAPP or higher order MAPP complex of the present disclosure is administered intravenously. In some cases, a MAPP or higher order MAPP complex of the present disclosure is administered intramuscularly. In some cases, a MAPP or higher order MAPP complex of the present disclosure is administered locally. In some cases, a MAPP or higher order MAPP complex of the present disclosure is administered intratumorally. In some cases, a MAPP or higher order MAPP complex of the present disclosure is administered peritumorally. In some cases, a MAPP or higher order MAPP complex of the present disclosure is administered intracranially. In some cases, a MAPP or higher order MAPP complex of the present disclosure is administered subcutaneously. In some cases, a MAPP or higher order MAPP complex of the present disclosure is administered intralymphatically.

A MAPP or higher order MAPP complex of the present disclosure, a nucleic acid of the present disclosure, or a recombinant expression vector of the present disclosure can be administered to a host using any available conventional methods and routes suitable for delivery of conventional drugs, including systemic or localized routes. In general, routes of administration contemplated for use in a method of the present disclosure include, but are not necessarily limited to, enteral, parenteral, and inhalational routes.

Parenteral routes of administration other than inhalation administration include, but are not necessarily limited to, topical, transdermal, subcutaneous, intramuscular, intraorbital, intracapsular, intraspinal, intrasternal, intratumoral, intralymphatic, peritumoral, and intravenous routes, i.e., any route of administration other than through the alimentary canal. Parenteral administration can be carried out to effect systemic or local delivery of a MAPP or higher order MAPP complex of the present disclosure by administration of a nucleic acid of the present disclosure, or a recombinant expression vector of the present disclosure. Where systemic delivery is desired, administration typically involves invasive or systemically absorbed topical or mucosal administration of pharmaceutical preparations.

### K. Subjects Suitable for Treatment

Subjects suitable for treatment, e.g., by selectively delivering a MOD to a T cell or by modulating their T cell activity, include those with a neoplasm, such as benign neoplasm or a malignant neoplasm (e.g., a cancer in the form of a solid malignant tumor).

Subjects suitable for treatment who have a cancer include, but are not limited to, individuals who have been provided other treatments for the cancer but who failed to respond to the treatment. Cancers and neoplasms that can be treated with a method of the present disclosure include, but are not limited to, those displaying any of the KRAS cancer epitopes including those recited herein. Such cancers include, but are not limited to, non-small cell lung cancer, lung adenocarcinoma, mucinous adenoma, ductal carcinoma of the pancreas, colorectal cancer, or leukemia.

### IV. Certain Aspects

Certain aspects, including embodiments/aspects of the present subject matter described above, may be beneficial alone or in combination, with one or more other aspects recited hereinbelow. In addition, while the present invention has been disclosed with reference to certain aspects recited below and in the claims, numerous modifications, alterations, and changes to the described aspects/embodiments are possible without departing from the sphere and scope of the present invention. Accordingly, it is intended that the present invention not be limited to the described embodiments, aspects and claims, but that it has the full scope defined by the language of this disclosure and equivalents thereof.
1. A multimeric antigen-presenting polypeptide complex (MAPP) comprising:
   a framework polypeptide comprising (e.g., from N-terminus to C-terminus) a dimerization sequence and a multimerization sequence;
   a dimerization polypeptide comprising a counterpart dimerization sequence complementary to the dimerization sequence of the framework polypeptide, and dimerizing therewith through covalent (e.g., disulfide bonds) and/or non-covalent interactions to form a MAPP heterodimer; and
   at least one (e.g., at least two) presenting sequence and/or presenting complex,
   wherein each presenting sequence comprises KRAS epitope, MHC class I heavy chain (“MHC-H”), and β2M polypeptide sequences;
   wherein each presenting complex comprises a presenting complex 1^{st} sequence and a presenting complex 2^{nd} sequence that together comprise KRAS epitope, MHC-H, and β2M polypeptide sequences (the KRAS epitope is part of the presenting complex 1^{st} sequence or presenting complex 2^{nd} sequence along with either the MHC-H or β2M polypeptide sequence);
   wherein one or both of the dimerization polypeptide and/or the framework polypeptide (e.g., either the framework polypeptide, dimerization polypeptide, or both polypeptides) comprise a presenting sequence or a presenting complex 1st sequence (e.g., located on the N-terminal side of the framework polypeptide dimerization sequence, or the N-terminal side of the dimerization polypeptide counterpart dimerization sequence);
   wherein optionally at least one (e.g., one, two, or more) of the framework polypeptide, dimerization polypeptide, presenting sequence(s), presenting complex 1st sequence and/or presenting complex 2nd sequence comprises one, two, three or more independently selected MOD and/or variant MOD polypeptide sequences (e.g., located at their N-terminus, C-terminus, or on the N-terminal or C-terminal side of the dimerization sequences), wherein the one or more MOD polypeptide sequences optionally may be polypeptides such as wt. IL-2 or a variant of wt. IL-2 (e.g., that comprises an H16A or T substitution and an F42A substitution) that result in T cell activation, wherein T cell activation may result in one or more of the following: an increase in the activity of ZAP70 protein kinase activity, induction in the proliferation of the T-cell(s), granule-dependent effector actions (e.g., the release of granzymes, perforin, and/or granulysin from cytotoxic T-cells), and/or release of T cell cytokines (e.g., interferon γ from CD8+ cells), and
   wherein the framework polypeptide, dimerization polypeptide, presenting sequence(s), presenting complex 1^{st} sequence and/or presenting complex 2^{nd} sequence optionally comprise one or more linker sequences selected independently. (See, e.g., FIGS. 1A and 1B.)
      It is understood that the dimerization sequence and multimerization sequence are different polypeptide sequences and do not bind in any substantial manner to each other, e.g., the framework polypeptides do not, to any substantial extent, form hair pin structures, self-polymerize, or self-aggregate. Similarly, such aspects may be subject to the proviso that neither the dimerization sequence nor the multimerization sequence of the framework polypeptide comprises an MHC-H polypeptide sequence having at least 85% (e.g. 90%, 95% or 98%) sequence identity to at least 20 (e.g., at least 30, 40, 50, 60 or 70) contiguous aas of a β2M or MHC-H polypeptide in any of FIG. 2 or 3A through 3H.
2. The MAPP of aspect 1, wherein the MHC-H polypeptide sequence comprises a human class I MHC-H chain polypeptide sequence selected from HLA-A, HLA-B, HLA-C, HLA-E, HLA-F, and HLA-G MHC-H polypeptide sequences or a portion thereof.
3. The MAPP of aspect 1, wherein at least one presenting sequence or presenting complex (e.g., at least two or all presenting sequences and/or complexes) comprises:
   an MHC-H sequence having at least 85% (e.g., at least 90%, at least 95% or 98%) or 100% sequence identity to at least 125 (e.g., at least 150, 175, 200, 250, or 275) contiguous aas of an MHC-H polypeptide provided in any of FIGS. 3A to 3H, wherein the MHC-H sequences do not include the MHC-H transmembrane domain, or a portion thereof, that will anchor the MAPP in a cell membrane; and/or
   a β2M sequence having at least 90% (e.g., at least 95% or 98%) or 100% sequence identity to at least 50 (e.g., 60, 70, 80, or 89) contiguous aas of a mature β2M polypeptide provided in FIG. 2.
4. The MAPP of any of aspects 1 to 3, wherein the MHC-H polypeptide sequences have at least 90% (e.g., at least 95% or 98%) or 100% sequence identity to at least 125 (e.g., at least 150, 175, 200, 250, or 275) contiguous aas of an HLA-A allele that does not include the MHC-H transmembrane domain or a portion thereof that will anchor the MAPP in a cell membrane.
5. The MAPP of any of aspects 1 to 4, wherein the MHC-H polypeptide sequences have at least 90% (e.g., at least 95% or 98%) or 100% sequence identity to at least 125 (e.g., at least 150, 175, 200, 250, or 275) contiguous aas of an HLA-A*0101, HLA-A*0201, HLA-A*0301, HLA-A*1101, HLA-A*2301, HLA-A*2402, HLA-A*2407, HLA-A*3303, or HLA-A*3401 polypeptide sequence provided e.g., in FIG. 3E.
6. The MAPP of any of aspects 1 to 5, wherein the MHC-H polypeptide sequences have at least 90% (e.g., at least 95% or 98%) or 100% sequence identity to at least 125 (e.g., at least 150, 175, 200, 250, or 275) contiguous aas of an HLA-A*0101, HLA-A*0201, HLA-A*1101, HLA-A*2402, or HLA-A*3303 polypeptide sequence (e.g., as provided in FIG. 3E).
7. The MAPP of any of aspects 1 to 3, wherein the MHC-H polypeptide sequences have at least 90% (e.g., at least 95% or 98%) or 100% sequence identity to at least 125 (e.g., at least 150, 175, 200, 250, or 275) contiguous aas of an HLA-B allele that does not include the MHC-H transmembrane domain or a portion thereof that will anchor the MAPP in a cell membrane.
8. The MAPP of any of aspects 1 to 3 or 7, wherein the MHC-H polypeptide sequences have at least 90% (e.g., at least 95% or 98%) or 100% sequence identity to at least 125 (e.g., at least 150, 175, 200, 250, or 275) contiguous aas of an HLA-B*0702, HLA-B*0801, HLA-B*1502, B27 (subtypes HLA-B*2701-2759), HLA-B*3802, HLA-B*4001, HLA-B*4601, or HLA-B*5301 polypeptide sequence (e.g., as provided in FIG. 3F).
9. The MAPP of any of aspects 1 to 3 or 7, wherein the MHC-H polypeptide sequences have at least 90% (e.g., at least 95% or 98%) or 100% sequence identity to at least 125 (e.g., at least 150, 175, 200, 250, or 275) contiguous aas of HLA-B*0702.
10. The MAPP of any of aspects 1 to 3, wherein the MHC-H polypeptide sequences have at least 90% (e.g., at least 95% or 98%) or 100% sequence identity to at least 125 (e.g., at least 150, 175, 200, 250, or 275) contiguous aas of an HLA-C allele that does not include the MHC-H transmembrane domain or a portion thereof that will anchor the MAPP in a cell membrane.
11. The MAPP of any of aspects 1 to 3 or 10, wherein the MHC-H polypeptide sequences have at least 90% (e.g., at least 95% or 98%) or 100% sequence identity to at least 125 (e.g., at least 150, 175, 200, 250, or 275) contiguous aas of an HLA-C*0102, HLA-C*0303, HLA-C*0304, HLA-C*0401, HLA-C*0602, HLA-C*0701, HLA-C*0702, HLA-C*0801, or HLA-C*1502 polypeptide sequence (e.g., as provided in FIG. 3G).
12. The MAPP of any of aspects 1 to 3 or 10, wherein the MHC-H polypeptide sequences have at least 90% (e.g., at least 95% or 98%) or 100% sequence identity to at least 125 (e.g., at least 150, 175, 200, 250, or 275) contiguous aas of HLA-C*0701.
13. The MAPP of any of aspects 1 to 3, wherein the MHC-H polypeptide sequences have at least 90% (e.g., at least 95% or 98%) or 100% sequence identity to at least 125 (e.g., at least 150, 175, 200, 250, or 275) contiguous aas of an HLA-E allele that does not include the MHC-H transmembrane domain or a portion thereof that will anchor the MAPP in a cell membrane.
14. The MAPP of any of aspects 1 to 3 or 13, wherein the MHC-H polypeptide sequences have at least 90% (e.g., at least 95% or 98%) or 100% sequence identity to at least 125 (e.g., at least 150, 175, 200, 250, or 275) contiguous aas of an HLA-E*0101, HLA-E*01:03, HLA-E*01:04, HLA-E*01:05, HLA-E*01:06, HLA-E*01:07, HLA-E*01:09, or HLA-E*01:10 polypeptide sequence (e.g., as provided in FIG. 3H).
15. The MAPP of any of aspects 1 to 3 or 13, wherein the MHC-H polypeptide sequences have at least 90% (e.g., at least 95% or 98%) or 100% sequence identity to at least 125 (e.g., at least 150, 175, 200, 225, or all) of the non-variable aas of the HLA-E allele consensus sequence:

GSHSLKYFHT SVSRPGRGEP RFISVGYVDD TQFVRFDNDA
ASPRMVPRAP WMEQEGSEYW DRETRSARDT AQIFRVNLRT
LRG [symbol] YNQSX1A GSHTLQWMHG CELGPDX2RFL RGYEQFAYDG
KDYLTLNEDL RSWTAVDT [symbol] A QISEQKSNDA SEAEHQX3X4YL
EDTCVEWLHK YLEKGKETLL HLEPPKTHVT HHPISDHEAT
LRCWALGFYP AEITLTWQQD GEGHTQDTEL VETRP [symbol] GDGT
FQKWAAVVVP SGEEX5RYTCH VQHEGLX6EPV TLRWKPASQP
TIPI,

wherein X1=K or E, X2=R or G, X3=R or G, X4=A or V, X5=Q or P, and X6=P or S.
16. The MAPP of any of aspects 1 to 3, wherein the MHC-H polypeptide sequences have at least 90% (e.g., at least 95% or 98%) or 100% sequence identity to at least 125 (e.g., at least 150, 175, 200, 250, or 275) contiguous aas of an HLA-F allele that does not include the MHC-H transmembrane domain or a portion thereof that will anchor the MAPP in a cell membrane.
17. The MAPP of any of aspects 1 to 3 or 16, wherein the MHC-H polypeptide sequences have at least 90% (e.g., at least 95% or 98%) or 100% sequence identity to at least 125 (e.g., at least 150, 175, 200, 250, or 275) contiguous aas of an HLA-F*0101 (HLA-F*01:01:01:01), HLA-F*01:02, HLA-F*01:03 (HLA-F*01:03:01:01), HLA-F*01:04, HLA-F*01:05, or HLA-F*01:06 polypeptide sequence (e.g., as provided in FIG. 3H).
18. The MAPP of any of aspects 1 to 3 or 16, wherein the MHC-H polypeptide sequences have at least 90% (e.g., at least 95% or 98%) or 100% sequence identity to at least 125 (e.g., at least 150, 175, 200, 225, or all) of the non-variable aas of the HLA-F allele consensus sequence:

GSHSLRX1FST AVSRPGRGEP RYIAVEYVDD TQFLRFDSDA
AIPRMEPREX2 WVEQEGPQYW EWTTGYAKAN AQTDRVALRN
LLRRYNQSEA GSHTLQGMNG CDMGPDGRLL RGYHQHAYDG
KDYISLNEDL RSWTAADTVA QITQRFYEAE EYAEEFRTYL
EGECLELLRR YLENGKETLQ RADPPKAHVA HHPISDHEAT
LRCWALGFYP AEITLTWQRD GEEQTQDTEL VETRPAGDGT
FQKWAAVVVP X3GEEQRYTCH VQHEGLPQPL ILRWEQSX4QP
TIPI,

wherein X1=Y or F; X2=P or Q; X3=S or P; and X4=P or L.
19. The MAPP of any of aspects 1 to 3, wherein the MHC-H polypeptide sequences have at least 90% (e.g., at least 95% or 98%) or 100% sequence identity to at least 125 (e.g., at least 150, 175, 200, 250, or 275) contiguous aas of an HLA-G allele that does not include the MHC-H transmembrane domain or a portion thereof that will anchor the MAPP in a cell membrane.
20. The MAPP of any of aspects 1 to 3 or 19, wherein the MHC-H polypeptide sequences have at least 90% (e.g., at least 95% or 98%) or 100% sequence identity to at least 125 (e.g., at least 150, 175, 200, 250, or 275) contiguous aas of an HLA-G*01:04 (HLA-G*01:04:01:01), HLA-G*01:06, HLA-G*01:07, HLA-G*01:08, HLA-G*01:09, HLA-G*01:10, HLA-G*01:11, HLA-G*01:12, HLA-G*01:14, HLA-G*01:15, HLA-G*01:16, HLA-G*01:17, HLA-G*01:18, HLA-G*01:19, HLA-G*01:20, or HLA-G*01:22 polypeptide sequence (e.g., as provided in FIG. 3H).
21. The MAPP of any of aspects 1 to 3 or 19, wherein the MHC-H polypeptide sequences have at least 90% (e.g., at least 95% or 98%) or 100% sequence identity to at least 125 (e.g., at least 150, 175, 200, 225, or all) of the non-variable aas of the HLA-G allele consensus sequence:

GSHSMRYFSA AVX1RPGRGEP RFIAMGX2VDD X3QFX4RFDSDS ACPRMEPRAP
WVEX5EGPEYW EEETRNTKAH AQTDRMNLQT X6RG [symbol] YNQSEA SSHTLQWMIX7
CDLX8X9DGRLX10 RGYEQYAYDG KDYLALNEDL RSWTAADT [symbol] A QISKRKCEAA
NVAEQRRAX11L EGTCVEWLX12R X13LENGKEX14LQ RADPX15KTHVT HHPVFDYEAT
LRCWALGFYP AEIILTWQX16D GEDQTQDVEL VETRP [symbol] GDGT FQKWAAVVVP
SGEEQRYX17CH VQHEGLPEPL MLRWX18QSSLP TIPI,

wherein X1=S or F, X2=Y or H, X3=T, S, or M, X4=L or V; X5=Q or R, X6=P or L, X7=G or D, X8=G or V, X9=S or C, X10=L or I, X11=Y or H, X12=H or R, X13=Y or H, X14=M or T, X15=P or A, X16=R, W, or Q, X17=T or M, X18=K or E.
22. The MAPP of any preceding aspect, wherein the β2M sequence has at least 90% (e.g., at least 95% or 98%) or 100% sequence identity to at least 50 (e.g., 60, 70, 80, or 89) contiguous aas of a mature human β2M polypeptide (e.g., aas 21-119 of NCBI accession number NP_004039.1 provided in FIG. 2).
23. The MAPP of any of aspects 1 to 22, wherein at least one of the MHC-H polypeptide sequences comprises at least one mutation (e.g., two or three mutations) selected from the group consisting of: an alanine at position 84 (e.g., Y84A or R84A in the case of HLA-F), a cysteine at position 84 (e.g., Y84C or R84C in the case of HLA-F), a cysteine at position 139 (e.g., A139C or V139C in the case of HLA-F), and a cysteine at position 236 (e.g., A236C). See FIG. 3I for the location of those aa positions.
24. The MAPP of any of aspects 1 to 23, wherein at least one of the MHC class I polypeptide sequences comprises a combination of mutations selected from the group consisting of: Y84A and A139C; Y84A and A236C; Y84C and A139C; Y84C and A236C; and Y84C, A139C and A236C.
25. The MAPP of any of aspects 1 to 24, wherein the at least one presenting sequence/complex (e.g., two, three, four, or each) comprises:
   a β2M sequence having a cysteine at position 12 of the mature β2M peptide (e.g., an R12C substitution, see FIG. 2 for numbering) and an MHC-H sequence with a cysteine at position 236 (e.g., an A236C substitution, see, e.g., FIG. 3I for numbering) with the cysteines forming a disulfide bond; and/or
   a β2M sequence having an amino terminal KRAS epitope and cysteine-containing peptide linker at its amino terminus (e.g., with the linker comprising a cysteine as the second, third, fourth, or fifth aa from the epitope's C-terminal aa) and an MHC-H sequence with a cysteine at position 84 (e.g., a Y84C substitution, see, e.g., FIG. 3I for numbering) with the cysteines forming a disulfide bond.
26. The MAPP of any preceding aspect, wherein the MAPP comprises at least one linker that comprises one or more sequences selected from (e.g., combinations of): polyG (e.g., polyglycine), GA, AG, AS, SA, GS, GSGGS (SEQ ID NO:101), GGGS (SEQ ID NO:102), GGSG (SEQ ID NO:103), GGSGG (SEQ ID NO:104), GSGSG (SEQ ID NO:105), GSGGG (SEQ ID NO:106), GGGSG (SEQ ID NO:107), GSSSG (SEQ ID NO:108), GSSSS (SEQ ID NO:109), GGGGS (SEQ ID NO:110), or AAAGG (SEQ ID NO:111), any of which may be repeated 1, 2, 3, 4, 5, 6, 7, 8, 9, or 10 times.
27. The MAPP of any preceding aspect, wherein the MAPP comprises at least one aa sequence independently selected from GCGASGGGGSGGGGS (SEQ ID NO:113), GCGGSGGGGSGGGGSGGGGS (SEQ ID NO:93), GCGGSGGGGSGGGGS (SEQ ID NO:94), and GCGGS(G₄S) (SEQ ID NO:87) where the G₄S unit may be repeated from 1 to 10 times (e.g., repeated 1, 2, 3, 4, 5, 6, 7, 8, 9, or 10 times); wherein the linker cysteine residue optionally forms a disulfide bond (e.g., with another peptide sequence of the MAPP).
28. The MAPP of any preceding aspect, wherein:
   a) the at least one presenting sequence (e.g., two or each presenting sequence) comprises (e.g., from N-terminus to C-terminus), either
      (i) the KRAS epitope sequence, the β2M sequence, and the MHC-H polypeptide sequence, or
      (ii) the KRAS epitope sequence, the MHC-H polypeptide sequence, and the β2M polypeptide sequence (see, e.g., FIG. 12); and/or
   b) the at least one presenting complex (e.g., two, three, four, or each presenting complex) comprises
      (i) the presenting complex 1^{st} sequence comprising the MHC-H polypeptide, its associated presenting complex 2^{nd} sequence comprising the KRAS epitope sequence and the β2M polypeptide (e.g., the epitope is placed N-terminal to the β2M) (see, e.g., FIG. 13, structures A, C, and E, and FIG. 14, structures A, C, and E), or
      (ii) the presenting complex 1^{st} sequence comprising (e.g., from N-terminus to C-terminus) the KRAS epitope sequence and the β2M sequence (e.g., the KRAS epitope is placed N-terminal to the β2M), the associated presenting complex 2^{nd} sequence comprising the MHC-H polypeptide sequence (see, e.g., FIG. 13, structures B, D, and F, and FIG. 14, structures B, D and F),
      wherein the presenting complex 1^{st} sequence and its associated presenting complex 2^{nd} sequence are optionally joined by at least one disulfide bond (see, e.g., FIG. 15);
      wherein any one or more of the KRAS epitope sequence, the β2M sequence, and the MHC-H polypeptide are joined by optional linkers; and
      wherein the at least one presenting sequence and/or the at least one presenting complex optionally comprise one or more, or two or more, independently selected MODs or variant MODs.
29. The MAPP of aspect 28, wherein a linker cysteine residue forms a disulfide bond between a presenting sequence and another polypeptide of the MAPP, or between the presenting complex 1st sequence and another polypeptide of the MAPP (e.g., with a presenting complex 2nd sequence).
30. The MAPP of any preceding aspect, wherein the dimerization and/or multimerization sequences are independently selected from non-interspecific sequences or interspecific sequences.
31. The MAPP of aspect 30, wherein the interspecific and non-interspecific sequences are selected from the group consisting of: immunoglobulin heavy chain constant regions (Ig Fc, e.g., CH2-CH3), collectin polypeptides, coiled-coil domains (see, e.g., SEQ ID NOs:71-77), leucine-zipper domains (see, e.g., SEQ ID NOs:79-82 and 100), Fos polypeptides, Jun polypeptides, Ig CH1, Ig C_{L}κ, Ig C_{L} λ, knob-in-hole without disulfide (KiH), knob-in hole with a stabilizing disulfide bond (KiHs-s), HA-TF, ZW-1, 7.8.60, DD-KK, EW-RVT, EW-RVTs-s, and A107 sequences.
32. The MAPP of any preceding aspect, complexed to form a duplex or higher order MAPP comprising at least a first MAPP heterodimer and a second MAPP heterodimer of any of aspects 1-31, wherein:
   (i) the first heterodimer comprises a first framework polypeptide having a first multimerization sequence and a first dimerization sequence, and a first dimerization polypeptide having a first counterpart dimerization sequence complementary to the first dimerization sequence; and
   (ii) the second heterodimer comprises a second framework polypeptide having a second multimerization sequence and a second dimerization sequence, and a second dimerization polypeptide having a second counterpart dimerization sequence complementary to the second dimerization sequence; and
wherein the first and second framework polypeptides are associated by binding interactions between the first and second multimerization sequences optionally including one or more interchain covalent bonds (e.g, one or two disulfide bonds), and the multimerization sequences are not the same (e.g., not the same type and/or not identical to) as, and do not substantially associate with or bind to, the dimerization or counterpart dimerization sequences. See, e.g., the duplexes in FIGS. 6 to 10.
33. The duplex MAPP of aspect 32, wherein the first and second dimerization sequence are identical, and the first and second counterpart dimerization sequences are identical. See, e.g., FIG. 6, structure A.
34. The duplex MAPP of aspect 33, wherein the first and second dimerization sequences do not substantially associate with or bind to each other.
35. The duplex MAPP of aspect 32, wherein the first and second multimerization sequences are interspecific multimerization sequences that form an interspecific pair, the first and second dimerization sequence are identical, and the first and second counterpart dimerization sequences are identical. See, e.g., FIG. 6, structure B.
36. The duplex MAPP of aspect 35, wherein the first and second dimerization sequences do not substantially associate with or bind to each other.
37. The duplex MAPP of any of aspects 35 to 36, wherein the first or second framework polypeptide comprises at least one MOD or variant MOD (e.g., two or three MODs or variant MODs) not present on the other framework polypeptide.
38. The duplex MAPP of aspect 32, wherein the first and second multimerization sequences are identical, the first dimerization sequence and the first counterpart dimerization sequence are interspecific dimerization sequences forming a first interspecific pair, and the second dimerization sequence and second counterpart dimerization sequence are interspecific dimerization sequences forming a second interspecific pair. See, e.g., FIG. 6, structure C.
39. The duplex MAPP of aspect 38, wherein the first and second dimerization sequences are identical and the first and second counterpart dimerization sequences are identical. See, e.g., FIG. 8, structure A.
40. The duplex MAPP of aspect 38, wherein the first and second dimerization sequences are not identical and do not bind in any substantial manner to each other.
41. The duplex MAPP of aspect 38, wherein the polypeptides of the first interspecific pair are different from (not identical to), and do not bind or interact with the polypeptides of the second interspecific pair.
42. The duplex MAPP of any of aspects 40 to 41, wherein the first or second dimerization polypeptide comprises at least one MOD (e.g., two or three MODs) not present on the other dimerization polypeptide.
43. The duplex MAPP of aspect 32, wherein
   the first and second multimerization sequences are interspecific multimerization sequences that form an interspecific multimerization pair,
   the first dimerization sequence and the first counterpart dimerization sequence are interspecific dimerization sequences forming a first interspecific pair, and
   the second dimerization sequence and second counterpart dimerization sequence are interspecific dimerization sequences forming a second interspecific pair. See, e.g., FIG. 6, structure D.
44. The duplex MAPP of aspect 43, wherein the first and second dimerization sequences are identical and the first and second counterpart dimerization sequences are identical. See, e.g., FIG. 8, structure D.
45. The duplex MAPP of aspect 43, wherein the first and second dimerization sequences do not substantially associate with or bind with each other.
46. The duplex MAPP of any of aspects 38 to 45, wherein the polypeptides of the first interspecific pair are different from (not identical to), and do not bind or interact with the polypeptides of the second interspecific pair.
47. The duplex MAPP of any of aspects 45 to 46, wherein the first or second dimerization polypeptide comprises at least one MOD or variant MOD (e.g., two or three MODs or variant MODs) not present on the other dimerization polypeptide.
48. The duplex MAPP of any of aspects 43 to 47, wherein the first or second framework polypeptide comprises at least one MOD or variant MOD (e.g., two or three MODs or variant MODs) not present on the other framework polypeptide.
49. The duplex MAPP of any of aspects 32 to 48, wherein, when the multimerization sequences are not an interspecific multimerization pair, the multimerization sequences are selected from the group consisting of immunoglobulin heavy chain constant regions (e.g., IgFc CH2-CH3, see, e.g., SEQ ID NOs:54-65), collectin family dimerization sequences, coiled-coil domains, and leucine-zipper domains; and
   wherein, when the multimerization sequences are an interspecific multimerization pair, the multimerization sequences are selected from the group consisting of a Fos and Jun polypeptide pair, Ig CH1 and Ig C_{L} κ or λ constant region polypeptide pair, a knob-in-hole without disulfide (KiH) pair, a knob-in hole with a stabilizing disulfide bond (KiHs-s) pair, a HA-TF polypeptide pair, a ZW-1 polypeptide pair, a 7.8.60 polypeptide pair, a DD-KK polypeptide pair, an EW-RVT polypeptide pair, an EW-RVTs-s polypeptide pair, and an A107 polypeptide pair.
50. The duplex MAPP of aspect 49, wherein the multimerization sequences, the first dimerization sequence and its counterpart first dimerization sequence, and the second dimerization sequence and its counterpart dimerization sequence are each selected from the group consisting of: immunoglobulin heavy chain constant regions (e.g., IgFc CH2-CH3, see, e.g., SEQ ID NOs:54-65), collectin family dimerization sequences, coiled-coil domains, and leucine-zipper domains; and
wherein the first and second dimerization sequences, are selected independently and may be the same or different.
51. The duplex MAPP of aspect 49, wherein the multimerization sequences are selected from the group consisting of immunoglobulin heavy chain constant regions (e.g., Ig CH2-CH3), collectin family dimerization sequences, coiled-coil domains, and leucine-zipper domains;
   wherein a pair comprising the first dimerization sequence and its counterpart dimerization sequence, and a pair comprising the second dimerization sequence and its counterpart dimerization sequence, are independently selected from the group consisting of a Fos and Jun polypeptide pair, Ig CH1 and Ig C_{L} κ or λ constant region polypeptide pair, a knob-in-hole without disulfide (KiH) pair, a knob-in hole with a stabilizing disulfide bond (KiHs-s) pair, an HA-TF polypeptide pair, a ZW-1 polypeptide pair, a 7.8.60 polypeptide pair, a DD-KK polypeptide pair, an EW-RVT polypeptide pair, an EW-RVTs-s polypeptide pair, and an A107 polypeptide pair; and
   wherein the pairs may be the same or different.
52. The duplex MAPP of aspect 49, wherein the multimerization sequences are selected from the group consisting of a Fos and Jun polypeptide pair, Ig CH1 and Ig C_{L} κ or λ constant region polypeptide pair, a knob-in-hole without disulfide (KiH) pair, a knob-in hole with a stabilizing disulfide bond (KiHs-s) pair, an HA-TF polypeptide pair, a ZW-1 polypeptide pair, a 7.8.60 polypeptide pair, a DD-KK polypeptide pair, an EW-RVT polypeptide pair, an EW-RVTs-s polypeptide pair, and an A107 polypeptide pair;
   wherein the first and second dimerization sequences, which may be the same or different, and their counterpart dimerization sequences are independently selected from the group consisting of immunoglobulin heavy chain constant regions (e.g., Ig CH2-CH3), collectin family dimerization sequences, coiled-coil domains, and leucine-zipper domains.
53. The duplex MAPP of aspect 49, wherein the multimerization sequences, the first dimerization sequence and its counterpart first dimerization sequence, and the second dimerization sequence and its counterpart dimerization sequence are each selected as a pair from the group consisting of: Fos and Jun polypeptide pairs, Ig CH1 and Ig C_{L} κ or λ constant region polypeptide pairs, knob-in-hole without disulfide (KiH) pairs, knob-in hole with a stabilizing disulfide bond (KiHs-s) pairs, HA-TF polypeptide pairs, ZW-1 polypeptide pairs, 7.8.60 polypeptide pairs, DD-KK polypeptide pairs, EW-RVT polypeptide pairs, EW-RVTs-s polypeptide pairs, and A107 polypeptide pairs; and
   wherein the pairs comprising the first and second dimerization sequences may be the same or different.
54. The duplex MAPP of aspect 49, wherein the multimerization sequences comprise Ig Fc regions and the first and second dimerization sequences comprise independently selected Ig CH1, Ig C_{L} κ or λ, leucine zipper, Fos or Jun domains.
55. The duplex MAPP of aspect 49, wherein the multimerization sequences comprise Ig Fc regions and the first and second dimerization sequences comprise independently selected Ig CH1 or Ig C_{L} κ or λ domains.
56. The duplex MAPP of any of aspects 54 to 55, wherein the Ig CH2-CH3 domains are selected from the group consisting of IgA, IgD, IgE, IgG and IgM Fc regions.
57. The duplex MAPP of aspects 54 to 56, wherein the Ig Fc regions are selected from IgG1, IgG2, IgG3, and IgG4 CH2-CH3 domains.
58. The duplex MAPP of any of aspects 54 to 56, wherein the Ig Fc regions are IgG1CH2-CH3 domains.
59. The duplex MAPP of aspect 49, wherein the multimerization sequences are a pair of interspecific immunoglobulin sequences.
60. The duplex MAPP of aspect 59, wherein the pair of interspecific immunoglobulin sequences are selected from the group consisting of knob-in-hole without disulfide (KiH) pairs, knob-in hole with a stabilizing disulfide bond (KiHs-s) pairs, HA-TF polypeptide pairs, ZW-1 polypeptide pairs, 7.8.60 polypeptide pairs, DD-KK polypeptide pairs, EW-RVT polypeptide pairs, EW-RVTs-s polypeptide pairs, and A107 polypeptide pairs
61. The duplex MAPP of any of aspects 59 to 60, wherein the multimerization sequences are a pair of interspecific immunoglobulin sequences comprising a knob-in-hole without disulfide (KiH).
62. The duplex MAPP of any of aspects 59 to 60, wherein the multimerization sequences are a pair of interspecific immunoglobulin sequences comprising a knob-in hole pair that comprises at least one stabilizing disulfide bond (e.g., a KiHs-s pair).
63. The duplex MAPP of any of aspects 59 to 62, wherein the first and second dimerization sequences comprise independently selected Ig CH1, Ig C_{L} κ or λ, leucine zipper, Fos or Jun domains.
64. The duplex MAPP of any of aspects 59 to 63, wherein the first and second dimerization sequences comprise independently selected Ig CH1 or Ig CL κ or λ domains.
65. The duplex MAPP of any of aspects 59 to 64, wherein the first and second dimerization sequences comprise Ig CH1 domains.
66. The duplex MAPP of any of aspects 59 to 64, wherein the first and/or second dimerization sequences do not comprise Ig CH1 domains.
67. The MAPPs of any of aspects 1 to 66, wherein the dimerization polypeptide and framework polypeptide are covalently linked by at least one (e.g., two) disulfide bond(s).
68. The duplex MAPPs of any of aspects 32 to 67, wherein the first MAPP heterodimer and/or the second MAPP heterodimer are covalently linked by at least one (e.g., two) disulfide bond(s).
69. The duplex MAPPs of any of aspects 32 to 68, wherein the multimerization sequences of the first and second framework polypeptides are covalently linked by at least one (e.g., two) disulfide bond(s).
70. The duplex MAPPs of any of aspects 32 to 66, wherein the first dimerization sequence and its counterpart dimerization sequence and/or the second dimerization sequence and its counterpart dimerization sequence are covalently linked by at least one (e.g., two) disulfide bond(s); and the multimerization sequences of the first and second framework polypeptides are covalently linked by at least one (e.g., two) disulfide bond(s).
71. The MAPP or duplex MAPP of any preceding aspect, wherein each MAPP comprises only one presenting sequence or one presenting complex. See, e.g., the 1st or 2nd heterodimer in FIG. 1.
72. The MAPP or duplex of aspect 71, wherein the one presenting sequence or the presenting complex 1^{st} sequence of the presenting complex is part of the dimerization polypeptide (e.g., located on the N-terminal side of the counterpart dimerization sequence). See, e.g., the 1st or 2nd heterodimer in FIG. 1.
73. The MAPP or duplex of aspect 71, wherein the one presenting sequence or the presenting complex 1^{st} sequence of the presenting complex is part of the framework polypeptide (e.g., located on the N-terminal side of the dimerization sequence).
74. The duplex MAPP of any of aspects 32-70, wherein the duplex MAPP comprises only one presenting sequence or one presenting complex.
75. The duplex MAPP of aspect 74, wherein the one presenting sequence or the presenting complex 1^{st} sequence of the presenting complex is part of one dimerization polypeptide (e.g., located on the N-terminal side of the counterpart dimerization sequence).
76. The duplex MAPP of aspect 74, wherein the one presenting sequence or the presenting complex 1^{st} sequence of the presenting complex is part of one framework polypeptide (e.g., located on the N-terminal side of the dimerization sequence).
77. The duplex MAPP of any of aspects 32 to 70, wherein the duplex MAPP comprises at least two presenting sequences or at least two presenting complexes. See, e.g., the duplex in FIG. 1.
78. The duplex MAPP of aspect 77, wherein one of the at least two presenting sequences or presenting complex 1^{st} sequences of the at least two presenting complexes is part of the first dimerization polypeptide, and the second of the at least two presenting sequences or presenting complex 1^{st} sequences is part of the second dimerization polypeptides (e.g., located on the N-terminal side of their counterpart dimerization sequences). See, e.g., the duplex in FIG. 1.
79. The duplex MAPP of aspect 77, wherein one of the at least two presenting sequences or each of the presenting complex 1^{st} sequences of the at least two presenting complexes is part of the first framework polypeptide, and the second of the at least two presenting sequences or presenting complex 1^{st} sequences is part of the second framework polypeptide (e.g., located on the N-terminal side of their dimerization sequence).
80. The duplex MAPP of any of aspects 32 to 70, wherein the duplex MAPP comprises at least four presenting sequences and/or presenting complexes. See, e.g., FIG. 7, structures A-D.
81. The duplex MAPP of aspect 80, wherein each one of the four presenting sequences or each one of the presenting complex 1^{st} sequences of the four presenting complexes are each part of a different one of the first dimerization polypeptide, second dimerization polypeptide, first framework polypeptide and second framework polypeptide (e.g., located on the N-terminal side of their dimerization sequence or counterpart dimerization sequence). See, e.g., FIG. 20, structures A-D.
82. The MAPP or duplex MAPP of any preceding aspect, wherein, when a framework or dimerization polypeptide of the MAPP or duplex MAPP comprises one or more IgFc regions (see, e.g., SEQ ID NOs:54-65), at least one of the one or more IgFc regions comprises one or more substitutions that limit complement activation.
83. The MAPP or duplex MAPP of any preceding aspect, wherein, when a framework or dimerization polypeptide of the MAPP or duplex MAPP comprises one or more IgFc regions (see, e.g., SEQ ID NOs:54-65), at least one of the one or more IgFc regions comprises one or more substitutions at L234, L235, G236, G237, P238, S239, D270, N297, K322, P329, and/or P331 (respectively, aas L14, L15, G16, G17, P18, S19, D50, N77, K102, P109, and P111 of the wt. IgG1 aa sequence in FIG. 4D, see, e.g., SEQ ID NOs:57-61).
84. The MAPP or the duplex MAPP of aspect 83, wherein the framework or dimerization polypeptide comprises an IgFc region having a substitution at N297 (e.g., N297A, see, e.g., SEQ ID NO:60).
85. The MAPP or the duplex MAPP of aspect 83, wherein the framework or dimerization polypeptide comprises an IgFc region having a substitution at L234 and/or L235 (e.g., L234A and/or L235A, see, e.g., SEQ ID NO:61).
86. The MAPP or the duplex MAPP of aspect 83, wherein the framework or dimerization polypeptide comprises an IgFc region having a substitution at P331 (e.g., P331A or P331S, see, e.g., SEQ ID NO:59).
87. The MAPP or the duplex MAPP of aspect 83, wherein the framework or dimerization polypeptide comprises an IgFc region having a substitution at: (i) L234, L235, and/or P331 (e.g., L234F, L235E, and P331S), or (ii) D270, K322, and/or P329 (e.g., D270, K322, and/or P329).
88. The MAPP of any of aspects 1 to 32, complexed to form a triplex MAPP of three heterodimers, a quadraplex MAPP of four heterodimers, a pentaplex MAPP of five heterodimers, or a hexaplex MAPP of six heterodimers.
89. The MAPP or duplex MAPP of any preceding aspect, comprising at least one MOD, at least one variant MOD, or at least one pair of MODs and/or variant MODs in tandem, located at one or more of positions 1, 1′, 2, 2′, 3, 3′, 4, 4′, 4″, 4′″, 5, and/or 5′ (see FIGS. 1A and 1B).
90. The MAPP or duplex MAPP of aspect 89, comprising:
   (a) at least one MOD, at least one variant MOD, or at least one pair of MODs and/or variant MODs in tandem located:
      (i) on the N-terminal side (e.g., at the N-terminus) of at least one framework polypeptide dimerization sequence (see, e.g., positions 1 and 1′ in any of FIGS. 6 and 8),
      (ii) on the N-terminal side (e.g., at the N-terminus) of at least one framework polypeptide dimerization sequence and any MHC-H or β2M polypeptide sequences that may be part of the framework polypeptide (see, e.g., positions 4″ and 4′″ in FIGS. 7 and 9), and/or
      (iii) on the C-terminal side (e.g., at the C-terminus) of at least one framework polypeptide multimerization sequence (see, e.g., positions 3 and 3′ in any of FIGS. 1 and 6 to 10); and/or
   (b) at least one MOD, at least one variant MOD, or at least one pair of MODs and/or variant MODs in tandem located:
      (i) on the N-terminal side (e.g., at the N-terminus) of each framework polypeptide dimerization sequence (see, e.g., positions 1 and 1′ in any of FIGS. 6 and 8);
      (ii) on the N-terminal side (e.g., at the N-terminus) of each framework polypeptide dimerization sequence and any MHC-H or β2M polypeptide sequences that may be part of the framework polypeptide (see, e.g., positions 4″ and 4′″ in FIGS. 7 and 9); and/or
      (iii) on the C-terminal side (e.g., at the C-terminus) of each framework polypeptide multimerization sequence (see, e.g., positions 3 and 3′ in any of FIGS. 1 and 6 to 10).
91. The MAPP or duplex MAPP of aspect 90 comprising:
   (i) at least one MOD or variant MOD, or pair of MODs and/or variant MODs in tandem located on the N-terminal side (e.g., at the N-terminus) of at least one (e.g., each) framework polypeptide dimerization sequence (see, e.g., position 1 and 1′ in any of FIGS. 6 and 8), or on the N-terminal side (e.g., at the N-terminus) of at least one (e.g., each) framework polypeptide dimerization sequence and any MHC-H or β2M polypeptide sequences that may be part of the framework polypeptide (see, e.g., positions 4″ and 4′″ in FIGS. 7 and 9); and/or
   (ii) at least one MOD or variant MOD, or pair of MODs and/or variant MODs in tandem located on the C-terminal side (e.g., at the C-terminus) of at least one (e.g., each) framework polypeptide multimerization sequence (see, e.g., positions 3 and 3′ in any of FIGS. 1 and 6 to 10).
92. The MAPP or duplex MAPP of any preceding aspect, comprising at least one MOD or variant MOD, or pair of MODs and/or variant MODs in tandem located:
   (i) on the N-terminal side (e.g., at the N-terminus) of at least one (e.g., two or each) dimerization polypeptide counterpart dimerization sequence (see, e.g., positions 4 and 4′ in FIGS. 1, and 7 to 9); and/or
   (ii) on the C-terminal side (e.g., at the C-terminus) of at least one (e.g., two or each) dimerization polypeptide counterpart dimerization sequence (see, e.g., positions 5 and 5′ in any of FIGS. 1 and 6 to 9).
93. The MAPP or duplex MAPP of any of aspects 89 to 92, wherein, when the at least one (e.g., two or each) dimerization polypeptide comprises a presenting sequence, the at least one MOD or variant MOD, or pair of MODs and/or variant MODs in tandem may be located:
   (i) between the counterpart dimerization sequence and the MHC-H, β2M and KRAS epitope sequences:
   (ii) between the MHC-H and β2M sequences;
   (iii) between the KRAS epitope and either the MHC-H or β2M sequence; and/or
   (iv) at the N-terminus of the presenting sequence. See FIG. 12.
94. The MAPP or duplex MAPP of any of aspects 89 to 92, wherein, when the dimerization polypeptide comprises a presenting complex, the at least one MOD, or variant MOD, or pair of MODs and/or variant MODs in tandem may be located:
   (i) between the counterpart dimerization sequence and any of the MHC-H, β2M or KRAS epitope sequences present in the presenting complex 1^{st} sequence;
   (ii) at the N-terminus of the presenting complex 1^{st} sequence;
   (iii) at the N-terminus of the presenting complex 2^{st} sequence;
   (iv) between either the MHC-H and KRAS epitope sequences, or between the β2M and KRAS epitope sequences of the presenting complex 2^{st} sequence; and/or
   (v) at the C-terminus of the presenting complex 2^{st} sequence. See, e.g., FIGS. 13 to 15.
95. The duplex MAPP of any of aspects 32 to 94, comprising at least one MOD, or variant MOD, or pair of MODs and/or variant MODs in tandem at positions 1 and/or 1′.
96. The duplex MAPP of any of aspects 32 to 94, comprising at least one MOD or variant MOD, or pair of MODs and/or variant MODs in tandem at positions 1 and/or 1′, and at least one MOD or variant MOD, or pair of MODs and/or variant MODs in tandem at positions 3 and/or 3′.
97. The duplex MAPP of any of aspects 32 to 94, comprising: at least one MOD or variant MOD, or pair of MODs and/or variant MODs in tandem at positions 1 and/or 1′, and at least one MOD or variant MOD, or pair of MODs and/or variant MODs in tandem at positions 5 and/or 5′.
98. The MAPP or duplex MAPP of any preceding aspect, comprising: at least one (e.g., at least two, or at least three) wt. MOD and/or variant MOD polypeptide sequence selected independently from the group consisting of: IL-1, IL-2, IL-4, IL-6, IL-7, IL-10, IL-12, IL-15, IL-17, IL-21, IL-23, CD7, CD30L, CD40, CD70, CD80 (B7-1), CD83, CD86 (B7-2), HVEM (CD270), ILT3 (immunoglobulin-like transcript 3), ILT4 (immunoglobulin-like transcript 4), Fas ligand (FasL), ICAM (intercellular adhesion molecule), ICOS-L (inducible costimulatory ligand), JAG1 (CD339), lymphotoxin beta receptor, 3/TR6, OX40L (CD252), PD-L1, PD-L2, TGF-β1, TGF-β2, TGF-β3, and 4-1BBL polypeptide sequences.
99. The MAPP or duplex MAPP of any preceding aspect, comprising at least one (e.g., at least two, or at least three) wt. MOD or variant MOD polypeptide sequence selected independently from the group consisting of: 4-1BBL, IL-2, CD80 and CD86.
100. The MAPP or duplex MAPP of any preceding aspect, comprising at least one (e.g., at least two, or at least three) wt. MOD or variant MOD polypeptide sequence selected independently from the group consisting of 4-1BBL, IL-2, CD80 and CD86 wt. MOD or variant MOD polypeptide sequences. For example, the MAPP or duplex MAPP may comprise at least one IL-2 MOD or variant MOD polypeptide sequence, and at least one CD80, CD86, variant CD80 or variant CD86 polypeptide sequence.
101. The MAPP or duplex MAPP of any preceding aspect, comprising at least one IL-2 MOD or variant MOD polypeptide sequence, or at least one pair of IL-2 MOD or variant MOD polypeptide sequences in tandem (optionally located at position 1 or 1′), and/or at least one CD80 and/or CD86 MOD or variant MOD polypeptide sequence.
102. The MAPP or duplex MAPP of aspect 100, further comprising at least one CD80 and/or CD86 MOD or variant MOD polypeptide sequence, optionally located at position 1 or 1′.
103. The MAPP or duplex MAPP of aspect 100, further comprising at least one variant IL-2 MOD polypeptide sequence, optionally located at position 1 or 1′.
104. The MAPP or duplex MAPP of aspect 100, further comprising at least one variant IL-2 MOD polypeptide sequence comprising an H16A or T substitution and an F42A substitution.
105. The MAPP or duplex MAPP of any preceding aspect further comprising an additional peptide, or a payload covalently attached to one or more framework polypeptides and/or dimerization polypeptides.
106. The MAPP or duplex MAPP of aspect 105, wherein the additional peptide is an epitope tag or an affinity domain.
107. The MAPP or duplex MAPP of aspect 105, wherein the additional peptide is a targeting sequence.
108. The MAPP or duplex MAPP of aspect 107, wherein the targeting sequence is an antibody or an antigen binding fragment thereof, or a single chain T cell receptor.
109. The MAPP or duplex MAPP of any of aspects 1 to 108, wherein the KRAS epitope is a KRAS peptide set forth in Section IV.C.7 of this disclosure.
110. The MAPP or duplex MAPP of any of aspects 1-31, wherein the KRAS epitope comprises the aa sequence: VVGADGVGK (SEQ ID NO:119), VVGACGVGK (SEQ ID NO:120), or VVGAVGVGK (SEQ ID NO:121), and has a length of 9 amino acids.
111. The MAPP or duplex MAPP of any of aspects 1-31, wherein the KRAS epitope comprises the aa sequence: VVVGADGVGK (SEQ ID NO:122), VVVGACGVGK (SEQ ID NO:124), or VVVGAVGVGK (SEQ ID NO:123), and has a length of 10 amino acids.
112. The MAPP or duplex MAPP of any of aspects 1-31, wherein the KRAS epitope comprises the aa sequence: VTGADGVGK (SEQ ID NO:125), VTGACGVGK (SEQ ID NO:127), or VTGAVGVGK (SEQ ID NO:126), and has a length of 9 amino acids.
113. The MAPP or duplex MAPP of any of aspects 1-31, wherein the KRAS epitope comprises the aa sequence: VTVGADGVGK (SEQ ID NO:128), VTVGACGVGK (SEQ ID NO:130), or VTVGAVGVGK (SEQ ID NO:129), and has a length of 10 amino acids.
114. The MAPP or duplex MAPP of any of aspects 109-113, wherein each MHC-H polypeptide comprises a sequence having at least 95% (e.g., at least 96%, 97%, 98%, 99%, or 100%) sequence identity to HLA-A*1101 polypeptide sequence SEQ ID NOs:20 or 26).
115. The MAPP or duplex MAPP of any of aspects 1-31, wherein the KRAS epitope comprises the aa sequence: LVVVGADGV (SEQ ID NO:135), LVVVGAVGV (SEQ ID NO:136), LVVVGACGV (SEQ ID NO:137), KLVVGADGV (SEQ ID NO:163), KLVVGAVGV (SEQ ID NO:164), KLVVVAVGV (SEQ ID NO:165), or KLVVVADGV (SEQ ID NO:166), and has a length of 9 amino acids.
116. The MAPP or duplex MAPP of any of aspects 1-31, wherein the KRAS epitope comprises the aa sequence: KLVVVGADGV (SEQ ID NO:138), KLVVVGAVGV (SEQ ID NO:139), or KLVVVGACGV (SEQ ID NO:140), and has a length of 10 amino acids.
117. The MAPP or duplex MAPP of any of aspects 1-31, wherein the KRAS epitope comprises the aa sequence: LLVVGADGV (SEQ ID NO:141), LLVVGAVGV (SEQ ID NO:142), or LLVVGACGV (SEQ ID NO:143), and has a length of 9 amino acids.
118. The MAPP or duplex MAPP of any of aspects 1-31, wherein the KRAS epitope comprises the aa sequence: FLVVVGADGV (SEQ ID NO:144), FLVVVGAVGV (SEQ ID NO:145), or FLVVVGACGV (SEQ ID NO:146), and has a length of 10 amino acids.
119. The MAPP or duplex MAPP any of aspects 1 to 118, wherein the epitope peptide is from about 4 aas (aa) to about 25 aa (e.g., the epitope can have a length of from about 4 aa to about 10 aa, from about 6 aa to about 12 aa, from about 10 aa to about 15 aa, from about 15 aa to about 20 aa, or from about 20 aa to about 25 aa).
120. The MAPP or duplex MAPP of aspect 119, wherein the epitope peptide is from about 6 aa to about 12 aa.
121. A method of treatment or prophylaxis of a disease (e.g., a benign (non-malignant) neoplasm or cancer) comprising:
   (i) administering to a patient/subject (e.g., a patient in need thereof) an effective amount of one or more MAPPs or duplex MAPPs of any of aspects 1 to 120;
   (ii) administering to a patient/subject (e.g., a patient in need thereof) an effective amount of one or more nucleic acids encoding a MAPP or duplex MAPP according to any of aspects 1 to 120;
   (iii) contacting a cell or tissue in vitro or in vivo with one or more MAPPs or duplex MAPPs of any of aspects 1 to 120 and administering the cell, tissue, or progeny thereof to the patient/subject; or
   (iv) contacting a cell or tissue in vitro or in vivo with one or more nucleic acids encoding a MAPP or duplex MAPP of any of aspects 1 to 120 and administering the cell, tissue, or progeny thereof to the patient/subject.
122. The method of aspect 121, wherein the MAPP or duplex MAPP further comprises at least one targeting sequence (e.g., a targeting sequence specific for an antigen associated with a cell or tissue).
123. The method of any of aspects 121 to 122, wherein the one or more MAPPs or duplex MAPPs are administered to a mammalian patient or subject.
124. The method of any of aspects 121 to 123, wherein the patient or subject is human.
125. The method of any of aspects 121 to 123, wherein the subject is non-human (e.g., rodent, lagomorph, bovine, canine, feline, rodent, murine, caprine, simian, ovine, equine, lappine, porcine, etc.).
126. The method of any of aspects 121 to 125, wherein the disease or condition is a cancer.
127. The method of aspect 126, wherein the cancer is a colorectal cancer, pancreatic cancer, lung cancer, bile duct carcinoma, gall bladder carcinoma, adenocarcinoma, rectal adenocarcinoma, endometrial carcinoma, or hematopoietic neoplasm.
128. The method of aspect 126, wherein the cancer is non-small cell lung cancer, lung adenocarcinoma, mucinous adenoma, ductal carcinoma of the pancreas, colorectal cancer, or leukemia.
129. The method of aspect 126, wherein the cancer is a colorectal cancer.
130. The method of aspect 126, wherein the cancer is a lung cancer.
131. The method of aspect 126, wherein the cancer is a pancreatic cancer.
132. The method of aspect 126, wherein the cancer is leukemia.
133. The method any of aspects 121 to 125, wherein the disease or condition is a non-malignant neoplasm.
134. The method of any of aspects 121 to 133, further comprising administering one or more therapeutic agents that enhance CD 8+ T cell functions (e.g., effector function) and/or treat the disease or condition before, during (concurrent or combined administration) or after administering the one or more MAPPs, duplexed MAPPs or one or more nucleic acids encoding the one or more MAPPs or duplexed MAPPs.
135. The method of aspect 134, wherein the one or more therapeutic agents that enhance CD 8+ function and/or treat the disease or condition comprise an antiTGF-β antibody such as Metelimumab (CAT192) directed against TGF-β1 and Fresolimub directed against TGF-β1 and TGF-β2, or a TGF-β trap (subject to the proviso that the MAPP or duplexed MAPP does not comprise an aa sequence to which the antibodies or TGF-β trap bind such as a TGF-β1 or TGF-β2 MOD or variant MOD aa sequence).
136. The method of any of aspects 134 to 135, wherein the one or more therapeutic agents that enhance CD 8+ function and/or treat the disease or condition comprise one or more antibodies directed against: B lymphocyte antigens (e.g., ibritumomab tiuxetan, obinutuzumab, ofatumumab, rituximab to CD20, brentuximab vedotin directed against CD30, and alemtuzumab to CD52); EGFR (e.g., cetuximab, panitumumab, and necitumumab); VEGF (e.g., bevacizumab); VEGFR2 (e.g., ramucirumab); HER2 (e.g., pertuzumab, trastuzumab, and ado-trastuzumab); PD-1 (e.g., nivolumab and pembrolizumab targeting a check point inhibition); RANKL (e.g., denosumab); CTLA-4 (e.g., ipilimumab targeting check point inhibition); IL-6 (e.g., siltuximab); disialoganglioside (GD2), (e.g., dinutuximab); CD38 (e.g., daratumumab); SLAMF7 (Elotuzumab); both EpCAM and CD3 (e.g., catumaxomab); or both CD19 and CD3 (blinatumomab) (subject to the proviso that the MAPP or duplexed MAPP does not comprise an aa sequence to which the antibodies bind).
137. The method of any of aspects 121 to 136, wherein the disease is a cancer and the method further comprises administering one or more chemotherapeutic agents, antibiotics and/or chemotherapeutics.
138. The method of any of aspects 121 to 137, further comprising administering one or more chemotherapeutic agent selected from the group consisting of: alkylating agents, cytoskeletal disruptors (taxane), epothilones, histone deacetylase inhibitors, topoisomerase I inhibitors, topoisomerase II inhibitors, kinase inhibitors, nucleotide analog or precursor analogs, peptide antineoplastic antibiotics (e.g. bleomycin or actinomycin), platinum-based agents, retinoids, or vinca alkaloids and their derivatives.
139. The method of any of aspects 121 to 137, further comprising administering one or more chemotherapeutic agents selected from the group consisting of: actinomycin all-trans retinoic acid, azacytidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, and vindesine.
140. The method of any of aspects 121 to 139, wherein the MAPP or duplex MAPP, or the nucleic acid encoding a MAPP or duplex MAPP, is administered in a composition comprising the MAPP or duplex MAPP and at least one pharmaceutical acceptable excipient.
141. A framework polypeptide of a MAPP or duplex MAPP according to any of aspects 1 to 120, optionally comprising an additional polypeptide.
142. A dimerization polypeptide of a MAPP or duplex MAPP, according to any of aspects 1 to 120, optionally comprising an additional polypeptide.
143. A nucleic acid sequence encoding the framework polypeptide of any of aspects 1 to 120, wherein the framework polypeptide optionally comprises an additional polypeptide.
144. A nucleic acid sequence encoding the dimerization polypeptide of any of aspects 1 to 120, wherein the dimerization polypeptide optionally comprises an additional polypeptide.
145. One or more nucleic acids comprising one or more nucleic acid sequences encoding a MAPP or duplex MAPP according to any of aspects 1 to 120.
146. The nucleic acid of any of aspects 143 to 145, wherein the nucleic acid sequences encoding the framework polypeptide and/or the dimerization polypeptide are operably linked to a promoter.
147. A method of producing cells expressing a MAPP or duplex MAPP, the method comprising introducing one or more nucleic acids according to aspect 146 into the cells in vitro; selecting for cells that produce the MAPP or duplex MAPP; and optionally selecting for cells comprising all or part of the one or more nucleic acids either unintegrated or integrated into at least one cellular chromosome.
148. The method of aspect 147, wherein the cell is a cell of a mammalian cell line selected from HeLa cells, CHO cells, 293 cells, Vero cells, NIH 3T3 cells, Huh-7 cells, BHK cells, PC12 cells, COS cells, COS-7 cells, RAT1 cells, mouse L cells, human embryonic kidney (HEK) cells, and HLHepG2 cells.
149. A cell transiently or stably expressing a MAPP or duplex MAPP prepared by the method of any of aspects 147 or 148.
150. The cell of aspect 149, wherein cells express from about 25 to about 350 (e.g., 20-50, 50-100, 100-200, 200-300, 300-350) mg/liter or more of the MAPP or duplex MAPP without a substantial reduction (e.g., less than a 5%, 10%, or 15% reduction) in cell viability relative to otherwise identical cells not expressing the MAPP or duplex MAPP.
151. A method of selectively delivering one or more MOD polypeptides and/or variant MOD polypeptides to a cell, tissue, patient or subject, the method comprising:
   (i) administering to a patient/subject (e.g., a patient in need thereof) an effective amount of one or more MAPPs or duplex MAPPs of any of aspects 1 to 120;
   (ii) administering to a patient/subject (e.g., a patient in need thereof) an effective amount of one or more nucleic acids encoding a MAPP or duplex MAPP according to any of aspects 1 to 120;
   (iii) contacting a cell or tissue in vitro, in vivo, or ex vivo with one or more MAPPs or duplex MAPPs of any of aspects 1 to 120 and optionally administering the cell, tissue, or progeny thereof to the patient/subject; or
   (iv) contacting a cell or tissue in vitro, in vivo, or ex vivo with one or more nucleic acids encoding a MAPP or duplex MAPP of any of aspects 1 to 120 and optionally administering the cell, tissue, or progeny thereof to the patient/subject;
   wherein the MAPP or duplex MAPP comprises one or more MODs or variant MODs.
152. The method of aspect 151, wherein the one or more MOD polypeptides and/or variant MOD polypeptides are selected independently from the group consisting of: 4-1BBL, PD-L1, IL-2, CD80, CD86, OX40L (CD252), Fas ligand (FasL), ICOS-L, ICAM, CD30L, CD40, CD83, HVEM (CD270), JAG1 (CD339), CD70, TGF-β1, TGF-β2, and TGF-β3 MOD or variant MOD polypeptide sequences.
153. The method of aspect 151, wherein the one or more MOD polypeptides and/or variant MOD polypeptides are selected independently from the group consisting of: 4-1BBL, IL-2, CD80 and CD86 MODs, and variant MOD polypeptide sequences of any thereof.
154. The method of aspect 151, wherein the MAPP or duplex MAPP comprises at least one IL-2 MOD or variant MOD polypeptide sequence, and at least one CD80, CD86, variant CD80 or variant CD86 polypeptide sequence.
155. The method of aspect 151, wherein the MAPP or duplex MAPP comprises at least one IL-2 MOD or variant IL-2 MOD (e.g., comprising an H16T substitution and an F42A substitution) polypeptide sequence, or at least one pair of IL-2 MOD (wt. IL-2 and/or variant IL-2 MOD) polypeptide sequences in tandem.
156. The method of aspect 151, wherein the MAPP or duplex MAPP comprises at least one CD80 and/or CD86 MOD or variant MOD polypeptide sequence.

### V. EXAMPLES

### Example 1

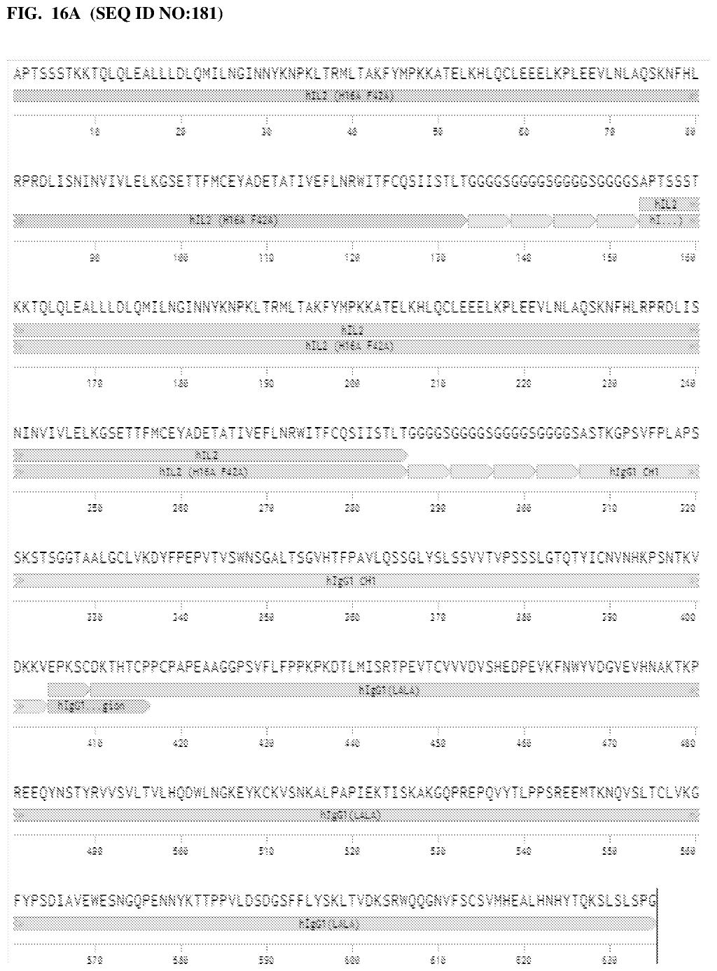

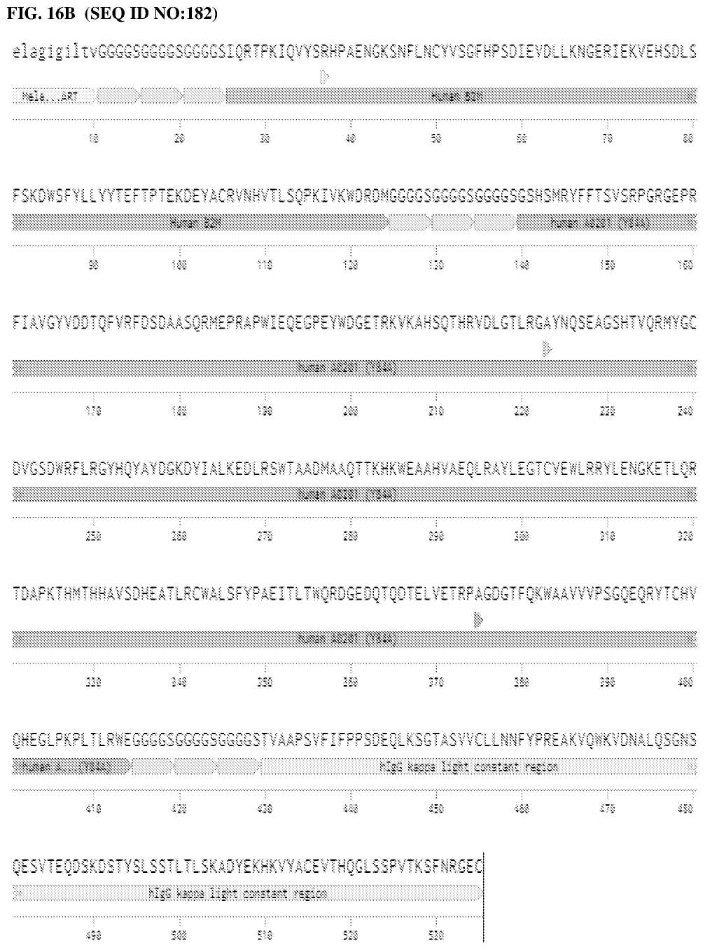

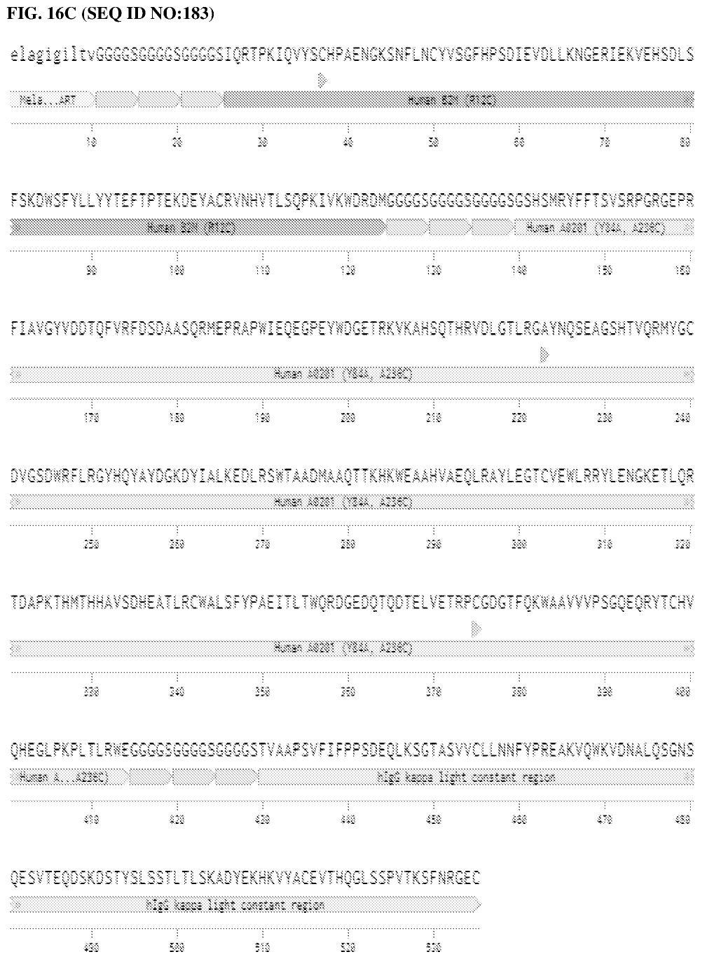

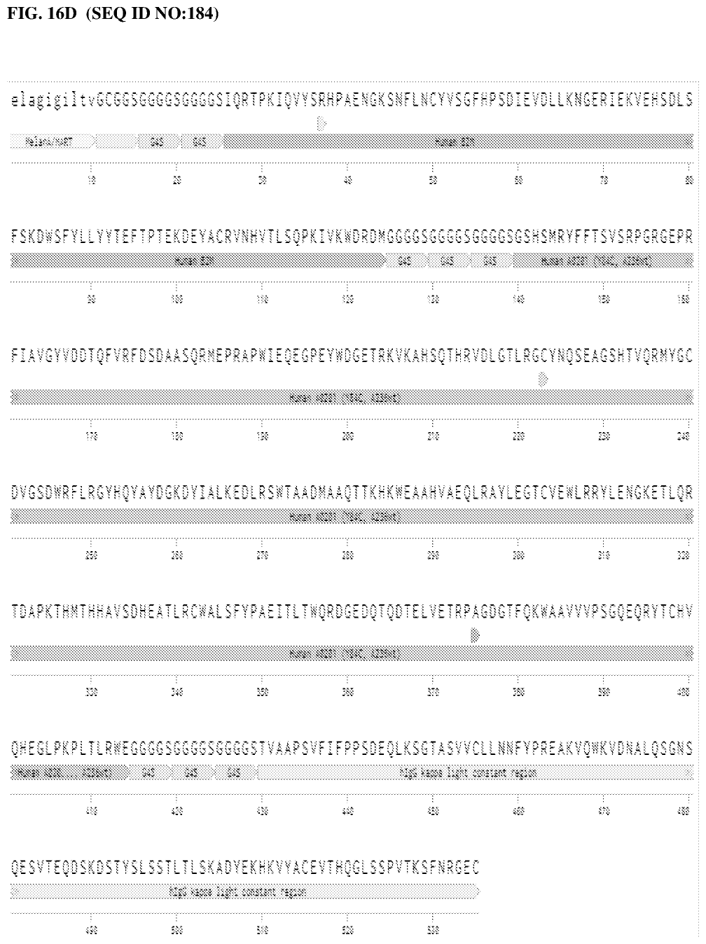

Example 1 illustrates three related MAPP heterodimer constructs that have multimerized into duplexes with the overall structures given in FIG. 11 as structures A, B, C, and D. Each of the heterodimers share a common framework polypeptide that comprises human IgG heavy chain constant region (CH2-CH3 domain) multimerization sequences and a CH1 dimerization sequence provide in FIG. 16A. Three dimerization polypeptides that can form heterodimers with the framework polypeptide are provided in FIGS. 16B, 16C, and 16D. The heterodimers each comprise a framework and dimerization polypeptide and pair to form duplex MAPP structures through interactions between IgG heavy elements in each of the framework polypeptides as shown in FIG. 11 structures A, B, C, and D.

FIG. 16A, construct 3777 (SEQ ID NO:181), provides the aa sequence and location of elements of the framework polypeptide, which comprises from N-terminus to C-terminus: IL-2 leader sequence; tandem IL-2 sequences each having H16A, F42A substitutions separated by a linker comprising four repeats of GGGGS (SEQ ID NO:110); a human IgG1 CH1 domain that acts as dimerization sequence, and a human IgG1 Fc region comprising “LALA” substitutions that acts as a multimerization sequence.

FIG. 16B, construct 3781 (SEQ ID NO:182), provides the aa sequence and location of elements in a dimerization polypeptide that comprises, from N-terminus to C-terminus: an epitope peptide (melanocyte-melanoma tumor-antigen or “MART” peptide ELAGIGILTV shown in lower case letters); three repeats of the linker sequence GGGGS (SEQ ID NO:110), a human β2M polypeptide sequence; three repeats of the linker sequences GGGGS (SEQ ID NO:110); a human HLA-A*0201 (HLA-A*02:01) polypeptide comprising a Y84A substitution; three repeats of the linker sequences GGGGS (SEQ ID NO:110); and a human IgG κ light chain constant region dimerization sequence.

FIG. 16C, construct 3782 (SEQ ID NO:183), provides the aa sequence and location of elements in a dimerization polypeptide that comprises, from N-terminus to C-terminus: an epitope peptide (melanocyte-melanoma tumor-antigen or “MART” peptide ELAGIGILTV shown in lower case letters); three repeats of the linker sequence GGGGS (SEQ ID NO:110), a human β2M polypeptide sequence comprising an R12C substitution; three repeats of the linker sequences GGGGS (SEQ ID NO:110); a human HLA-A*0201 (HLA-A*02:01) polypeptide comprising Y84A and A236C substitutions; three repeats of the linker sequences GGGGS (SEQ ID NO:110); and a human IgG κ light chain constant region dimerization sequence.

FIG. 16D, construct 378 (SEQ ID NO:184), provides the aa sequence and location of elements in a dimerization polypeptide that comprises, from N-terminus to C-terminus: an epitope peptide (melanocyte-melanoma tumor-antigen or “MART” peptide ELAGIGILTV shown in lower case letters); a linker comprising the sequence GCGGS followed by two repeats of the sequence GGGGS (SEQ ID NO:110), a human β2M polypeptide sequence; three repeats of the linker sequences GGGGS (SEQ ID NO:110); a human HLA-A*0201 (HLA-A*02:01) polypeptide comprising a Y84A substitution; three repeats of the linker sequences GGGGS (SEQ ID NO:110); and a human IgG κ light chain constant region dimerization sequence.

FIG. 11 structure A shows a duplex MAPP formed from constructs 3777 and 3781. FIG. 11 structure B shows a duplex MAPP formed from constructs 3777 and 3782 with an additional disulfide bond between the R12C cysteine of the β2M polypeptide and the Y84C cysteine of the HLA-A*02:01 sequence. FIG. 11 structure C shows a duplex MAPP formed from constructs 3777 and 3783. FIG. 11 structure D shows modification of structure C in which the MHC-H sequence has a internal Y84C A139C disulfide bond (denoted Y84C-SS-A139C), and the same substitutions may be made in structures A and B. Structures A and C of FIG. 11 may also include a disulfide bond between position 84 of the MHC-H (Y84C) and a cysteine the linker between the the epitope and β2M polypeptide.

The three duplex MAPPs in FIG. 11 are prepared by cellular expression of the framework and dimerization sequences in Expi-CHO cells by transient transfection using an expression vector containing a nucleic acid construct encoding the polypeptides. The assembled duplex MAPPs are purified over Protein A (MabSelect SuRe™; GE), followed by further purification by size exclusion chromatography.

Testing, conducted by contacting the MAPPs with a population of PBMCs obtained from one or more individuals that have not been exposed to the MART epitope peptide, demonstrates an expansion in the number of MART specific T cells as demonstrated by MART-tetramer staining. In contrast, exposure of PBMC to MAPPs bearing an unrelated epitope does not increase MART specific T cell numbers.

1. A multimeric antigen-presenting polypeptide complex (MAPP) comprising:
a framework polypeptide comprising a dimerization sequence and a multimerization sequence; and
a dimerization polypeptide comprising a counterpart dimerization sequence complementary to the dimerization sequence of the framework polypeptide, and dimerizing therewith through covalent and/or non-covalent interactions to form a MAPP heterodimer; and at least one presenting sequence and/or presenting complex,
wherein each presenting sequence comprises a KRAS epitope, an MHC class I heavy chain (MHC-H), and a β2M polypeptide sequence;
wherein each presenting complex comprises a presenting complex 1^{st} sequence and a presenting complex 2^{nd} sequence that together comprise a KRAS epitope, an MHC-H, and a β2M polypeptide sequence;
wherein one or both of the dimerization polypeptide and/or the framework polypeptide comprises a presenting sequence or a presenting complex 1st sequence;
wherein optionally at least one of the framework polypeptide, dimerization peptide, presenting sequence(s), presenting complex 1^{st} sequence and/or presenting complex 2^{nd}sequence comprises one, two, three or more independently selected MOD and/or variant MOD polypeptide sequences; and
wherein the framework polypeptide, dimerization polypeptide, presenting sequence, presenting complex 1^{st} sequence and/or presenting complex 2^{nd} sequence optionally comprise one or more linker sequences selected independently,
optionally wherein the one or more MOD polypeptide sequences may be polypeptides such as wt. IL-2 or a variant of wt. IL-2 that result in T cell activation, wherein T cell activation may result in one or more of the following: an increase in the activity of ZAP70 protein kinase activity, induction in the proliferation of the T-cell(s), granule-dependent effector actions and/or release of T cell cytokines.

2. (canceled)

3. The MAPP of claim 1, wherein at least one presenting sequence or presenting complex comprises:
an MHC-H sequence that does not include the MHC-H transmembrane domain, or a portion thereof, that will anchor the MAPP in a cell membrane; and
a β2M sequence having at least 90% sequence identity to at least 0 contiguous aas of a mature β2M polypeptide provided in SEQ ID NO:1 through SEQ ID NO:5.

4. (canceled)

5. The MAPP of claim 3, wherein the MHC-H polypeptide sequence has at least 90% or 100% sequence identity to at least 125 contiguous aas of an MHC-as sequence selected from the group consisting of: HLA-A*1101 (SEQ ID NOs:20 or 26), HLA-A*0101 (SEQ ID NOs: 12 or 23), HLA-A*0201 (SEQ ID NO:15 or 24), HLA-A*0203, HLA-A*0301 (SEQ ID NO:25), HLA-A*2301 (SEQ ID NO: 27), HLA-A*2402 (SEQ ID NOs:21 or 28), HLA-A*2407 (SEQ ID NO: 29), HLA-A*3101, HLA-A*3303 (SEQ ID NOs: 22 or 30), HLA-A*3401 SEQ ID NO:31), HLA-A*6801, HLA-B*0702 (SEQ ID NO:13 or 33), HLA-B*0801 (SEQ ID NO:341 HLA-B*1502 (SEQ ID NO:35), HLA-B*2705, HLA-B*3802 (SEQ ID NO:36L HLA-B*3901, HLA-B*3902, HLA-B*4001 (SEQ ID NO:37), HLA-B*4601 (SEQ ID NO:38), HLA-B*5101, or HLA-B*5301 (SEQ ID NO:39), HLA-C*0102 (SEQ ID NO:41), HLA-C*0303 (SEQ ID NQ:42), HLA-C*0304 (SEQ ID NO:43), HLA-C*0401 (SEQ ID NO:44), HLA-C*0602 (SEQ ID NO:45), HLA-C*0701 (SEQ ID NO:46), HLA-C*0702 (SEQ ID NO:47), HLA-C*0801 (SEQ ID NO:48), or HLA-C*1502 (SEQ ID NO:49), HLA-E*01:01, HLA-E*01:03, HLA-E*01:04, HLA-E*01:05, HLA-E*01:06, HLA-E*01:07, HLA-E*01:09, or HLA-E*01:10, HLA-F*0101 (HLA-F*01:01:01:01), HLA-F*01:02, HLA-F*01:03 (HLA-F*01:03:01:01), HLA-F*01:04, HLA-F*01:05, and HLA-F*01:06, HLA-G*01:04 (HLA-G*01:04:01:01), HLA-G*01:06, HLA-G*01:07, HLA-G*01:08, HLA-G*01:09, HLA-G*01:10, HLA-G*01:11, HLA-G*01:12, HLA-G*01:14, or HLA-G*01:15.

6.-13. (canceled)

14. The MAPP of claim 5, wherein the β2M sequence has at least 90% sequence identity to at least 80 contiguous aas of a mature human β2M polypeptide of NCBI accession number NP_004039.1 (SEQ ID NO:1).

15. The MAPP of claim 14, wherein:
A) at least one of the MHC class I polypeptide heavy chain sequences comprises a cysteine at positions 84 and 139; or
B) at least one presenting sequence or presenting complex comprises:
a B2M sequence having a cysteine at position 12 of the mature B2M sequence and an MHC-H sequence with a cysteine at position 236, with the cysteines forming a disulfide bond; and/or
a β2M sequence having a KRAS epitope and cysteine-containing peptide linker at its amino terminus and an MHC-H sequence with a cysteine at position 84, with the cysteines forming a disulfide bond.

16.-19. (canceled)

20. The MAPP of claim 15, wherein the dimerization and/or multimerization sequences are independently selected from non-interspecific sequences or interspecific sequences.

21. The MAPP of claim 20, wherein the interspecific and non-interspecific sequences are selected from the group consisting of: immunoglobulin heavy chain constant regions, collectin polypeptides, coiled-coil domains, leucine-zipper domains, Fos polypeptides, Jun polypeptides, Ig CH1, Ig CL c, Ig CL X, knob-in-hole without disulfide (KiH), knob-in hole with a stabilizing disulfide bond (KiHs-s), HA-TF; ZW-1, 7.8.60, DD-KK, EW-RVT, EW-RVTs-s, and A107 sequences.

22. The MAPP of claim 20, complexed to form a duplex or higher order MAPP comprising at least a first MAPP heterodimer and a second MAPP heterodimer of claim 20, wherein:
(i) the first heterodimer comprises a first framework polypeptide having a first multimerization sequence and a first dimerization sequence, and a first dimerization polypeptide having a first counterpart dimerization sequence complementary to the first dimerization sequence; and
(ii) the second heterodimer comprises a second framework polypeptide having a second multimerization sequence and a second dimerization sequence, and a second dimerization polypeptide having a second counterpart dimerization sequence complementary to the second dimerization sequence; and
wherein the first and second framework polypeptides are associated by binding interactions between the first and second multimerization sequences optionally including one or more interchain covalent bonds, and the multimerization sequences are not the same as, and do not substantially associate with or bind to, the dimerization or counterpart dimerization sequences.

23. The duplex MAPP of claim 22, wherein:
the first dimerization polypeptide and first framework polypeptide are covalently linked by at least one disulfide bond: or
the first MAPP heterodimer and/or the second MAPP heterodimer are covalently linked by at least one disulfide bond, and the multimerization sequences of the first and second framework polypeptides are covalently linked by at least one disulfide bond.

24.-26. (canceled)

27. The MAPP or duplex MAPP of claim 22, wherein, when a framework or dimerization polypeptide of the MAPP or duplex MAPP comprises one or more IgFc regions, at least one of the one or more IgFc regions comprises one or more substitutions that limit complement activation.

28. The MAPP or duplex MAPP of claim 22, comprising at least one MOD, at least one variant MOD, or at least one pair of MODs and/or variant MODs in tandem.

29. The MAPP or duplex MAPP of claim 22, comprising at least one MOD and/or variant MOD polypeptide sequence selected independently from the group consisting of: IL-1, IL-2, IL-4, IL-6, IL-7, IL-10, IL-12, IL-15, IL-17, IL-21, IL-23, CD7, CD30L, CD40, CD70, CD80 (B7-1), CD83, CD86 (B7-2), HVEM (CD270), ILT3 (immunoglobulin-like transcript 3), ILT4 (immunoglobulin-like transcript 4), Fas ligand (FasL), ICAM (intercellular adhesion molecule), ICOS-L (inducible costimulatory ligand), JAG1 (CD339), lymphotoxin beta receptor, 3/TR6, OX40L (CD252), PD-L1, PD-L2, TGF-β1, TGF-β2, TGF-β3, and 4-1BBL polypeptide sequences.

30. The MAPP or duplex MAPP of claim 22, comprising at least one MOD or variant MOD polypeptide sequence selected independently from the group consisting of 4-1BBL, IL-2, CD80 and CD86 wt. MOD or variant MOD polypeptide sequences.

31. (canceled)

32. The MAPP or duplex MAPP of claim 29, wherein the KRAS epitope comprises an aa sequence selected from the group consisting of: VVGADGVGK (SEQ ID NO: 119), VVGACGVGK (SEQ ID NO:120), VVGAVGVGK (SEQ ID NO:121), VVVGADGVGK (SEQ ID NO:122), VVVGACGVGK (SEQ ID NO:124), VVVGAVGVGK (SEQ ID NO:123), VTGADGVGK (SEQ ID NO:125), VTGACGVGK (SEQ ID NO:127), VTGAVGVGK (SEQ ID NO:126), VTVGADGVGK (SEQ ID NO:128), VTVGACGVGK (SEQ ID NO:130), and VTVGAVGVGK (SEQ ID NO:129).

33. The MAPP or duplex MAPP of claim 30, wherein each MHC-H polypeptide comprises a sequence having at least 95% sequence identity to HLA-A*1101 polypeptide sequence (SEQ ID NO:20 or SEQ ID NO:26).

34. The MAPP or duplex MAPP of claim 29, wherein the KRAS epitope comprises an aa sequence selected from the group consisting of: LVVVGADGV (SEQ ID NO:135), LVVVGAVGV (SEQ ID NO:136), LVVVGACGV (SEQ ID NO:137), KLVVGADGV (SEQ ID NO:163), KLVVGAVGV (SEQ ID NO:164), KLVVVAVGV (SEQ ID NO:165), KLVVVADGV (SEQ ID NO:166), KLVVVGADGV (SEQ ID NO:138), KLVVVGAVGV (SEQ ID NO:139), KLVVVGACGV (SEQ ID NO:140), LLVVGADGV (SEQ ID NO:141), LLVVGAVGV (SEQ ID NO:142), LLVVGACGV (SEQ ID NO:143), FLVVVGADGV (SEQ ID NO:144), FLVVVGAVGV (SEQ ID NO:145), and FLVVVGACGV (SEQ ID NO:146).

35. The MAPP or duplex MAPP of claim 3, wherein the epitope peptide is from about 4 aa to about 25 aa.

36. (canceled)

37. A method of treatment or prophylaxis of a disease comprising:
(i) administering to a patient/subject an effective amount of one or more MAPPs or duplex MAPPs of claim 29;
(ii) administering to a patient/subject an effective amount of one or more nucleic acids encoding a MAPP or duplex MAPP of claim 29;
(iii) contacting a cell or tissue in vitro or in vivo with one or more MAPPs or duplex MAPPs of claim 29 and administering the cell, tissue, or progeny thereof to the patient/subject; or
(iv) contacting a cell or tissue in vitro or in vivo with one or more nucleic acids encoding a MAPP or duplex MAPP of claim 29 and administering the cell, tissue, or progeny thereof to the patient/subject.

38. The method of claim 37, wherein the disease or condition is a cancer.

39. The method of claim 38, wherein the cancer is a colorectal cancer, pancreatic cancer, lung cancer, bile duct carcinoma, gall bladder carcinoma, adenocarcinoma, rectal adenocarcinoma, endometrial carcinoma, hematopoietic neoplasm, non-small cell lung cancer, lune adenocarcinoma, mucinous adenoma, ductal carcinoma of the pancreas, colorectal cancer, or leukemia.

40.-46. (canceled)

47. One or more nucleic acids comprising one or more nucleic acid sequences encoding a MAPP or duplex MAPP according to claim 3.

48. A method of producing cells expressing a MAPP or duplex MAPP, the method comprising introducing one or more nucleic acids according to claim 47 into the cells in vitro; selecting for cells that produce the MAPP or duplex MAPP; and optionally selecting for cells comprising all or part of the one or more nucleic acids either unintegrated or integrated into at least one cellular chromosome.

49. (canceled)


SEQUENCE LISTING (185 sequences)

SEQ ID NO: 1 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 2 - protein, 119 aa, Pan troglodytes; identical to SEQ ID NO: 1

SEQ ID NO: 3 - protein, 119 aa, Macaca mulatta
MSRSVALAVLALLSLSGLEAIQRTPKIQVYSRHPPENGKPNFLNCYVSGFHPSDIEVDLLKNGEKMGKVEHSDLSFSKDWSFYLLYYTEFTPNEKDEYACRVNHVTLSGPRTVKWDRDM

SEQ ID NO: 4 - protein, 118 aa, Bos taurus
MARFVALVLLGLLSLSGLDAIQRPPKIQVYSRHPPEDGKPNYLNCYVYGFHPPQIEIDLLKNGEKIKSEQSDLSFSKDWSFYLLSHAEFTPNSKDQYSCRVKHVTLEQPRIVKWDRDL

SEQ ID NO: 5 - protein, 119 aa, Mus musculus
MARSVTLVFLVLVSLTGLYAIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDM

SEQ ID NO: 6 - protein, 365 aa, Homo sapiens
MAVMAPRTLLLLLSGALALTQTWAGSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 7 - protein, 365 aa, Homo sapiens
MAVMAPRTLLLLLSGALALTQTWAGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 8 - protein, 365 aa, Homo sapiens [sequence omitted]

SEQ ID NO: 9 - protein, 365 aa, Homo sapiens [sequence omitted]

SEQ ID NO: 10 - protein, 362 aa, Homo sapiens
MLVMAPRTVLLLLSAALALTETWAGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 11 - protein, 365 aa, Homo sapiens
MRVMAPRALLLLLSGGLALTETWACSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCK

SEQ ID NO: 12 - protein, 275 aa, Homo sapiens
GSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 13 - protein, 275 aa, Homo sapiens
GSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWE

SEQ ID NO: 14 - protein, 275 aa, Homo sapiens
CSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWE

SEQ ID NO: 15 - protein, 276 aa, Homo sapiens [sequence omitted]

SEQ ID NO: 16 - protein, 274 aa, Mus musculus
GPHSLRYFVTAVSRPGLGEPRFIAVGYVDDTQFVRFDSDADNPRFEPRAPWMEQEGPEYWEEQTQRAKSDEQWFRVSLRTAQRYYNQSKGGSHTFQRMFGCDVGSDWRLLRGYQQFAYDGRDYIALNEDLKTWTAADTAALITRRKWEQAGDAEYYRAYLEGECVEWLRRYLELGNETLLRTDSPKAHVTYHPRSQVDVTLRCWALGFYPADITLTWQLNGEDLTQDMELVETRPAGDGTFQKWAAVVVPLGKEQNYTCHVHHKGLPEPLTLRW

SEQ ID NO: 17 - protein, 275 aa, Homo sapiens
GSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWE

SEQ ID NO: 18 - protein, 275 aa, Homo sapiens
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWE

SEQ ID NO: 19 - protein, 276 aa, Homo sapiens
GSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEP

SEQ ID NO: 20 - protein, 276 aa, Homo sapiens
GSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 21 - protein, 276 aa, Homo sapiens
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEP

SEQ ID NO: 22 - protein, 276 aa, Homo sapiens
GSHSMRYFTTSVSRPGRGEPRFIAVGYVDDTQFVRFDSDAASQRMEPRAPWIEQEGPEYWDRNTRNVKAHSQIDRVDLGTLRGYYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 23 - protein, 275 aa, Homo sapiens; identical to SEQ ID NO: 12

SEQ ID NO: 24 - protein, 276 aa, Homo sapiens [sequence omitted]

SEQ ID NO: 25 - protein, 276 aa, Homo sapiens
GSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 26 - protein, 276 aa, Homo sapiens; identical to SEQ ID NO: 20

SEQ ID NO: 27 - protein, 276 aa, Homo sapiens
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITQRKWEAARVAEQLRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEP

SEQ ID NO: 28 - protein, 276 aa, Homo sapiens; identical to SEQ ID NO: 21

SEQ ID NO: 29 - protein, 276 aa, Homo sapiens
GSHSMRYFSTSVSRPGRGEPRFIAVGYVDDTQFVRFDSDAASQRMEPRAPWIEQEGPEYWDEETGKVKAQSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEP

SEQ ID NO: 30 - protein, 276 aa, Homo sapiens; identical to SEQ ID NO: 22

SEQ ID NO: 31 - protein, 276 aa, Homo sapiens
GSHSMRYFYTSVSRPGRGEPRFIAVGYVDDTQFVRFDSDAASQRMEPRAPWIEQEGPEYWDRNTRKVKAQSQTDRVDLGTLRGYYNQSEDGSHTIQRMYGCDVGPDGRFLRGYQQDAYDGKDYIALNEDLRSWTAADMAAQITQRKWETAHEAEQWRAYLEGTCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 32 - protein, 276 aa, Homo sapiens
  VARIANT (9)..(9): X1 is F, Y, S, or T
  VARIANT (44)..(44): X2 is K or R
  VARIANT (62)..(62): X3 is Q, G, E, or R
  VARIANT (63)..(63): X4 is N or E
  VARIANT (65)..(65): X5 is R or G
  VARIANT (66)..(66): X6 is N or K
  VARIANT (67)..(67): X7 is M or V
  VARIANT (70)..(70): X8 is H or Q
  VARIANT (73)..(73): X9 is T or I
  VARIANT (74)..(74): X10 is D or H
  VARIANT (76)..(76): X11 is A, V, or E
  VARIANT (77)..(77): X12 is N or D
  VARIANT (79)..(79): X13 is G or R
  VARIANT (80)..(80): X14 is T or I
  VARIANT (81)..(81): X15 is L or A
  VARIANT (82)..(82): X16 is R or L
  VARIANT (83)..(83): X17 is G or R
  VARIANT (90)..(90): X18 is A or D
  VARIANT (95)..(95): X19 is I, L, or V
  VARIANT (97)..(97): X20 is I, R or M
  VARIANT (99)..(99): X21 is F or Y
  VARIANT (105)..(105): X22 is S or P
  VARIANT (107)..(107): X23 is W or G
  VARIANT (114)..(114): X24 is R, H, or Q
  VARIANT (116)..(116): X25 is D or Y
  VARIANT (127)..(127): X26 is N or K
  VARIANT (142)..(142): X27 is T or I
  VARIANT (144)..(144): X28 is K or Q
  VARIANT (145)..(145): X29 is R or H
  VARIANT (149)..(149): X30 is A or T
  VARIANT (150)..(150): X31 is A or V
  VARIANT (151)..(151): X32 is H or R
  VARIANT (156)..(156): X33 is R, L, Q, or W
  VARIANT (158)..(158): X34 is V or A
  VARIANT (161)..(161): X35 is D or E
  VARIANT (163)..(163): X36 is R or T
  VARIANT (166)..(166): X37 is D or E
  VARIANT (167)..(167): X38 is W or G
  VARIANT (184)..(184): X39 is P or A
  VARIANT (193)..(193): X40 is P or A
  VARIANT (194)..(194): X41 is V or I
  VARIANT (207)..(207): X42 is S or G
  VARIANT (246)..(246): X43 is A or S
  VARIANT (253)..(253): X44 is Q or E
  VARIANT (276)..(276): X45 is P or L
GSHSMRYFXTSVSRPGRGEPRFIAVGYVDDTQFVRFDSDAASQXMEPRAPWIEQEGPEYWDXXTXXXKAXSQXXRXXLXXXXXYYNQSEXGSHTXQXMXGCDVGXDXRFLRGYXQXAYDGKDYIALXEDLRSWTAADMAAQXTXXKWEXXXEAEQXRXYLXGXCVXXLRRYLENGKETLQRTDXPKTHMTHHXXSDHEATLRCWALXFYPAEITLTWQRDGEDQTQDTELVETRPAGDGTFQKWAXVVVPSGXEQRYTCHVQHEGLPKPLTLRWEX

SEQ ID NO: 33 - protein, 276 aa, Homo sapiens [sequence omitted]

SEQ ID NO: 34 - protein, 276 aa, Homo sapiens
GSHSMRYFDTAMSRPGRGEPRFISVGYVDDTQFVRFDSDAASPREEPRAPWIEQEGPEYWDRNTQIFKTNTQTDRESLRNLRGYYNQSEAGSHTLQSMYGCDVGPDGRLLRGHNQYAYDGKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 35 - protein, 276 aa, Homo sapiens
GSHSMRYFYTAMSRPGRGEPRFIAVGYVDDTQFVRFDSDAASPRMAPRAPWIEQEGPEYWDRNTQISKTNTQTYRESLRNLRGYYNQSEAGSHIIQRMYGCDVGPDGRLLRGYDQSAYDGKDYIALNEDLSSWTAADTAAQITQRKWEAAREAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 36 - protein, 276 aa, Homo sapiens
GSHSMRYFYTSVSRPGRGEPRFISVGYVDDTQFVRFDSDAASPREEPRAPWIEQEGPEYWDRNTQICKTNTQTYRENLRTALRYYNQSEAGSHTLQRMYGCDVGPDGRLLRGHNQFAYDGKDYIALNEDLSSWTAADTAAQITQRKWEAARVAEQLRTYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 37 - protein, 276 aa, Homo sapiens
GSHSMRYFHTAMSRPGRGEPRFITVGYVDDTLFVRFDSDATSPRKEPRAPWIEQEGPEYWDRETQISKTNTQTYRESLRNLRGYYNQSEAGSHTLQRMYGCDVGPDGRLLRGHNQYAYDGKDYIALNEDLRSWTAADTAAQISQRKLEAARVAEQLRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 38 - protein, 276 aa, Homo sapiens
GSHSMRYFYTAMSRPGRGEPRFIAVGYVDDTQFVRFDSDAASPRMAPRAPWIEQEGPEYWDRETQKYKRQAQTDRVSLRNLRGYYNQSEAGSHTLQRMYGCDVGPDGRLLRGHDQSAYDGKDYIALNEDLSSWTAADTAAQITQRKWEAAREAEQWRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 39 - protein, 276 aa, Homo sapiens
GSHSMRYFYTAMSRPGRGEPRFIAVGYVDDTQFVRFDSDAASPRTEPRAPWIEQEGPEYWDRNTQIFKTNTQTYRENLRIALRYYNQSEAGSHIIQRMYGCDLGPDGRLLRGHDQSAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEP

SEQ ID NO: 40 - protein, 276 aa, Homo sapiens
  VARIANT (9)..(9): X1 is H, Y, or D
  VARIANT (11)..(11): X2 is A or S
  VARIANT (12)..(12): X3 is M or V
  VARIANT (24)..(24): X4 is A, S, or T
  VARIANT (32)..(32): X5 is Q or L
  VARIANT (41)..(41): X6 is A or T
  VARIANT (45)..(45): X7 is E, M K, or T
  VARIANT (46)..(46): X8 is A or T
  VARIANT (63)..(63): X9 is E or N
  VARIANT (66)..(66): X10 is I or K
  VARIANT (67)..(67): X11 is Y, F, S, or C
  VARIANT (70)..(70): X12 is N or Q
  VARIANT (71)..(71): X13 is A or T
  VARIANT (74)..(74): X14 is D or Y
  VARIANT (76)..(76): X15 is E or V
  VARIANT (77)..(77): X16 is S or N
  VARIANT (80)..(80): X17 is T, N, or I
  VARIANT (81)..(81): X18 is A or L
  VARIANT (82)..(82): X19 is L, or R
  VARIANT (82)..(82): X19 is L, or R
  misc_feature (83)..(83): Xaa can be any naturally occurring amino acid
  VARIANT (91)..(91): X21 is T or I
  misc_feature (94)..(94): Xaa can be any naturally occurring amino acid
  VARIANT (95)..(95): X22 is L or I
  VARIANT (97)..(97): X23 is R or S
  VARIANT (131)..(131): X24 is R or S
  VARIANT (143)..(143): X25 is S or T
  VARIANT (147)..(147): X26 is L or W
  VARIANT (152)..(152): X27 is E OR V
  VARIANT (156)..(156): X28 is R, D, L or W
  VARIANT (158)..(158): X29 is A or T
  VARIANT (163)..(163): X30 is L, E or T
  VARIANT (177)..(177): X31 is E or D
  VARIANT (178)..(178): X32 is K or T
  VARIANT (180)..(180): X33 is E or Q
  VARIANT (194)..(194): X34 is I or V
GSHSMRYFXTXXSRPGRGEPRFIXVGYVDDTXFVRFDSDAXSPRXXPRAPWIEQEGPEYWDRXTQXXKTXXTQXYXXNLXXXXYYNQSEAGSHXXQXMYGCDLGPDGRLLRGHDQSAYDGKDYIALNEDLXSWTAADTAAQIXQRKXEAARXAEQXRXYLEGXCVEWLRRYLENGKXXLXRADPPKTHVTHHPXSDHEATLRCWALGFYPAEITLTWQRDGEDQTQDTELVETRPAGDRTFQKWAAVVVPSGEEQRYTCHVQHEGLPKPLTLRWEP

SEQ ID NO: 41 - protein, 276 aa, Homo sapiens
CSHSMKYFFTSVSRPGRGEPRFISVGYVDDTQFVRFDSDAASPRGEPRAPWVEQEGPEYWDRETQKYKRQAQTDRVSLRNLRGYYNQSEAGSHTLQWMCGCDLGPDGRLLRGYDQYAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVMVPSGEEQRYTCHVQHEGLPEPLTLRWEP

SEQ ID NO: 42 - protein, 276 aa, Homo sapiens
GSHSMRYFYTAVSRPGRGEPHFIAVGYVDDTQFVRFDSDAASPRGEPRAPWVEQEGPEYWDRETQKYKRQAQTDRVSLRNLRGYYNQSEARSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEP

SEQ ID NO: 43 - protein, 276 aa, Homo sapiens
GSHSMRYFYTAVSRPGRGEPHFIAVGYVDDTQFVRFDSDAASPRGEPRAPWVEQEGPEYWDRETQKYKRQAQTDRVSLRNLRGYYNQSEAGSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEP

SEQ ID NO: 44 - protein, 276 aa, Homo sapiens
GSHSMRYFSTSVSWPGRGEPRFIAVGYVDDTQFVRFDSDAASPRGEPREPWVEQEGPEYWDRETQKYKRQAQADRVNLRKLRGYYNQSEDGSHTLQRMFGCDLGPDGRLLRGYNQFAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWKP

SEQ ID NO: 45 - protein, 276 aa, Homo sapiens
CSHSMRYFDTAVSRPGRGEPRFISVGYVDDTQFVRFDSDAASPRGEPRAPWVEQEGPEYWDRETQKYKRQAQADRVNLRKLRGYYNQSEDGSHTLQWMYGCDLGPDGRLLRGYDQSAYDGKDYIALNEDLRSWTAADTAAQITQRKWEAAREAEQWRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEP

SEQ ID NO: 46 - protein, 276 aa, Homo sapiens
CSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEP

SEQ ID NO: 47 - protein, 276 aa, Homo sapiens
CSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEP

SEQ ID NO: 48 - protein, 276 aa, Homo sapiens
CSHSMRYFYTAVSRPGRGEPRFIAVGYVDDTQFVQFDSDAASPRGEPRAPWVEQEGPEYWDRETQKYKRQAQTDRVSLRNLRGYYNQSEAGSHTLQRMYGCDLGPDGRLLRGYNQFAYDGKDYIALNEDLRSWTAADTAAQITQRKWEAARTAEQLRAYLEGTCVEWLRRYLENGKKTLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWGP

SEQ ID NO: 49 - protein, 276 aa, Homo sapiens
CSHSMRYFYTAVSRPGRGEPHFIAVGYVDDTQFVRFDSDAASPRGEPRAPWVEQEGPEYWDRETQNYKRQAQTDRVNLRKLRGYYNQSEAGSHIIQRMYGCDLGPDGRLLRGHDQLAYDGKDYIALNEDLRSWTAADTAAQITQRKWEAAREAEQLRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEP

SEQ ID NO: 50 - protein, 276 aa, Homo sapiens
  VARIANT (1)..(1): X1 is C or G
  VARIANT (6)..(6): X2 is R or K
  VARIANT (9)..(9): X3 is F, Y, S, or D
  VARIANT (14)..(14): X4 is R or W
  VARIANT (21)..(21): X5 is H or R
  VARIANT (24)..(24): X6 is A or S
  VARIANT (35)..(35): X7 is Q or R
  VARIANT (49)..(49): X8 is A or E
  VARIANT (66)..(66): X9 is N or K
  VARIANT (73)..(73): X10 is T or A
  VARIANT (77)..(77): X11 is S or N
  VARIANT (80)..(80): X12 is N or K
  VARIANT (90)..(90): X13 is A or D
  VARIANT (91)..(91): X14 is G or R
  VARIANT (94)..(94): X15 is T or I
  VARIANT (95)..(95): X16 is L or I
  VARIANT (97)..(97): X17 is W or R
  VARIANT (99)..(99): X18 is C, Y, F, or S
  VARIANT (103)..(103): X19 is L, or V
  VARIANT (113)..(113): X20 is Y or H
  VARIANT (114)..(114): X21 is D or N
  VARIANT (116)..(116): X22 is Y, F, S, or L
  VARIANT (147)..(147): X23 is L or W
  VARIANT (152)..(152): X24 is E, A, or T
  VARIANT (156)..(156): X25 is R, L, or W
  VARIANT (163)..(163): X26 is L or T
  VARIANT (173)..(173): X27 is E or K
  VARIANT (177)..(177): X28 is E or K
  VARIANT (184)..(184): X29 is H or P
  VARIANT (194)..(194): X30 is R or V
  VARIANT (219)..(219): X31 is W or R
  VARIANT (248)..(248): X32 is V or M
  VARIANT (253)..(253): X33 is E or Q
  VARIANT (261)..(261): X34 is M or V
  VARIANT (267)..(267): X35 is P or Q
  VARIANT (273)..(273): X36 is R or S
  VARIANT (275)..(275): X37 is P or G
XSHSMXYFXTAVSXPGRGEPXFIXVGYVDDTQFVXFDSDAASPRGEPRXPWVEQEGPEYWDRETQXYKRQAQXDRVXLRXLRGYYNQSEXXSHXXQXMXGCDXGPDGRLLRGXXQXAYDGKDYIALNEDLRSWTAADTAAQITQRKXEAARXAEQXRAYLEGXCVEWLRRYLXNGKXTLQRAEXPKTHVTHHPXSDHEATLRCWALGFYPAEITLTWQXDGEDQTQDTELVETRPAGDGTFQKWAAVXVPSGXEQRYTCHXQHEGLXEPLTLXWXP

SEQ ID NO: 51 - protein, 284 aa, Homo sapiens
  VARIANT (89)..(89): X1= K or E
  VARIANT (107)..(107): X2= R or G
  VARIANT (157)..(157): X3= R or G
  VARIANT (158)..(158): X3= R or G
  VARIANT (255)..(255): X5= Q or P
  VARIANT (267)..(267): X6= P or S
GSHSLKYFHTSVSRPGRGEPRFISVGYVDDTQFVRFDNDAASPRMVPRAPWMEQEGSEYWDRETRSARDTAQIFRVNLRTLRGYYNQSXAGSHTLQWMHGCELGPDXRFLRGYEQFAYDGKDYLTLNEDLRSWTAVDTAAQISEQKSNDASEAEHQXXYLEDTCVEWLHKYLEKGKETLLHLEPPKTHVTHHPISDHEATLRCWALGFYPAEITLTWQQDGEGHTQDTELVETRPAGDGTFQKWAAVVVPSGEEXRYTCHVQHEGLXEPVTLRWKPASQPTIPI

SEQ ID NO: 52 - protein, 284 aa, Homo sapiens
  VARIANT (7)..(7): X1= Y or F
  VARIANT (50)..(50): X2= P or Q
  VARIANT (251)..(251): X3= S or P
  VARIANT (278)..(278): X4= P or L
GSHSLRXFSTAVSRPGRGEPRYIAVEYVDDTQFLRFDSDAAIPRMEPREXWVEQEGPQYWEWTTGYAKANAQTDRVALRNLLRRYNQSEAGSHTLQGMNGCDMGPDGRLLRGYHQHAYDGKDYISLNEDLRSWTAADTVAQITQRFYEAEEYAEEFRTYLEGECLELLRRYLENGKETLQRADPPKAHVAHHPISDHEATLRCWALGFYPAEITLTWQRDGEEQTQDTELVETRPAGDGTFQKWAAVVVPXGEEQRYTCHVQHEGLPQPLILRWEQSXQPTIPI

SEQ ID NO: 53 - protein, 284 aa, Homo sapiens
  VARIANT (13)..(13): X1= S or
  VARIANT (27)..(27): X2= Y or H
  VARIANT (31)..(31): X3= T, S, or M
  VARIANT (34)..(34): X4= L or V
  VARIANT (54)..(54): X5= Q or R
  VARIANT (81)..(81): X6= P or L
  VARIANT (100)..(100): X7= G or D
  VARIANT (104)..(104): X8= G or V
  VARIANT (105)..(105): X9= S or C
  VARIANT (110)..(110): X10= L or I
  VARIANT (159)..(159): X11= Y or H
  VARIANT (169)..(169): X12= H or R
  VARIANT (171)..(171): X13= Y or H
  VARIANT (178)..(178): X14= M or T
  VARIANT (185)..(185): X15= P or A
  VARIANT (219)..(219): X16= R, W, or Q
  VARIANT (258)..(258): X17= T or M
  VARIANT (275)..(275): X18= K or E
GSHSMRYFSAAVXRPGRGEPRFIAMGXVDDXQFXRFDSDSACPRMEPRAPWVEXEGPEYWEEETRNTKAHAQTDRMNLQTXRGYYNQSEASSHTLQWMIXCDLXXDGRLXRGYEQYAYDGKDYLALNEDLRSWTAADTAAQISKRKCEAANVAEQRRAXLEGTCVEWLXRXLENGKEXLQRADPXKTHVTHHPVFDYEATLRCWALGFYPAEIILTWQXDGEDQTQDVELVETRPAGDGTFQKWAAVVVPSGEEQRYXCHVQHEGLPEPLMLRWXQSSLPTIPI

SEQ ID NO: 54 - protein, 352 aa, Homo sapiens
ASPTSPVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 55 - protein, 383 aa, Homo sapiens
PTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 56 - protein, 222 aa, Homo sapiens
ADPCDSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 57 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 227 aa, Homo sapiens
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 227 aa, Homo sapiens
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 325 aa, Homo sapiens
STKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 63 - protein, 246 aa, Homo sapiens
HKPSNTKVDKRVELKTPLGDTTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 65 - protein, 223 aa, Homo sapiens
PPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

SEQ ID NO: 66 - protein, 453 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 67 - protein, 112 aa, Homo sapiens
FTVRETASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKT

SEQ ID NO: 68 - protein, 159 aa, Homo sapiens
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 69 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 71 - protein, 33 aa, Artificial Sequence
  Leucine-zipper polypeptide
RMKQIEDKIEEILSKIYHIENEIARIKKLIGER

SEQ ID NO: 72 - protein, 32 aa, Artificial Sequence
  Leucine-zipper polypeptide
LSSIEKKQEEQTSWLIWISNELTLIRNELAQS

SEQ ID NO: 73 - protein, 31 aa, Artificial Sequence
  Leucine-zipper polypeptide
LSSIEKKLEEITSQLIQISNELTLIRNELAQ

SEQ ID NO: 74 - protein, 31 aa, Artificial Sequence
  Leucine-zipper polypeptide
LSSIEKKLEEITSQLIQIRNELTLIRNELAQ

SEQ ID NO: 75 - protein, 31 aa, Artificial Sequence
  Leucine-zipper polypeptide
LSSIEKKLEEITSQLQQIRNELTLIRNELAQ

SEQ ID NO: 76 - protein, 31 aa, Artificial Sequence
  Leucine-zipper polypeptide
LSSLEKKLEELTSQLIQLRNELTLLRNELAQ

SEQ ID NO: 77 - protein, 31 aa, Artificial Sequence
  Leucine-zipper polypeptide
ISSLEKKIEELTSQIQQLRNEITLLRNEIAQ

SEQ ID NO: 78 - protein, 47 aa, Artificial Sequence
  Leucine-zipper polypeptide
LEIEAAFLERENTALETRVAELRQRVQRLRNRVSQYRTRYGPLGGGK

SEQ ID NO: 79 - protein, 31 aa, Artificial Sequence
  Coiled-coil polypeptide
LKSVENRLAVVENQLKTVIEELKTVKDLLSN

SEQ ID NO: 80 - protein, 31 aa, Artificial Sequence
  Coiled-coil polypeptide
VSRLEEKVKTLKSQVTELASTVSLLREQVAQ

SEQ ID NO: 81 - protein, 30 aa, Artificial Sequence
  Coiled-coil polypeptide
IQSEKKIEDISSLIGQIQSEITLIRNEIAQ

SEQ ID NO: 82 - protein, 31 aa, Artificial Sequence
  Coiled-coil polypeptide
LMSLEKKLEELTQTLMQLQNELSMLKNELAQ

SEQ ID NO: 83 - protein, 18 aa, Artificial Sequence
  Framework/dimerization polypeptide
VDLEGSTSNGRQCAGIRL

SEQ ID NO: 84 - protein, 27 aa, Artificial Sequence
  Framework/dimerization polypeptide
EDDVTTTEELAPALVPPPKGTCAGWMA

SEQ ID NO: 85 - protein, 27 aa, Artificial Sequence
  Framework/dimerization polypeptide
GHDQETTTQGPGVLLPLPKGACTGQMA

SEQ ID NO: 86 - protein, 64 aa, Artificial Sequence
  Collagen Oligomerization Sequence
VTAFSNMDDMLQKAHLVIEGTFIYLRDSTEFFIRVRDGWKKLQLGELIPIPADSPPPPALSSNP

SEQ ID NO: 87 - protein, 10 aa, Artificial Sequence
  Cysteine Containing Linker
GCGGSGGGGS

SEQ ID NO: 88 - protein, 276 aa, Artificial Sequence
  HLA Class I heavy chain variant with Y84C, and A139C, substitutions
  VARIANT (79)..(86): Any of residues 79-83 may be present or absent, when present they are each selected independently from proteinogenic amino acids or any proteinogenic amino acid amino acid other than glycine, in an embodiment it is the sequence GTLRG
  DISULFID (84)..(84): Residue 84 can form a disulfide bond with residue 139
  VARIANT (85)..(89): Any of residues 85-89 may be present or absent, when present they are each selected independently from proteinogenic amino acids or any proteinogenic amino acid amino acid other than glycine, in an embodiment it is the sequence YNQSE
  VARIANT (134)..(138): Any of residues 134-138 may be present or absent, when present they are each selected independently from proteinogenic amino acids or any proteinogenic amino acid amino acid other than glycine, in an embodiment it is the sequence TAADM
  DISULFID (139)..(139): Residue 139 can form a disulfide bond with residue 84
  VARIANT (140)..(144)
GSHSMRYFFTSVSRPGRGEPRFIAVGYVDDTQFVRFDSDAASQRMEPRAPWIEQEGPEYWDGETRKVKAHSQTHRVDLXXXXXCXXXXXAGSHTVQRMYGCDVGSDWRFLRGYHQYAYDGKDYIALKEDLRSWXXXXXCXXXXXHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEP

SEQ ID NO: 89 - protein, 99 aa, Artificial Sequence
  Beta-2 microglobulin polypeptide variant with a cysteine substitution at position 12
  VARIANT (12)..(12): Beta-2 microglobulin polypeptide variant with a cysteine substitution at position 12
IQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 90 - protein, 276 aa, Artificial Sequence
  HLA Class I heavy chain variant with Y84C, A139C, and A236C substitutions
  VARIANT (79)..(83): Any of residues 79-83 may be present or absent, when present they are each selected independently from proteinogenic amino acids or any proteinogenic amino acid amino acid other than glycine, in an embodiment it is the sequence GTLRG
  VARIANT (84)..(84): Cysteine substitution
  VARIANT (85)..(89): Any of residues 85-89 may be present or absent, when present they are each selected independently from proteinogenic amino acids or any proteinogenic amino acid amino acid other than glycine, in an embodiment it is the sequence YNQSE
  VARIANT (134)..(138): Any of residues 134-138 may be present or absent, when present they are each selected independently from proteinogenic amino acids or any proteinogenic amino acid amino acid other than glycine, in an embodiment it is the sequence TAADM
  VARIANT (139)..(139): Cysteine substitution
  VARIANT (140)..(144): Any of residues 140-144 may be present or absent, when present they are each selected independently from proteinogenic amino acids or any proteinogenic amino acid amino acid other than glycine, in an embodiment it is the sequence AQITK
  VARIANT (231)..(235): Any of residues 231-235 may be present or absent, when present they are each selected independently from proteinogenic amino acids or any proteinogenic amino acid amino acid other than glycine, in an embodiment it is the sequence VETRP
  VARIANT (236)..(236): cysteine substitution
  VARIANT (237)..(241): Any of residues 237-241 may be present or absent, when present they are each selected independently from proteinogenic amino acids or any proteinogenic amino acid amino acid other than glycine, in an embodiment it is the sequence GDGTF
GSHSMRYFFTSVSRPGRGEPRFIAVGYVDDTQFVRFDSDAASQRMEPRAPWIEQEGPEYWDGETRKVKAHSQTHRVDLXXXXXCXXXXXAGSHTVQRMYGCDVGSDWRFLRGYHQYAYDGKDYIALKEDLRSWXXXXXCXXXXXHKWEAAHVAEQLRAYLEGTCVEWLRRYLENGKETLQRTDAPKTHMTHHAVSDHEATLRCWALSFYPAEITLTWQRDGEDQTQDTELXXXXXCXXXXXQKWAAVVVPSGQEQRYTCHVQHEGLPKPLTLRWEP

SEQ ID NO: 91 - protein, 133 aa, Artificial Sequence
  IL-2 polypeptide with H16A and F42A substitutions
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 92 - protein, 10 aa, Artificial Sequence
  Cysteine containing  linker where the GGGGS portion may be repeated 1-9 times
GCGGSGGGGS

SEQ ID NO: 93 - protein, 20 aa, Artificial Sequence
  cysteine containing linker
GCGGSGGGGSGGGGSGGGGS

SEQ ID NO: 94 - protein, 15 aa, Artificial Sequence
  cysteine containing linker
GCGGSGGGGSGGGGS

SEQ ID NO: 95 - protein, 133 aa, Homo sapiens [sequence omitted]

SEQ ID NO: 96 - protein, 251 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 97 - protein, 524 aa, Homo sapiens
VNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 98 - protein, 347 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 99 - protein, 133 aa, Artificial Sequence
  IL-2 variant polypeptide with substitutions at postions 16 and 42,, e.g., an H16A F42A variant
  VARIANT (16)..(16): X is any aa other than His. In some embodiments it may be Ala or Thr.
  VARIANT (42)..(42): X is any aa other than Phe. In some embodiments it may be Ala or Thr.
APTSSSTKKTQLQLEXLLLDLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 100 - protein, 31 aa, Artificial Sequence
  Coiled Coil polypeptide
LARIEEKLKTIKAQLSEIASTLNMIREQLAQ

SEQ ID NO: 101 - protein, 5 aa, Artificial Sequence
  Linker sequence
GSGGS

SEQ ID NO: 102 - protein, 4 aa, Artificial Sequence
  Linker sequence
GGGS

SEQ ID NO: 103 - protein, 4 aa, Artificial Sequence
  Linker sequence
GGSG

SEQ ID NO: 104 - protein, 5 aa, Artificial Sequence
  Linker sequence
GGSGG

SEQ ID NO: 105 - protein, 5 aa, Artificial Sequence
  Linker sequence
GSGSG

SEQ ID NO: 106 - protein, 5 aa, Artificial Sequence
  Linker sequence
GSGGG

SEQ ID NO: 107 - protein, 5 aa, Artificial Sequence
  Linker sequence
GGGSG

SEQ ID NO: 108 - protein, 5 aa, Artificial Sequence
  Linker sequence
GSSSG

SEQ ID NO: 109 - protein, 5 aa, Artificial Sequence
  Linker sequence
GSSSS

SEQ ID NO: 110 - protein, 5 aa, Artificial Sequence
  Linker sequence
GGGGS

SEQ ID NO: 111 - protein, 5 aa, Artificial Sequence
  Linker sequence
AAAGG

SEQ ID NO: 112 - protein, 5 aa, Artificial Sequence
  Linker sequence
GCGGS

SEQ ID NO: 113 - protein, 15 aa, Artificial Sequence
  Linker sequence
GCGASGGGGSGGGGS

SEQ ID NO: 114 - protein, 20 aa, Artificial Sequence
  Linker sequence
GCGGSGGGGSGGGGSGGGGS

SEQ ID NO: 115 - protein, 15 aa, Artificial Sequence
  Linker sequence
GCGGSGGGGSGGGGS

SEQ ID NO: 116 - protein, 188 aa, Homo sapiens [sequence omitted]

SEQ ID NO: 117 - protein, 189 aa, Homo sapiens [sequence omitted]

SEQ ID NO: 118 - protein, 188 aa, Artificial Sequence
  KRAS variant
  VARIANT (5)..(5): X1 is Lys, Phe, or Leu
  VARIANT (7)..(7): X2 is Val or Leu
  VARIANT (8)..(8): X3 is Val or Thr
  VARIANT (9)..(9): X4 is Val or Thr
  VARIANT (12)..(12): X5 is Gly, Asp, Cys, Val, or Ser
  VARIANT (13)..(13): X6 is Gly, Cys, or Asp; where one or both of X5 and X6 is not a Cys
MTEYXLXXXGAXXVGKSALTIQLIQNHFVDEYDPTIEDSYRKQVVIDGETCLWDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 119 - protein, 9 aa, Artificial Sequence
  KRAS peptide
VVGADGVGK

SEQ ID NO: 120 - protein, 9 aa, Artificial Sequence
  KRAS peptide
VVGACGVGK

SEQ ID NO: 121 - protein, 9 aa, Artificial Sequence
  KRAS peptide
VVGAVGVGK

SEQ ID NO: 122 - protein, 10 aa, Artificial Sequence
  KRAS peptide
VVVGADGVGK

SEQ ID NO: 123 - protein, 10 aa, Artificial Sequence
  KRAS peptide
VVVGAVGVGK

SEQ ID NO: 124 - protein, 10 aa, Artificial Sequence
  KRAS peptide
VVVGACGVGK

SEQ ID NO: 125 - protein, 9 aa, Artificial Sequence
  KRAS peptideKRAS peptide
VTGADGVGK

SEQ ID NO: 126 - protein, 9 aa, Artificial Sequence
  KRAS peptide
VTGAVGVGK

SEQ ID NO: 127 - protein, 9 aa, Artificial Sequence
  KRAS peptide
VTGACGVGK

SEQ ID NO: 128 - protein, 10 aa, Artificial Sequence
  KRAS peptide
VTVGADGVGK

SEQ ID NO: 129 - protein, 10 aa, Artificial Sequence
  KRAS peptide
VTVGAVGVGK

SEQ ID NO: 130 - protein, 10 aa, Artificial Sequence
  KRAS peptide
VTVGACGVGK

SEQ ID NO: 131 - protein, 9 aa, Artificial Sequence
  KRAS peptide
VVVGAGDVG

SEQ ID NO: 132 - protein, 9 aa, Artificial Sequence
  KRAS peptide
VVGAGDVGK

SEQ ID NO: 133 - protein, 10 aa, Artificial Sequence
  KRAS peptide
VVVGARGVGK

SEQ ID NO: 134 - protein, 9 aa, Artificial Sequence
  KRAS peptide
VVGARGVGK

SEQ ID NO: 135 - protein, 9 aa, Artificial Sequence
  KRAS peptide
LVVVGADGV

SEQ ID NO: 136 - protein, 9 aa, Artificial Sequence
  KRAS peptide
LVVVGAVGV

SEQ ID NO: 137 - protein, 9 aa, Artificial Sequence
  KRAS peptide
LVVVGACGV

SEQ ID NO: 138 - protein, 10 aa, Artificial Sequence
  KRAS peptide
KLVVVGADGV

SEQ ID NO: 139 - protein, 10 aa, Artificial Sequence
  KRAS peptide
KLVVVGAVGV

SEQ ID NO: 140 - protein, 10 aa, Artificial Sequence
  KRAS peptide
KLVVVGACGV

SEQ ID NO: 141 - protein, 9 aa, Artificial Sequence
  KRAS peptide
LLVVGADGV

SEQ ID NO: 142 - protein, 9 aa, Artificial Sequence
  KRAS peptide
LLVVGAVGV

SEQ ID NO: 143 - protein, 9 aa, Artificial Sequence
  KRAS peptide
LLVVGACGV

SEQ ID NO: 144 - protein, 10 aa, Artificial Sequence
  KRAS peptide
FLVVVGADGV

SEQ ID NO: 145 - protein, 10 aa, Artificial Sequence
  KRAS peptide
FLVVVGAVGV

SEQ ID NO: 146 - protein, 10 aa, Artificial Sequence
  KRAS peptide
FLVVVGACGV

SEQ ID NO: 147 - protein, 9 aa, Artificial Sequence
  KRAS peptide
GAGDVGKSA

SEQ ID NO: 148 - protein, 9 aa, Artificial Sequence
  KRAS peptide
AGDVGKSAL

SEQ ID NO: 149 - protein, 9 aa, Artificial Sequence
  KRAS peptide
DVGKSALTI

SEQ ID NO: 150 - protein, 10 aa, Artificial Sequence
  KRAS peptide
GAVGVGKSAL

SEQ ID NO: 151 - protein, 9 aa, Artificial Sequence
  KRAS peptide
AVGVGKSAL

SEQ ID NO: 152 - protein, 9 aa, Artificial Sequence
  KRAS peptide
YKLVVVGAV

SEQ ID NO: 153 - protein, 9 aa, Artificial Sequence
  KRAS peptide
ARGVGKSAL

SEQ ID NO: 154 - protein, 10 aa, Artificial Sequence
  KRAS peptide
GARGVGKSAL

SEQ ID NO: 155 - protein, 10 aa, Artificial Sequence
  KRAS peptide
EYKLVVVGAR

SEQ ID NO: 156 - protein, 10 aa, Artificial Sequence
  KRAS peptide
RGVGKSALTI

SEQ ID NO: 157 - protein, 9 aa, Artificial Sequence
  KRAS peptide
LVVVGARGV

SEQ ID NO: 158 - protein, 9 aa, Artificial Sequence
  KRAS peptide
ACGVGKSAL

SEQ ID NO: 159 - protein, 10 aa, Artificial Sequence
  KRAS peptide
GACGVGKSAL

SEQ ID NO: 160 - protein, 10 aa, Artificial Sequence
  KRAS peptide
GADGVGKSAL

SEQ ID NO: 161 - protein, 10 aa, Artificial Sequence
  KRAS peptide
KLVVVGAGDV

SEQ ID NO: 162 - protein, 10 aa, Artificial Sequence
  KRAS peptide
KLVVVGARGV

SEQ ID NO: 163 - protein, 9 aa, Artificial Sequence
  KRAS peptide
KLVVGADGV

SEQ ID NO: 164 - protein, 9 aa, Artificial Sequence
  KRAS peptide
KLVVGAVGV

SEQ ID NO: 165 - protein, 9 aa, Artificial Sequence
  KRAS peptide
KLVVVAVGV

SEQ ID NO: 166 - protein, 9 aa, Artificial Sequence
  KRAS peptide
KLVVVADGV

SEQ ID NO: 167 - protein, 9 aa, Artificial Sequence
  KRAS peptide
VGAVGVGKS

SEQ ID NO: 168 - protein, 10 aa, Artificial Sequence
  KRAS peptide
VGAVGVGKSA

SEQ ID NO: 169 - protein, 10 aa, Artificial Sequence
  KRAS peptide
AVGVGKSALT

SEQ ID NO: 170 - protein, 9 aa, Artificial Sequence
  KRAS peptide
GAVGVGKSA

SEQ ID NO: 171 - protein, 9 aa, Artificial Sequence
  KRAS peptide
KLVVVGAVG

SEQ ID NO: 172 - protein, 9 aa, Artificial Sequence
  KRAS peptide
KLVVVGAVG

SEQ ID NO: 173 - protein, 9 aa, Artificial Sequence
  epitope tag
YPYDVPDYA

SEQ ID NO: 174 - protein, 8 aa, Artificial Sequence
  epitope tag
DYKDDDDK

SEQ ID NO: 175 - protein, 10 aa, Artificial Sequence
  epitope tag
EQKLISEEDL

SEQ ID NO: 176 - protein, 5 aa, Artificial Sequence
  affinity domain
HHHHH

SEQ ID NO: 177 - protein, 6 aa, Artificial Sequence
  affinity domain
HHHHHH

SEQ ID NO: 178 - protein, 5 aa, Artificial Sequence
  affinity domain
RYIRS

SEQ ID NO: 179 - protein, 4 aa, Artificial Sequence
  affinity domain
FHHT

SEQ ID NO: 180 - protein, 17 aa, Artificial Sequence
  C-end RNA tag/affinity domain
WEAAAREACCRECCARA

SEQ ID NO: 181 - protein, 635 aa, Artificial Sequence
  Construct 3777
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 182 - protein, 535 aa, Artificial Sequence
  Construct 3781
ELAGIGILTVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEGGGGSGGGGSGGGGSTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 183 - protein, 535 aa, Artificial Sequence
  Construct 3782
ELAGIGILTVGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEGGGGSGGGGSGGGGSTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 184 - protein, 535 aa, Artificial Sequence
  Construct 3783
ELAGIGILTVGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEGGGGSGGGGSGGGGSTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 185 - protein, 8 aa, Artificial Sequence
  Strep. Tag
WSHPQFEK